(19) United States
(12) Patent Application Publication
(10) Pub. No.: US 2022/0411388 A1
(43) Pub. Date: Dec. 29, 2022

(54) SUBSTITUTED IMIDAZOLE CARBOXAMIDES AND THEIR USE IN THE TREATMENT OF MEDICAL DISORDERS

(71) Applicant: Bial - R&D Investments, S.A., Coronado (S.Romão e S.Mamede) (PT)

(72) Inventors: Renato T. Skerlj, West Newton, MA (US); Elyse Marie Josee Bourque, L'etang-Du-Nord (CA); Soumya Ray, Quincy, MA (US); Peter T. Lansbury, Brookline, MA (US)

(21) Appl. No.: 17/760,871

(22) PCT Filed: Sep. 17, 2020
(86) PCT No.: PCT/US2020/051282

§ 371 Date: Mar. 16, 2022

Related U.S. Application Data
(60) Provisional application No. 62/901,382, filed on Sep. 17, 2019.

(51) Int. Cl. C07D 295/088

(52) U.S. Cl. CPC C07D 295/088

(57) ABSTRACT

The invention provides substituted imidazole carboxamides and related compounds, compositions containing such compounds, medical kits, and methods for using such compounds and compositions to treat a medical disorder, e.g., cancer, lysosomal storage disorder, neurodegenerative disorder, inflammatory disorder, in a patient.

### CROSS-REFERENCE TO RELATED APPLICATIONS

This application claims the benefit of U.S. Provisional Patent Application No. 62/901,382 filed on Sep. 17, 2019, the entire contents of which are incorporated by reference herein.

### FIELD OF THE INVENTION

The invention provides substituted imidazole carboxamides and related compounds, compositions containing such compounds, medical kits, and methods for using such compounds and compositions to treat medical disorders in a patient.

### BACKGROUND

Sphingolipids, in addition to serving roles in cell membrane structure and dynamics, also serve important signaling functions, for example, in the control of cell growth, cell differentiation, and cell death, and so are important for cell homeostasis and development. Zeidan et al. (2010) CURR. MOL. MED. 10, 454, Proksch et al. (2011) J. LIPIDS Article ID 971618. Ceramide, a key member of this lipid class, has attracted attention in view of its impact on the replication and differentiation of neoplastic cells. Furuya et al. (2011) CANCER METASTASIS REV. 30, 567. For example, lower levels of ceramide have been discovered in several types of human tumors relative to normal tissue, where the level of ceramide appears to correlate inversely with the degree of malignant progression. Realini et al. (2013) J. MOL. BIOL. 56, 3518.

Acid ceramidase (AC, also known as N-acylsphingosine amidohydrolase-1, and ASAH-1) is a cysteine amidase that catalyzes the hydrolysis of ceramide into sphingosine and fatty acid. Acid ceramidase is believed to be involved in the regulation of ceramide levels in cells and modulates the ability of this lipid messenger to influence the survival, growth and death of certain tumor cells. Doan et al. (2017) ONCOTARGET 8(68), 112662-74. Furthermore, acid ceramidase enzymes are abnormally expressed in various types of human cancer (e.g., prostate, head and neck, and colon) and serum AC levels are elevated in patients with melanoma relative to control subjects. Realini et al. (2015) J. BIOL. CHEM. 291 (5), 2422-34.

In addition, acid ceramidase enzymes have been implicated in a number of other disorders, including, inflammation (for example, rheumatoid arthritis and psoriasis), pain, inflammatory pain, and various pulmonary disorders. See, International Application Publication No. WO2015/173169. Furthermore, acid ceramidase enzymes have been identified as a target for the treatment of certain lysosomal storage disorders (for example, Gaucher's, Fabry's, Krabbe, Tay Sachs), and neurodegenerative disorders (for example, Alzheimer's, Parkinson's, Huntington's, and amyotrophic lateral sclerosis). See, International Application Publication Nos. WO2016/210116 and WO2016/210120.

Despite the efforts to develop acid ceramidase inhibitors for use in the treatment of various disorders there is still a need for new acid ceramidase inhibitors.

### SUMMARY

The invention provides substituted benzimidazole carboxamides and related compounds, compositions containing such compounds, medical kits, and methods for using such compounds and compositions to treat medical disorders, for example, cancer (such as glioblastoma), a lysosomal storage disorder (such as Krabbe disease, Fabry disease, Tay-Sachs disease, Pompe disease, Hunter's syndrome, Niemann Pick disease Types A and B, Gaucher disease), a neurodegenerative disease (such as Alzheimer's disease, Parkinson's disease, Huntington's disease, amyotrophic lateral sclerosis, and Lewy body disease), an inflammatory disorder, and pain. Various aspects and embodiments of the invention are described in further detail below.

In one aspect, provided herein is a compound embraced by formula (I):

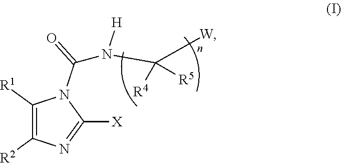

or a pharmaceutically acceptable salt thereof, wherein:

one of R¹ and R² is selected from the group consisting of hydrogen, C₁₋₆alkyl, halogen, cyano, phenyl, 3-12 membered heterocyclyl, C₃₋₇cycloalkyl, C₅₋₁₀bicyclic carbocyclyl, 5-6 membered heteroaryl, C₁₋₆alkylene-cyano, C₁₋₆alkylene-N(R^{a})₂, —O—R^{b}, C₁₋₆alkylene-OR, C₁₋₆alkylene-(5-6 membered heteroaryl), C₁₋₆alkylene-(3-12 membered heterocyclyl), C₁₋₆alkylene-phenyl, C₁₋₆alkylene-C₃₋₇cycloalkyl, (3-7 membered heterocyclylene)-(3-7 membered heterocyclyl), (5-6 membered heteroarylene)-(3-7 membered heterocyclyl), phenylene-(3-7 membered heterocyclyl), phenylene-(3-7 membered heterocyclylene)-(3-7 membered heterocyclyl) and (5-6 membered heteroarylene)-(3-7 membered heterocyclylene)-(3-7 membered heterocyclyl), and the other is selected from the group consisting of hydrogen, C₁₋₆ alkyl, and C₁₋₆alkylene-N(R^{a})₂; R⁴ and R⁵ are independently, for each occurrence, selected from the group consisting of hydrogen, C₁₋₆alkyl, C₁₋₆haloalkyl, and halogen, or R⁴ and R⁵ can be taken together to form C₃₋₇cycloalkylene; n is integer selected from 0 to 6; X is selected from the group consisting of hydrogen, —OR^{c}, —S—C₁₋₆alkyl, C₁₋₆alkyl, and phenyl; R^{a} is independently, for each occurrence, hydrogen or C₁₋₆alkyl; R^{b} is independently, for each occurrence, selected from the group consisting of C₁₋₆alkyl, C₁₋₆alkylene-NR^{a}₂, C₁₋₆haloalkyl, C₃₋₇cycloalkyl, 3-7 membered heterocyclyl, 5-6 membered heteroaryl, and phenyl; R^{c} is independently, for each occurrence, selected from the group consisting of C₁₋₆alkyl, —C₁₋₆alkylene-O—R^{a}, —C₁₋₆alkylene-N(R^{a})₂, C₁₋₆alkylene-(3-7 membered heterocyclyl), C₁₋₆haloalkyl, C₃₋₇cycloalkyl, 3-7 membered heterocyclyl, 5-6 membered heteroaryl, phenyl, and phenylene-(3-7 membered heterocyclyl); and W is selected from the group consisting of methyl, —CF₃, halogen, phenyl, phenylene-phenyl, C₃₋₇cycloalkyl, 3-7 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₆alkynylene, —(C₂₋₆alkynylene)-phenyl, and —(C₂₋₆alkynylene)-C₃₋₇cycloalkyl;

wherein any aforementioned phenyl, C₃₋₇cycloalkyl, 3-12 or 3-7 membered heterocyclyl, or 5-6 membered heteroaryl is optionally substituted (e.g., with one or more substituents each independently selected from the group consisting of C₁₋₆alkyl, C₁₋₆haloalkyl, —CN, halogen, C₁₋₆alkylene-N(R^{a})₂, —O—C₁₋₆alkyl, and oxo, wherein R^{a} is as defined herein); and
when (i) n is 0, or (ii) each of R⁴ and R⁵ is hydrogen, W is not methyl; and when each of R⁴ and R⁵ is independently selected from hydrogen and halogen and W is halogen, R² is not pyridyl.

In some embodiments, the compound is a compound of formula (I-a):

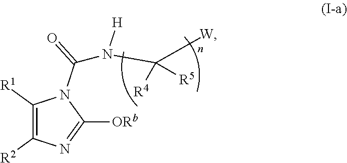

or a pharmaceutically acceptable salt thereof, wherein the variables are as defined herein.

In some embodiments, the compound is a compound of formula (I-b):

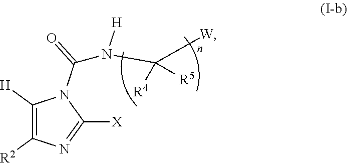

or a pharmaceutically acceptable salt thereof, wherein the variables are as defined herein.

In other embodiments, the compound is a compound of formula (I-c):

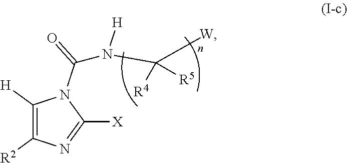

or a pharmaceutically acceptable salt thereof, wherein the variables are as defined herein.

In another aspect, provided herein is a pharmaceutical composition comprising a compound disclosed herein (e.g., a compound of Formula (I), e.g., a compound of Formula (I-a), (I-b), or (I-c)) and a pharmaceutically acceptable carrier.

In another aspect, provided herein is a method of treating a subject with cancer and in need thereof, the method comprising administering to the subject a therapeutically effective amount of a compound (e.g., a compound of Formula (I), e.g., a compound of Formula (I-a), (I-b), or (I-c)) or a pharmaceutical composition disclosed herein.

In another aspect, provided herein is a method of treating a subject with a lysosomal storage disorder and in need thereof, the method comprising administering to the subject a therapeutically effective amount of a compound (e.g., a compound of Formula (I), e.g., a compound of Formula (I-a), (I-b), or (I-c)) or a pharmaceutical composition disclosed herein.

In another aspect, provided herein is a method of treating a subject with a neurodegenerative disorder and in need thereof, the method comprising administering to the subject a therapeutically effective amount of a compound (e.g., a compound of Formula (I), e.g., a compound of Formula (I-a), (I-b), or (I-c)) or a pharmaceutical composition disclosed herein.

In another aspect, provided herein is a method of treating a subject with an inflammatory disorder and in need thereof, the method comprising administering to the subject a therapeutically effective amount of a compound (e.g., a compound of Formula (I), e.g., a compound of Formula (I-a), (I-b), or (I-c)) or a pharmaceutical composition disclosed herein.

In another aspect, the invention provides a compound (e.g., a compound of Formula (I), e.g., a compound of Formula (I-a), (I-b), or (I-c)) or a pharmaceutical composition as disclosed herein for use in a method of treating a subject with cancer and in need thereof, the method comprising administering to the subject a therapeutically effective amount of the compound or the pharmaceutical composition.

In another aspect, the invention provides a compound (e.g., a compound of Formula (I), e.g., a compound of Formula (I-a), (I-b), or (I-c)) or a pharmaceutical composition as disclosed herein for use in a method of treating a subject with a lysosomal storage disorder and in need thereof, the method comprising administering to the subject a therapeutically effective amount of the compound or the pharmaceutical composition.

In another aspect, the invention provides a compound (e.g., a compound of Formula (I), e.g., a compound of Formula (I-a), (I-b), or (I-c)) or a pharmaceutical composition as disclosed herein for use in a method of treating a subject with a neurodegenerative disorder and in need thereof, the method comprising administering to the subject a therapeutically effective amount of the compound or the pharmaceutical composition.

In another aspect, the invention provides a compound (e.g., a compound of Formula (I), e.g., a compound of Formula (I-a), (I-b), or (I-c)) or a pharmaceutical composition as disclosed herein for use in a method of treating a subject with an inflammatory disorder and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), or (I-c)) or a pharmaceutical composition as disclosed herein for the manufacture of a medicament for treating a subject with cancer and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), or (I-c)) or a pharmaceutical composition as disclosed herein for the manufacture of a medicament for treating a subject with a lysosomal storage disorder and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), or (I-c)) or a pharmaceutical composition as disclosed herein for the manufacture of a medicament for treating a subject with a neurodegenerative disorder and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), or (I-c)) or a pharmaceutical composition as disclosed herein for the manufacture of a medicament for treating a subject with an inflammatory disorder and in need thereof, the method comprising administering to the subject a therapeutically effective amount of the compound or the pharmaceutical composition.

### DETAILED DESCRIPTION

The invention provides substituted imidazole carboxamides and related compounds, compositions containing such compounds, medical kits, and methods for using such compounds and compositions to treat medical disorders in a patient. The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein should be construed according to the standard rules of chemical valency known in the chemical arts.

The terms “a” and “an” as used herein mean “one or more” and include the plural unless the context is inappropriate.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). The heteroalkyl may be, for example, an —O—C₁-C₁₀alkyl group, an —C₁-C₆alkylene-O—C₁-C₆alkyl group, or a C₁-C₆ alkylene-OH group. In certain embodiments, the “heteroalkyl” may be 2-8 membered heteroalkyl, indicating that the heteroalkyl contains from 2 to 8 atoms selected from the group consisting of carbon, oxygen, nitrogen, and sulfur. In yet other embodiments, the heteroalkyl may be a 2-6 membered, 4-8 membered, or a 5-8 membered heteroalkyl group (which may contain for example 1 or 2 heteroatoms selected from the group oxygen and nitrogen). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, C₂-C₁₀alkenyl, and C₂-C₆alkenyl, respectively. Exemplary alkenyl groups include vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, and the like.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂alkynyl, C₂-C₁₀alkynyl, and C₂-C₆alkynyl, respectively. Exemplary alkynyl groups include ethynyl, prop-1-yn-1-yl, and but-1-yn-1-yl.

The term “cycloalkyl” refers to a monovalent saturated cyclic, bicyclic, bridged cyclic (e.g., adamantyl), or spirocyclic hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group. An exemplary cycloalkylene group is

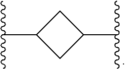

The term “cycloalkenyl” as used herein refers to a monovalent unsaturated cyclic, bicyclic, or bridged cyclic (e.g., adamantyl) hydrocarbon group of 3-12, 3-8, 4-8, or 4-6 carbons containing one carbon-carbon double bond, referred to herein, e.g., as “C₄₋₈cycloalkenyl,” derived from a cycloalkane. Exemplary cycloalkenyl groups include, but are not limited to, cyclohexenes, cyclopentenes, and cyclobutenes. Unless specified otherwise, cycloalkenyl groups are optionally substituted at one or more ring positions with, for example, alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkenyl group is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The term “aralkyl” refers to an alkyl group substituted with an aryl group.

The term “bicyclic carbocyclyl that is partially unsaturated” refers to a bicyclic carbocyclic group containing at least one double bond between ring atoms and at least one ring in the bicyclic carbocyclic group is not aromatic. Representative examples of a bicyclic carbocyclyl that is partially unsaturated include, for example:

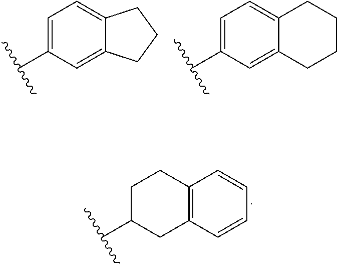 [SMILES: *c1ccc2c(c1)CCC2 ; *c1ccc2c(c1)CCCC2 ; *C1CCc2ccccc2C1]

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may be, for example, mono-, bi-, or other multi-cyclic ring systems. A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “bicyclic heterocyclyl” refers to a fused, spiro, or bridged heterocyclyl group that contains two rings. Representative examples of a bicyclic heterocyclyl include, for example:

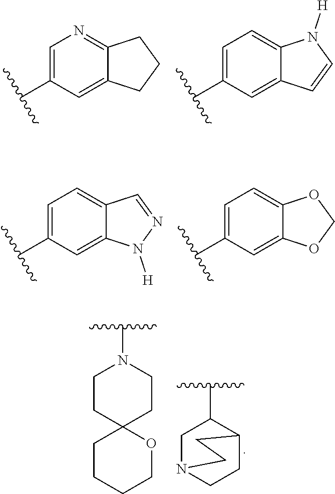

In certain embodiments, the bicyclic heterocyclyl is a carbocyclic ring fused to partially unsaturated heterocyclic ring, that together form a bicyclic ring structure having 8-10 ring atoms (e.g., where there are 1, 2, 3, or 4 heteroatoms selected from the group consisting of nitrogen, oxygen, and sulfur).

The term “heterocyclylene” refers to a diradical of a heterocyclyl group. An exemplary heterocyclylene group is

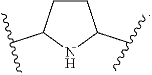

The heterocyclylene may contain, for example, 3-6 ring atom (i.e., a 3-6 membered heterocyclylene). In certain embodiments, the heterocyclylene is a 3-6 membered heterocyclylene containing 1, 2, or 3 three heteroatoms selected from the group consisting of oxygen, nitrogen, and sulfur.

The term “bicyclic heterocyclylene” refers to a diradical of a bicyclic heterocyclyl group.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The terms “amine” and “amino” are art-recognized and refer to both unsubstituted and substituted amines, e.g., a moiety represented by the general formula —N(R⁵⁰)(R⁵¹), wherein R⁵⁰ and R⁵¹ each independently represent hydrogen, alkyl, cycloalkyl, heterocyclyl, alkenyl, aryl, aralkyl, or —(CH₂)ₘ—R⁶¹; or R⁵⁰ and R⁵¹, taken together with the N atom to which they are attached complete a heterocycle having from 4 to 8 atoms in the ring structure; R^{6′} represents an aryl, a cycloalkyl, a cycloalkenyl, a heterocycle or a polycycle; and m is zero or an integer in the range of 1 to 8. In certain embodiments, R⁵⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R₆₁, where m and R₆₁ are described above. The term “haloalkoxyl” refers to an alkoxyl group that is substituted with at least one halogen. For example, —O—CH₂F, —O—CHF₂, —O—CF₃, and the like. In certain embodiments, the haloalkoxyl is an alkoxyl group that is substituted with at least one fluoro group. In certain embodiments, the haloalkoxyl is an alkoxyl group that is substituted with from 1-6, 1-5, 1-4, 2-4, or 3 fluoro groups.

Any aryl (e.g., phenyl), cycloalkyl (e.g., C₃₋₇cycloalkyl), heterocyclyl (e.g., 3-12 membered heterocyclyl), heteroaryl (e.g., 5-6 membered heteroaryl) may be optionally substituted unless otherwise states. In some embodiments, any aryl (e.g., phenyl), cycloalkyl (e.g., C₃₋₇cycloalkyl), heterocyclyl (e.g., 3-12 membered heterocyclyl), heteroaryl (e.g., 5-6 membered heteroaryl) may be optionally substituted with 1-4 substituents independently for each occurrence selected from the group consisting of halogen, C₁₋₆alkyl, C₁₋₆haloalkyl, C₁₋₆alkoxy, cyano, N(R^{aa})₂, —CH₂N(R^{aa})₂, and hydroxyl, wherein R^{aa} is independently for each occurrence hydrogen or C₁₋₆alkyl.

The term “carbamate” as used herein refers to a radical of the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently alkoxy, aryloxy, alkyl, alkenyl, alkynyl, amide, amino, aryl, arylalkyl, carboxy, cyano, cycloalkyl, ester, ether, formyl, halogen, haloalkyl, heteroaryl, heterocyclyl, hydroxyl, ketone, nitro, sulfide, sulfonyl, or sulfonamide. Exemplary carbamates include arylcarbamates and heteroaryl carbamates, e.g., wherein at least one of R_{g}, Rₕ and Rᵢ are independently aryl or heteroaryl, such as phenyl and pyridinyl.

The term “carbonyl” as used herein refers to the radical —C(O)—.

The term “carboxamido” as used herein refers to the radical —C(O)NRR′, where R and R′ may be the same or different. R and R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —RₐC(O)N(R_{b})—, —RₐC(O)N(R_{b})R_{c}—, —C(O)NR_{b}R_{c}, or —C(O)NH₂, wherein Rₐ, R_{b} and R_{c} are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro. The amide can be attached to another group through the carbon, the nitrogen, R_{b}, R_{c}, or Rₐ. The amide also may be cyclic, for example R_{b} and R_{c}, Rₐ and R_{b}, or Rₐ and R_{c} may be joined to form a 3- to 12-membered ring, such as a 3- to 10-membered ring or a 5- to 6-membered ring.

The term “amidino” as used herein refers to a radical of the form —C(═NR)NR′R″ where R, R′, and R″ are each independently alkyl, alkenyl, alkynyl, amide, aryl, arylalkyl, cyano, cycloalkyl, haloalkyl, heteroaryl, heterocyclyl, hydroxyl, ketone, or nitro.

The term “alkanoyl” as used herein refers to a radical —O—CO-alkyl.

The term “oxo” is art-recognized and refers to a “═O” substituent. For example, a cyclopentane substituted with an oxo group is cyclopentanone.

The term “sulfonamide” or “sulfonamido” as used herein refers to a radical having the structure —N(Rᵣ)—S(O)₂—Rₛ— or —S(O)₂—N(Rᵣ)Rₛ, where Rᵣ, and Rₛ can be, for example, hydrogen, alkyl, aryl, cycloalkyl, and heterocyclyl. Exemplary sulfonamides include alkylsulfonamides (e.g., where Rₛ is alkyl), arylsulfonamides (e.g., where Rₛ is aryl), cycloalkyl sulfonamides (e.g., where Rₛ is cycloalkyl), and heterocyclyl sulfonamides (e.g., where Rₛ is heterocyclyl), etc.

The term “sulfonyl” as used herein refers to a radical having the structure RᵤSO₂—, where Rᵤ can be alkyl, aryl, cycloalkyl, and heterocyclyl, e.g., alkylsulfonyl. The term “alkylsulfonyl” as used herein refers to an alkyl group attached to a sulfonyl group.

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. In some embodiments, an optional substituent may be selected from the group consisting of: C₁₋₆alkyl, cyano, halogen, —O—C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂, wherein R^{a} is hydrogen or C_{1-b}alkyl. In some embodiments, an optional substituent may be selected independently for each occurrence from the group consisting of CH₂N(R^{a})₂, cyano, C₁₋₆alkyl, halogen, and —O—C₁₋₆alkyl, wherein R^{a} is hydrogen or C₁₋₆alkyl.

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(f)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the compounds and compositions of the present invention. Such organisms are preferably mammals (e.g., murines, simians, equines, bovines, porcines, canines, felines, and the like), and more preferably humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Abbreviations as used herein include diisopropylethylamine (DIPEA); dimethylformamide (DMF); methylene chloride (DCM); tetrahydrofuran (THF); trifluoroacetic acid (TFA); dimethylsulfoxide (DMSO); diisopropylethylamine (DIEA); ethyl acetate (EtOAc or EA); petroleum ether (PE); p-methoxybenzyl (PMB); flash column chromatography (FCC); supercritical fluid chromatography (SFC); acetonitrile (ACN); acetic acid (AcOH); ammonium acetate (NH₄OAc); ethylene bridged hybrid (BEH); broadband inverse (BBI); cyclohexane (Cy); dichloroethane (DCE); dimethylamine (NHMe₂); dimethylcyclohexanedicarboxylate (DMCD); ethanol (EtOH); in situ chemical oxidation (ISCO); high-performance liquid chromatography (HPLC); methanol (MeOH); methylmagnesium bromide (MeMgBr); mass spectrometry, electrospray (MS (ES)); methyl tert-butyl ether (MTBE); methyl iodide (MeI); nuclear magnetic resonance spectroscopy (NMR); [1,1′-Bis(diphenylphosphino)ferrocene]dichloropalladium(II), complex with dichloromethane (PdCl₂(dppf)-DCM); photodiode array (PDA); p-toluenesulfonic acid (p-TsOH); potassium acetate (KOAc); room temperature (RT); sodium acetate (NaOAc); sodium methoxide (NaOMe); sodium triacetoxyborohydride (NaBH(AcO)₃); solid phase extraction (SPE); thin layer chromatography (TLC); triethylamine (Et₃N); 2-(trimethylsilyl)ethoxymethyl chloride (SEMCl); and ultra-performance liquid chromatography/mass spectrometry (UPLC/MS).

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a 10% variation from the nominal value unless otherwise indicated or inferred.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. Additional examples include that the phrase “optionally substituted with 1-5 substituents” is specifically intended to individually disclose a chemical group that can include 0, 1, 2, 3, 4, 5, 0-5, 0-4, 0-3, 0-2, 0-1, 1-5, 1-4, 1-3, 1-2, 2-5, 2-4, 2-3, 3-5, 3-4, and 4-5 substituents.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Substituted Imidazole Carboxamides and Related Compounds

It has been discovered that the active site (binding site) of human acid ceramidase (ASAH-1), as determined by x-ray crystallography, contains a plurality of hydration sites, each of which is occupied by a single molecule of water and whose position and energetics (which incorporates the enthalpy, entropy, and free energy values associated with each water molecule) have been calculated. Each of these water molecules has a stability rating (namely, a numerical calculation which incorporates the enthalpy, entropy, and free energy values associated with each water molecule), which provides a measurable value associated with the relative stability of water molecules that occupy hydration sites in the binding pocket of the acid ceramidase enzyme. Water molecules occupying hydration sites in the binding pocket of acid ceramidase having a stability rating of >2.5 kcal/mol are referred to as unstable waters. It is contemplated that the displacement or disruption of an unstable water molecule (i.e., a water molecule having a stability rating of greater than 2.5 kcal/mol), or replacement of a stable water molecule (i.e., a water molecule having a stability rating of less than 1 kcal/mol), by an inhibitor results in tighter binding of that inhibitor. Accordingly, it is contemplated that an inhibitor designed to displace one or more unstable water molecules (namely, a water molecule having a stability rating of greater than 2.5 kcal/mol) may bind more tightly to the binding pocket and, therefore, will be a more potent inhibitor as compared to an inhibitor that does not displace unstable water molecules. Certain of the compounds described herein were designed to displace one or more unstable water molecules in the binding pocket.

### Compounds

One aspect of the invention provides substituted imidazole carboxamides and related compounds. The substituted imidazole carboxamides and related organic compounds are contemplated to be useful in the methods, compositions, and kits described herein. In certain embodiments, the substituted imidazole carboxamides or related organic compound is a compound embraced by formula (I):

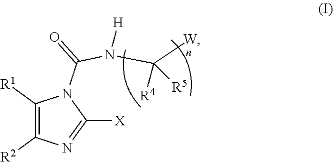

or a pharmaceutically acceptable salt thereof, wherein:

one of R¹ and R² is selected from the group consisting of hydrogen, C₁₋₆alkyl, halogen, cyano, phenyl, 3-12 membered heterocyclyl, C₃₋₇cycloalkyl, C₅₋₁₀bicyclic carbocyclyl, 5-6 membered heteroaryl, C₁₋₆alkylene-cyano, C₁₋₆alkylene-N(R^{a})₂, —O—R^{b}, C₁₋₆alkylene-OR^{b}, C₁₋₆alkylene-(5-6 membered heteroaryl), C₁₋₆alkylene-(3-12 membered heterocyclyl), C₁₋₆alkylene-phenyl, C₁₋₆alkylene-C₃₋₇cycloalkyl, (3-7 membered heterocyclylene)-(3-7 membered heterocyclyl), (5-6 membered heteroarylene)-(3-7 membered heterocyclyl), phenylene-(3-7 membered heterocyclyl), phenylene-(3-7 membered heterocyclylene)-(3-7 membered heterocyclyl), and (5-6 membered heteroarylene)-(3-7 membered heterocyclylene)-(3-7 membered heterocyclyl), and the other is selected from the group consisting of hydrogen, C₁₋₆alkyl, and C₁₋₆alkylene-N(R^{a})₂;

R⁴ and R⁵ are independently, for each occurrence, selected from the group consisting of hydrogen, C₁₋₆alkyl, C₁₋₆haloalkyl, and halogen, or R⁴ and R⁵ can be taken together to form C₃₋₇cycloalkylene;

n is integer selected from 0 to 6;

X is selected from the group consisting of hydrogen, —OR^{c}, —S—C₁₋₆alkyl, C₁₋₆alkyl, and phenyl;

R^{a} is independently, for each occurrence, hydrogen or C₁₋₆alkyl;

R^{b} is independently, for each occurrence, selected from the group consisting of C₁₋₆alkyl, C₁₋₆alkylene-NR′2, C₁₋₆haloalkyl, C₃₋₇cycloalkyl, 3-7 membered heterocyclyl, 5-6 membered heteroaryl, and phenyl;

R^{c} is independently, for each occurrence, selected from the group consisting of C₁₋₆alkyl, —C₁₋₆alkylene-O—R^{a}, —C₁₋₄alkylene-N(R^{a})₂, C₁₋₆alkylene-(3-7 membered heterocyclyl), C₁₋₆haloalkyl, C₃₋₇cycloalkyl, 3-7 membered heterocyclyl, 5-6 membered heteroaryl, phenyl, and phenylene-(3-7 membered heterocyclyl); and

W is selected from the group consisting of methyl, —CF₃, halogen, phenyl, phenylene-phenyl, C₃₋₇cycloalkyl, 3-7 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₄alkynylene, —(C₂₋₆alkynylene)-phenyl, and —(C₂₋₆alkynylene)-C₃₋₇cycloalkyl;

wherein any aforementioned phenyl, C₃₋₆cycloalkyl, 3-12 or 3-7 membered heterocyclyl, or 5-6 membered heteroaryl is optionally substituted (e.g., with one or more substituents each independently selected from the group consisting of C₁₋₆alkyl, C₁₋₆haloalkyl, —CN, halogen, C₁₋₆alkylene-N(R^{a})₂, —O—C₁₋₆alkyl, and oxo, wherein R^{a} is as defined herein); and
when (i) n is 0, or (ii) each of R⁴ and R⁵ is hydrogen, W is not methyl; and when each of R⁴ and R⁵ is independently selected from hydrogen and halogen and W is halogen, R² is not pyridyl.

In some embodiments, X is selected from the group consisting of hydrogen, methyl, —OR^{c}, and —SCH₃. In some embodiments, X is —OR^{c}.

In some embodiments, the compound is a compound of formula (I-a):

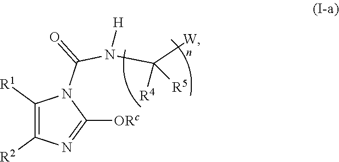

or a pharmaceutically acceptable salt thereof, wherein the variables are as defined herein.

In each of the foregoing compounds of formula (I) and formula (I-a), R^{c} is selected from the group consisting of methyl, ethyl, —CH₂(CH₃)₂, phenyl,

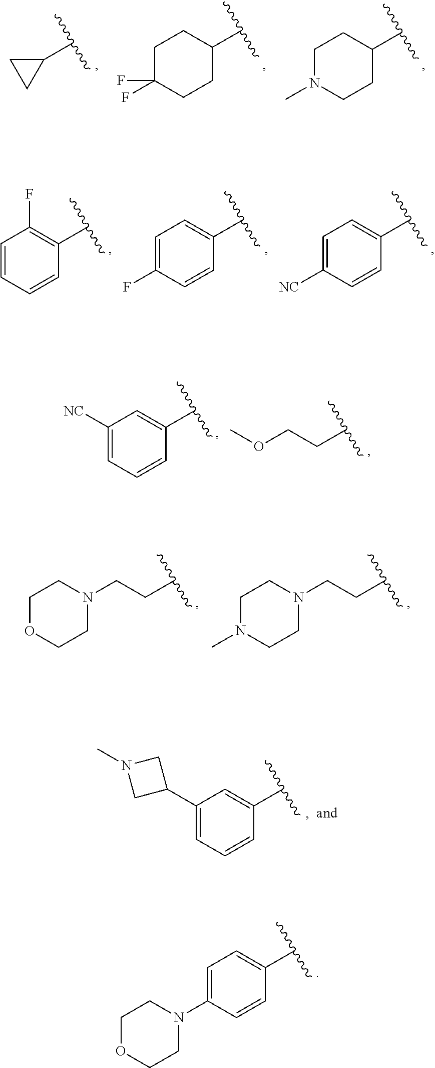

In some embodiments, R^{c} is ethyl. In some embodiments, R^{c} is methyl. In some embodiments, R^{c} is phenyl.

In each of the foregoing compounds of formula (I) and formula (I-a), R² is selected from the group consisting of hydrogen, C₁₋₆alkyl, halogen, cyano, —O—R^{b}, phenyl, 3-12 membered heterocyclyl, C₃₋₇cycloalkyl, C₅₋₁₀ bicyclic carbocyclyl, 5-6 membered heteroaryl, C₁₋₆alkylene-cyano, C₁₋₄alkylene-OR^{b}, C₁₋₆alkylene-N(R^{a})₂, C₁₋₆alkylene-(5-6 membered heteroaryl), C₁₋₆alkylene-(3-12 membered heterocyclyl), C₁₋₆alkylene-phenyl, C₁₋₆alkylene-C₃₋₇cycloalkyl, (3-7 membered heterocyclylene)-(3-7 membered heterocyclyl), phenylene-(3-7 membered heterocyclyl), phenylene-(3-7 membered heterocyclylene)-(3-7 membered heterocyclyl), (5-6 membered heteroarylene)-(3-7 membered heterocyclyl), and (5-6 membered heteroarylene)-(3-7 membered heterocyclylene)-(3-7 membered heterocyclyl), and R¹ is selected from the group consisting of hydrogen, C₁₋₆alkyl, and C₁₋₆alkylene-N(R^{a})₂, wherein the 3-12 membered heterocyclyl, C₃₋₇cycloalkyl, phenyl, 5-6 membered heteroaryl, C₁₋₆alkylene-(3-12 membered heterocyclyl) (5-6 membered heteroarylene)-(3-7 membered heterocyclyl), and (5-6 membered heteroarylene)-(3-7 membered heterocyclylene)-(3-7 membered heterocyclyl) are optionally substituted.

In each of the foregoing compounds of formula (I) and formula (I-a), R² is selected from the group consisting of hydrogen, C₁₋₆alkyl, halogen, phenyl, 3-12 membered heterocyclyl, C₃₋₇cycloalkyl, 5-6 membered heteroaryl, C₁₋₆alkylene-cyano, C₁₋₆alkylene-(3-12 membered heterocyclyl), (5-6 membered heteroarylene)-(3-7 membered heterocyclyl), and (5-6 membered heteroarylene)-(3-7 membered heterocyclylene)-(3-7 membered heterocyclyl), and R¹ is hydrogen or methyl, wherein the 3-12 membered heterocyclyl, C₃₋₇cycloalkyl, phenyl, 5-6 membered heteroaryl, C₁₋₆alkylene-(3-12 membered heterocyclyl), (5-6 membered heteroarylene)-(3-7 membered heterocyclyl), and (5-6 membered heteroarylene)-(3-7 membered heterocyclylene)-(3-7 membered heterocyclyl) are optionally substituted with 1-3 substituents independently, for each occurrence, selected from the group consisting of —CH₂N(CH₃)₂, cyano, C₁₋₆alkyl, halogen, methoxy, oxo, and combinations thereof.

In each of the foregoing compounds of formula (I) and formula (I-a), R² is selected from the group consisting of hydrogen, —O—R^{b}, phenyl, C₁₋₆alkyl, C₁₋₆alkylene-O—C₁₋₆alkylene, C₁₋₆alkylene-NMe₂, 3-12 membered heterocyclyl, and 5-6 membered heteroaryl, and R¹ is selected from the group consisting of hydrogen, methyl, and —CH₂NMe₂.

In each of the foregoing compounds of formula (I) and formula (I-a), R² is selected from the group consisting of hydrogen, phenyl, C₁₋₆alkyl, 3-12 membered heterocyclyl, and 5-6 membered heteroaryl.

In each of the foregoing compounds of formula (I) and formula (I-a), R² is selected from the group consisting of hydrogen, methyl, ethyl, isopropyl, t-butyl, —C(CH₃)₂CN, bromine, chlorine, phenyl,

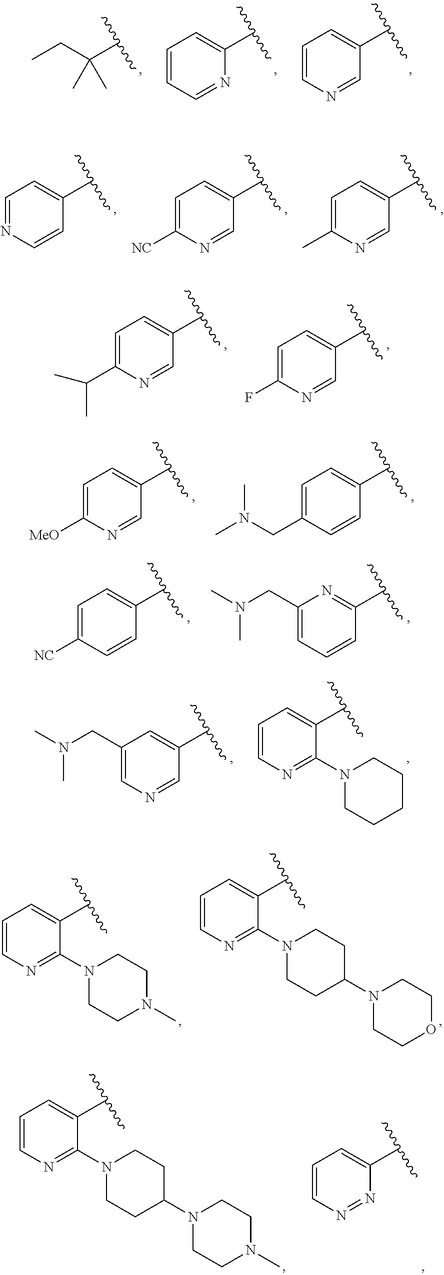

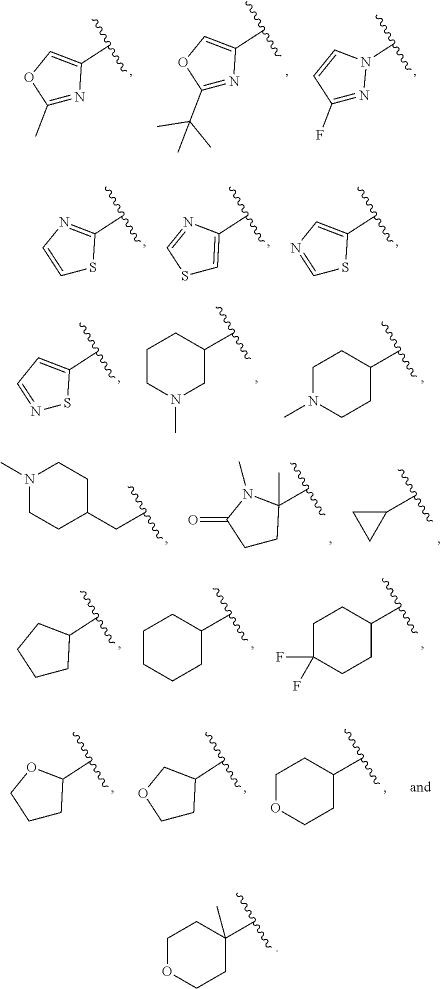

In some embodiments R² is selected from the group consisting of phenyl, t-butyl,

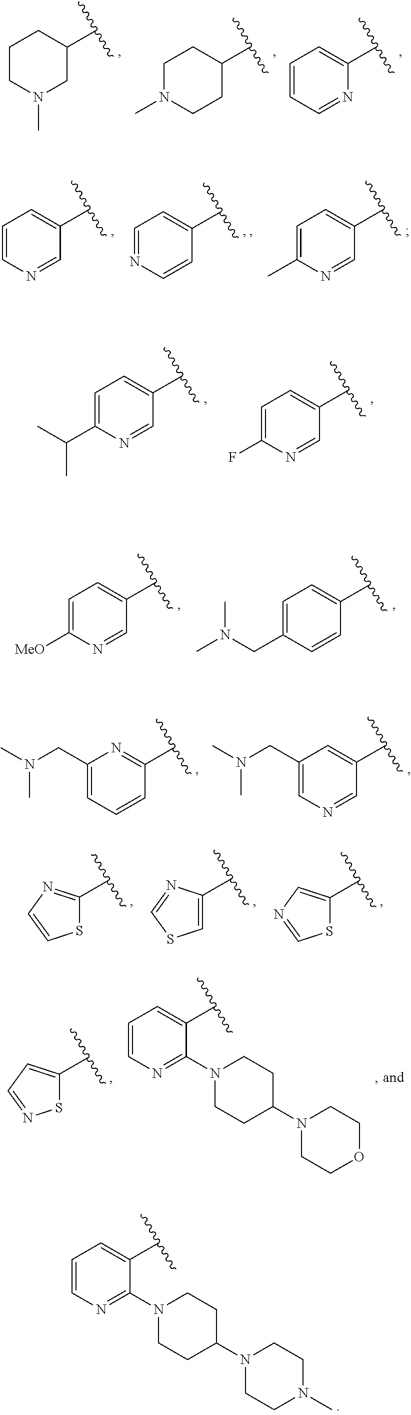

In some embodiments, R² is selected from the group consisting of phenyl, t-butyl,

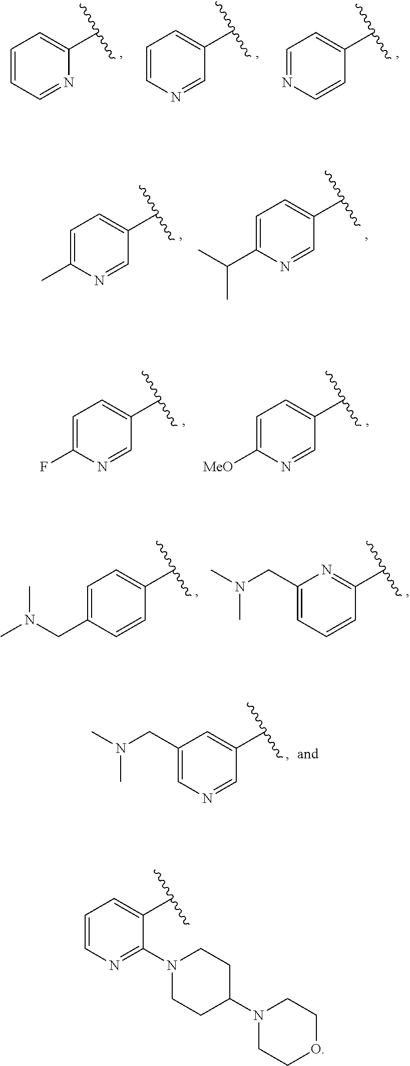

In each of the foregoing compounds of formula (I) and formula (I-a), R² is

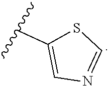 [SMILES: *c1cncs1]

In each of the foregoing compounds of formula (I) and formula (I-a), R² is

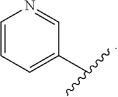 [SMILES: *c1cccnc1]

In each of the foregoing compounds of formula (I) and formula (I-a), R² is i-butyl.

In each of the foregoing compounds of formula (I) and formula (I-a), R² is 5-6 membered heteroaryl.

In each of the foregoing compounds of formula (I) and formula (I-a), R¹ is hydrogen.

In each of the foregoing compounds of formula (I) and formula (I-a), R⁴ and R⁵ are independently, for each occurrence, hydrogen or methyl, or R⁴ and R⁵ can be taken together to form a cyclopropylene.

In each of the foregoing compounds of formula (I) and formula (I-a), R⁴ and R⁵ are independently, for each occurrence, hydrogen or methyl.

In each of the foregoing compounds of formula (I) and formula (I-a), R⁴ and R⁵ are hydrogen.

In each of the foregoing compounds of formula (I) and formula (I-a), n is 0, 1, 2, 3, 4, or 5.

In each of the foregoing compounds of formula (I) and formula (I-a), n is 0. In each of the foregoing compounds of formula (I) and formula (I-a), n is 1. In each of the foregoing compounds of formula (I) and formula (I-a), n is 2. In each of the foregoing compounds of formula (I) and formula (I-a), n is 3. In each of the foregoing compounds of formula (I) and formula (I-a), n is 4. In each of the foregoing compounds of formula (I) and formula (I-a), n is 5. In each of the foregoing compounds of formula (I) and formula (I-a), n is 6.

In each of the foregoing compounds of formula (I) and formula (I-a), W is selected from the group consisting of methyl, CF₃, halogen, —O—C₁₋₆alkyl, —O—C₁₋₆haloalkyl, C₂₋₆alkynylene, phenyl, C₃₋₇cycloalkyl, 5-6 membered heteroaryl, —(C₂₋₆alkynylene)-phenyl, —(C₂₋₆alkynylene)-C₃₋₇cycloalkyl, and —O-phenyl, wherein phenyl, C₃₋₇cycloalkyl, 5-6 membered heteroaryl, and —(C₂₋₆alkynylene)-C₃₋₇cycloalkyl is optionally substituted with 1-3 substituents independently, for each occurrence, selected from the group consisting of C₁₋₆alkyl, halogen, CF₃, phenyl, and combinations thereof.

In each of the foregoing compounds of formula (I) and formula (I-a), W is selected from the group consisting of methyl, —CF₃, —OCF₃,

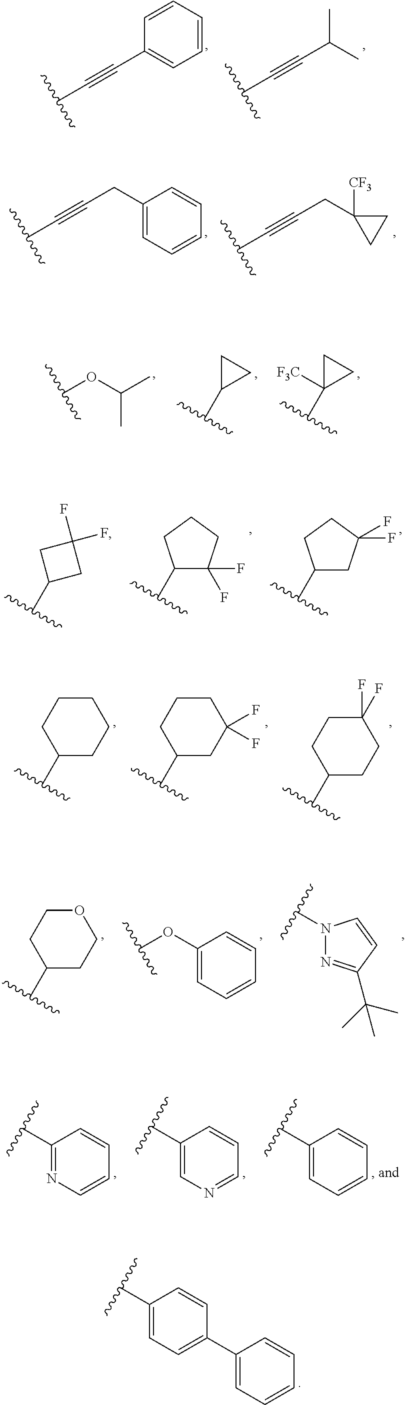

In each of the foregoing compounds of formula (I) and formula (I-a), W is selected from the group consisting of methyl,

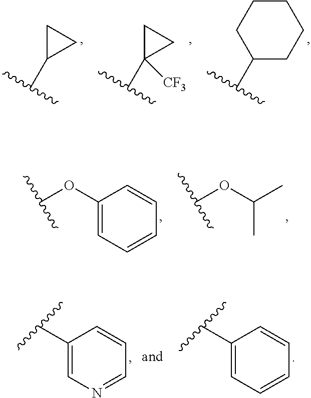

In each of the foregoing compounds of formula (I) and formula (I-a), W is selected from the group consisting of methyl,

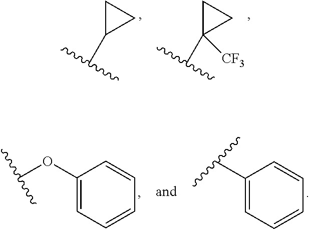

In some embodiments, W is methyl or phenyl.

In some embodiments, W is methyl.

In some embodiments, W is phenyl.

In some embodiments, W is

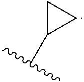 [SMILES: *C1CC1]

In each of the foregoing compounds of formula (I) and formula (I-a), any aforementioned phenyl, 3-12 membered heterocyclyl, or 5-6 membered heteroaryl is independently, for each occurrence, optionally substituted with 1-3 substituents independently, for each occurrence, selected from the group consisting of C₁₋₆alkyl, C₁₋₆haloalkyl, —CN, halogen, C₁₋₆alkylene-N(R^{a})₂, —O—C₁₋₆alkyl, and oxo, wherein R^{a} is as defined herein).

In each of the foregoing compounds of formula (I) and formula (I-a), any aforementioned phenyl is optionally substituted with 1-3 of —CH₂N(CH₃)₂, halogen, or —CN, any aforementioned 3-12 membered heterocyclyl is independently for each occurrence optionally substituted with 1-3 substituents each independently selected from methyl and oxo, any aforementioned 5-6 membered heteroaryl is independently for each occurrence optionally substituted with 1-3 substituents independently, for each occurrence, selected from the group consisting of —CH₂N(CH₃)₂, cyano, C₁₋₆alkyl, halogen, and methoxy, and any aforementioned C₃₋₇cyclohexyl is independently for each occurrence optionally substituted with 1-3 substituents each independently halogen or trifluoromethyl.

In each of the foregoing compounds of formula (I) and formula (I-a), any phenyl at R² is independently, for each occurrence, optionally substituted with 1-2 of —CH₂N(CH₃)₂, any 3-12 membered heterocyclyl of R² is independently, for each occurrence, optionally substituted with 1-3 substituents each independently selected from methyl or oxo, any 5-6 membered heteroaryl of R² is independently, for each occurrence, optionally substituted with 1 or 2 substituents independently, for each occurrence, selected from the group consisting of —CH₂N(CH₃)₂, cyano, C₁₋₆alkyl, halogen, and methoxy, and any C₃₋₇cyclohexyl at R² is independently for each occurrence optionally substituted with 1-3 halogen.

In some embodiments, the compound is a compound of formula (I-b):

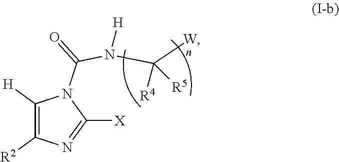

or a pharmaceutically acceptable salt thereof, wherein:

R² is selected from the group consisting of hydrogen, C₁₋₆alkyl, halogen, cyano, phenyl, 3-12 membered heterocyclyl, C₃₋₇cycloalkyl, C₅₋₁₀ bicyclic carbocyclyl, 5-6 membered heteroaryl, C_{1-f}alkylene-cyano, C₁₋₆alkylene-N(R^{a})₂, —O—R^{b}, C₁₋₆alkylene-OR^{b}, C₁₋₆alkylene-(5-6 membered heteroaryl), C₁₋₆alkylene-(3-12 membered heterocyclyl), C₁₋₆alkylene-phenyl, C₁₋₆alkylene-C₃₋₇cycloalkyl, (3-7 membered heterocyclylene)-(3-7 membered heterocyclyl), (5-6 membered heteroarylene)-(3-7 membered heterocyclyl), phenylene-(3-7 membered heterocyclyl), phenylene-(3-7 membered heterocyclylene)-(3-7 membered heterocyclyl), and (5-6 membered heteroarylene)-(3-7 membered heterocyclylene)-(3-7 membered heterocyclyl);

n is an integer selected from 0 to 6; wherein,

when n is selected from 2 to 6, R⁴ and R⁵ are independently, for each occurrence, selected from the group consisting of hydrogen, C₁₋₆alkyl, C₁₋₆haloalkyl, and halogen, or R⁴ and R⁵ can be taken together to form C₃₋₇cycloalkylene;

when n is 1, R⁴ and R⁵ are independently selected from the group consisting of hydrogen, C₁₋₆alkyl, C₁₋₆haloalkyl, and halogen;

X is selected from the group consisting of hydrogen, —OR^{c}, —S—C₁₋₆alkyl, C₁₋₆alkyl, and phenyl;

R^{a} is independently, for each occurrence, hydrogen or C₁₋₆alkyl;

R^{b} is independently, for each occurrence, selected from the group consisting of C₁₋₆alkyl, C₁₋₆alkylene-NR^{a}₂, C₁₋₆haloalkyl, C₃₋₇cycloalkyl, 3-7 membered heterocyclyl, 5-6 membered heteroaryl, and phenyl;

R^{c} is independently, for each occurrence, selected from the group consisting of C₁₋₆alkyl, —C₁₋₆alkylene-O—R^{a}, —C₁₋₆alkylene-N(R^{a})₂, C₁₋₆alkylene-(3-7 membered heterocyclyl), C₁₋₆haloalkyl, C₃₋₇cycloalkyl, 3-7 membered heterocyclyl, 5-6 membered heteroaryl, phenyl, and phenylene-(3-7 membered heterocyclyl); and

W is selected from the group consisting of methyl, halogen, phenyl, phenylene-phenyl, C₃₋₇cycloalkyl, 3-7 membered heterocyclyl, 5-6 membered heteroaryl, —O—C₁₋₆alkyl, —O—C₁₋₆haloalkyl, —O-phenyl, —O—(C₁₋₄alkylene)-phenyl, C₂₋₆alkynyl, —(C₂₋₆alkynylene)-phenyl, and —(C₂₋₆alkynylene)-C₃₋₇cycloalkyl; and

wherein any aforementioned phenyl, C₃₋₆cycloalkyl, 3-12 or 3-7 membered heterocyclyl, or 5-6 membered heteroaryl is optionally substituted with one or more substituents each independently selected from the group consisting of C₁₋₆alkyl, C₁₋₆haloalkyl, —CN, halogen, C₁₋₆alkylene-N(R^{a})₂, —O—C₁₋₆alkyl, and oxo, wherein R^{a} is hydrogen or C₁₋₆alkyl; wherein

when (i) n is 0, or (ii) each of R⁴ and R⁵ is hydrogen, W is not methyl, and when each of R⁴ and R⁵ is independently selected from hydrogen and halogen and W is halogen, R² is not pyridyl.

In some embodiments, R² is selected from the group consisting of hydrogen, —O—R^{b}, phenyl, C₁₋₆alkyl, C₁₋₆alkylene-O—C₁₋₆alkylene, C₁₋₆alkylene-NMe₂, 3-12 membered heterocyclyl, and 5-6 membered heteroaryl, and R¹ is selected from the group consisting of hydrogen, methyl, and —CH₂NMe₂.

In some embodiments, R² is selected from the group consisting of hydrogen, phenyl, C₁₋₆alkyl, 3-12 membered heterocyclyl, and 5-6 membered heteroaryl.

In some embodiments, R² is selected from the group consisting of hydrogen, methyl, ethyl, isopropyl, t-butyl, —C(CH₃)₂CN, bromine, chlorine, phenyl,

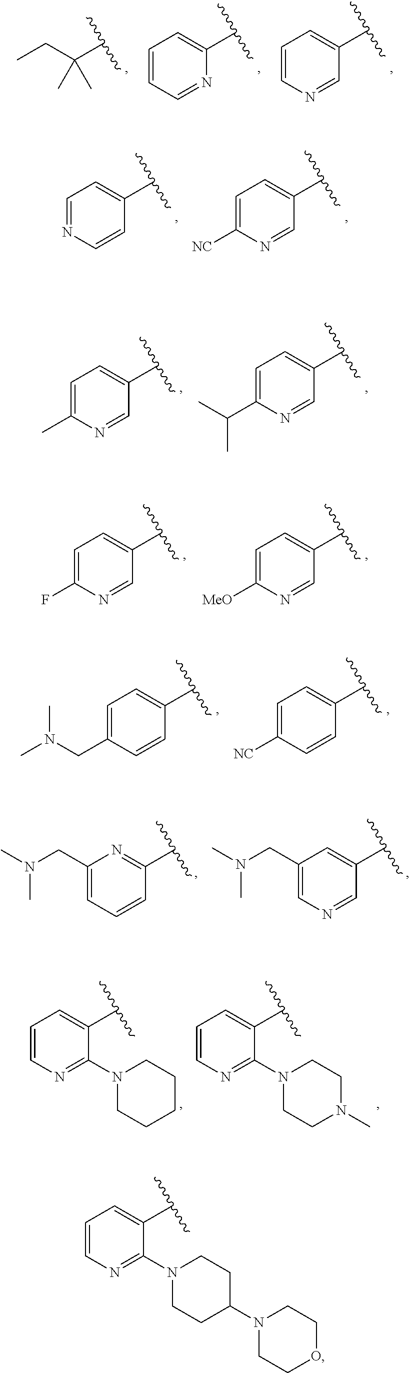

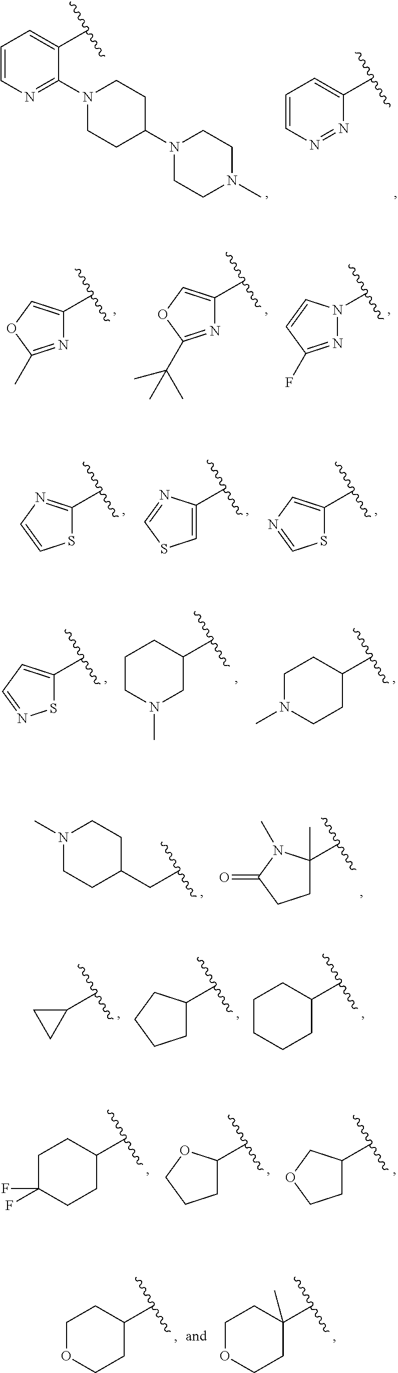

In other embodiments, R² is selected from the group consisting of phenyl, t-butyl,

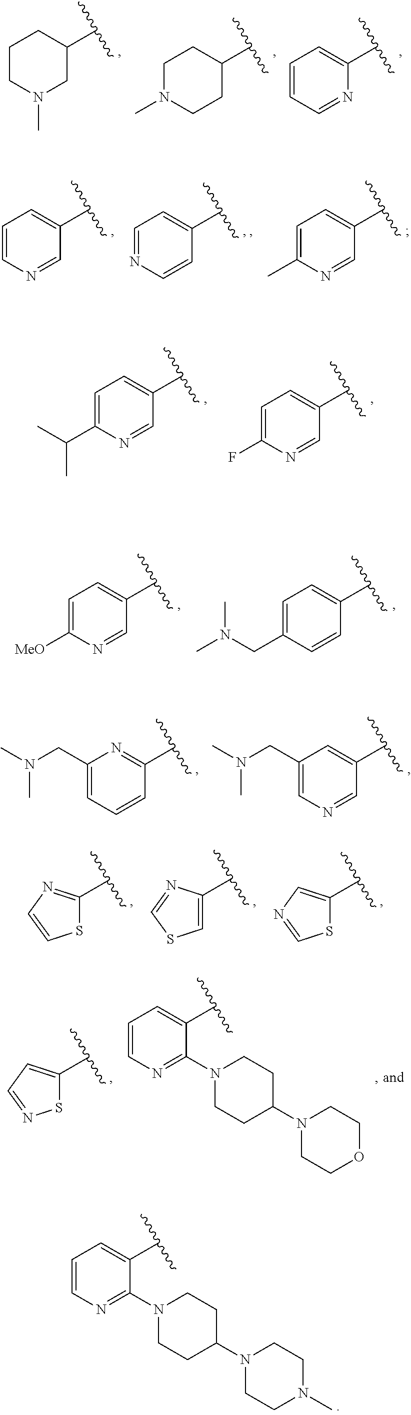

In some embodiments, R² is selected from the group consisting of phenyl, t-butyl,

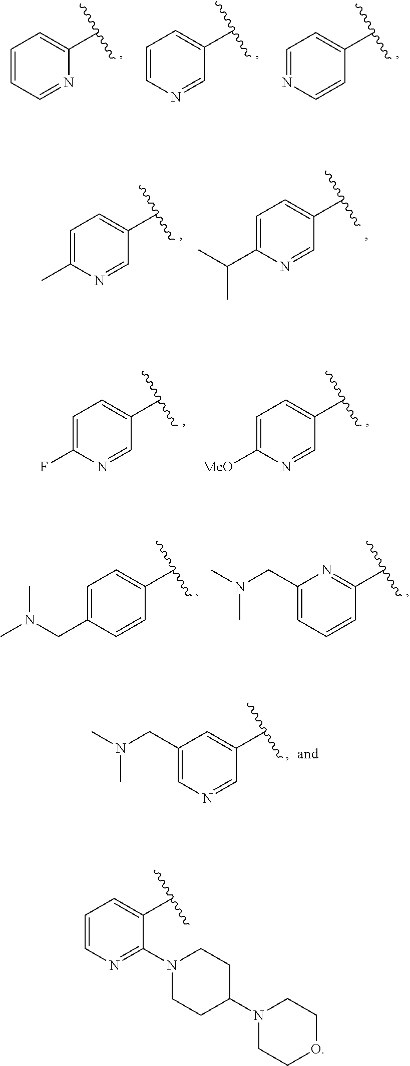

In some embodiments, R² is

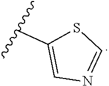 [SMILES: *c1cncs1]

In other embodiments, R² is

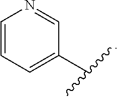 [SMILES: *c1cccnc1]

In some embodiments, R² is t-butyl.

In some embodiments, R² is 5-6 membered heteroaryl.

In some embodiments, R⁴ and R⁵ are independently, for each occurrence, hydrogen or methyl, or R⁴ and R⁵ can be taken together to form a cyclopropylene.

In some embodiments, R⁴ and R⁵ are independently, for each occurrence, hydrogen or methyl.

In some embodiments, R⁴ and R⁵ are hydrogen.

In some embodiments, n is 0, 1, 2, 3, 4, or 5.

In some embodiments, n is 0. In some embodiments, n is 1. In some embodiments, n is 2. In some embodiments, n is 3. In some embodiments, n is 4. In some embodiments, n is 5. In some embodiments, n is 6.

In other embodiments, the compound is a compound of formula (I-c):

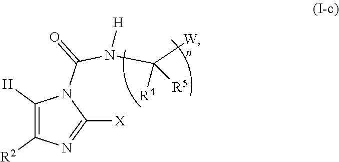

or a pharmaceutically acceptable salt thereof, wherein:

R² is selected from the group consisting of C₁₋₆alkyl, phenyl, and 3-7 membered heterocyclyl;

R⁴ and R⁵ are hydrogen;

n is an integer selected from 1 to 3;

X is hydrogen or —O—C₁₋₆alkylene-(3-7 membered heterocyclyl); and

W is selected from the group consisting of phenylene-phenyl, C₃₋₇cycloalkyl, and —(C₂₋₆alkynylene)-phenyl; and

wherein any aforementioned C₃₋₇cycloalkyl or 3-7 membered heterocyclyl is optionally substituted with one or more substituents each independently selected from C₁₋₆alkyl and C₁₋₆haloalkyl.

In some embodiments, R² is methyl. In other embodiments, R² is phenyl. In certain embodiments, R² is

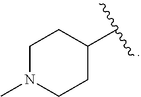 [SMILES: *C1CCN(C)CC1]

In some embodiments, n is 1. In other embodiments, n is 3.

In other embodiments, X is hydrogen. In some embodiments, X is

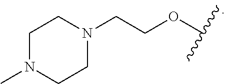 [SMILES: *OCCN1CCN(C)CC1]

In certain embodiments, W is

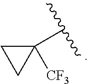 [SMILES: *C1(C(F)(F)F)CC1]

In other embodiments, W is

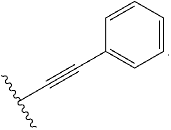 [SMILES: *C#Cc1ccccc1]

In some embodiments, W is

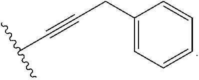 [SMILES: *C#CCc1ccccc1]

In certain embodiments, W is

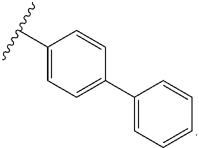 [SMILES: *c1ccc(-c2ccccc2)cc1]

In certain embodiments, the compound is a compound described in the Examples, or a pharmaceutically acceptable salt thereof.

In certain other embodiments, the compound is one of the compounds listed in Table 1 or a pharmaceutically acceptable salt thereof.

### Methods of Preparing Compounds

Methods for preparing compounds described herein are illustrated in the following synthetic schemes. These schemes are given for the purpose of illustrating the invention, and should not be regarded in any manner as limiting the scope or the spirit of the invention. Starting materials shown in the schemes can be obtained from commercial sources or can be prepared based on procedures described in the literature.

The synthetic route illustrated in Scheme 1 depicts an exemplary procedure for preparing substituted imidazole carboxamides. In the first step, compound A (wherein the variables are as described herein) is treated with triphosgene with triethylamine in DCM and the resulting intermediate is treated with amine B (wherein the variables are as described herein) to afford carboxamide C (a compound of formula (I)).

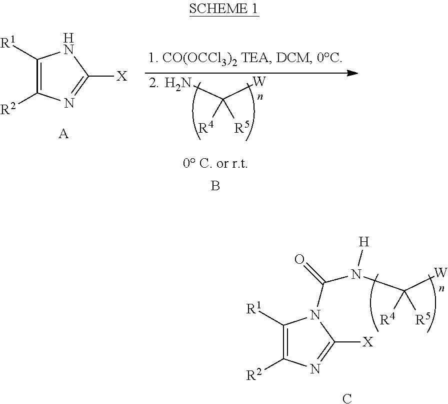

The synthetic route illustrated in Scheme 2 depicts another exemplary procedure for preparing substituted imidazole carboxamides. In the first step, compound A (wherein the variables are as described herein) is treated with 4-nitrophenyl chloroformate with triethylamine in DCM and the resulting intermediate is treated with amine B (wherein the variables are as described herein) to afford carboxamide C (a compound of formula (I)).

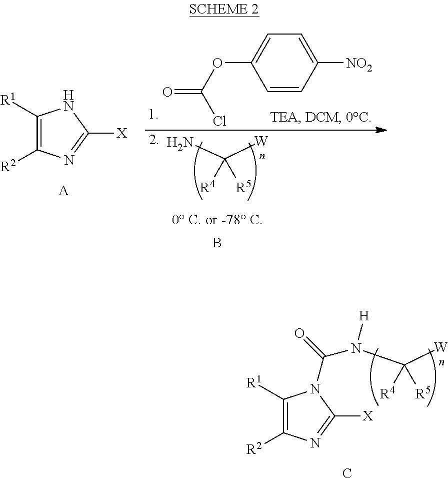

The synthetic route illustrated in Scheme 3 depicts another exemplary procedure for preparing substituted imidazole carboxamides. Compound A (wherein the variables are as described herein) is treated with isocyanate B (wherein the variables are as described herein) in the presence of triethylamine in DCM followed by reflux to afford carboxamide C (a compound of formula (I)).

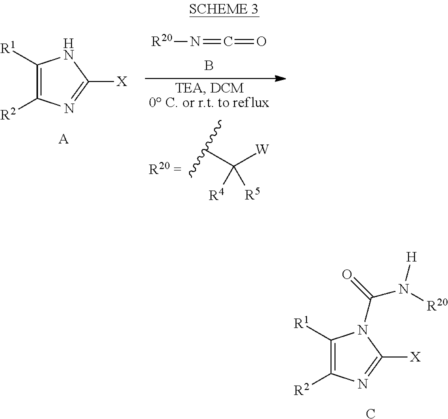

The reaction procedures in Schemes 1 to 3 are contemplated to be amenable to preparing a wide variety of substituted imidazole carboxamide compounds having different substituents. Furthermore, if a functional group that is part of the substituents would not be amenable to a reaction condition described in Schemes 1-3, it is contemplated that the functional group can first be protected using standard protecting group chemistry and strategies, and then the protecting group is removed after completing the desired synthetic transformation. See, for example, Greene, T. W.; Wuts, P. G. M. Protective Groups in Organic Synthesis, 2^{nd} ed.; Wiley: New York, 1991, for further description of protecting chemistry and strategies.

### III. Pharmaceutical Compositions

The invention provides pharmaceutical compositions comprising a compound described herein (e.g., a compound of Formula (I), e.g., a compound of Formula (I-a), (I-b), or (I-c)) or related organic compound described herein. In certain embodiments, the pharmaceutical compositions preferably comprise a therapeutically-effective amount of one or more of a compound described herein, e.g., a compound of Formula (I), e.g., a compound of Formula (I-a), (I-b), or (I-c), formulated together with one or more pharmaceutically acceptable carriers. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration.

The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention, e.g., a compound of Formula (I), e.g., a compound of Formula (I-a), (I-b), or (I-c).

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

### IV. Methods of Use

Sphingolipids are a family of membrane lipids derived from the aliphatic amino alcohol sphingosine and its related sphingoid bases. They are present in eukaryote membranes, where they exert important structural roles in the regulation of fluidity and subdomain structure of the lipid bilayer. In addition to serving roles in cell membrane structure and dynamics, sphingolipids also serve important signaling functions, for example, in the control of cell growth, cell differentiation, and cell death, and can be important for cell homeostasis and development. Zeidan et al. (2010) supra, Proksch et al. (2011) supra. Ceramide, a key member of this lipid class, has attracted attention in view of its impact on the replication and differentiation of neoplastic cells. Furuya et al. (2011) supra. For example, lower levels of ceramide have been discovered in several types of human tumors relative to normal tissue, where the level of ceramide appears to correlate inversely with the degree of malignant progression. Realini et al. (2013) supra.

Acid ceramidase is a cysteine amidase that catalyzes the hydrolysis of ceramide into sphingosine and fatty acid and is believed to be involved in the regulation of ceramide levels in cells and modulates the ability of this lipid messenger to influence the survival, growth and death of certain tumor cells. Id. Furthermore, acid ceramidase enzymes are abnormally expressed in various types of human cancer (e.g., prostate, head and neck, and colon) and serum AC levels are elevated in patients with melanoma relative to control subjects. Id.

In addition, acid ceramidase enzymes have been implicated in a number of other disorders, including, inflammation (for example, rheumatoid arthritis and psoriasis), pain, inflammatory pain, and various pulmonary disorders. See, International Application Publication No. WO2015/173169. Furthermore, acid ceramidase enzymes have been identified as a target for the treatment of certain lysosomal storage disorders (for example, Gaucher's, Fabry's, Krabbe, Tay Sachs), and neurodegenerative disorders (for example, Alzheimer's, Parkinson's, Huntington's, and amyotrophic lateral sclerosis). See, International Application Publication Nos. WO2016/210116 and WO2016/210120.

It is contemplated that the compounds, compositions, and methods disclosed herein can be used to treat various disorders associated or correlated with elevated levels of acid ceramidase activity. The invention provides administering to a subject in need thereof an effective amount of a compound or composition disclosed herein, either alone or in a combination with another therapeutic agent to treat the disorder.

In certain embodiments, the compound or composition used in one or more of the methods described herein is one of the generic or specific compounds described in Section II, such as a compound of Formula (I), a compound embraced by one of the further embodiments describing definitions for certain variables of Formula (I), a compound of Formula (I-a), (I-b), or (I-c), or a compound embraced by one of the further embodiments describing definitions for certain variables of Formula (I-a), (I-b), or (I-c).

In certain embodiments, a method or composition described herein, is administered in combination with one or more additional therapies, e.g., surgery, radiation therapy, or administration of another therapeutic preparation. In certain embodiments, the additional therapy may include an additional therapeutic agent. The invention embraces combination therapy, which includes the administration of a compound described herein, e.g., a compound of Formula (I), e.g., a compound of Formula (I-a), (I-b), or (I-c), or composition described herein and a second treatment and/or agent as part of a specific treatment regimen intended to provide the beneficial effect from the co-action of the foregoing. The beneficial effect of the combination may include pharmacokinetic or pharmacodynamic co-action resulting from the foregoing combination of agents and/or treatments.

The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

I. Cancer, Inflammation and Other Disorders

The compositions and methods disclosed herein can be used to treat various disorders associated or otherwise correlated with elevated levels of acid ceramidase activity. Exemplary disorders include cancer, inflammation, pain and inflammatory pain, or a pulmonary disease.

In certain embodiments, the compositions and methods disclosed herein can be used to treat cancer or inhibit cancer growth in a subject in need thereof. The invention provides a method of treating a cancer in a subject. The method comprises administering to the subject an effective amount of a compound (e.g., a compound of Formula (I), e.g., a compound of Formula (I-a), (I-b), or (I-c)) or a pharmaceutical composition disclosed herein, either alone or in a combination with another therapeutic agent to treat the cancer in the subject.

Exemplary cancers include, but are not limited to, pre-malignant conditions, for example hyperplasia, metaplasia or dysplasia, cancer metastasis, benign tumors, angiogenesis, hyperproliferative disorders and benign dysproliferative disorders. The treatment may be prophylactic or therapeutic. The subject to be treated may be human or a non-human animal (e.g., a non-human primate or a non-human mammal).

In certain embodiments, a compound disclosed herein, e.g., a compound of Formula (I), e.g., a compound of Formula (I-a), (I-b), or (I-c), or a pharmaceutical composition containing such a compound, can be used to treat a disorder involving primary and/or metastatic neoplastic disease.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma)

In certain embodiments, the present invention provides a compound disclosed herein, e.g., a compound of Formula (I), e.g., a compound of Formula (I-a), (I-b), or (I-c), or a pharmaceutical composition disclosed herein for the use in the treatment and/or prevention of brain cancer, breast cancer, colon cancer, head and neck cancer, liver cancer, lung cancer (e.g., alveolar cancer), pancreatic cancer, prostate cancer, skin cancer (e.g., melanoma).

It is contemplated that the compounds disclosed can be used in combination with other treatments and/or therapeutic agents. The invention embraces combination therapy, which includes the administration of a compound described herein, e.g., a compound of Formula (I), e.g., a compound of Formula (I-a), (I-b), or (I-c), or related compound described herein and a second treatment and/or agent as part of a specific treatment regimen intended to provide the beneficial effect from the co-action of these therapeutic agents. The beneficial effect of the combination may include pharmacokinetic or pharmacodynamic co-action resulting from the combination of therapeutic agents.

In certain embodiments, a compound or pharmaceutical composition described herein, is administered in combination with one or more additional cancer therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodialator, a statin, an anti-inflammatory agent (e.g. methotrexate), or an NSAID. In certain embodiments, the additional therapy may include a combination of therapeutics of different classes.

In certain embodiments, a method or pharmaceutical composition described herein is administered in combination with a checkpoint inhibitor. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody-based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), duvalumab (AstraZeneca), MED14736, avelumab, and BMS 936559 (Bristol Myers Squibb Co.).

In certain embodiments, a compound or pharmaceutical composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 BL. Exemplary CTLA-4 antibodies include ipilimumab or tremelimumab.

Exemplary cytotoxic agents that can be administered in combination with a compound or pharmaceutical composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a compound or pharmaceutical composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

In certain embodiments, a compound disclosed herein, e.g., a compound of Formula (I), e.g., a compound of Formula (I-a), (I-b), or (I-c), or a pharmaceutical composition containing such a compound, can be used to treat an inflammatory condition, such as rheumatoid arthritis and ulcerative cholitis. The invention provides a method of treating an inflammatory condition. The method comprises administering to the subject an effective amount of a compound (e.g., a compound of Formula (I), e.g., a compound of Formula (I-a), (I-b), or (I-c)) or a pharmaceutical composition disclosed herein, either alone or in a combination with another therapeutic agent to treat the inflammatory condition in the subject.

As used herein, an inflammatory condition is a disease or condition characterized, in whole or in part, by inflammation or an inflammatory response in the patient. Typically, one or more of the symptoms of the inflammatory disease or condition is caused or exacerbated by an inappropriate, misregulated, or overactive inflammatory response. Inflammatory diseases or conditions may be chronic or acute. In certain embodiments, the inflammatory disease or condition is an autoimmune disorder.

Inflammatory conditions treatable using a compound or pharmaceutical composition disclosed herein may be characterized, for example, based on the primary tissue affected, the mechanism of action underlying the condition, or the portion of the immune system that is misregulated or overactive. Examples of inflammatory conditions, as well categories of diseases and conditions are provided herein. In certain embodiments, examples of inflammatory conditions that may be treated include inflammation of the lungs, joints, connective tissue, eyes, nose, bowel, kidney, liver, skin, central nervous system, vascular system, heart, or adipose tissue. In certain embodiments, inflammatory conditions which may be treated include inflammation due to the infiltration of leukocytes or other immune effector cells into affected tissue. In certain embodiments, inflammatory conditions which may be treated include inflammation mediated by IgE antibodies. Other relevant examples of inflammatory conditions which may be treated by the present disclosure include inflammation caused by infectious agents, including but not limited to viruses, bacteria, fungi, and parasites. In certain embodiments, the inflammatory condition that is treated is an allergic reaction. In certain embodiments, the inflammatory condition is an autoimmune disease.

Inflammatory lung conditions include asthma, adult respiratory distress syndrome, bronchitis, pulmonary inflammation, pulmonary fibrosis, and cystic fibrosis (which may additionally or alternatively involve the gastro-intestinal tract or other tissue(s)). Inflammatory joint conditions include rheumatoid arthritis, rheumatoid spondylitis, juvenile rheumatoid arthritis, osteoarthritis, gouty arthritis and other arthritic conditions. Inflammatory eye conditions include uveitis (including iritis), conjunctivitis, scleritis, and keratoconjunctivitis sicca. Inflammatory bowel conditions include Crohn's disease, ulcerative colitis, inflammatory bowel disease, and distal proctitis. Inflammatory skin conditions include conditions associated with cell proliferation, such as psoriasis, eczema, and dermatitis (e.g., eczematous dermatitides, topic and seborrheic dermatitis, allergic or irritant contact dermatitis, eczema craquelee, photoallergic dermatitis, phototoxicdermatitis, phytophotodermatitis, radiation dermatitis, and stasis dermatitis). Inflammatory conditions of the endocrine system include, but are not limited to, autoimmune thyroiditis (Hashimoto's disease), Type I diabetes, inflammation in liver and adipose tissue associated with Type II diabetes, and acute and chronic inflammation of the adrenal cortex. Inflammatory conditions of the cardiovascular system include, but are not limited to, coronary infarct damage, peripheral vascular disease, myocarditis, vasculitis, revascularization of stenosis, atherosclerosis, and vascular disease associated with Type II diabetes. Inflammatory conditions of the kidney include, but are not limited to, glomerulonephritis, interstitial nephritis, lupus nephritis, nephritis secondary to Wegener's disease, acute renal failure secondary to acute nephritis, Goodpasture's syndrome, post-obstructive syndrome and tubular ischemia. Inflammatory conditions of the liver include, but are not limited to, hepatitis (arising from viral infection, autoimmune responses, drug treatments, toxins, environmental agents, or as a secondary consequence of a primary disorder), obesity, biliary atresia, primary biliary cirrhosis and primary sclerosing cholangitis. In certain embodiments, the inflammatory condition is an autoimmune disease, for example, rheumatoid arthritis, lupus, alopecia, autoimmune pancreatitis, Celiac disease, Behcet's disease, Cushing syndrome, and Grave's disease. In certain embodiments, the inflammatory condition is a rheumatoid disorder, for example, rheumatoid arthritis, juvenile arthritis, bursitis, spondylitis, gout, scleroderma, Still's disease, and vasculitis.

In certain embodiments, the present invention provides a compound disclosed herein, e.g., a compound of Formula (I), e.g., a compound of Formula (I-a), (I-b), or (I-c), or a pharmaceutical composition containing a compound disclosed herein for use in the treatment of a pain syndrome, disorder, disease or condition characterized by nociceptive pain, neuropathic pain, inflammatory pain, non-inflammatory pain, pain associated with acute conditions such as post-operative or post-traumatic stress disorders, pain associated with chronic conditions such as diabetes. The invention provides a method of treating pain. The method comprises administering to the subject an effective amount of a compound (e.g., a compound of Formula (I), e.g., a compound of Formula (I-a), (I-b), or (I-c)) or a pharmaceutical composition disclosed herein, either alone or in a combination with another therapeutic agent to treat the pain in the subject.

A compound or composition described herein can be useful for the treatment (including prevention and/or alleviation) of chronic and/or acute pain, in particular non-inflammatory musculoskeletal pain such as back pain, fibromyalgia and myofascial pain, more particularly for reduction of the associated muscular hyperalgesia or muscular allodynia. Non-limiting examples of types of pain that can be treated by a compound or composition disclosed includes chronic conditions such as musculoskeletal pain, including fibromyalgia, myofascial pain, back pain, pain during menstruation, pain during osteoarthritis, pain during rheumatoid arthritis, pain during gastrointestinal inflammation, pain during inflammation of the heart muscle, pain during multiple sclerosis, pain during neuritis, pain during AIDS, pain during chemotherapy, tumor pain, headache, CPS (chronic pain syndrome), central pain, neuropathic pain such as trigeminal neuralgia, shingles, stamp pain, phantom limb pain, temporomandibular joint disorder, nerve injury, migraine, post-herpetic neuralgia, neuropathic pain encountered as a consequence of injuries, amputation infections, metabolic disorders or degenerative diseases of the nervous system, neuropathic pain associated with diabetes, pseudesthesia, hypothyroidism, uremia, vitamin deficiency or alcoholism; and acute pain such as pain after injuries, postoperative pain, pain during acute gout or pain during operations, such as jaw surgery.

In certain embodiments, the present invention provides a compound disclosed herein, e.g., a compound of Formula (I), e.g., a compound of Formula (I-a), (I-b), or (I-c), or a pharmaceutical composition disclosed herein for use in the treatment of a pulmonary disease, such as asthma, chronic obstructive pulmonary disease (COPD), adult respiratory disease, acute respiratory distress syndrome, chronic bronchitis, and emphysema. The invention provides a method of treating a pulmonary disease. The method comprises administering to the subject an effective amount of a compound (e.g., a compound of Formula (I), e.g., a compound of Formula (I-a), (I-b), or (I-c)) or a pharmaceutical composition disclosed herein, either alone or in a combination with another therapeutic agent to treat the pulmonary disease in the subject.

II. Lysosomal Storage Disorders

Lysosomal storage disorders (LSDs) are a group of more than 50 clinically-recognized, rare inherited metabolic disorders that result from defects in lysosomal function (Walkley, J. (2009) INHERIT. METAB. DIS., 32(2): 181-9). LSDs are caused by dysfunction of the cell's lysosomes, which are heterogeneous subcellular organelles containing specific hydrolases that allow targeted processing or degradation of proteins, nucleic acids, carbohydrates, and lipids (HARRISON'S PRINCIPLES OF INTERNAL MEDICINE, 16^{th} Edition, vol. II, Chapter 20, pp. 2315-2319). The lysosome encloses an acidic environment and contains enzymes that catalyze the hydrolysis of biological macromolecules.

Individually, LSDs occur with incidences of less than 1:100,000, however, as a group the incidence is as high as 1 in 1,500 to 7,000 live births (Staretz-Chacham, et al. (2009) PEDIATRICS, 123(4): 1191-207). LSDs typically are caused by inborn genetic errors. Affected individuals generally appear normal at birth, however the diseases are progressive. The development of clinical disease may not occur until years or decades later, but is typically fatal.

It is believed that sphingosine-containing analogs (for example, glucosylsphingosine, galactosphingosine, lactosylsphingosine, GB3-sphingosine, and GM2-sphingosine) may accumulate in cells of subjects with certain lysosomal storage disorders or LSDs (for example, Gaucher's disease, Krabbe disease, multiple sclerosis, Fabry's disease, and Tay Sachs disease, respectively) and that the accumulation of these sphingosine-containing analogs may contribute to the disease phenotype. See, e.g., International Application Publication No. WO2016/210116. Given that such sphingosine-containing analogs are often produced by acid ceramidase enzymes in the lysosomal compartments of cells in subjects with LSDs, the accumulation of the sphingosine-containing analogs to detrimental levels can be prevented or reduced by the use of an effective amount of one or more of the acid ceramidase inhibitors described herein.

In certain embodiments, a compound (e.g., a compound of Formula (I), e.g., a compound of Formula (I-a), (I-b), or (I-c)) or pharmaceutical composition containing a compound disclosed herein can be used to treat a LSD in a subject in need thereof. The invention provides a method of treating a LSD in a subject. The method comprises administering to the subject an effective amount of a compound (e.g., a compound of Formula (I), e.g., a compound of Formula (I-a), (I-b), or (I-c)) or a pharmaceutical composition disclosed herein, either alone or in a combination with another therapeutic agent to treat the LSD in the subject.

Exemplary LSDs include, for example, Krabbe disease, Fabry disease, Tay-Sachs disease, Sandhoff Variant A, or B, Pompe disease, Hunter's syndrome, Niemann Pick disease Types A and B, and Gaucher's disease.

It is contemplated that the compounds disclosed can be used in combination with other treatments and/or therapeutic agents. The invention embraces combination therapy, which includes the administration of a compound described herein, e.g., a compound of Formula (I), e.g., a compound of Formula (I-a), (I-b), or (I-c), or related compound described herein and a second treatment and/or agent as part of a specific treatment regimen intended to provide the beneficial effect from the co-action of these therapeutic agents. Exemplary second agents for use in treating Gaucher disease include, for example, imiglucerase (CEREZYME®), taliglucerase alfa (ELELYSO®), velaglucerase alfa (VPRIV®), eliglustat (CERDELGA®), and miglustat (ZAVESCA®) or a glucocerebrosidase activator such as one or more of the compounds described in International Application Publication No. WO2012/078855. Exemplary second agents for use in treating Fabry disease include, for example, alpha-galactosidase A (FABRAZYME®). Additional acid ceramidase inhibitors for use in combination therapies include, for example, those described in International Patent Application Publications WO 2015/173168 and WO 2015/173169, each of which are hereby incorporated by reference.

III. Neurodegenerative Disorders

Neurodegenerative disorders often are associated with reduction in the mass and/or volume of the brain, which may be due to the atrophy and/or death of brain cells, which are far more profound than those in a healthy subject that are attributable to aging. Neurodegenerative disorders can evolve gradually, after a long period of normal brain function, due to progressive degeneration (e.g., nerve cell dysfunction and death) of specific brain regions. Alternatively, neurodegenerative disorders can have a quick onset, such as those associated with trauma or toxins. The actual onset of brain degeneration may precede clinical expression by many years.

Examples of neurodegenerative disorders include, for example, Alzheimer's disease, Parkinson's disease, Huntington's disease, amyotrophic lateral sclerosis (ALS; also known as Lou Gehrig's disease or motor neuron disease), multiple sclerosis, and diffuse Lewy body disease. Once clinical expression occurs, the neurodegenerative disorder may be associated with impairment of motor function, for example, as observed in subjects with Parkinson's disease, Huntington's disease multiple sclerosis, or ALS. Alternatively or in addition, neurodegenerative disorders may be associated with cognitive impairment and/or the loss of cognitive function, for example, as observed in subjects with Alzheimer's disease.

Alzheimer's disease is a central nervous system (CNS) disorder that results in memory loss, unusual behavior, personality changes, and a decline in thinking abilities. These losses are related to the death of specific types of brain cells and the breakdown of connections and their supporting network (e.g., glial cells) between them. The earliest symptoms include loss of recent memory, faulty judgment, and changes in personality. Parkinson's disease is a CNS disorder that results in uncontrolled body movements, rigidity, tremor, and dyskinesia, and is associated with the death of brain cells in an area of the brain that produces dopamine. ALS (motor neuron disease) is a CNS disorder that attacks the motor neurons, components of the CNS that connect the brain to the skeletal muscles. Huntington's disease is another neurodegenerative disease that causes uncontrolled movements, loss of intellectual faculties, and emotional disturbance.

It has been observed that subjects with certain mutant alleles in genes encoding β-glucocerebrosidase activity (the GBA gene; Aharon-Peretz (2004) NEW. ENG. J. MED. 351: 1972-1977; Gan-Or et al. (2008) NEUROLOGY 70:2277-2283; Gan-Or et al. (2015) NEUROLOGY 3:880-887) and sphinomyelinase activity (the SMPD1 gene, Gan-Or et al. (2013) NEUROLOGY 80:1606-1610) have been associated with, and identified as a risk factor for, Parkinson's Disease. As a result, defects with, or deficiencies in the activities of these enzymes, as in the case of Gaucher's disease and Niemann Pick types A and B, can cause an accumulation of glucosylceramide and sphingomyelin, which can then be converted to glucosylsphingosine or lyso-sphingomyelin, respectively, via acid ceramidase activity. The accumulation of glucosylsphingosine or lyso-sphingomyelin may thus be implicated in the development of Parkinson's disease. It is contemplated that the administration of an acid ceramidase inhibitor, which slows down, stops or reverses the accumulation of glucosylsphingosine and/or lyso-sphingomyelin can be used to treat Parkinson's Disease. For example, an acid ceramidase inhibitor can be used to improve motor and/or memory impairments symptomatic of Parkinson's disease.

Similarly, it has been observed that lactosylceramide (LacCer) is upregulated in the central nervous system of mice during chronic experimental autoimmune encephalomyelitis (EAE), a model of multiple sclerosis (Lior et al. (2014) NATURE MEDICINE 20:1147-1156.). It is contemplated that the increase in LacCer may also result in an increase in lactosylsphingosine (LacSph) via conversion by an acid ceramidase (a lactosylceramide to lactosylsphingosine converting enzyme). Given the accumulation of lactosylsphingosine to a toxic or otherwise detrimental level or concentration in the lysosomal compartments of cells in subjects with multiple sclerosis, it is contemplated that the administration of an acid ceramidase inhibitor can reduce the accumulation of lactosylsphingosine thereby treating multiple sclerosis, which includes ameliorating a symptom associated with multiple sclerosis.

It has been observed that the level and activity of acid ceramidase can be elevated in subjects with Alzheimer's disease (Huang et al. (2004) EUROPEAN J. NEUROSCI. 20:3489-3497). Given that the accumulation of sphingosine or sphingosine analogs to a toxic or otherwise detrimental level or concentration in the lysosomal compartments of cells in subjects with Alzheimer's disease, it is contemplated that the administration of an acid ceramidase inhibitor can reduce the accumulation of the sphingosine or sphingosine analogs thereby treating Alzheimer's disease, which includes ameliorating a symptom associated with Alzheimer's disease.

Furthermore, given that a number of the foregoing neurodegenerative disorders, for example, Alzheimer's disease, are associated with a level of cognitive impairment and/or some decrease or loss of cognitive function, it is contemplated that the administration of an effective of an acid ceramidase inhibitor to a subject in need thereof may be reduce, stabilize, or reverse cognitive impairment and/or the loss of cognitive function. Cognitive function generally refers to the mental processes by which one becomes aware of, perceives, or comprehends ideas. Cognitive function involves all aspects of perception, thinking, learning, reasoning, memory, awareness, and capacity for judgment. Cognitive impairment generally refers to conditions or symptoms involving problems with thought processes. This may manifest itself in one or more symptoms indicating a decrease in cognitive function, such as impairment or decrease of higher reasoning skills, forgetfulness, impairments to memory, learning disabilities, concentration difficulties, decreased intelligence, and other reductions in mental functions.

Cognitive function and cognitive impairment may be readily evaluated using tests well known in the art. Performance in these tests can be compared over time to determine whether a treated subject is improving or whether further decline has stopped or slowed, relative to the previous rate of decline of that patient or compared to an average rate of decline. Tests of cognitive function, including memory and learning for evaluating human patients are well known in the art and regularly used to evaluate and monitor subjects having or suspected of having cognitive disorders such as Alzheimer's disease including the clock-drawing test (Agrell & Dehlin (1998) AGE & AGING 27:399-403). Even in healthy individuals, these and other standard tests of cognitive function can be readily used to evaluate beneficial affects over time.

In certain embodiments, a compound (e.g., a compound of Formula (I), e.g., a compound of Formula (I-a), (I-b), or (I-c)) or a pharmaceutical composition containing a compound disclosed herein can be used to treat a neurodegenerative disorder in a subject in need thereof. The invention provides a method of treating a neurodegenerative disorder in a subject. The method comprises administering to the subject an effective amount of a compound (e.g., a compound of Formula (I), e.g., a compound of Formula (I-a), (I-b), or (I-c)) or a pharmaceutical composition disclosed herein, either alone or in a combination with another therapeutic agent to treat the neurodegenerative disorder in the subject.

Exemplary neurodegenerative disorders include, for example, Alzheimer's disease, Parkinson's disease, Huntington's disease, amyotrophic lateral sclerosis, Lewy body disease, dementia (e.g., frontotemporal dementia), multisystem atrophy, multiple sclerosis, epilepsy, bipolar disorder, schizophrenia, anxiety disorders (e.g., a panic disorder, social anxiety disorder or generalized anxiety disorder) or progressive supranuclear palsy.

It is contemplated that the compounds disclosed can be used in combination with other treatments and/or therapeutic agents. The invention embraces combination therapy, which includes the administration of a compound described herein, e.g., a compound of Formula (I), e.g., a compound of Formula (I-a), (I-b), or (I-c), or related compound described herein and a second treatment and/or agent as part of a specific treatment regimen intended to provide the beneficial effect from the co-action of these therapeutic agents.

During the treatment of Parkinson's disease, the acid ceramidase inhibitor can be administered in combination with carbidopa and/or levadopa, a dopamine agonist, a monoamine oxidase B inhibitor, a catchetol O-methyltransferase inhibitor, an anticholingeric, or amantadine. During the treatment of Alzheimer's disease, the acid ceramidase inhibitor can be administered in combination with a cholinesterase inhibitor and/or memantine. During the treatment of Huntington's disease, the acid ceramidase inhibitor can be administered in combination with tetrabenazine; an antipsychotic drug such as haloperidol, chlorpromazine, quetiapine, risperidone, and olanzapine; a chorea-suppressing medication such as amantadine, levetiracetam, and clonazempam; an antidepressant such as citalopram, fluoxetine, and sertraline; and a mood-stabilizing drug such as valproate, carbamazepine, and lamotrigine.

During the treatment of amyotrophic lateral sclerosis, the acid ceramidase inhibitor can be administered in combination with riluzole; an agent for ameliorating muscle cramps and spasms such as cyclobenzaprine HCl, metaxalone, and robaxin; an agent for ameliorating spasticity such as tizanidine HCl, baclofen, and dantrolene; an agent for ameliorating fatigue such as caffeine, caffeine citrate, or caffeine benzoate injection; an agent for ameliorating excessive salivation such as glycopyrrolate, propantheline, amitriptyline, nortriplyline HCl and scopolamine; an agent for ameliorating excessive phlegm such as guaifenesin, albuterol inhalation, and acetylcysteine; an agent for ameliorating pain such as an opioid; an anticonvulsant or antiepileptic; a serotonin reuptake inhibitor; an antidepressant; an agent for ameliorating sleep disorders such as a benzodiazepine, a non-benzodiazepine hypnotic, a melatonin receptor stimulator, an anti-narcoleptic, and an orexin receptor antagonist; and an agent pseudobulbar affect such as dextromethorphan/quinidine.

During the treatment of multiple sclerosis, the acid ceramidase inhibitor can be administered in combination with a corticosteroid, β interferon, glatiramer acetate, dimethyl fumarate, fingolimod, teriflunomide, natalizumab, mitoxantrone, baclofen, and tizanidine. During the treatment of diffuse Lewy body disease, the acid ceramidase inhibitor can be administered in combination with a cholinesterase inhibitor, a Parkinson's disease medication such as carbidopa and/or levodopa, and an anti-psychotic medication such as quetiapine and olanzapine.

During the treatment of multisystem atrophy, the acid ceramidase inhibitor can be administered in combination with a medication to raise blood pressure such as fludrocortisone, psyridostigmine, midodrine, and droxidopa; and a Parkinson's disease medication such as carbidopa and/or levodopa. During the treatment of frontotemporal dementia, the acid ceramidase inhibitor can be administered in combination with an antidepressant, a selective serotonin reuptake inhibitor, and an antipsychotic. During the treatment of progressive upranuclear palsy, the acid ceramidase inhibitor can be administered in combination with a Parkinson's disease medication such as carbidopa and/or levodopa. It is understood that other combinations would be known be those skilled in the art.

### V. Kits for Use in Medical Applications

Another aspect of the invention provides a kit for treating a disorder. The kit comprises: i) instructions for treating a medical disorder, such as, cancer (such as melanoma), a lysosomal storage disorder (such as Krabbe disease, Fabry disease, Tay-Sachs disease, Pompe disease, Hunter's syndrome, Niemann Pick disease Types A and B, Gaucher disease), a neurodegenerative disease (such as Alzheimer's disease, Parkinson's disease, Huntington's disease, and amyotrophic lateral sclerosis), an inflammatory disorder, and pain; and ii) a compound described herein or related organic compound described herein, such as a compound of Formula (I), e.g., a compound of Formula (I-a), (I-b), or (I-c), or a composition described herein. The kit may comprise one or more unit dosage forms containing an amount of a compound described herein or related organic compound described herein, such as a compound of Formula (I), e.g., a compound of Formula (I-a), (I-b), or (I-c), that is effective for treating said medical disorder, cancer (such as melanoma), lysosomal storage diseases (such as Krabbe disease, Fabry disease, Tay-Sachs disease, Pompe disease, Hunter's syndrome, Niemann Pick disease Types A and B, Gaucher disease), and neurodegenerative diseases (such as Alzheimer's disease, Parkinson's disease, Huntington's disease, and amyotrophic lateral sclerosis), an inflammatory disorder, and pain.

The description above describes multiple aspects and embodiments of the invention, including substituted benzimidazole carboxamides and related organic compounds, compositions comprising a substituted benzimidazole carboxamides or related organic compounds, methods of using the substituted benzimidazole carboxamides or related organic compounds, and kits. The patent application specifically contemplates all combinations and permutations of the aspects and embodiments. For example, the invention contemplates treating a medical disorder such as Gaucher disease, Parkinson's disease, Lewy body disease, dementia, or multiple system atrophy in a human patient by administering a therapeutically effective amount of a compound described herein, e.g., a compound of Formula (I), e.g., a compound of Formula (I-a), (I-b), or (I-c), or a composition comprising such a compound. Further, for example, the invention contemplates a kit for treating a medical disorder, for example, cancer (such as melanoma), lysosomal storage disorder (such as Krabbe disease, Fabry disease, Tay-Sachs disease, Pompe disease, Hunter's syndrome, Niemann Pick disease Types A and B, Gaucher disease), and neurodegenerative disease (such as Alzheimer's disease, Parkinson's disease, Huntington's disease, and amyotrophic lateral sclerosis), inflammatory disorder, and pain and ii) a compound described herein or related organic compound described herein, such as a compound of Formula (I), e.g., a compound of Formula (I-a), (I-b), or (I-c), or a composition comprising such a compound.

In another aspect, the invention provides a compound (e.g., a compound of Formula (I), e.g., a compound of Formula (I-a), (I-b), or (I-c)) or a pharmaceutical composition as disclosed herein for use in a method of treating a subject with cancer and in need thereof, the method comprising administering to the subject a therapeutically effective amount of the compound or the pharmaceutical composition.

In another aspect, the invention provides a compound (e.g., a compound of Formula (I), e.g., a compound of Formula (I-a), (I-b), or (I-c)) or a pharmaceutical composition as disclosed herein for use in a method of treating a subject with a lysosomal storage disorder and in need thereof, the method comprising administering to the subject a therapeutically effective amount of the compound or the pharmaceutical composition.

In another aspect, the invention provides a compound (e.g., a compound of Formula (I), e.g., a compound of Formula (I-a), (I-b), or (I-c)) or a pharmaceutical composition as disclosed herein for use in a method of treating a subject with a neurodegenerative disorder and in need thereof, the method comprising administering to the subject a therapeutically effective amount of the compound or the pharmaceutical composition.

In another aspect, the invention provides a compound (e.g., a compound of Formula (I), e.g., a compound of Formula (I-a), (I-b), or (I-c)) or a pharmaceutical composition as disclosed herein for use in a method of treating a subject with an inflammatory disorder and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), or (I-c)) or a pharmaceutical composition as disclosed herein for the manufacture of a medicament for treating a subject with cancer and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), or (I-c)) or a pharmaceutical composition as disclosed herein for the manufacture of a medicament for treating a subject with a lysosomal storage disorder and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), or (I-c)) or a pharmaceutical composition as disclosed herein for the manufacture of a medicament for treating a subject with a neurodegenerative disorder and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), or (I-c)) or a pharmaceutical composition as disclosed herein for the manufacture of a medicament for treating a subject with an inflammatory disorder and in need thereof, the method comprising administering to the subject a therapeutically effective amount of the compound or the pharmaceutical composition.

### ExampleS

The invention now being generally described, will be more readily understood by reference to the following examples, which are included merely for purposes of illustration of certain aspects and embodiments of the present invention, and are not intended to limit the invention. In certain instances, the amount of compound produced by the procedure is stated along with the yield, which may be presented in the format of the procedure produced the title compound (10 mg; 90%) which means that 10 mg of the title compound was obtained and that corresponds to a yield of 90%.

### Example 1—Preparation of Imidazole Carboxamide Compounds and Substituted Imidazole Compounds

Imidazole carboxamide compounds were prepared based on general procedures described in Part I below. Specific imidazole carboxamide compounds prepared according to the general procedures are provided in Part II below.

### Part I—General Procedures

### General Procedure A for the Preparation of Substituted Imidazoles

A bromo alkyl/heteroaryl alkanone formamide (20.0 eq) was stirred at 180° C. for 8-hrs. The reaction mixture was cooled to room temperature (RT), diluted with saturated NaHCO₃ (20 mL) and extracted with DCM (50 mL×2). The resulting organic phases were washed with water (20 mL×1), dried over anhydrous Na₂SO₄, filtered and concentrated to give the substituted imidazole. In some cases the crude was purified by silica gel chromatography (DCM:MeOH; 10:1 to 4:1) to afford the substituted imidazole.

### General Procedure B for the Preparation of Imidazole Carboxamides

Method 1: To a solution of a substituted imidazole (1.0 eq) and Et₃N (2.0-5.0 eq) in DCM or CH₃CN (5-20 mL/mmol) was added isocyanate (1.2-4.0 eq) (e.g., (2-isocyanatoethyl)benzene) at 0° C. or at RT. The resulting mixture was stirred at RT or at reflux for 2 h to overnight. The reaction mixture was poured into water and extracted with DCM. The combined organic layers were washed with brine, dried over Na₂SO₄, filtered and concentrated to give a residue which was purified by silica gel column chromatography or Prep-HPLC to give an imidazole carboxamide which was further triturated with organic solvents if needed to increase the purity.

Method 2: To a solution of a substituted imidazole (1.0 eq) and triphosgene (0.5-1.0 eq) in DCM (8-20 mL/mmol) at 0° C. or −78° C. was added Et₃N (3.0 eq). The reaction mixture was stirred at 0° C. for 10 min-2 h. The amine (1.2-3.0 eq) was added at 0° C. or −78° C. and the reaction mixture was stirred at 0° C. or RT for 1 h-4 h. The solution was diluted with DCM, washed with H₂O, brine, dried over Na₂SO₄ and purified by silica gel column chromatography or Prep-HPLC to give an imidazole carboxamide.

### General Procedure C for the Preparation of Imidazole Carboxamides

To a solution of a substituted imidazole (1.0 eq) and Et₃N (2.0-5.0 eq) in DCM (5-20 mL/mmol) was added a solution of 4-nitrophenyl chloroformate (1.2-2.0 eq) in DCM (2-10 mL/mmol) at 0° C. The mixture was then stirred at 0° C. for 10-30 min and then the amine (1.5-5.0 eq) was added. The resulting mixture was stirred at 0° C. for 30-60 min. The reaction mixture was poured into water and extracted with DCM. The combined organic layers were washed with brine, dried over Na₂SO₄, filtered and concentrated to give a residue which was purified by silica gel column chromatography to give an imidazole carboxamide which was further triturated with organic solvents if needed to increase the purity.

### General Procedure D to Afford Ring Coupled Imidazoles Using Suzuki Cross-Coupling

A suspension of a bromo imidazole compound (1 equivalent), organoboronic acid or organoboronic ester (1.1 equivalents), Na₂CO₃ (3.0 equivalents) and Pd(dppf)Cl₂.DCM (5 mol %) or Pd(PPh₃)₄ (5 mol %) in 1,4-dioxane/H₂O (40 mL/mmol; 5/1) was stirred at 90-120° C. for 12-16 h under N₂. The reaction mixture was quenched with water (30 mL/mmol) and the resulting mixture extracted with EA (30 mL/mmol×3). The organic phases were washed with water (30 mL/mmol) and brine (30 mL/mmol), dried over anhydrous Na₂SO₄ and filtered. The filtrate was concentrated in vacuo, and the resulting residue was purified by silica gel column chromatography to afford the coupled ring system.

### General Procedure E for the O-Alkylation of a 2-Bromo Substituted Imidazole

To a solution of 2-bromo-substituted imidazole (1.0 eq) in a substituted alcohol (0.75 M) was added t-BuONa (5.0 eq). The mixture was stirred at 120° C. for 2 h or 150° C. for 2 h under microwave conditions and then poured into H₂O (20 mL). The mixture was extracted with EA (20 mL×2). The combined organic layers were concentrated, and the residue was purified by column (DCM:MeOH; 10:1 to 5:1) or (PE:EA; 10:1 to 5:1) to give the O-substituted imidazole.

### General Procedure F for the Deprotection of a SEM Protecting Group

Method 1: To a solution of a substituted SEM protected imidazole (1.0 eq) in DCM (2 M) was added TFA (2.5 M). The mixture was stirred at RT for 2 h and then concentrated. Then H₂O was added, and the pH was adjusted to 8 with saturated aq. NaHCO₃ solution. After extraction with EA the organic layers were dried over Na₂SO₄, filtered and concentrated and used directly in the subsequent reaction. In other examples the residue was purified by silica gel chromatography (DCM:MeOH=10:1 to 5:1) to give the deprotected substituted imidazole.

Method 2: A solution of a substituted SEM protected imidazole (1.0 eq) in 37% HCl/dioxane (4.0 mol/L) was stirred at 60° C. for 18 h. The reaction mixture was cooled, concentrated and H₂O was added, and the pH was adjusted to 8 with saturated aq. NaHCO₃ solution. After extraction with EA the organic layers were dried over Na₂SO₄, filtered and concentrated to give the deprotected substituted imidazole.

### Part II—Preparation of Specific Imidazole Compounds and Intermediates

Exemplary procedures for preparing specific imidazole carboxamides and intermediates are provided below. The following examples describe the multistep synthesis of imidazole carboxamides and intermediates. The various steps, including the synthesis of intermediates, are discussed in more detail below.

### 2-(1-(Trifluoromethyl)cyclopropyl)ethan-1-amine

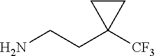

A solution of 1-(trifluoromethyl)cyclopropanecarboxylic acid (10 g, 64.9 mmol) in BH₃-THF (1M, 100 mL) was stirred at 40° C. overnight under N₂. The reaction was quenched with saturated NH₄Cl solution. The solid was filtered and the liquid was extracted with EA (200 mL×3). The combined organic layers were dried over Na₂SO₄ and concentrated under vacuum to give (1-(trifluoromethyl)cyclopropyl)methanol as a colorless oil (7.0 g, 77.0%). ¹H NMR (400 MHz, DMSO-d₆): δ 4.94 (t, J=6.0 Hz, 1H), 3.54 (d, J=6.0 Hz, 2H), 0.87-0.84 (m, 2H), 0.81-0.79 (m, 2H).

To a solution of (1-(trifluoromethyl)cyclopropyl)methanol (2 g, 14.28 mmol) in DCM (30 mL) were added TsCl (3.27 g, 17.14 mmol) and Et₃N (4.34 g, 42.84 mmol) and the resulting reaction mixture was stirred at RT overnight under N₂. The mixture was filtered and concentrated to give a residue which was purified by silica gel column chromatography (PE:EA=5:1) to give (1-(trifluoromethyl)cyclopropyl)methyl4-methylbenzenesulfonate (2 g, 47.6%) as a colorless oil. LC-MS m/z: 295.1 [M+H]⁺. Purity (214 nm): 90%; t_{R}=1.13 min.

To a solution of (1-(trifluoromethyl)cyclopropyl)methyl4-methylbenzenesulfonate (2 g, 6.8 mmol) in DMF (20 mL) were added 18-crown-6 (2.7 g, 10.2 mmol) and KCN (664 mg, 10.2 mmol) and the resulting reaction mixture was stirred at 55° C. for 48 h under N₂. The reaction mixture was diluted with EA (100 mL×3), washed with brine (50 mL×3), dried over Na₂SO₄, and concentrated under vacuum to give 2-(1-(trifluoromethyl)cyclopropyl)acetonitrile (800 mg, 78.9%) as a light-yellow oil. ¹H NMR (400 MHz, CDCl₃): δ 2.81 (s, 2H), 1.24-1.18 (m, 2H), 0.95-0.92 (m, 2H).

A solution of 2-(1-(trifluoromethyl)cyclopropyl)acetonitrile (800 mg, 5.4 mmol) in BH₃-THF (1M, 10 mL) under N₂, was heated to 70° C. and stirred overnight. The reaction mixture was cooled to 0° C. and methanolic HCl (2 mL) was added dropwise. The mixture was concentrated, and EA was added. The resulting solid was collected via filtration to afford the title compound (300 mg, 29.5%) as a white solid. ¹H NMR (400 MHz, MeOH-d₄): δ 3.13-3.07 (m, 2H), 1.98-1.92 (m, 2H), 1.98-1.93 (m, 2H), 1.09-1.05 (m, 2H), 0.82-0.81 (m, 2H).

### 3-(1-(Trifluoromethyl)cyclopropyl)propan-1-amine

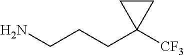 [SMILES: NCCCC1(C(F)(F)F)CC1]

A solution of 1-(trifluoromethyl)cyclopropanecarboxylic acid (6.0 g, 38.9 mmol) in BH₃-THF (70 mL) was stirred at 40° C. overnight. The mixture was quenched with saturated NH₄Cl and extracted with EA (200 mL×3). The organics were washed with saturated NaHCO₃ aqueous solution, brine, dried over Na₂SO₄, filtered and concentrated to afford (1-(trifluoromethyl)cyclopropyl)methanol as a colorless oil (5.0 g, 91.7%). ¹H NMR (400 MHz, DMSO-d₆) δ 4.93 (t, J=6.0 Hz, 1H), 3.52 (d, J=6.0 Hz, 2H), 0.86-0.75 (m, 4H).

A solution of (1-(trifluoromethyl)cyclopropyl)methanol (5.0 g, 35.7 mmol), Et₃N (7.2 g, 71.4 mmol) and TsCl (7.5 g, 39.3 mmol) in DCM (60 mL) was stirred at RT overnight. The mixture was concentrated under vacuum and the residue was purified by silica gel column chromatography (PE:EA=10:1) to give (1-(trifluoromethyl)cyclopropyl)methyl 4-methylbenzenesulfonate (7.9 g, 75.2%) as a colorless oil. ¹H NMR (400 MHz, DMSO-d₆) δ 7.77 (m, 2H), 7.48 (m, 2H), 4.16 (s, 2H), 2.41 (s, 3H), 1.04 (m, 2H), 0.92 (m, 2H).

At 0° C. to a solution of diisopropyl malonate (12.3 g, 65.5 mmol) in DMF (80 mL) was added NaH (60% in mineral, 3.1 g, 78.6 mmol). After the addition, the reaction mixture was warmed to RT and NaI (3.9 g, 26.2 mmol) was added followed by the drop-wise addition of a solution of (1-(trifluoromethyl)cyclopropyl)methyl 4-methylbenzenesulfonate (7.7 g, 26.2 mmol) in DMF (80 mL). The reaction mixture was heated at 80° C. overnight. The mixture was cooled to RT quenched with saturated NH₄Cl aqueous solution and extracted with DCM (200 mL×3). The organics were dried over Na₂SO₄, filtered and concentrated. The residue was purified by silica gel column chromatography (DCM) to give diisopropyl 2-((1-(trifluoromethyl)cyclopropyl) methyl)malonate (5.0 g, 61.6%) as a colorless oil. ¹H NMR (400 MHz, DMSO-d₆) δ 4.91 (m, 2H), 3.47 (t, J=7.2 Hz, 1H), 2.10 (d, J=7.2 Hz, 2H), 1.17 (dd, J=7.3 Hz, 6.3 Hz, 12H), 0.89 (m, 2H), 0.76 (m, 2H).

To a solution of 2-((1-(trifluoromethyl)cyclopropyl)methyl)malonate (4.9 g, 15.8 mmol) in MeOH/dioxane/H₂O (1/1/1, 80 mL) was added NaOH (3.8 g, 94.8 mmol) and the resulting reaction mixture was heated to 40° C. and stirred overnight. The mixture was cooled to RT and the organic solvents were removed under reduced pressure. The aqueous layer was acidified with 3M HCl and the mixture was extracted with DCM (200 mL×3). The combined organics were dried over Na₂SO₄, filtered and concentrated to afford 2-((1-(trifluoromethyl)cyclopropyl)methyl)malonic acid (3.6 g, 100%) as a yellow oil. ¹H NMR (400 MHz, DMSO-d₆) δ 12.91 (s, 2H), 3.36-3.34 (m, 1H), 2.08 (d, J=7.0 Hz, 2H), 0.88 (m, 2H), 0.77 (m, 2H).

A solution of 2-((1-(trifluoromethyl)cyclopropyl)methyl)malonic acid (3.6 g, 15.9 mmol) in pyridine (50 mL) was heated to 100° C. and stirred overnight, then cooled to RT and concentrated to dryness. The residue was dissolved in 3N HCl and the mixture was extracted with DCM (200 mL×3). The organics were dried over Na₂SO₄, filtered and concentrated to afford 3-(1-(trifluoromethyl)cyclopropyl)propanoic acid (2.4 g, 82.8%) as a yellow oil. ¹H NMR (400 MHz, DMSO-d₆) δ 12.18 (s, 1H), 2.32 (t, J=8.0 Hz, 2H), 1.79 (t, J=8.0 Hz, 2H), 0.86 (m, 2H), 0.75 (m, 2H).

A solution of 3-(1-(trifluoromethyl)cyclopropyl)propanoic acid (2.3 g, 12.6 mmol) and oxalyl chloride (2.4 g, 18.9 mmol) in DCM (30 mL) and DMF (10 drops) was stirred at RT for 1 h, then concentrated to dryness. The residue was co-evaporated with DCM, then dissolved in THF (30 mL). The mixture was added drop-wise to a solution of NH₄OH (15M, 12.6 mL, 189 mmol) in THF (30 mL) and the resulting reaction mixture was stirred at RT for 30 min. The mixture was treated with brine and extracted with EA (200 mL×3). The organics were dried over Na₂SO₄, filtered and concentrated to dryness to afford 3-(1-(trifluoromethyl)cyclopropyl)propanamide (2.0 g, 87.4%) as a yellow solid. ¹H NMR (400 MHz, DMSO-d₆) δ 7.30 (s, 1H), 6.77 (s, 1H), 2.16 (m, 2H), 1.75 (m, 2H), 0.85 (m, 2H), 0.71 (m, 2H).

A solution of 3-(1-(trifluoromethyl)cyclopropyl)propanamide (500 mg) in BH₃-THF (10 mL) was stirred at 70° C. overnight, then cooled to RT, quenched with H₂O and extracted with EA (100 mL×3). The organics were dried over Na₂SO₄, filtered and concentrated to dryness to afford the title compound (300 mg, 65%). LC-MS m/z: 168.2 [M+H]⁺.

### 5-Bromo-2-methoxy-1-((2-(trimethylsilyl)ethoxy)methyl)-1H-imidazole

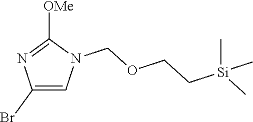 [SMILES: COc1nc(Br)cn1COCC[Si](C)(C)C]

To a solution of 2,4,5-tribromo-1H-imidazole (5.0 g, 16.56 mmol) in DMF (10 mL) was added NaH (795 mg, 33.12 mmol) and the mixture was stirred at 0° C. for 10 min. Then SEMCl (4.08 g, 24.59 mmol) was added dropwise at a rate that maintained the internal temperature between 0° C.˜5° C. The mixture was concentrated and purified by silica gel column chromatography (PEEA=20/1) to give 2,4,5-tribromo-1-((2-(trimethylsilyl)ethoxy)methyl)-1H-imidazole (6.0 g, 83.9%) as a yellow oil. LC-MS m/z: 407.1 [M+H]⁺. Purity (214 nm): >99%; t_{R}=1.60 min.

To a solution of 2,4,5-tribromo-1-((2-(trimethylsilyl)ethoxy)methyl)-1H-imidazole (6.0 g, 13.9 mmol) in MeOH (12 mL) was added NaOMe (3.75 g, 69.5 mmol) and the mixture was stirred at 110° C. for 2 h under microwave conditions. The mixture was concentrated and purified by silica gel column chromatography (PE/EA=5/1) to give 4,5-dibromo-2-methoxy-1-((2-(trimethylsilyl)ethoxy)methyl)-1H-imidazole (3.0 g, 56.6%) as a yellow oil. LC-MS m/z: 385.1. [M+H]r. Purity (214 nm): >99%; t_{R}=2.34 min.

To a solution of 4,5-dibromo-2-methoxy-1-((2-(trimethylsilyl)ethoxy)methyl)-1H-imidazole (3.0 g, 7.8 mmol) in THF (30 mL) was added n-BuLi (2.5 mol/L, 3.1 mL) and the mixture was stirred at −78° C. for 3 h. The mixture was then poured into 20 mL ice-water and extracted with EA (30 mL×3). The combined organic layers were dried over anhydrous Na₂SO₄, filtered and concentrated. The residue was purified by silica gel column chromatography (PE/EA=5/1) to give the title compound (1.7 g, 71.1%) as a pale-yellow oil. ¹H NMR (400 MHz, CDCl₃) δ 6.63 (s, 1H), 5.04 (s, 2H), 4.04 (s, 3H), 3.51 (t, J=8.4 Hz, 2H), 0.91 (t, J=8.4 Hz, 2H). LC-MS m/z: 307.0 [M+H]⁺. Purity (214 nm): >94%; t_{R}=2.18 min.

### 4-tert-Butyl-2-methoxy-1H-imidazole

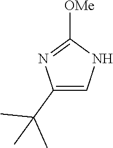

A solution of 1-bromo-3,3-dimethylbutan-2-one (1.00 g, 11.1 mmol), o-methylisourea sulfate (1.44 g, 16.7 mmol) and sodium bicarbonate (1.40 g, 33.3 mmol) in EtOH (10 ml) was stirred for 16 h at 65° C. The mixture was concentrated and purified by silica gel column chromatography (PE:EA=2:1) to give the title compound as a colorless oil (140 mg, 27.9%).

### 2-Methoxy-1H-imidazole

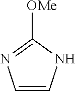

To a solution of 2,4,5-tribromo-1H-imidazole (5.0 g, 16.56 mmol) in DMF (10 mL) was added NaH (795 mg, 33.12 mmol), then the mixture was stirred at 0° C. for 10 min. Then SEMCl (4.08 g, 24.59 mmol) was added dropwise to the cold slurry at a rate that maintained the internal temperature between 0° C.˜5° C. The mixture was concentrated and purified by silica gel column chromatography (PE/EA=20/1) to give 2,4,5-tribromo-1-((2-(trimethylsilyl)ethoxy)methyl)-1H-imidazole (6.0 g, 83.9%) as a yellow oil. LC-MS m/z: 407.1 [M+H]⁺. HPLC Purity (214 nm): >99%; t_{R}=1.60 min.

To a solution of 2,4,5-tribromo-1-((2-(trimethylsilyl)ethoxy)methyl)-1H-imidazole (6.0 g, 13.9 mmol) in MeOH (12 mL) was added sodium methanolate (3.75 g, 69.5 mmol), then the mixture was stirred at 110° C. for 2 h under microwave condition. The mixture was concentrated and purified by silica gel column chromatography (PE/EA=5/1) to give 4,5-dibromo-2-methoxy-1-((2-(trimethylsilyl)ethoxy)methyl)-1H-imidazole (3.0 g, 56.6%) as a yellow oil. LC-MS m/z: 385.1 [M+H]⁺. Purity (214 nm): >99%; t_{R}=2.34 min.

To a solution of 4,5-dibromo-2-methoxy-1-((2-(trimethylsilyl)ethoxy)methyl)-1H-imidazole (3.0 g, 7.8 mmol) in THF (30 mL) was added n-BuLi (2.5 mol/L, 6.2 mL), then the mixture was stirred at −78° C. for 2 h. The mixture was poured into 20 mL ice-water and extracted with EA (30 mL×3). The combined organic layers were dried over anhydrous Na₂SO₄ and concentrated. The residue was purified by silica gel column chromatography (PE/EA=5/1) to give 5-bromo-2-methoxy-1-((2-(trimethylsilyl) ethoxy)methyl)-1H-imidazole (1.2 g, 67.4%) as a yellow oil. LC-MS m/z: 229.1 [M+H]⁺. Purity (214 nm): >99%; t_{R}=1.14 min.

A solution of 5-bromo-2-methoxy-1-((2-(trimethylsilyl)ethoxy)methyl)-1H-imidazole (1.2 mg, 5.26 mmol) in conc. HC/THF (6 ml/6 ml) was stirred at RT for 3 h. The pH value adjusted to 9 with 20 ml saturated NaHCO₃ aqueous solution. Extracted with DCM (50 ml×3) and the combined organic layers were dried over anhydrous Na₂SO₄ and concentrated to give 2-methoxy-1H-imidazole (360 mg, 69.8%) as a yellow oil. LC-MS m/z: 99.1 [M+H]⁺.

### Example 2—N-Phenethyl-4-phenyl-1H-imidazole-1-carboxamide

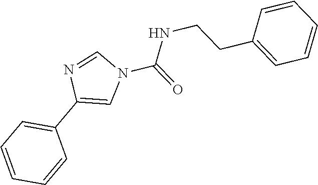 [SMILES: O=C(NCCc1ccccc1)n1cnc(-c2ccccc2)c1]

Following general procedure B (method 1), 4-phenyl-1H-imidazole (0.20 g, 1.39 mmol) and (2-isocyanatoethyl)benzene (0.31 g, 2.08 mmol) in DCM afforded the title compound (0.14 g, 35%) as a white solid. ¹H NMR (400 MHz, DMSO-di) δ 8.67 (brs, 1H), 8.29 (s, 1H), 8.11 (s, 1H), 7.81 (d, J=7.2 Hz, 2H), 7.42-7.22 (m, 8H), 3.51 (br m, 2H), 2.89 (t, J=7.6 Hz, 1H). LC-MS m/z: 292.2 [M+H]⁺. HPLC Purity (254 nm): 100%; t_{R}=1.95 min.

### Example 3—4-tert-Butyl-N-phenethyl-1H-imidazole-1-carboxamide

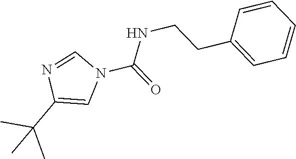 [SMILES: CC(C)(C)c1cn(C(=O)NCCc2ccccc2)cn1]

Following general procedure A, 1-bromo-3,3-dimethylbutan-2-one (0.20 g, 11.24 mmol) afforded 4-tert-butyl-1H-imidazole (0.50 g, 35%) as a yellow oil. LC-MS m/z: 125.1 [M+H]⁺. HPLC Purity (254 nm): 65%; t_{R}=1.14 min.

Following general procedure B (method 1), 4-tert-butyl-1H-imidazole (0.20 g, 1.39 mmol) and (2-isocyanatoethyl)benzene (0.20 g, 1.39 mmol) in DCM afforded the title compound (39 mg, 9.0%) as a white solid. ¹H NMR (400 MHz, DMSO-d₆) δ 8.46 (s, 1H), 8.09 (s, 1H), 7.32-7.21 (m, 6H), 3.46-3.44 (m, 2H), 2.84 (t, J=7.2 Hz, 2H), 1.20 (s, 9H). LC-MS m/z: 272.3 [M+H]⁺. HPLC Purity (254 nm): 100%; t_{R}=1.95 min.

### Example 4—N-Phenethyl-4-(pyridin-2-yl)-1H-imidazole-1-carboxamide

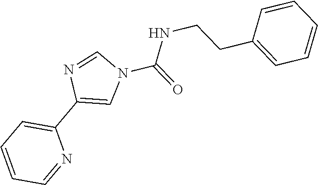 [SMILES: O=C(NCCc1ccccc1)n1cnc(-c2ccccn2)c1]

Following general procedure A, 2-bromo-1-(pyridin-2-yl)ethanone hydrobromide (400 mg, 1.43 mmol) afforded 2-(1H-imidazol-4-yl)pyridine (180 mg, 86%) as a yellow oil. LC-MS m/z: 146.0 [M+H]⁺. Purity (254 nm): >97%; t_{R}=1.38 min.

Following general procedure B (method 1), 2-(1H-imidazol-4-yl)pyridine (80 mg, 0.55 mmol) and (2-isocyanatoethyl)benzene (162 mg, 1.1 mmol) in CH₃CN afforded the title compound (43 mg, 12%) as a yellow solid. ¹H NMR (400 MHz, CDCl₃) δ 8.52 (d, J=4.6 Hz, 1H), 8.20 (s, 1H), 7.99 (d, J=7.9 Hz, 1H), 7.87 (s, 1H), 7.76 (t, 0.1=8.5 Hz, 1H), 7.34 (t, J=7.3 Hz, 2H), 7.29-7.15 (m, 4H), 5.95 (s, 1H), 3.72 (dd, J=12.9, 6.7 Hz, 2H), 2.96 (t, J=6.9 Hz, 2H). LC-MS m/z: 293.2 [M+H]⁺. HPLC Purity (214 nm): 98%; t_{R}=6.42 min.

### Example 5—N-Phenethyl-4-(pyridin-3-yl-1H-imidazole-1-carboxamide

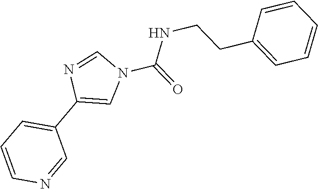 [SMILES: O=C(NCCc1ccccc1)n1cnc(-c2cccnc2)c1]

Following general procedure A, 2-bromo-1-(pyridin-3-yl)ethanone hydrobromide (2.00 g, 7.17 mmol) afforded 3-(1H-imidazol-4-yl)pyridine (0.70 g, 67.9%) as a yellow oil. LC-MS m/z: 145.1 [M+H]⁺. HPLC Purity (254 nm): 78%; t_{R}=0.72 min.

Following general procedure B (method 1), 3-(1H-imidazol-4-yl)pyridine (0.50 g, 3.45 mmol) and (2-isocyanatoethyl)benzene (0.61 g, 4.14 mmol) in DCM afforded the title compound (0.13 g, 13%) as a white solid. ¹H NMR (400 MHz, DMSO-d₆) δ 9.02 (d, J=2.0 Hz, 1H), 8.74 (t, J=5.6 Hz, 1H), 8.47 (dd, J=4.8, 1.6 Hz, 1H), 8.35 (s, 1H), 8.25 (s, 1H), 8.14 (dt, J=8.5, 2.0 Hz, 1H), 7.2 (dd, J=8.0, 5.2 Hz, 1H), 7.33-7.20 (m, 5H), 3.53-3.48 (q, J=7.6 Hz, 2H), 2.89 (t, J=7.2 Hz, 2H). LC-MS m/z: 293.3 [M+H]⁺. HPLC Purity (254 nm): 100%; t_{R}=1.75 min.

### Example 6—N-Phenethyl-4-(pyridin-4-yl)-1H-imidazole-1-carboxamide

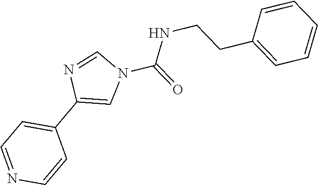 [SMILES: O=C(NCCc1ccccc1)n1cnc(-c2ccncc2)c1]

Following general procedure A, 2-bromo-1-(pyridin-4-yl)ethanone hydrobromide (558 mg, 2 mmol) afforded 4-(1H-imidazol-4-yl)pyridine (250 mg, 86%) as a yellow oil. LC-MS m/z: 146.0 [M+H]⁺. HPLC Purity (254 nm): >97%; t_{R}=1.10 min.

Following general procedure B (method 1), 4-(1H-imidazol-4-yl)pyridine (145 mg, 1 mmol) and (2-isocyanatoethyl)benzene (194 mg, 2 mmol) in CH₃CN afforded the title compound (14 mg, 5%) as a white solid. ¹H NMR (400 MHz, CDCl₃) δ 8.60 (d, J=5.7 Hz, 2H), 8.06 (s, 1H), 7.71 (s, 1H), 7.65 (d, J=5.9 Hz, 2H), 7.37 (t, J=7.3 Hz, 2H), 7.32-7.20 (m, 3H), 5.81 (s, 1H), 3.74 (dd, J=12.8, 6.6 Hz, 2H), 2.99 (t, J=6.8 Hz, 2H). LC-MS m/z: 293.1 [M+H]⁺. HPLC Purity (214 nm): 99%; t_{R}=6.72 min.

### Example 7—2-Methyl-N-phenethyl-4-phenyl-1H-imidazole-1-carboxamide

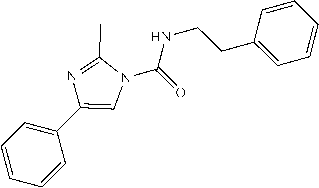 [SMILES: Cc1nc(-c2ccccc2)cn1C(=O)NCCc1ccccc1]

Following general procedure B (method 1), 2-methyl-4-phenyl-1H-imidazole (0.20 g, 1.27 mmol) and (2-isocyanatoethyl)benzene (0.22 g, 1.52 mmol) DCM afforded the title compound (14.0 mg, 3.6%) as a white solid. ¹H NMR (400 MHz, CDCl₃) δ 7.71 (d, J=8.4 Hz, 2H), 7.38-7.36 (m, 4H), 7.30-7.20 (m, 5H), 5.55 (br, 1H), 3.70 (dd, J=12.8 Hz, 6.8 Hz, 2H), 2.97 (t, J=6.8 Hz, 2H), 2.63 (s, 3H). LC-MS m/z: 306.1 [M+H]⁺. HPLC Purity (254 nm): 95%; t_{R}=1.65 min.

### Example 8—2-Methoxy-N-phenethyl-4-phenyl-1H-imidazole-1-carboxamide

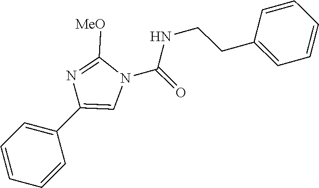 [SMILES: COc1nc(-c2ccccc2)cn1C(=O)NCCc1ccccc1]

A mixture of 2-bromo-1-phenylethanone (2.0 g, 7.14 mmol), methyl carbamimidate hydrochloride (1.17 g, 10.7 mmol) and NaHCO₃ (1.80 g, 21.4 mmol) in EtOH (100 mL) was heated to 60° C. and stirred for 6 h. The reaction mixture was concentrated and purified by silica gel column chromatography (EA:PE=10:1) to afford 2-methoxy-4-phenyl-1H-imidazole (800 mg, 66%) as a yellow solid. LC-MS m/z: 175.1 [M+H]⁺. HPLC Purity (214 nm): 95%; t_{R}=0.58 min.

Following general procedure B (method 1), 2-methoxy-4-phenyl-1H-imidazole (200 mg, 1.15 mmol) and (2-isocyanatoethyl)benzene (253 mg, 1.73 mmol) in DCM afforded the title compound (80 mg, 22%) as a white solid. ¹H NMR (400 MHz, CDCl₃) δ 7.71 (d, J=8.4 Hz, 2H), 7.53 (s, 1H), 7.36 (t, J=6.8 Hz, 4H), 7.29 (d, J=7.2 Hz, 1H), 7.26-7.21 (m, 3H), 6.95 (t, J=4.8 Hz, 1H), 4.08 (s, 3H), 3.69 (q, J=6.2 Hz, 2H), 2.93 (t, J=6.8 Hz, 2H). LC-MS m/z: 322.3 [M+H]⁺. HPLC Purity (214 nm): >99%; t_{R}=9.86 min.

### Example 9—4-Phenyl-N-(3-phenylpropyl-1H-imidazole-1-carboxamide

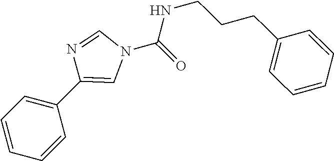 [SMILES: O=C(NCCCc1ccccc1)n1cnc(-c2ccccc2)c1]

Following general procedure C, 4-phenyl-1H-imidazole (0.30 g, 2.08 mmol) and 3-phenylpropan-1-amine (0.31 g, 2.29 mmol) afforded the title compound (242 mg, 38%) as a white solid. ¹H NMR (400 MHz, CDCl₃) δ 7.95 (s, 1H), 7.75 (d, J=7.2 Hz, 2H), 7.41-7.23 (m, 9H), 5.56 (br s, 1H), 3.51 (dd, J=12.8, 6.4 Hz, 2H), 2.77 (t, J=7.2 Hz, 2H), 2.02 (t, J=6.8 Hz, 1H). LC-MS m/z: 306.1 [M+H]⁺. HPLC Purity (254 nm): 100%; t_{R}=2.01 min.

### Example 10—4-(6-((Dimethylamino)methyl)pyridin-2-yl)-N-phenethyl-1H-imidazole-1-carboxamide

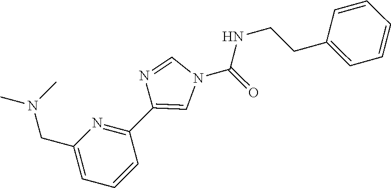 [SMILES: CN(C)Cc1cccc(-c2cn(C(=O)NCCc3ccccc3)cn2)n1]

A solution of 6-bromopicolinaldehyde (3.00 g, 16.22 mmol), NHMe₂ (1.10 g, 24.33 mmol) and NaBH₃CN (1.53 g, 24.33 mmol) in MeOH (30.0 mL) was stirred at RT for 48 h. The MeOH was removed and the residue was purified by FCC (DCM/MeOH=1/1) to give 1-(6-bromopyridin-2-yl)-N,N-dimethylmethanamine (1.3 g, 38.2%) as a brown oil. LC-MS m/z: 215.1 [M+H]⁺. HPLC Purity (254 nm): 84%; t_{R}=1.68 min.

A solution of 1-(6-bromopyridin-2-yl)-N,N-dimethylmethanamine (1.10 g, 5.14 mmol), 4-(tributylstannyl)-1-trityl-1H-imidazole (4.63 g, 7.71 mmol) and Pd(PPh₃)₄ (0.59 g, 0.51 mmol) in DMF (10.0 mL) was stirred at 100° C. for 2 h. The mixture was cooled to RT, diluted with water (20 mL), extracted with EA (100 mL×2), washed with water (20 mL×2), dried over anhydrous Na₂SO₄, filtered and concentrated. The residue was purified by silica gel column chromatography (MeOH) to give N,N-dimethyl-1-(6-(1-trityl-1H-imidazol-4-yl)pyridin-2-yl)methanamine (0.6 g, 26.3%) as a white solid. LC-MS m/z: 445.3 [M+H]⁺. HPLC Purity (254 nm): 84%; t_{R}=1.32 min.

N,N-dimethyl-1-(6-(1-trityl-1H-imidazol-4-yl)pyridin-2-yl)methanamine (0.50 g, 1.13 mmol) was dissolved in 4 N HCl-dioxane (5.0 ml) and the reaction mixture was stirred at RT for 3 h. Then the solid was filtered and dried to give 1-(6-(1H-imidazol-4-yl)pyridin-2-yl)-N,N-dimethylmethanamine (0.12 g, 54.5%) as a white solid. LC-MS m/z: 203.1 [M+H]⁺. HPLC Purity (254 nm): 75%; t_{R}=0.44 min.

Following general procedure B (method 1), 1-(6-(1H-imidazol-4-yl)pyridin-2-yl)-N,N-dimethylmethanamine (0.10 g, 0.49 mmol) and (2-isocyanatoethyl)benzene (0.09 g, 0.59 mmol) in DCM afforded the title compound (8.6 mg, 5.0%) as a white solid. ¹H NMR (400 MHz, DMSO-d₆) δ 10.01 (s, 1H), 9.01 (brs, 1H), 8.57 (s, 1H), 8.54 (s, 1H), 7.98-7.91 (m, 2H), 7.42 (d, J=7.6 Hz, 1H), 7.33-7.19 (m, 5H), 4.50 (d, J=5.2 Hz, 2H), 3.55-3.50 (m, 2H), 2.91 (t, J=7.6 Hz, 2H), 2.87 (s, 3H), 2.86 (s, 3H). LC-MS m/z: 350.3 [M+H]⁺. HPLC Purity (254 nm): 97%; t_{R}=6.34 min.

### Example 11—4-tert-Butyl-2-methoxy-N-phenethyl-1H-imidazole-1-carboxamide

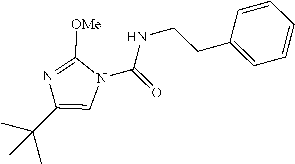 [SMILES: COc1nc(C(C)(C)C)cn1C(=O)NCCc1ccccc1]

Following general procedure B (method 1), 4-tert-butyl-2-methoxy-1H-imidazole (150 mg, 0.97 mmol) and (2-isocyanatoethyl)benzene (214 mg, 1.46 mmol) in DCM afforded the title compound (40 mg, 13%) as a white oil. ¹H NMR (400 MHz, CDCl₃) δ 7.38-7.32 (m, 2H), 7.28-7.20 (m, 3H), 6.89 (s, 2H), 3.98 (s, 3H), 3.64 (q, J=6.0 Hz, 2H), 2.89 (t, J=6.8 Hz, 2H), 1.30 (s, 9H). LC-MS m/z: 301.9 [M+H]⁺. HPLC Purity (214 nm): >990%; t_{R}=9.47 min.

### Example 12—2-Methoxy-4-phenyl-N-(3-phenylpropyl)-1H-imidazole-1-carboxamide

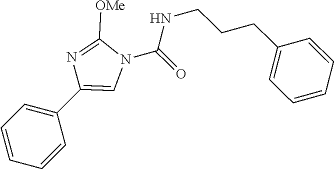 [SMILES: COc1nc(-c2ccccc2)cn1C(=O)NCCCc1ccccc1]

Following general procedure C, 2-methoxy-4-phenyl-1H-imidazole (170 mg, 1 mmol) and 2-phenylpropylamine (121 mg, 0.9 mmol) afforded the title compound (30 mg, 99%) as a white solid. ¹H NMR (400 MHz, CDCl₃) δ 7.89-7.66 (m, 2H), 7.54 (s, 1H), 7.42-7.28 (m, 8H), 6.94 (brs, 1H), 4.23 (s, 3H), 3.43 (dt, J=13.0, 7.1 Hz, 2H), 2.72 (t, J=7.6 Hz, 2H), 2.10-1.77 (quin, J=7.2 Hz, 2H). LC-MS m/z: 336 [M+H]⁺. HPLC Purity (214 nm): 99%; t_{R}=9.98 min.

### Example 13—N-(2-Methyl-2-phenylpropyl)-4-(pyridin-3-yl)-1H-imidazole-1-carboxamide

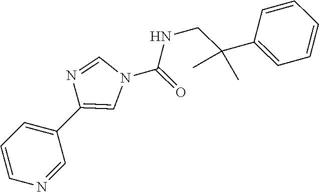 [SMILES: CC(C)(CNC(=O)n1cnc(-c2cccnc2)c1)c1ccccc1]

Following general procedure C, 3-(1H-imidazol-4-yl)pyridine (154 mg, 1.06 mmol) and 2-methyl-2-phenylpropan-1-amine (100 mg 0.67 mmol) afforded the title compound (27.9 mg 8.2%) as a white solid. ¹H NMR (400 MHz, CDCl₃) δ 8.92 (s, 1H), 8.51 (d, J=4.5 Hz, 1H), 8.06 (d, 0.1=8.0 Hz, 1H), 7.95 (s, 1H), 7.42 (s, 5H), 7.32 (dd, J=7.9, 4.5 Hz, 2H), 5.25 (s, 1H), 3.64 (d, J=6.0 Hz, 2H), 1.45 (s, 6H). LC-MS m/z: 321.3 [M+H]⁺. HPLC Purity (214 nm): >98%; t_{R}=1.33 min.

### Example 14—N-(3-Phenylpropyl)-4-(pyridin-3-yl)-1H-imidazole-1-carboxamide

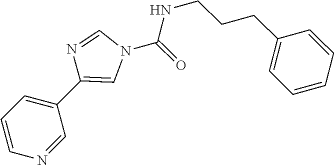 [SMILES: O=C(NCCCc1ccccc1)n1cnc(-c2cccnc2)c1]

Following general procedure C, 3-(1H-imidazol-4-yl)pyridine (200 mg, 1.38 mmol) and 3-phenylpropan-1-amine (223 mg, 1.66 mmol) afforded the title compound (75 mg, 19%) as a white solid. ¹H NMR (400 MHz, CDCl₃) δ 8.96 (d, J=1.6 Hz, 1H), 8.53 (dd, 0.1=4.8, 1.6 Hz, 1H), 8.10 (d, J=7.6 Hz, 1H), 7.96 (s, 1H), 7.40-7.26 (m, 7H), 5.46 (t, J=3.6 Hz, 1H), 3.55 (q, J=6.0 Hz, 2H), 2.80 (t, J=6.8 Hz, 2H), 2.06 (p, J=6.4 Hz, 2H). LC-MS m/z: 307.1 [M+H]⁺. HPLC Purity (214 nm): 99%; t_{R}=5.94 min.

### Example 15—N-(2-Methyl-3-phenylpropyl)-4-(pyridin-3-yl)-1H-imidazole-1-carboxamide

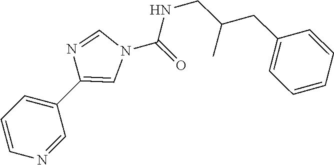 [SMILES: CC(CNC(=O)n1cnc(-c2cccnc2)c1)Cc1ccccc1]

A mixture of 2-methyl-3-phenylpropanoic acid (2.0 g, 12.2 mmol), HOBT (1.8 g, 13.4 mmol), EDCI (2.56 g, 13.4 mmol) and DIPEA (2.35, 13.4 mmol) in DMF (30 ml) was stirred at RT for 0.5 h followed by the addition of NH₄Cl (1.95 g, 37 mmol) and the reaction mixture was stirred at RT for 10 h. The solvent was removed under reduced pressure and the residue was dissolved in EA (50 ml), washed with NaOH (1 N, 40 ml×2) and HCl (1N, 40 ml×2) and brine (50 mL). The organic layer was dried over Na₂SO₄ and concentrated to afford 2-methyl-3-phenylpropanamide (1.2 g, 60%) as a white solid. LC-MS m/z: 164.4 [M+H]⁺. HPLC Purity (214 nm): 100%; t_{R}=1.62 min.

A solution of 2-methyl-3-phenylpropanamide (800 mg, 4.8 mmol) and LAH (240 mg, 6.4 mmol) in THF (20 ml) was stirred for 1 day at RT under N₂. Then H₂O (240 mg), NaOH (15%, 240 mg) and H₂O (720 mg) were added successively. The mixture was stirred for 10 min and filtered. The filtrate was concentrated and purified by silica gel column chromatography (DCM:MeOH=8:1) to give 2-methyl-3-phenylpropan-1-amine (500 mg, 68%) as a yellow oil. LC-MS m/z: 150 [M+H]⁺. HPLC Purity (214 nm): 90%; t_{R}=1.57 min.

Following general procedure C, 3-(1H-imidazol-4-yl)pyridine (400 mg, 2.7 mmol) and 2-methyl-3-phenylpropan-1-amine (530 mg, 3.6 mmol) afforded the title compound (26 mg, 2.4%) as a white solid. ¹H NMR (400 MHz, CDCl₃) δ 8.94 (dd, J=2.2, 0.7 Hz, 1H), 8.51 (dd, J=4.8, 1.6 Hz, 1H), 8.17-8.06 (m, 1H), 7.91 (d, J=1.3 Hz, 1H), 7.39-7.27 (m, 4H), 7.26-7.21 (m, 3H), 5.66 (s, 1H), 3.62-3.45 (m, 1H), 3.34-3.24 (m, 1H), 2.77 (dd, J=13.7, 6.1 Hz, 1H), 2.61 (dd, J=13.7, 8.3 Hz, 1H), 2.20 (dd, J=13.6, 6.9 Hz, 1H), 1.07 (d, J=6.8 Hz, 3H). LC-MS m/z: 321.0 [M+H]⁺. HPLC Purity (214 nm): 100%; t_{R}=1.93 min.

### Example 16—N-(3-Phenylbutyl)-4-(pyridin-3-yl)-1H-imidazole-1-carboxamide

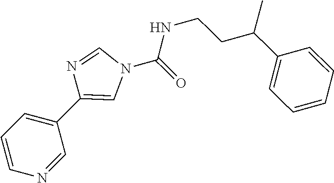 [SMILES: CC(CCNC(=O)n1cnc(-c2cccnc2)c1)c1ccccc1]

A mixture of 3-(pyridin-3-yl)butanal (3 mL, 20 mmol) and CH₃CO₂NH₄ (1.18 g, 20 mmol) in MeOH (30 mL) was stirred at RT for 1 h. Then NaBH₃CN (900 mg, 14 mmol) was added and the reaction mixture was stirred at RT for 10 h. Solvent was removed under reduced pressure and the residue was dissolved in EA (50 ml), washed with NaOH (1 N, 40 mL×2), HCl (1N, 40 mL×2) and brine (50 mL). The organic layer was dried over Na₂SO₄ and purified by silica gel column chromatography (DCM:MeOH=12:1) to give 3-(pyridin-3-yl)butan-1-amine (1.5 g, 60%) as a yellow oil. LC-MS m/z: 151 [M+H]⁺. HPLC Purity (214 nm): 90%; t_{R}=1.62 min.

Following general procedure C, 3-(1H-imidazol-4-yl)pyridine (200 mg, 1.4 mmol) and 3-phenylbutan-1-amine (210 mg, 1.4 mmol) afforded the title compound (18 mg, 4.1%) as a white solid. ¹H NMR (400 MHz, CDCl₃) δ 8.94 (s, 1H), 8.58-8.43 (m, 1H), 8.09 (d, J=7.9 Hz, 1H), 7.86 (s, 1H), 7.47-7.26 (m, 6H), 7.17 (s, 1H), 5.31 (s, 1H), 3.63 (td, J=12.6, 6.2 Hz, 1H), 3.41-3.20 (m, 1H), 2.97-2.75 (m, 1H), 2.15-1.83 (m, 2H), 1.34 (d, J=7.0 Hz, 3H). LC-MS m/z: 321.0 [M+H]⁺. HPLC Purity (214 nm): 98.5%; t_{R}=1.63 min.

### Example 17—N-(2-Phenoxyethyl)-4-(pyridin-3-yl)-1H-imidazole-1-carboxamide

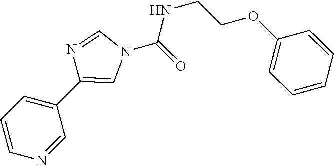 [SMILES: O=C(NCCOc1ccccc1)n1cnc(-c2cccnc2)c1]

Following general procedure C, 3-(1H-imidazol-4-yl)pyridine (200 mg, 1.38 mmol) and 2-phenoxyethanamine (223 mg, 1.66 mmol) afforded the title compound (67 mg, 17%) as a white solid. ¹H NMR (400 MHz, CDCl₃) δ 9.00 (d, J=2.0 Hz, 1H), 8.55 (dd, J=4.8, 2.0 Hz, 1H), 8.21 (s, 1H), 8.13 (dt, J=8.0, 2.0 Hz, 1H), 7.70 (s, 1H), 7.36-7.26 (m, 3H), 7.00 (t, J=7.6 Hz, 1H), 7.92 (d, J=4.0 Hz, 2H), 6.52 (t, J=5.2 Hz, 1H), 4.21 (t, J=4.8 Hz, 2H), 3.90 (q, J=5.2 Hz, 2H). LC-MS m/z: 309.1 [M+H]⁺. HPLC Purity (214 nm): 99%; t_{R}=5.62 min.

### Example 18—N-(2-Phenoxyethyl)-4-phenyl-1H-imidazole-1-carboxamide

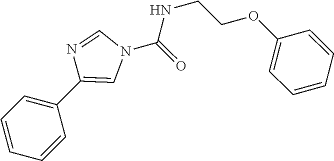 [SMILES: O=C(NCCOc1ccccc1)n1cnc(-c2ccccc2)c1]

Following general procedure C, 4-phenyl-1H-imidazole (0.30 g, 2.08 mmol) and 2-phenoxyethanamine (0.31 g, 2.29 mmol) afforded the title compound (18.5 mg, 4.3%) as a white solid. ¹H NMR (400 MHz, DMSO-d₆) δ 8.82 (s, 1H), 8.34 (s, 1H), 8.16 (s, 1H), 7.81 (d, J=8.0 Hz, 2H), 7.40 (t, J=7.6 Hz, 2H), 7.31-7.27 (m, 3H), 6.99-6.92 (m, 3H), 4.15 (t, J=5.6 Hz, 2H), 3.66 (t, J=5.6 Hz, 2H). LC-MS m/z: 308.1 [M+H]⁺. HPLC Purity (214 nm): 100%, t_{R}=7.68 min.

### Example 19—4-tert-Butyl-N-(2-phenoxyethyl)-1H-imidazole-1-carboxamide

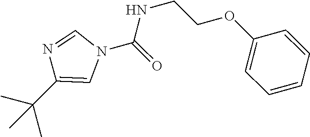 [SMILES: CC(C)(C)c1cn(C(=O)NCCOc2ccccc2)cn1]

Following general procedure C, 4-tert-butyl-1H-imidazole (0.30 g, 2.42 mmol) and 2-phenoxyethanamine (0.36 g, 2.66 mmol) afforded the title compound (76.8 mg, 11.1%) as a white solid. ¹H NMR (400 MHz, CDCl₃) δ 8.05 (s, 1H), 7.31 (t, J=8.0 Hz, 2H), 7.01-6.90 (m, 4H), 6.02 (brs, 1H), 4.16 (t, J=5.2 Hz, 2H), 3.85-3.81 (m, 2H), 1.28 (s, 9 Hz). LC-MS m/z: 288.1 [M+H]⁺. HPLC Purity (214 nm): 100%; t_{R}=6.59 min.

### Example 20—4-tert-Butyl-N-(2-(pyridin-3-yl)ethyl)-1H-imidazole-1-carboxamide

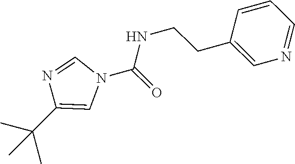 [SMILES: CC(C)(C)c1cn(C(=O)NCCc2cccnc2)cn1]

Following general procedure B (method 2), 4-t-butyl-1H-imidazole (200 mg, 1.61 mmol) and 2-(pyridin-3-yl)ethanamine (235.7 mg, 1.93 mmol) afforded the title compound (80 mg, 18%) as a white solid. ¹H NMR (400 MHz, CDCl₃) δ 8.46 (dd, J=4.8, 1.4 Hz, 1H), 8.41 (d, J=1.9 Hz, 1H), 7.99 (d, J=1.2 Hz, 1H), 7.57 (d, J=7.8 Hz, 1H), 7.28 (s, 1H), 6.98 (t, J=5.8 Hz, 1H), 6.26 (s, 1H), 3.76-3.57 (m, 2H), 3.09-2.79 (m, 2H), 1.26 (s, 9H). LC-MS m/z: 273.0 [M+H]⁺. HPLC Purity (214 nm): >97%; t_{R}=1.76 min.

### Example 21—N-iso-Pentyl-4-(pyridin-3-yl)-1H-imidazole-1-carboxamide

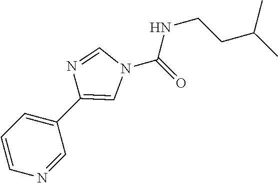 [SMILES: CC(C)CCNC(=O)n1cnc(-c2cccnc2)c1]

Following general procedure C, 3-(1H-imidazol-4-yl)pyridine (187 mg, 1.29 mmol) and 3-methylbutan-1-amine (90 mg 1.0 mmol) afforded the title compound (12.9 mg 3.9%) as a white solid. ¹H NMR (500 MHz, CDCl₃) δ 8.97 (d, J=1.5 Hz, 1H), 8.51 (dd, J=4.8, 1.4 Hz, 1H), 8.21 (s, 1H), 8.12 (d, J=8.0 Hz, 1H), 7.74 (s, 1H), 7.35 (dd, J=7.9, 4.9 Hz, 1H), 6.40 (s, 1H), 3.49 (dd, J=13.8, 6.8 Hz, 2H), 1.69 (td, J=13.3, 6.7 Hz, 1H), 1.56 (dd, J=14.8, 7.1 Hz, 2H), 0.97 (d, J=6.6 Hz, 6H). LC-MS m/z: 259.2 [M+H]⁺. HPLC Purity (214 nm): >99%; t_{R}=1.49 min.

### Example 22—N-(2-Cyclopropylethyl)-4-(pyridin-3-yl)-1H-imidazole-1-carboxamide

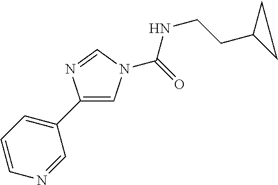 [SMILES: O=C(NCCC1CC1)n1cnc(-c2cccnc2)c1]

Following general procedure C, 3-(1H-imidazol-4-yl)pyridine (161 mg, 1.11 mmol) and 2-cyclopropylethanamine (132 mg, 1.08 mmol) afforded the title compound (25.3 mg 8.9%) as a white solid. ¹H NMR (500 MHz, CDCl₃) δ 8.99 (s, 1H), 8.57-8.47 (m, 1H), 8.20 (s, 1H), 8.13 (dt, J=7.9, 1.8 Hz, 1H), 7.72 (s, 1H), 7.35 (dd, J=7.9, 4.8 Hz, 1H), 6.35 (s, 1H), 3.62-3.49 (m, 2H), 1.58 (q, J=7.0 Hz, 2H), 0.83-0.66 (m, 1H), 0.53 (q, J=5.1 Hz, 2H), 0.18-0.05 (m, 2H). LC-MS m/z: 257.1 [M+H]⁺. HPLC Purity (254 nm): >99%; t_{R}=1.44 min.

### Example 23—4-(Pyridin-3-yl)-N-(2-(1-(trifluoromethyl)cyclopropyl)ethyl)-1H-imidazole-1-carboxamide

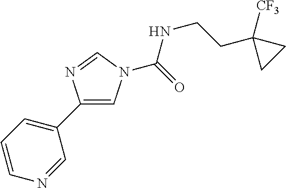 [SMILES: O=C(NCCC1(C(F)(F)F)CC1)n1cnc(-c2cccnc2)c1]

Following general procedure C, 3-(1H-imidazol-4-yl)pyridine (100 mg, 0.7 mmol) and 2-(1-(trifluoromethyl)cyclopropyl)ethanamine hydrochloride (182 mg, 0.8 mmol) afforded the title compound (4 mg, 1.8%) as a white solid. ¹H NMR (400 MHz, DMSO-d₆) δ 9.02 (t, J=1.6 Hz, 1H), 8.68 (brs, 1H), 8.48 (dd, J=4.8 Hz, 1.6 Hz, 1H), 8.35 (d, J=1.2 Hz, 1H), 8.25 (d, J=1.2 Hz, 1H), 8.15 (dt, J=8.4 Hz, 1.6 Hz, 1H), 7.49-7.40 (m, 1H), 3.42-3.36 (m, 2H), 1.88 (t, J=8 Hz, 2H), 0.97 (t, J=5.2 Hz, 2H), 0.84-0.79 (m, 2H) LC-MS m/z: 325.2 [M+H]⁺. HPLC Purity (214 nm): 100%; t_{R}=6.17 min.

### Example 24—N-(2-iso-Propoxyethyl)-4-(pyridin-3-yl)-1H-imidazole-1-carboxamide

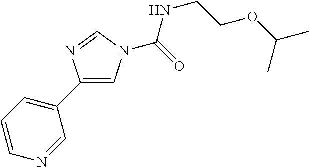 [SMILES: CC(C)OCCNC(=O)n1cnc(-c2cccnc2)c1]

Following general procedure C, 3-(1H-imidazol-4-yl)pyridine (200 mg, 1.38 mmol) and 2-isopropoxyethanamine (185 mg, 1.8 mmol) afforded the title compound as a yellow solid (16.2 mg, 4.2%). ¹H NMR (400 MHz, CDCl₃) δ 9.02 (s, 1H), 8.55 (d, J=4.8 Hz, 1H), 8.20 (s, 1H), 8.13 (d, J=8.0 Hz, 1H), 7.68 (s, 1H), 7.35 (dd, J=7.5, 4.9 Hz, 1H), 6.52 (s, 1H), 3.70-3.60 (m, 2H), 3.63 (s, 3H), 1.20 (d, 0.1=6.1 Hz, 6H). LC-MS m/z: 275.2 [M+H]⁺. HPLC Purity (214 nm): >96%; t_{R}=4.88 min.

### Example 25—4-Phenyl-N-(2-(1-(trifluoromethyl)cyclopropyl)ethyl)-1H-imidazole-1-carboxamide

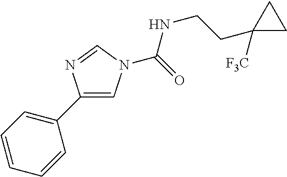 [SMILES: O=C(NCCC1(C(F)(F)F)CC1)n1cnc(-c2ccccc2)c1]

Following general procedure C, 4-phenyl-1H-imidazole (30 mg, 0.2 mmol) and 2-(1-(trifluoromethyl)cyclopropyl)ethanamine hydrochloride (40 mg, 0.2 mmol) afforded the title compound (12.3 mg, 18%) as a white solid. ¹H NMR (400 MHz, CDCl₃) δ 8.14 (s, 1H), 7.78 (d, J=6.8 Hz, 2H), 7.55 (s, 1H), 7.41 (t, J=7.6 Hz, 2H), 7.33-7.28 (m, 1H), 5.91 (s, 1H), 3.62 (q, J 6.4 Hz, 2H), 1.92 (t, J=7.2 Hz, 2H), 1.05 (t, J=5.6 Hz, 2H), 0.69 (s, 2H). LC-MS m/z: 324.1 [M+H]⁺. HPLC Purity (214 nm): 95%; t_{R}=8.54 min.

### Example 26—N-(2-Cyclopropylethyl)-4-phenyl-1H-imidazole-1-carboxamide

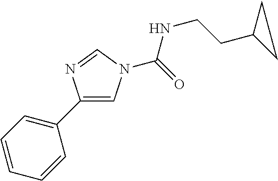 [SMILES: O=C(NCCC1CC1)n1cnc(-c2ccccc2)c1]

Following general procedure C, 4-phenyl-1H-imidazole (200 mg, 1.4 mmol) and 2-cyclopropylethanamine hydrochloride (182 mg, 1.5 mmol) afforded the title compound (118.1 mg, 33%) as a white solid. ¹H NMR (400 MHz, CDCl₃) δ 8.14 (s, 1H), 7.79 (d, J=7.6 Hz, 2H), 7.57 (s, 1H), 7.41 (t, J=7.6 Hz, 2H), 7.30 (t, J=7.2 Hz, 1H), 5.79 (s, 1H), 3.56 (q, J=6.4 Hz, 2H), 1.57 (t, J=7.2 Hz, 2H), 0.76-0.72 (m, 1H), 0.56-0.51 (m, 2H), 0.14 (q, J=4.8 Hz, 2H) LC-MS m/z: 256.1 [M+H]⁺. HPLC Purity (214 nm): 100%; t_{R}=7.30 min.

### Example 27—N-iso-Pentyl-4-phenyl-1H-imidazole-1-carboxamide

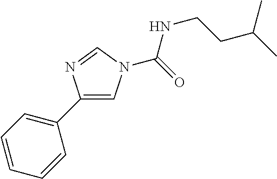 [SMILES: CC(C)CCNC(=O)n1cnc(-c2ccccc2)c1]

Following general procedure C, 4-phenyl-1H-imidazole (500 mg, 3.47 mmol) and 3-methylbutan-1-amine (335 mg, 3.85 mmol) afforded the title compound (40 mg, 4.7%) as a white solid. ¹H NMR (500 MHz, CDCl₃) δ 8.15 (s, 1H), 7.79 (d, J=7.3 Hz, 2H), 7.6 (d, J=1.1 Hz, 1H), 7.40 (t, J=7.6 Hz, 2H), 7.30 (t, J=7.4 Hz, 1H), 5.68 (s, 1H), 3.48 (dd, J=14.7, 6.0 Hz, 2H), 1.61 (m, 2H), 0.99 (s, 6H). LC-MS m/z: 258.1 [M+H]⁺. HPLC Purity (214 nm): 100%; t_{R}=7.67 min.

### Example 28—4-(6-Cyanopyridin-3-yl)-N-phenethyl-1H-imidazole-1-carboxamide

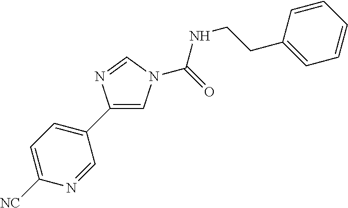 [SMILES: N#Cc1ccc(-c2cn(C(=O)NCCc3ccccc3)cn2)cn1]

Following general procedure D, 4-bromo-1H-imidazole (5.0 g, 34.0 mmol) and 6-fluoropyridin-3-ylboronic acid (4.8 g, 34.0 mmol) afforded 2-fluoro-5-(1H-imidazol-4-yl)pyridine (2.5 g, 94%) as a yellow oil. LC-MS m/z: 164.2 [M+H]⁺. HPLC Purity (214 nm): 94%; t_{R}=1.44 min.

A suspension of 2-fluoro-5-(1H-imidazol-4-yl)pyridine (2.5 g, 15.3 mmol) and NaCN (1.9 g, 38.3 mmol) in DMSO (20 mL) was stirred at 130° C. under N₂ for 16 h and then poured into water (20 mL) and extracted with DCM (20 mL×3). The combined organic layers were dried over Na₂SO₄, filtered and concentrated in vacuo to give a residue which was purified by prep-HPLC (FA) to afford 5-(1H-imidazol-4-yl)picolino-nitrile (83 mg, 98%) as a white solid. LC-MS m/z: 171.3 [M+H]⁺. HPLC Purity (214 nm): 98%; t_{R}=1.18 min.

Following general procedure B (method 1), 5-(1H-imidazol-4-yl)picolinonitrile (83 mg, 0.49 mmol) and (2-isocyanatoethyl)benzene (108 mg, 0.74 mmol) afforded the title compound (28.1 mg, 100%) as a white solid. ¹H NMR (400 MHz, DMSO-d₆) δ 9.20 (d, J=1.2 Hz, 1H), 8.81 (brs, 1H), 8.49 (s, 1H), 8.42 (s, 1H), 8.38 (dd, J=8.1, 2.0 Hz, 1H), 8.06 (d, J=8.1 Hz, 1H), 7.35-7.18 (m, 5H), 3.55-3.48 (m, 2H), 2.89 (t, J=7.3 Hz, 2H). LC-MS m/z: 318.0 [M+H]⁺. HPLC Purity (214 nm): 100%; t_{R}=7.50 min.

### Example 29—4-(6-Methylpyridin-3-yl)-N-phenethyl-1H-imidazole-1-carboxamide

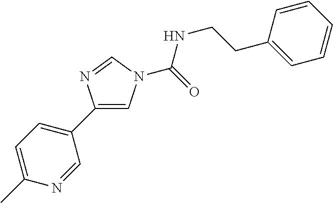 [SMILES: Cc1ccc(-c2cn(C(=O)NCCc3ccccc3)cn2)cn1]

To a solution of 1-(6-methylpyridin-3-yl)ethanone (500 mg, 3.7 mmol) in ether (10 mL) was added Br₂ (0.15 mL) at 0° C. and the mixture was stirred at RT overnight. The resulting mixture was filtered to afford 2-bromo-1-(6-methylpyridin-3-yl)ethanone (1 g, crude) as a yellow solid. LC-MS m/z: 214[M+H]⁺. HPLC Purity (214 nm): 67%; t_{R}=1.73 min.

A mixture of 2-bromo-1-(6-methylpyridin-3-yl)ethanone (1 g, 4.7 mmol) in methanamide (10 mL) was stirred at 180° C. for 2 h. The solution was then cooled, concentrated and purified by silica gel column chromatography (DCM:MeOH=20:1) to afford 5-(1H-imidazol-4-yl)-2-methylpyridine (150 mg, 26% over two steps) as a yellow solid. LC-MS m/z: 160[M+H]⁺. HPLC Purity (254 nm): 96%; t_{R}=1.48 min.

Following general procedure B (method 1), 5-(1H-imidazol-4-yl)-2-methylpyridine (150 mg, 0.94 mmol) and (2-isocyanatoethyl)benzene (208 mg, 1.41 mmol) afforded the title compound (149 mg, 52%) as a white solid. ¹H NMR (400 MHz, DMSO-d₆) δ 8.88 (s, 1H), 8.70 (t, J=5.2 Hz, 1H), 8.33 (s, 1H), 8.18 (s, 1H), 8.03 (dd, J=8.0, 2.4 Hz, 1H), 7.23-7.32 (m, 6H), 3.50-3.52 (m, 2H), 2.89 (t, J=7.2 Hz, 2H), 2.48 (s, 3H). LC-MS m/z: 307 [M+H]⁺. HPLC Purity (214 nm): 96%; t_{R}=8.35 min.

### Example 30—4-(6-Fluoropyridin-3-yl)-N-phenethyl-1N-imidazole-1-carboxamide

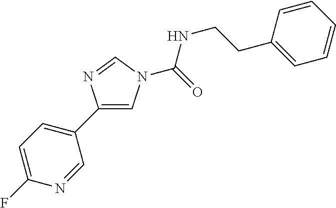 [SMILES: O=C(NCCc1ccccc1)n1cnc(-c2ccc(F)nc2)c1]

Following general procedure D, 4-bromo-1H-imidazole (1.30 g, 8.82 mmol) and 6-fluoropyridin-3-ylboronic acid (1.04 g, 7.35 mmol) afforded 2-fluoro-5-(1H-imidazol-4-yl)pyridine (500 mg, 41.7%) as a brown solid. LC-MS m/z: 164.0 [M+H]⁺. HPLC Purity (214 nm): >94%; t_{R}=1.06 min.

Following general procedure B (method 1), 2-fluoro-5-(1H-imidazol-4-yl)pyridine (300 mg, 1.84 mmol) and (2-isocyanatoethyl)benzene (298 mg, 2.02 mmol) in DCM afforded the title compound (57.4 mg 10.6%) as a white solid. ¹H NMR (500 MHz, CDCl₃) δ 8.57 (d, J=2.4 Hz, 1H), 8.18 (dt, J=9.4, 2.4 Hz, 1H), 8.06 (d, J=1.3 Hz, 1H), 7.53 (d, J=1.3 Hz, 1H), 7.37 (t, J=7.2 Hz, 2H), 7.30-7.21 (m, 3H), 6.98 (dd, J=8.5, 2.8 Hz, 1H), 5.70 (s, 1H), 3.74 (q, J=6.8 Hz, 2H), 2.98 (t, J=6.8 Hz, 2H). LC-MS m/z: 311.2 [M+H]⁺. HPLC Purity (214 nm): >99%; t_{R}=1.89 min.

### Example 31—4-(6-Methoxypyridin-3-yi)-N-phenethyl-1H-imidazole-1-carboxamide

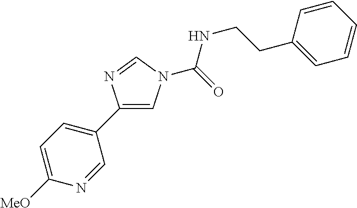 [SMILES: COc1ccc(-c2cn(C(=O)NCCc3ccccc3)cn2)cn1]

A solution of 1-(6-methoxypyridin-3-yl)ethanone (2.0 g, 13 mmol) and Br₂ (2.1 g, 13 mmol) in dioxane (20 mL) was stirred at 50° C. for 6 h and then concentrated in vac-no. The residue was purified by silica gel column chromatography (PE:EA=20:1) to afford 2-bromo-1-(6-methoxypyridin-3-yl)ethanone (500 mg, 17%) as a yellow solid. LC-MS m/z: 231.1 [M+H]⁺. HPLC Purity (214 nm): 99%; t_{R}=1.55 min.

Following general procedure A, 2-bromo-1-(6-methoxypyridin-3-yl)ethanone (500 mg, 2.17 mmol) afforded 5-(1H-imidazol-4-yl)-2-methoxypyridine (110 mg, 29%). LC-MS m/z: 176.1 [M+H]⁺. HPLC Purity (254 nm): >99%; t_{R}=1.35 min.

Following general procedure B (method 1), 5-(1H-imidazol-4-yl)-2-methoxypyridine (100 mg, 0.57 mmol) and (2-isocyanatoethyl)benzene (167 mg, 1.14 mmol) in DCM afforded the title compound (13 mg, 7.0%) as a white solid. ¹H NMR (400 MHz, CDCl₃) δ 8.53 (d, J=2.4 Hz, 1H), 8.03 (d, J=1.2 Hz, 1H), 7.92 (d, J=2.4 Hz, 1H), 7.40 (d, J=1.2 Hz, 1H), 7.38-7.21 (m, 5H), 6.78 (d, J=8.8 Hz, 1H), 5.65 (s, 1H), 3.96 (s, 3H), 3.72 (q, J=6.4 Hz, 2H), 2.98 (t, J=6.8 Hz, 2H). LC-MS m/z: 323.0 [M+H]⁺. HPLC Purity (214 nm): >97%; t_{R}=1.65 min.

### Example 32—N-(3-Cyclopropylpropyl)-4-(pyridin-3-yl)-1H-imidazole-1-carboxamide

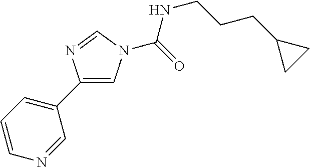 [SMILES: O=C(NCCCC1CC1)n1cnc(-c2cccnc2)c1]

To a solution of 3-cyclopropylpropanenitrile (300 mg, 3.2 mmol) in THF (14 mL) was added LAH (240 mg, 6.4 mmol) and AlCl₃ (850 mg, 6.4 mmol) at RT and then the mixture was stirred at 50° C. for 1 h. To the resulting mixture was added Na₂SO₄.10H₂O and the mixture was stirred at RT for 1 h, filtered and concentrated in vacuo to give 3-cyclopropylpropan-1-amine (300 mg) as a colorless oil, which was used directly in the next step. A solution of 2-bromo-1-(pyridin-3-yl) ethanone (3 g, 16 mmol) in acetamide (15 mL) was stirred at 180° C. for 3 h. The result mixture was concentrated in vacuo to give a residue which was purified by silica gel column chromatography (DCM/MeOH=10/1) to give 3-(1H-imidazol-4-yl) pyridine (1.82 g, 83.1%) as a brown oil. LC-MS m/z: 146.2 [M+H]⁺. HPLC Purity (254 nm): 100%; t_{R}=0.21 min.

Following general procedure C, 3-(1H-imidazol-4-yl)pyridine (100 mg, 0.7 mmol) and 3-cyclopropylpropan-1-amine (100 mg, 0.9 mmol) afforded the title compound (14.1 mg, 7.5%) as a yellow solid. ¹H NMR (400 MHz, CDCl₃) δ 8.97 (d, J=1.7 Hz, 1H), 8.51 (dd, J=4.8, 1.5 Hz, 1H), 8.20 (d, J=1.2 Hz, 1H), 8.12 (dt, J=7.9, 1.9 Hz, 1H), 7.73 (d, J=1.0 Hz, 1H), 7.34 (dd, J=7.6, 5.1 Hz, 1H), 6.32 (s, 1H), 3.50 (td, J=7.3, 5.9 Hz, 2H), 1.80-1.75 (m, 2H), 1.31 (dd, J=14.7, 7.0 Hz, 2H), 0.70 (tt, J=7.0, 3.5 Hz, 1H), 0.51-0.41 (m, 2H), 0.09-0.02 (m, 2H). LC-MS m/z: 271.0 [M+H]⁺. HPLC Purity (254 nm): 98.29%; t_{R}=8.01 min.

### Example 33—N-(4-Methylpentyl 4-(pyridin-3-yl)-1H-imidazole-1-carboxamide

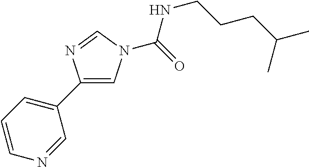 [SMILES: CC(C)CCCNC(=O)n1cnc(-c2cccnc2)c1]

Following general procedure C, 3-(1H-imidazol-4-yl)pyridine (100 mg, 0.7 mmol) and 4-methylpentan-1-amine (70 mg, 0.7 mmol) afforded the title compound (27.9 mg, 15.0%) as a white solid. ¹H NMR (400 MHz, CDCl₃) δ 9.00 (s, 1H), 8.53 (s, 1H), 8.19 (d, J=1.1 Hz, 1H), 8.13 (d, J=7.9 Hz, 1H), 7.70 (s, 1H), 7.35 (s, 1H), 6.02 (s, 1H), 3.45 (dd, J=13.2, 7.3 Hz, 2H), 1.68 (d, J=7.5 Hz, 1H), 1.59 (dd, J=13.4, 6.7 Hz, 2H), 1.33-1.23 (m, 2H), 0.91 (d, J=6.6 Hz, 6H). LC-MS m/z: 273.0 [M+H]⁺. HPLC purity (214 nm): 100%; t_{R}=8.43 min.

### Example 34—4-(4-((Dimethylamino)methyl)phenyl)-N-phenethyl-1H-imidazole-1-carboxamide

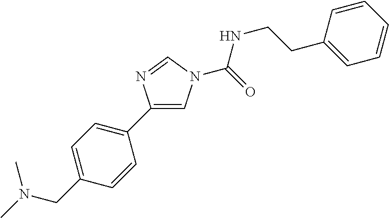 [SMILES: CN(C)Cc1ccc(-c2cn(C(=O)NCCc3ccccc3)cn2)cc1]

A solution of 1-bromo-4-(bromomethyl)benzene (1.2 g, 5.0 mmol) in dimethylamine/THF (10 mL, 4 N) was stirred at RT for 16 h and concentrated. The residue was basified by Na₂CO₃ solution to pH=9 and extracted with EA (30 mL). The organic layers were concentrated to afford 1-(4-bromophenyl)-N,N-dimethylmethanamine (640 mg, 60%) as a white solid. LC-MS m/z: 214.1 [M+H]⁺. HPLC Purity (214 nm): 99%; t_{R}=0.60 min.

A mixture of 1-(4-bromophenyl)-N,N-dimethylmethanamine (639 mg, 3.0 mmol), 4,4,4′,4′,5,5,5′,5′-octamethyl-2,2′-bi(1,3,2-dioxaborolane) (4.1 g, 16.0 mmol), Pd(dppf)Cl₂.DCM (915 mg, 3.6 mmol) and KOAc (600 mg, 6.0 mmol) in 1,4-dioxane (10 mL) was stirred at 100° C. for 12 h under N₂. The reaction mixture was cooled and concentrated in vacuo to give a residue which was purified by silica gel column chromatography (DCM/MeOH=10/1) to give N,N-dimethyl-1-(4-(4,4,5,5-tetramethyl-1,3,2-dioxaborolan-2-yl)phenyl)methanamine (530 mg, 67%) as a white solid. LC-MS m/z: 262.4 [M+H]⁺. HPLC Purity (214 nm): 79%; t_{R}=0.75 min.

Following general procedure D, 2-bromo-1-methyl-1H-imidazole (600 mg, 4.0 mmol) and N,N-dimethyl-1-(4-(4,4,5,5-tetramethyl-1,3,2-dioxaborolan-2-yl)phenyl) methanamine (522 mg, 2.0 mmol) afforded 1-(4-(1H-imidazol-4-yl)phenyl)-N,N-dimethylmethanamine (202 mg, 51%) as a yellow solid. LC-MS m/z: 202.1 [M+H]⁺. HPLC Purity (214 nm): 92%; t_{R}=1.11 min.

Following general procedure B (method 1), 1-(4-(1H-imidazol-4-yl)phenyl)-N,N-dimethylmethanamine (207 mg, 1.0 mmol) and (2-isocyanatoethyl)benzene (147 mg, 1.0 mmol) afforded the title compound (28.7 mg, 8.2%) as a white solid. ¹H NMR (400 MHz, CDCl₃) δ 8.05 (d, J=1.3 Hz, 1H), 7.73 (d, J=8.2 Hz, 2H), 7.50 (d, J=1.2 Hz, 1H), 7.41-7.32 (m, 5H), 7.25-7.20 (m, 2H), 5.78 (brs, 1H), 3.72 (q, J=6.4 Hz, 2H), 3.48 (s, 2H), 2.97 (t, J=6.8 Hz, 2H), 2.30 (s, 6H). LC-MS m/z: 349.2 [M+H]⁺. HPLC Purity (214 nm): 100%; t_{R}=8.54 min.

### Example 35—N-iso-Butyl-4-(pyridin-3-yl)-1H-imidazole-1-carboxamide

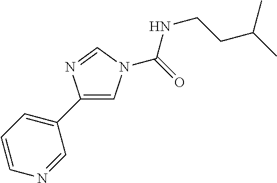 [SMILES: CC(C)CCNC(=O)n1cnc(-c2cccnc2)c1]

Following general procedure C, 3-(1H-imidazol-4-yl)pyridine (180 mg, 1.24 mmol) and 2-methylpropan-1-amine (108 mg, 1.86 mmol) afforded the title compound (43.0 mg, 14.2%) as a yellow solid. 1H NMR (400 MHz, CDCl₃) δ 9.00 (s, 1H), 8.54 (d, J=3.6 Hz, 1H), 8.18 (s, 1H), 8.13 (d, J=8.1 Hz, 1H), 7.67 (s, 1H), 7.35 (dd, J=7.6, 4.8 Hz, 1H), 5.77 (s, 1H), 3.31 (t, J=6.4 Hz, 2H), 1.95 (dd, J=13.8, 7.0 Hz, 1H), 1.02 (d, J=6.7 Hz, 6H). LC-MS m/z: 245.3[M+H]⁺. HPLC Purity (214 nm): 100%; t_{R}=4.41 min.

### Example 36—N-iso-Pentyl-4-(6-methoxypyridin-3-yl)-1H-imidazole-1-carboxamide

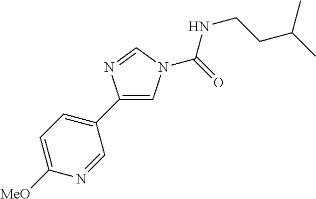 [SMILES: COc1ccc(-c2cn(C(=O)NCCC(C)C)cn2)cn1]

Following general procedure D, 4-bromo-1H-imidazole (576 mg, 3.92 mmol) and (6-methoxypyridin-3-yl)boronic acid (500 mg, 3.92 mmol) afforded crude 5-(1H-imidazol-4-yl)-2-methoxy pyridine (380 mg, 55%) as a white solid. LC-MS m/z: 176.7 [M+H]⁺. HPLC purity (214 nm): 95%; t_{R}=1.47 min.

Following general procedure B (method 2), 5-(1H-imidazol-4-yl)-2-methoxy pyridine (320 mg, 1.83 mmol) and methylbutan-1-amine (335 mg, 3.85 mmol) afforded the title compound (154.7 mg, 29.4%) as a white solid. ¹H NMR (400 MHz, CDCl₃) δ 8.50 (d, J=2 Hz, 1H), 8.10 (t, J=1.2 Hz, 1H), 7.95 (dd, J=8.4, 2.4 Hz, 1H), 7.48 (s, 1H), 6.79 (d, J=8.8 Hz, 1H), 5.67 (s, 1H), 4.63 (s, 3H), 3.54-3.59 (m, 2H), 1.81-1.82 (m, 1H), 1.66-1.71 (m, 2H), 1.12 (d, J=6.4 Hz, 6H). LC-MS m/z: 289.7 [M+H]⁺. HPLC Purity (214 nm): 96.27%; t_{R}=7.25 min.

### Example 37—N-iso-Pentyl-4-(6-methylpyridin-3-yl)-1H-imidazole-1-carboxamide

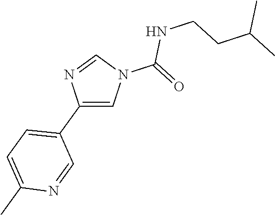 [SMILES: Cc1ccc(-c2cn(C(=O)NCCC(C)C)cn2)cn1]

To a solution of 4-bromo-1H-imidazole (2.9 g, 20 mmol) in THF (20 mL) was added NaH (1.6 g, 40 mmol) at 0° C. The mixture was stirred at 0° C. for 30 min and PMBCl (3.7 g, 24 mmol) was added. The solution was stirred at 50° C. for 16 h and poured into ice-water (20 ml). The mixture was extracted with EA (50 mL) and the organic layers were concentrated and purified by silica gel column chromatography (PEEA=1/1) to give 4-bromo-1-(4-methoxybenzyl)-1H-imidazole (3.3 g, 62%) as a yellow oil. LC-MS m/z: 268.8 [M+H]⁺. HPLC Purity (254 nm): 65.84%; t_{R}=1.61 min.

Following general procedure D, 4-bromo-1-(4-methoxybenzyl)-1H-imidazole (897 mg, 3.4 mmol) and 6-methylpyridin-3-ylboronic acid (600 mg, 4.4 mmol) afforded 5-(1-(4-methoxybenzyl)-1H-imidazol-4-yl)-2-methylpyridine (700 mg, 74%) as a yellow oil. LC-MS m/z: 280.1 [M+H]⁺. HPLC Purity (214 nm): 37.54%; t_{R}=1.58 min.

To a solution of 5-(1-(4-methoxybenzyl)-1H-imidazol-4-yl)-2-methylpyridine (600 mg, 2.2 mmol) in THF (10 mL) was added Ce(NH₄)₂(NO₃)₆ (2.4 g, 4.4 mmol) and the mixture was stirred at 50° C. for 16 h and purified by silica gel column chromatography (DCM/MeOH=5/1) to give 5-(1H-imidazol-4-yl)-2-methylpyridine (200 mg, 57%) as a yellow solid. LC-MS m/z: 160.0 [M+H]⁺. HPLC Purity (254 nm): 26.59%; t_{R}=1.20 min.

Following general procedure B (method 2), 5-(1H-imidazol-4-yl)-2-methylpyridine (80 mg, 0.5 mmol) and 3-methylbutylamine (52 mg, 0.6 mmol) afforded the title compound (19.4 mg, 14.2%) as a yellow solid. ¹H NMR (400 MHz, CDCl₃) δ 8.86 (d, J=1.9 Hz, 1H), 8.16 (d, J=1.3 Hz, 1H), 8.00 (dd, J=8.0, 2.3 Hz, 1H), 7.61 (d, J=1.3 Hz, 1H), 7.20 (d, J=8.0 Hz, 1H), 5.78 (s, 1H), 3.65-3.35 (m, 2H), 2.58 (s, 3H), 1.88-1.37 (m, 5H), 0.97 (d, J=6.6 Hz, 6H). LC-MS m/z: 273.1 [M+H]⁺. HPLC Purity (214 nm): 97.36%; t_{R}=5.97 min.

### Example 38—N-iso-Pentyl-4-(2-(1-methylpiperidin-4-yl)phenyl)-1H-imidazole-1-carboxamide

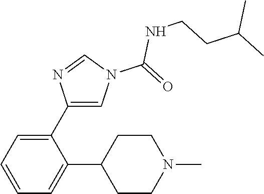 [SMILES: CC(C)CCNC(=O)n1cnc(-c2ccccc2C2CCN(C)CC2)c1]

A solution of 1-(2-bromophenyl)ethan-1-one (10.0 g, 50.2 mmol), NBS (8.94 g, 50.2 mmol) and TsOH (12.9 g, 75.3 mmol) in MeCN (100 mL) was stirred at 80° C. for 8 h. The reaction mixture was cooled and concentrated in vacuo to give a residue which was purified by silica gel column chromatography (PE:EA=9:1) to afford 2-bromo-1-(2-bromophenyl)ethan-1-one (11.6 g, 83.8%) as a brown oil. LC-MS m/z: 279.0 [M+H]⁺. Purity (214 nm): 94%; t_{R}=1.34 min.

Following general procedure A, 2-bromo-1-(2-bromophenyl)ethan-1-one (11.6 g, 42.0 mmol) in formamide (20 mL) afforded 4-(2-bromophenyl)-1H-imidazole (10.3 g, 91%) as a brown oil. LC-MS m/z: 225.1 [M+H]⁺. Purity (214 nm): 97%; t_{R}=0.65 min.

To a stirred solution of NaH (1.8 g, 45.0 mmol) in THF (20 mL) was added 4-(2-bromophenyl)-1H-imidazole (5.0 g, 22.5 mmol) at 0° C. and the mixture was stirred for 30 min. PMBCl (5.29 g, 33.7 mmol) was added and the reaction mixture was stirred at RT for 16 h. The reaction mixture was diluted with EA (100 mL), washed with water (30 mL×3) and brine (30 mL), dried over Na₂SO₄, filtered, concentrated and purified by silica gel column chromatography (EA:PE=1:3) to give 4-(2-bromophenyl)-1-(4-methoxybenzyl)-1H-imidazole (3.7 g, 48.1%) as a yellow solid. LC-MS m/z: 344.5 [M+H]⁺. Purity (214 nm): 99%; t_{R}=1.64 min.

A mixture of 4-(2-bromophenyl)-1-(4-methoxybenzyl)-1H-imidazole (600 mg, 1.75 mmol), 1-methyl-4-(4,4,5,5-tetramethyl-1,3,2-dioxaborolan-2-yl)-1,2,3,6-tetrahydropyridine (469 mg, 2.10 mmol), Na₂CO₃ (483 mg, 3.5 mmol) and Pd[(tBu)₃P]2 (89.6 mg, 0.18 mmol) in anhydrous DMF (5 mL) was stirred at 120° C. under N₂ for 3 h under microwave conditions. The reaction was cooled and concentrated in vacuo to give a residue which was purified by silica gel column chromatography (DCM:MeOH=6:1) to give 4-(2-(1-(4-methoxybenzyl)-1H-imidazol-4-yl)phenyl)-1-methyl-1,2,3,6-tetrahydropyridine (440 mg, 70.0%) as a brown oil. LC-MS m/z: 360.4 [M+H]⁺. Purity (214 nm): 99% t_{R}=0.68 min.

To a solution of 4-(2-(1-(4-methoxybenzyl)-1H-imidazol-4-yl)phenyl)-1-methyl-1,2,3,6-tetrahydropyridine (440 mg, 1.22 mmol) in MeOH (20.0 mL) was added PtO₂ (150 mg). The mixture was stirred at RT under H₂ for 16 h, filtered and concentrated to afford 4-(2-(1-(4-methoxybenzyl)-1H-imidazol-4-yl)phenyl)-1-methylpiperidine (400 mg, 90%) as a black oil. LC-MS m/z: 362.2 [M+H]⁺. Purity (214 nm): 87%; t_{R}=1.77 min.

To a solution of 4-(2-(1-(4-methoxybenzyl)-1H-imidazol-4-yl)phenyl)-1-methylpiperidine (300 mg, 0.83 mmol) in MeOH (10.0 mL) was added Pd/C (200 mg). The mixture was stirred at 55° C. under H₂ for 16 h. The mixture was filtered and concentrated to afford 4-(2-(1H-imidazol-4-yl)phenyl)-1-methylpiperidine (100 mg, 50%) as a yellow oil. LC-MS m/z: 242.2 [M+H]⁺. Purity (214 nm): 87%; t_{R}=1.31 min.

Following general procedure C, 4-(2-(1H-imidazol-4-yl)phenyl)-1-methylpiperidine (200 mg, 0.83 mmol) and 3-methylbutan-1-amine (108 mg, 1.25 mmol) afforded the title compound (40 mg, 14%) as a yellow solid. ¹H NMR (400 MHz, CDCl₃) δ 8.27 (d, J=1.2 Hz, 1H), 7.62 (d, 0.1=0.4 Hz, 1H), 7.49 (s, 1H), 7.37-7.28 (m, 3H), 7.20 (t, J=7.9 Hz, 1H), 3.47 (dd, J=14.7, 5.8 Hz, 2H), 3.33 (d, J=10.3 Hz, 2H), 2.59 (s, 3H), 2.54-2.44 (m, 3H), 2.15-2.05 (m, 2H), 1.79 (d, J=12.9 Hz, 2H), 1.75-1.68 (m, 1H), 1.58 (q, J=7.6 Hz, 2H), 0.97 (d, J=6.6 Hz, 6H). LC-MS m/z: 355.2 [M+H]⁺. HPLC Purity (214 nm): 100%; t_{R}=5.87 min.

### Example 39—N-(5-Methylhexyl)-4-(pyridin-3-yl)-1H-imidazole-1-carboxamide

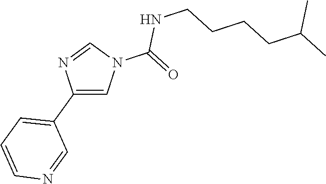 [SMILES: CC(C)CCCCNC(=O)n1cnc(-c2cccnc2)c1]

Following general procedure C, 3-(1H-imidazol-4-yl)pyridine (145 mg, 1.0 mmol) and 5-methylhexylamine (115 mg, 1 mmol) afforded the title compound (47 mg, 16.2%) as a yellow solid. ¹H NMR (400 MHz, CDCl₃) δ 8.97 (s, 1H), 8.51 (d, J=4 Hz, 1H), 8.20 (s, 1H), 8.12 (d, J=7.6 Hz, 1H), 7.72 (s, 1H), 7.36-7.33 (m, 1H), 6.30 (brs, 1H), 3.49-3.42 (m, 2H), 1.72-1.60 (m, 2H), 1.59-1.49 (m, 1H), 1.44-1.34 (m, 2H), 1.26-1.18 (m, 2H), 0.88 (d, J=6.4 Hz, 6H). LC-MS m/z: 272.7 [M+H]⁺. HPLC Purity (214 nm): 100%; t_{R}=5.95 min.

### Example 40—N-(4-Cyclopropylbutyl)-4-(thiazol-2-yl)-1H-imidazole-1-carboxamide

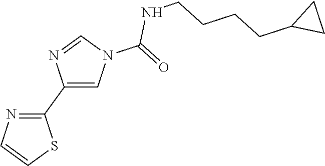 [SMILES: O=C(NCCCCC1CC1)n1cnc(-c2nccs2)c1]

Following general procedure C, 2-(1H-imidazol-4-yl)thiazole (0.10 g, 0.66 mmol) and 4-cyclopropylbutan-1-amine hydrochloride (0.11 g, 0.73 mmol) afforded the title compound (41.3 mg, 24.0%) as a white solid. ¹H NMR (400 MHz, CDCl₃) δ 8.21 (d, J=1.2 Hz, 1H), 7.91 (d, J=1.2 Hz, 1H), 7.81 (d, 0.1=3.2 Hz, 1H), 7.33 (d, 0.1=2.8 Hz, 1H), 5.96 (brs, 1H), 3.43 (q, J=7.2 Hz, 2H), 1.79-1.62 (m, 2H), 1.51-1.44 (m, 2H), 1.25 (q, J=7.2 Hz, 2H), 0.67-0.63 (m, 1H), 0.44-0.39 (m, 2H), 0.03-0.00 (m, 2H). LC-MS m/z: 291.0 [M+H]⁺. HPLC Purity (214 nm): >98%; t_{R}=6.85 min.

### Example 41—N-iso-Pentyl-4-(pyridin-4-yl)-1H-imidazole-1-carboxamide

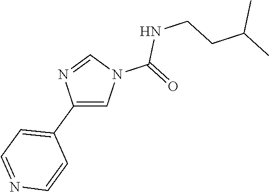 [SMILES: CC(C)CCNC(=O)n1cnc(-c2ccncc2)c1]

Following general procedure A, 2-bromo-1-(pyridin-4-yl)ethenone (300 mg, 1.5 mmol) afforded 4-(1H-imidazol-4-yl)pyridine as a brown oil (200 mg, 91%). LC-MS m/z: 146 [M+H]⁺. HPLC Purity (214 nm): 80%; t_{R}=0.38 min.

Following general procedure C, 4-(1H-imidazol-4-yl)pyridine (200 mg, 1.37 mmol) and 3-methylbutylamine (195 mg, 2.23 mmol) afforded the title compound (4.4 mg, 1.2%) as a white solid. ¹H NMR (400 MHz, CDCl₃) δ 8.61 (dd, J=4.6, 1.5 Hz, 2H), 8.17 (d, J=1.3 Hz, 1H), 7.81 (d, J=1.3 Hz, 1H), 7.67 (dd, J=4.6, 1.6 Hz, 2H), 5.97 (brs, 1H), 3.49 (dt, J=7.5, 5.8 Hz, 2H), 2.20-1.63 (m, 1H), 1.59-1.54 (m, 2H), 0.98 (d, J=6.4 Hz, 6H). LC-MS m/z: 259 [M+H]⁺. HPLC Purity (214 nm): 99%; t_{R}=4.48 min.

### Example 42—N-Benzyl-4-(pyridin-3-yl-1H-imidazole-1-carboxamide

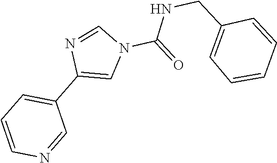 [SMILES: O=C(NCc1ccccc1)n1cnc(-c2cccnc2)c1]

Following general procedure B (method 1), 3-(1H-imidazol-4-yl)pyridine (200 mg, 1.38 mmol) and benzyl isocyanate (270 mg, 2.07 mmol) afforded the title compound as a white solid (44.5 mg, 10.1%). ¹H NMR (400 MHz, CDCl₃) δ 8.95 (d, J=1.6 Hz, 1H), 8.45 (dd, J=4.8, 1.6 Hz, 1H), 8.21 (d, J=1.3 Hz, 1H), 8.11 (dt, J=7.9, 1.9 Hz, 1H), 7.71 (d, J=1.3 Hz, 1H), 7.49-7.26 (m, 6H), 6.49 (brs, 1H), 4.65 (d, J=5.6 Hz, 2H). LC-MS m/z: 279.0 [M+H]⁺. HPLC Purity (214 nm): 100%; t_{R}=1.78 min.

### Example 43—4-(6-iso-Propylpyridin-3-yl)-N-phenethyl-1H-imidazole-1-carboxamide

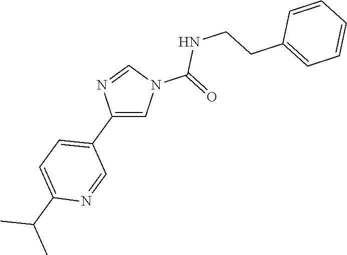 [SMILES: CC(C)c1ccc(-c2cn(C(=O)NCCc3ccccc3)cn2)cn1]

To a solution of 1-(6-bromopyridin-3-yl)ethan-1-one (2 g, 9.99 mmol) in DCM (10 mL) was added HBr (0.2 mL), followed by Br₂ (1.9 g, 11.998 mmol) and the mixture was stirred at RT for 2 days. The mixture was filtered and concentrated to give 2-bromo-1-(6-bromopyridin-3-yl)ethan-1-one (3 g, crude) as a gray solid. LC-MS m/z: 280.0 [M+H]⁺. HPLC Purity (214 nm): 79.29%; t_{R}=0.99 min.

Following general procedure A, 2-bromo-1-(6-bromopyridin-3-yl)ethan-1-one (2.7 g, 9.68 mmol) afforded 2-bromo-5-(1H-imidazol-4-yl) pyridine (670 mg, 31%) as a light brown semisolid. LC-MS m/z: 224.0 [M+H]⁺. HPLC Purity (214 nm): 80%; t_{R}=1.60 min.

Following general procedure D, 2-bromo-5-(1H-imidazol-4-yl)pyridine (520 mg, 2.32 mmol) and 4,4,5,5-tetramethyl-2-(prop-1-en-2-yl)-1,3,2-dioxaborolane (468 mg, 2.78 mmol) afforded 5-(1H-imidazol-4-yl)-2-(prop-1-en-2-yl)pyridine (320 mg, 74.5%) as a light brown oil. LC-MS m/z: 186.1 [M+H]⁺. HPLC Purity (214 nm): 80.79%; t_{R}=1.68 min.

To a solution of 5-(1H-imidazol-4-yl)-2-(prop-1-en-2-yl)pyridine (320 mg, 1.73 mmol) in MeOH (5 mL) was added Pd/C (10%) (50 mg). The mixture was stirred at RT under H₂ for 4 h and then the mixture was filtered and concentrated to give 5-(1H-imidazol-4-yl)-2-isopropylpyridine (285 mg, 88%) as a brown solid. LC-MS m/z: 188.1 [M+H]⁺. Purity (214 nm): 96.4%; t_{R}=1.675 min.

Following general procedure C, 5-(1H-imidazol-4-yl)-2-isopropylpyridine (285 mg, 1.522 mmol) and 2-phenylethan-1-amine (184 mg, 1.522 mmol) afforded the title compound (20.4 mg, 4.0%) as a white solid. ¹H NMR (400 MHz, CDCl₃) δ 8.84 (d, J=1.7 Hz, 1H), 8.06 (d, J=1.3 Hz, 1H), 8.01 (dd, J=8.1, 2.3 Hz, 1H), 7.50 (d, J=1.3 Hz, 1H), 7.38-7.32 (m, 2H), 7.30-7.20 (m, 4H), 5.80 (brs, 1H), 3.72 (dt, J=12.7, 6.8 Hz, 2H), 3.08 (septet, J=6.9 Hz, 1H), 2.98 (t, J=6.8 Hz, 2H), 1.32 (d, J=6.9 Hz, 6H). LC-MS m/z: 335.1 [M+H]⁺. HPLC Purity (214 nm): 100%; t_{R}=6.46 min.

### Example 44—4-(5-((Dimethylamino)methyl)pyridin-3-yl)-N-phenethyl-1H-imidazole-1-carboxamide

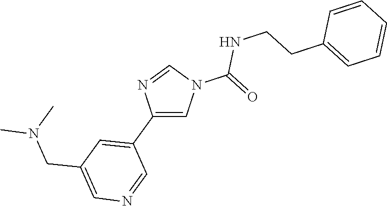 [SMILES: CN(C)Cc1cncc(-c2cn(C(=O)NCCc3ccccc3)cn2)c1]

To a solution of 5-bromonicotinaldehyde (2 g, 10.7 mmol) in toluene (30 mL) was added 4-bromo-1-((2-(trimethylsilyl)ethoxy)methyl)-1H-imidazole (2.9 g, 10.7 mmol), 1,1,1,2,2,2-hexabutyldistannane (6.2 g, 10.7 mmol), CuBr (1.5 g, 10.7 mmol) and Pd(PPh₃)₄ (1.8 g, 1.1 mmol). The mixture was stirred at 100° C. under N₂ for 16 h, cooled and purified by silica gel column chromatography (DCM/MeOH=20/1) to give 5-(1-((2-(trimethylsilyl)ethoxy)methyl)-1H-imidazol-4-yl)nicotinaldehyde (600 mg, 18.5%) as a yellow solid. LC-MS m/z: 304.3 [M+H]⁺. HPLC Purity (254 nm): 46.62%; t_{R}=0.835 min.

A mixture of 5-(1-((2-(trimethylsilyl)ethoxy)methyl)-1H-imidazol-4-yl)nicotinaldehyde (600 mg, 1.98 mmol), NHMe₂ hydrochloride (323 mg, 3.96 mmol) and TEA (400 mg, 3.96 mmol) was stirred at RT for 1 h and then NaBH₃CN (249 mg, 3.96 mmol) was added. The reaction was stirred at RT for 1 h and concentrated in vacuo to give N,N-dimethyl(5-(1-((2-(trimethylsilyl)ethoxymethyl)-1H-imidazol-4-yl)pyridin-3-yl)methanamine (1 g, crude) as a yellow solid. LC-MS m/z: 333.1 [M+H]⁺. HPLC Purity (254 nm): 57.85%; t_{R}=1.95 min.

A solution of N,N-dimethyl (5-(1-((2-(trimethylsilyl)ethoxy)methyl)-1H-imidazol-4-yl)pyridin-3-yl)methanamine (1.0 g, 3 mmol) in THF (2 mL) was added TBAF (1.56 g, 6 mmol). The mixture stirred at 60° C. for 40 h and purified by silica gel column chromatography (DCM/MeOH=10/1) to give (5-(1H-imidazol-4-yl)pyridin-3-yl)-N,N-dimethylmethanamine (600 mg, crude) as a yellow solid. LC-MS m/z: 203.0 [M+H]⁺. HPLC Purity (254 nm): 75.99%; t_{R}=1.43 min.

Following general procedure B (method 1), (5-(1H-imidazol-4-yl)pyridin-3-yl)-N,N-dimethyl methanamine (202 mg, 1 mmol) and (2-isocyanatoethyl)benzene (164 mg, 1.1 mmol) afforded the title compound (18 mg, 5.1%) as a white solid. ¹H NMR (400 MHz, CDCl₃) δ 8.88 (d, J=2.0 Hz, 1H), 8.44 (d, J=1.7 Hz, 1H), 8.09 (d, J=0.8 Hz, 1H), 8.06 (s, 1H), 7.59 (d, J=1.2 Hz, 1H), 7.36 (t, J=7.3 Hz, 2H), 7.30 (d, J=7.3 Hz, 1H), 7.25-7.23 (m, 2H), 5.82 (brs, 1H), 3.73 (dt, J=12.8, 6.7 Hz, 2H), 3.47 (s, 2H), 2.98 (t, J=6.8 Hz, 2H), 2.26 (s, 6H). LC-MS m/z: 350.0 [M+H]⁺. HPLC Purity (214 nm): 95.96%; t_{R}=1.79 min.

### Example 45—N-(3,3-Dimethylbutyl)-4-(pyridin-3-yl)-1H-imidazole-1-carboxamide

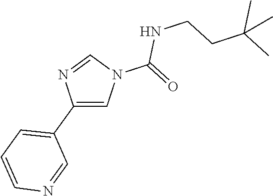 [SMILES: CC(C)(C)CCNC(=O)n1cnc(-c2cccnc2)c1]

Following general procedure C, 3-(1H-imidazol-4-yl)pyridine (145 mg, 1.0 mmol) and 3,3-dimethylbutan-1-amine (202 mg, 2 mmol) afforded the title compound (21.3 mg, 7.8%) as a yellow solid. ¹H NMR (400 MHz, CDCl₃) δ 8.98 (d, J=1.6 Hz, 1H), 8.51 (d, J=4.8 Hz, 1H), 8.17 (s, 1H), 8.12 (d, J=8.0 Hz, 1H), 7.67 (s, 1H), 7.36-7.33 (m, 1H), 5.90 (brs, 1H), 3.51-3.46 (m, 2H), 1.59-1.56 (m, 2H), 0.99 (s, 9H). LC-MS m/z: 272.7 [M+H]⁺. HPLC Purity (214 nm): 100%; t_{R}=5.95 min.

### Example 46—N-iso-Pentyl-4-(pyridin-2-yl)-1H-imidazole-1-carboxamide

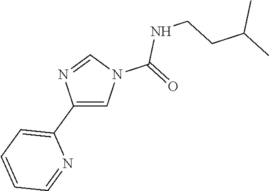 [SMILES: CC(C)CCNC(=O)n1cnc(-c2ccccn2)c1]

Following general procedure C, 2-(1H-imidazol-4-yl)pyridine (260 mg, 1.8 mmol) and 4-methylpentan-1-amine (235 mg, 2.7 mmol) afforded the title compound (42.4 mg, 9.1%) as a yellow solid. ¹H NMR (400 MHz, CDCl₃) δ 8.54 (d, J=4.9 Hz, 1H), 8.25 (d, J=1.3 Hz, 1H), 8.02 (d, J=7.9 Hz, 1H), 7.95 (d, J=1.3 Hz, 1H), 7.77 (td, J=7.8, 1.7 Hz, 1H), 7.24-7.18 (m, 1H), 5.86 (brs, 1H), 3.50-3.41 (m, 2H), 1.68 (dq, J=13.2, 6.7 Hz, 1H), 1.52 (q, J=7.2 Hz, 2H), 0.95 (d, J=7.7 Hz, 6H). LC-MS m/z: 259.1 [M+H]⁺. HPLC Purity (214 nm): 100%; t_{R}=7.97 min.

### Example 47—N-iso-Pentyl-4-(1-methylpiperidin-3-yl)-1H-imidazole-1-carboxamide

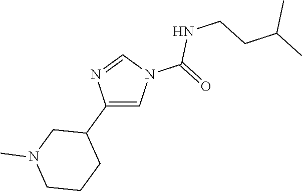 [SMILES: CC(C)CCNC(=O)n1cnc(C2CCCN(C)C2)c1]

Following general procedure D, 4-bromo-1H-imidazole (438 mg, 3.0 mmol and tert-butyl-5-(4,4,5,5-tetramethyl-1,3,2-dioxaborolan-2-yl)-3,6-dihydropyridine-1(2H)-carboxylate (927 mg, 3.0 mmol) afforded tert-butyl 5-(1H-imidazol-4-yl)-3,6-dihydropyridine-1(2H)-carboxylate (590 mg, 79%) as a yellow solid. LC-MS m/z: 250.1 [M+H]⁺. HPLC Purity (214 nm): 79%; t_{R}=1.51 min.

A suspension of tert-butyl 5-(1H-imidazol-4-yl)-3,6-dihydropyridine-1(2H)-carboxylate (500 mg, 2.0 mmol), and PtO₂ (80 mg, 0.2 mmol) in MeOH (10 mL) was stirred at 50° C. for 48 h under H₂ (1 atm) and then filtered. The filtrate was concentrated to afford tert-butyl 3-(1H-imidazol-4-yl)piperidine-1-carboxylate (460 mg, crude) as a yellow solid. LC-MS m/z: 252.4 [M+H]⁺. HPLC Purity (214 nm): 80%; t_{R}=0.80 min.

To a solution of tert-butyl 3-(1H-imidazol-4-yl)piperidine-1-carboxylate (500 mg, 2.0 mmol) in THF (10 mL) was added LAH (30 mL, 1N in THF) at 0° C. The mixture was stirred at RT for 24 h and then filtered. The filtrate was concentrated and purified by silica gel column chromatography (DCM/MeOH=10/1) to afford 3-(1H-imidazol-4-yl)-1-methylpiperidine (450 mg, crude) as a yellow oil. LC-MS m/z: 166.2 [M+H]⁺. HPLC Purity (214 nm): 39%; t_{R}=1.21 min.

Following general procedure C, 3-(1H-imidazol-4-yl)-1-methylpiperidine (82.0 mg, 0.5 mmol) and 3-methylbutylamine (52.0 mg, 0.5 mmol) afforded the title compound (23.0 mg, 17%) as a white solid. ¹H NMR (400 MHz, CDCl₃) δ 8.15 (s, 1H), 7.32 (s, 2H), 3.40-3.35 (m, 3H), 3.23-3.05 (m, 2H), 2.60-2.52 (m, 5H), 2.03-1.80 (m, 3H), 1.70-1.67 (m, 2H), 1.55-1.49 (m, 2H), 1.05-0.87 (m, 6H). LC-MS m/z: 279.1 [M+H]⁺. HPLC Purity (214 nm): 100%; t_{R}=4.88 min.

### Example 48—N-iso-Pentyl-4-(1-methylpiperidin-4-yl)-1H-imidazole-1-carboxamide

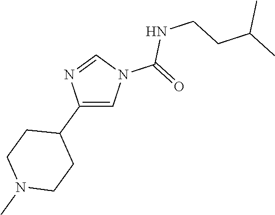 [SMILES: CC(C)CCNC(=O)n1cnc(C2CCN(C)CC2)c1]

A mixture of 4-bromo-1H-imidazole (1.3 g, 8.84 mmol), 1-methyl-4-(4,4,5,5-tetramethyl-1,3,2-dioxaborolan-2-yl)-1,2,3,6-tetrahydropyridine (2.367 g, 10.61 mmol), Pd[(t-Bu)₃P]2 (453 mg, 0.88 mmol) and Na₂CO₃ (1.874 g, 17.69 mmol) in 1,4-dioxane/H₂O (10 mL, 2/1) was heated to 100° C. under N₂ for 4 h under microwave conditions. The mixture was concentrated to give a residue which was purified by silica gel column chromatography (DCM:MeOH=1:1) to give 4-(1H-imidazol-4-yl)-1-methyl-1,2,3,6-tetrahydropyridine (220 mg, 15%) as a yellow oil. LC-MS m/z: 164.7 [M+H]⁺. HPLC Purity (254 nm): 99%; t_{R}=0.98 min.

To a solution of 4-(1H-imidazol-4-yl)-1-methyl-1,2,3,6-tetrahydropyridine (167 mg, 1.04 mmol) in MeOH (40 mL) was added PtO₂ (67 mg) and the mixture was stirred at RT under H₂ overnight. The mixture was filtered and concentrated to afford 4-(1H-imidazol-4-yl)-1-methylpiperidine (160 mg, 94%) as a yellow oil. LC-MS m/z: 166.7 [M+H]⁺. HPLC Purity (214 nm): 86%; t_{R}=0.66 min.

Following general procedure C, 4-(1H-imidazol-4-yl)-1-methylpiperidine (150 mg, 0.91 mmol) and 3-methylbutan-1-amine (95 mg, 1.09 mmol) afforded the title compound (24.1 mg, 10%) as a yellow oil. ¹H NMR (400 MHz, CDCl₃) δ 8.11 (s, 1H), 7.20 (s, 1H), 6.65 (brs, 1H), 3.46-3.29 (m, 4H), 2.78-2.60 (m, 1H), 2.62 (s, 5H), 2.15-2.02 (m, 4H), 1.72-1.60 (m, 1H), 1.55-1.48 (m, 2H), 0.95 (d, J=6.6 Hz, 6H). LC-MS m/z: 279.7 [M+H]⁺. HPLC Purity (254 nm): 92.24%; t_{R}=4.90 min.

### Example 49—N-iso-Pentyl-2-methyl-4-(pyridin-3-yl)-1H-imidazole-1-carboxamide

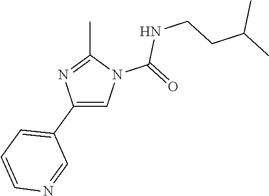 [SMILES: Cc1nc(-c2cccnc2)cn1C(=O)NCCC(C)C]

Following general procedure D, 4-bromo-2-methyl-1H-imidazole (500 mg, 3.12 mmol) and pyridin-3-ylboronic acid (500 mg, 4.06 mmol) afforded 3-(2-methyl-1H-imidazol-4-yl)pyridine (260 mg, 52%) as a yellow solid. LC-MS m/z: 160.0 [M+H]⁺. HPLC Purity (254 nm): >90%; t_{R}=0.33 min.

Following general procedure C, 3-(2-methyl-1H-imidazol-4-yl)pyridine (160 mg, 1.0 mmol) and 3-methylbutan-1-amine (96 mg, 1.1 mmol) afforded the title compound (24.6 mg, 9.0%) as a yellow solid. ¹H NMR (400 MHz, CDCl₃) δ 8.94-8.90 (m, 1H), 8.50-8.47 (m, 1H), 8.09 (d, J=8.0 Hz, 1H), 7.49-7.47 (m, 1H), 7.37-7.29 (m, 1H), 6.22 (brs, 1H), 3.51-3.44 (m, 2H), 2.70 (s, 3H), 1.81-1.54 (m, 3H), 1.01-0.98 (m, 6H). LC-MS m/z: 273.0 [M+H]⁺. HPLC Purity (214 nm): >99%; t_{R}=1.49 min.

### Example 50—N-iso-Pentyl-4-(thiazol-2-yl)-1H-imidazole-1-carboxamide

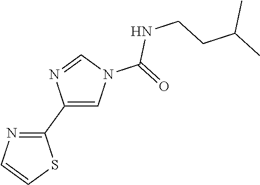 [SMILES: CC(C)CCNC(=O)n1cnc(-c2nccs2)c1]

Following general procedure A, 2-bromo-1-(thiazol-2-yl)ethanone (1.00 g, 4.83 mmol) afforded 2-(1H-imidazol-5-yl)thiazole (0.50 g, 68.4%) as a yellow solid. LC-MS m/z: 152.1 [M+H]⁺. HPLC Purity (254 nm): 95%; t_{R}=1.27 min.

Following general procedure C, 2-(1H-imidazol-5-yl)thiazole (0.10 g, 0.66 mmol) and 3-methylbutylamine (0.06 g, 0.70 mmol) afforded the title compound (29.6 mg, 17.0%) as a white solid. ¹H NMR (400 MHz, CDCl₃) δ 8.18 (s, 1H), 7.86 (s, 1H), 7.81 (d, J=3.2 Hz, 1H), 7.33 (d, J=3.2 Hz, 1H), 5.68 (brs, 1H), 3.47 (dd, J=14 Hz, 6.8 Hz, 2H), 1.71-1.66 (m, 1H), 1.53 (dd, J=14.4 Hz, 6.8 Hz, 2H), 0.96 (d, J=6.4 Hz, 6H). LC-MS m/z: 265.1 [M+H]⁺. HPLC Purity (214 nm): 100%; t_{R}=6.99 min.

### Example 51—N-iso-Pentyl-4-(thiazol-5-yl)-1H-imidazole-1-carboxamide

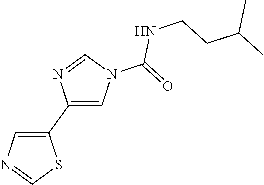 [SMILES: CC(C)CCNC(=O)n1cnc(-c2cncs2)c1]

To a solution 1-(thiazol-5-yl)ethanone (0.5 g, 4 mmol) in Et₂O (15 mL) was added Br₂ (8 ml, 22.8 mmol) and the mixture was stirred at RT for 2 h. the precipitate was filtered to give 2-bromo-1-(thiazol-5-yl)ethanone (0.3 g, 37.5%) as a grey solid. LC-MS m/z: 207.1 [M+H]⁺. HPLC Purity (214 nm): 90.93%; t_{R}=0.72 min.

Following general procedure A, 2-bromo-1-(thiazol-5-yl)ethanone (0.3 g, 1.5 mmol) afforded 5-(1H-imidazol-4-yl)thiazole (0.2 g, 91%). LC-MS m/z: 152.1 [M+H]⁺. HPLC Purity (214 nm): 46.40%; t_{R}=1.04 min.

Following general procedure C, 5-(1H-imidazol-4-yl)thiazole (200 mg, 1.3 mmol) and 3-methylbutylamine (136 mg, 1.5 mmol) afforded the title compound (14.3 mg, 4.1%) as a white solid. ¹H NMR (400 MHz, CDCl₃) δ 8.73 (s, 1H), 8.17 (s, 1H), 8.08 (s, 11H), 7.61 (s, 1H), 6.35 (s, 1H), 3.47 (dd, J=13.8, 6.5 Hz, 2H), 1.75-1.61 (m, 1H), 1.54 (dd, J=14.6, 7.1 Hz, 2H), 0.95 (d, J=6.6 Hz, 6H). LC-MS m/z: 265.3 [M+H]⁺. HPLC Purity (254 nm): 100%; t_{R}=6.76 min.

### Example 52—N-iso-Pentyl-2-methoxy-4-(pyridin-3-yl)-1H-imidazole-1-carboxamide

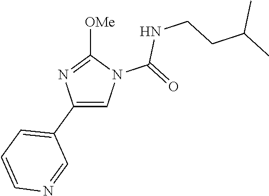 [SMILES: COc1nc(-c2cccnc2)cn1C(=O)NCCC(C)C]

A mixture of 2-bromo-1-(pyridin-3-yl)ethanone (1.0 g, 5.03 mmol), methyl carbamimidate (3.72 g, 50.3 mmol) and NaHCO₃ (8.45 g, 100.6 mmol) in THF (100 mL) was heated to 60° C. and stirred for 3 h. The reaction was concentrated and purified by silica gel column chromatography (DCM:MeOH=5:1) to afford 3-(2-methoxy-1H-imidazol-4-yl)pyridine (320 mg, 36%) as a yellow solid. LC-MS m/z: 176.1 [M+H]⁺. HPLC Purity (214 nm): 95%; t_{R}=1.22 min.

Following general procedure C, 3-(2-methoxy-1H-imidazol-4-yl)pyridine (150 mg, 0.86 mmol) and 3-methylbutylamine (90 mg, 1.03 mmol) afforded the title compound (42.2 mg, 16.8%) as a white solid. ¹H NMR (400 MHz, CDCl₃) δ 8.97 (s, 1H), 8.48 (d, J=4.8 Hz, 1H), 7.99 (d, J=10.8 Hz, 1H), 7.61 (s, 1H), 7.30-7.26 (m, 1H), 6.89 (t, J=5.2 Hz, 1H), 4.25 (s, 3H), 3.43 (q, J=6.8 Hz, 2H), 1.73-1.67 (m, 1H), 1.59-1.49 (m, 2H), 0.97 (d, J=6.8 Hz, 6H). LC-MS m/z: 289.0 [M+H]⁺. HPLC Purity (214 nm): >99%; t_{R}=6.11 min.

### Example 53—N-(2-Cyclopropylethyl)-2-methoxy-4-(pyridin-3-yl)-1H-imidazole-1-carboxamide

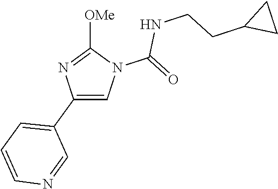 [SMILES: COc1nc(-c2cccnc2)cn1C(=O)NCCC1CC1]

Following general procedure C, 3-(2-methoxy-1H-imidazol-4-yl)pyridine (150 mg, 0.86 mmol) and 2-cyclopropylethanamine (90 mg, 1.03 mmol) afforded the title compound (61.6 mg, 24.2%) as a white solid. ¹H NMR (400 MHz, CDCl₃) δ 9.08 (s, 1H), 8.79 (d, J=4.0 Hz, 1H), 8.10 (d, J=8.0 Hz, 1H), 7.72 (s, 1H), 7.41-7.37 (m, 1H), 7.22 (brs, 1H), 4.37 (s, 3H), 3.62 (q, J=6.0 Hz, 2H), 1.65 (q, J=6.8 Hz, 2H), 1.89-1.78 (m, 1H), 0.66-0.60 (m, 2H), 0.37-0.05 (m, 2H). LC-MS m/z: 287.0 [M+H]⁺. HPLC Purity (214 nm): >99%; t_{R}=5.72 min.

### Example 54—N-(2-Cyclopropylethyl)-4-(2-(4-morpholinopiperidin-1-yl)pyridin-3-yl)-1H-imidazole-1-carboxamide

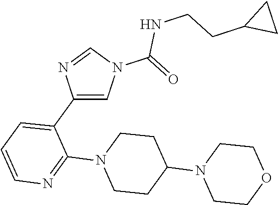 [SMILES: O=C(NCCC1CC1)n1cnc(-c2cccnc2N2CCC(N3CCOCC3)CC2)c1]

To a solution of 4-bromo-1H-imidazole (12.0 g, 81.7 mmol) in THF (100 mL) was added NaH (4.0 g, 163.4 mmol) and 4-methoxybenzylchloride (2.0 g, 122.5 mmol) at 0° C. and the resulting reaction mixture was heated to 50° C. and stirred overnight. The reaction mixture was then concentrated in vacuo to give a residue which was purified by silica gel column chromatography (PE:EA=1/1) to afford 4-bromo-1-(4-methoxybenzyl)-1H-imidazole (8.8 g, 40%) as a faint yellow solid. LC-MS m/z: 268.0 [M+H]⁺. t_{R}=1.80 min.

Following general procedure D, 4-bromo-1-(4-methoxybenzyl)-1H-imidazole (2.8 g, 20.0 mmol) and 2-fluoropyridin-3-ylboronic acid (5.4 g, 20.0 mmol) afforded 2-fluoro-3-(1-(4-methoxybenzyl)-1H-imidazol-4-yl)pyridine as a yellow solid (600 mg, 12.0%). LC-MS m/z: 284.2 [M+H]⁺. HPLC Purity (214 nm): 90%; t_{R}=1.33 min.

To a solution of 2-fluoro-3-(1-(4-methoxybenzyl)-1H-imidazol-4-yl)pyridine (0.50 g, 1.87 mmol) and K₂CO₃ (0.38 g, 2.80 mmol) in DMA (10.0 mL) was added 4-(piperidin-4-yl)morpholine (0.32 g, 1.87 mmol). The mixture was stirred overnight at 90° C. and then poured into water (30 ml) and extracted with EA (25 mL×3). The combined extracts were washed with water (20 mL×2), dried over Na₂SO₄ and concentrated to give a residue which was purified by silica gel chromatography (DCM/MeOH=10/1) to give 4-(1-(3-(1-(4-methoxybenzyl)-1H-imidazol-4-yl)pyridin-2-yl) piperidin-4-yl)morpholine (500 mg, 61%) as a yellow liquid. LC-MS m/z: 434.0 [M+H]⁺. HPLC Purity (254 nm): 900%; t_{R}=0.69 min.

A solution of 4-(1-(3-(1-(4-methoxybenzyl)-1H-imidazol-4-yl)pyridin-2-yl)piperidin-4-yl)morpholine (500 mg, 1.15 mmol) in TFA (1.5 mL) was stirred under microwave at 140° C. for 3 h. The mixture was concentrated in vacuo to give a residue which was purified by silica gel column chromatography (DCM:MeOH=10:1) to give 4-(1-(3-(1H-imidazol-4-yl)pyridin-2-yl)piperidin-4-yl)morpholine (250 mg, 52%) as a colorless liquid. LC-MS m/z: 314.3.0 [M+H]⁺. HPLC Purity (254 nm): 92%; t_{R}=0.70 min.

Following general procedure C, 4-(1-(3-(1H-imidazol-4-yl) pyridin-2-yl)piperidin-4-yl)morpholine (100 mg, 0.32 mmol) and 2-cyclopropylethanamine (32 mg, 0.38 mmol) afforded the title compound (19 mg, 14.0%) as a white solid. ¹H NMR (500 MHz, CDCl₃) δ 8.27 (dd, J=7.6, 1.8 Hz, 1H), 8.24-8.17 (m, 2H), 8.03 (s, 1H), 7.02 (dd, J=7.6, 4.9 Hz, 1H), 6.25 (brs, 1H), 3.75 (s, 4H), 3.59-3.50 (m, 4H), 2.78 (t, J=12.0 Hz, 2H), 2.61 (s, 4H), 2.31-2.25 (m, 1H), 2.06-1.98 (m, 2H), 1.63-1.56 (m, 4H), 0.77-0.70 (m, 1H), 0.56-0.49 (m, 2H), 0.16-0.11 (m, 2H). LC-MS m/z: 425.2 [M+H]⁺. HPLC Purity (214 nm): 100%; t_{R}=6.73 min.

### Example 55—N-(2-Cyclopropylethyl)-4-(2-(4-(4-methylpiperazin-1-yl)piperidin-1-yl)pyridin-3-yl)-1H-imidazole-1-carboxamide

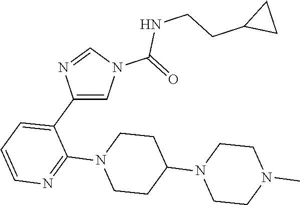 [SMILES: CN1CCN(C2CCN(c3ncccc3-c3cn(C(=O)NCCC4CC4)cn3)CC2)CC1]

A mixture of 2-fluoro-3-(1-(4-methoxybenzyl)-1H-imidazol-4-yl)pyridine (400 mg, 1.41 mmol), 1-methyl-4-(piperidin-4-yl)piperazine (309 mg, 1.69 mmol) and K₂CO₃ (389 mg, 2.82 mmol) in NMP (8 ml) was stirred at 180° C. for 13 h under microwave conditions. The reaction mixture was cooled, concentrated and the residue was purified by silica gel column chromatography (DCM/MeOH=5/1) to afford 1-(1-(3-(1-(4-methoxybenzyl)-1H-imidazol-4-yl)pyridin-2-yl)piperidin-4-yl)-4-methylpiperazine (350 mg, 55.7%) as a yellow oil. LC-MS m/z: 447.1 [M+H]⁺. HPLC Purity (254 nm): 97.17%; t_{R}=1.37 min.

A mixture of 1-(1-(3-(1-(4-methoxybenzyl)-1H-imidazol-4-yl)pyridin-2-yl)piperidin-4-yl)-4-methylpiperazine (180 mg, 0.4 mmol) in TFA (10 ml) was stirred at 130° C. for 5 h under microwave conditions. The reaction mixture was cooled, concentrated and the residue purified by silica gel column chromatography (DCM/MeOH=20/1) to give 1-(1-(3-(1H-imidazol-4-yl)pyridin-2-yl) piperidin-4-yl)-4-methylpiperazine (100 mg, 76.7%) as a yellow oil. LC-MS m/z: 327.2 [M+H]⁺. HPLC Purity (214 nm): 90.71%; t_{R}=1.52 min.

Following general procedure C, 1-(1-(3-(1H-imidazol-4-yl)pyridine-2-yl)piperidin-4-yl)-4-methylpiperazine (100 mg, 0.31 mmol) and 2-cyclopropylethanamine (45 mg, 0.37 mmol) afforded the title compound (6.6 mg, 4.9%) as a yellow solid. ¹H NMR (400 MHz, CDCl₃) δ 8.27 (dd, J=7.6, 1.9 Hz, 1H), 8.24 (d, J=1.4 Hz, 1H), 8.22 (dd, J=4.8, 1.9 Hz, 1H), 8.04 (d, J=1.4 Hz, 1H), 7.01 (dd, J=7.6, 4.8 Hz, 1H), 6.47 (s, 1H), 3.62-3.46 (m, 4H), 2.77 (t, J=11.7 Hz, 3H), 2.76-2.40 (m, 6H), 2.32 (s, 5H), 2.03 (d, J=9.6 Hz, 2H), 1.69-1.59 (m, 4H), 0.80-0.73 (m, 1H), 0.58-1.50 (m, 2H), 0.18-0.13 (m, 2H). LC-MS m/z: 438.1[M+H]⁺. HPLC Purity (214 nm): 92.96%; t_{R}=1.41 min.

### Example 56—N-iso-Pentyl-4-((1-methylpiperidin-4-yl)methyl)-1H-imidazole-1-carboxamide

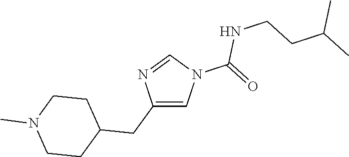 [SMILES: CC(C)CCNC(=O)n1cnc(CC2CCN(C)CC2)c1]

A solution of (1H-imidazol-4-yl)methanol (3.0 g, 30 mmol) in SOCl₂ (30 mL) was stirred at 80° C. for 16 h and then concentrated to give 4-(chloromethyl)-1H-imidazole (3.5 g, crude) as a yellow solid. LC-MS m/z: 117.2 [M+H]⁺. HPLC Purity (214 nm): 85%; t_{R}=0.19 min.

To a solution of diethyl phosphonate (4.97 g, 36 mmol) in THF (20 mL) was added LiHMDS (75 mL, 75.0 mmol) at −50° C. The mixture was stirred at −50° C. for 30 min and 4-(chloromethyl)-1H-imidazole (3.4 g, 30 mmol) was added and the solution was stirred at RT for 2 h and poured into ice-water (20 mL). The mixture was extracted with EA (30 mL) and the organic layers were concentrated and the residue was purified by silica gel column chromatography (DCM/MeOH=10/1) to give diethyl((1H-imidazol-4-yl)methyl)phosphonate (4.4 g, crude) as a yellow solid. LC-MS m/z: 219.0 [M+H]⁺. HPLC Purity (214 nm): 69%; t_{R}=1.51 min.

To a solution of diethyl((1H-imidazol-4-yl)methyl)phosphonate (4.3 g, 20.0 mmol) in THF (35 mL) was added NaH (1200 mg, 30.0 mmol) at 0° C. The mixture was stirred at 0° C. for 30 min and then PMBCl (3.7 g, 24.0 mmol) was added. The solution was stirred at 50° C. for 16 h and poured into ice-water (20 mL). The mixture was extracted with EA (50 mL) and the organic layers were concentrated and the residue was purified by silica gel column chromatography (DCM/MeOH=10/1) to give diethyl((1-(4-methoxybenzyl)-1H-imidazol-4-yl)methyl)phosphonate (1.1 g, 16%) as a yellow solid. LC-MS m/z: 339.1 [M+H]⁺. HPLC Purity (214 nm): 19%; t_{R}=1.50 min.

A mixture of diethyl((1-(4-methoxybenzyl)-1H-imidazol-4-yl)methyl)phosphonate (1.0 g, 3.0 mmol), 1-methylpiperidin-4-one (678 mg, 6.0 mmol) and NaH (600 mg, 15.0 mmol) in THF (15 mL) was stirred at 60° C. for 15 h under N₂. The reaction was cooled and concentrated in vacuo to give a residue which was purified by silica gel column chromatography (DCM/MeOH=10/1) to give 5-4-((1-(4-methoxybenzyl)-1H-imidazol-4-yl)methylene)-1-methylpiperidine (400 mg, 45%) as a yellow oil. LC-MS m/z: 339.2 [M+H]⁺. HPLC Purity (214 nm): 85%; t_{R}=0.74 min.

A suspension of 4-((1-(4-methoxybenzyl)-1H-imidazol-4-yl)methylene)-1-methylpiperidine (356 mg, 1.2 mmol), Pd(OH)₂ (80 mg, 0.6 mmol) in MeOH (10 mL) was stirred at 50° C. for 15 h under H₂ (1 atm) and filtered. The filtrate was concentrated to afford 4-((1-(4-methoxybenzyl)-1H-imidazol-4-yl)methyl)-1-methylpiperidine (340 mg, crude) as a yellow solid. LC-MS m/z: 300.2 [M+H]⁺. HPLC Purity (214 nm): 95%, t_{R}=1.771 min.

To a solution of 4-((1-(4-methoxybenzyl)-1H-imidazol-4-yl)methyl)-1-methyl piperidine (300 mg, 1.0 mmol) in CH₃CN/H₂O (8 mL/2 mL) was added CAN (1.6 g, 3.0 mmol). The mixture was stirred at RT for 16 h and purified by silica gel column chromatography (DCM/MeOH=1/1) to give 4-((1H-imidazol-4-yl)methyl)-1-methylpiperidine (200 mg, crude) as a yellow solid. LC-MS m/z: 180.1 [M+H]⁺. HPLC Purity (214 nm): 38%; t_{R}=0.37 min.

Following general procedure C, 4-((1H-imidazol-4-yl)methyl)-1-methylpiperidine (179 mg, 1.0 mmol) and 3-methylbutylamine (87 mg, 1.0 mmol) afforded the title compound (6.2 mg, 2.1%) as a white solid. ¹H NMR (400 MHz, CDCl₃) δ 8.14 (d, J=13.6 Hz, 1H), 7.25 (s, 1H), 7.02 (brs, 1H), 3.43-3.38 (m, 2H), 3.31 (d, J=11.1 Hz, 2H), 2.57 (s, 3H), 2.53 (d, J=6.8 Hz, 2H), 2.49-2.40 (m, 2H), 1.95-1.63 (m, 6H), 1.53 (dd, J=14.8, 7.1 Hz, 2H), 0.94 (d, J=8.0 Hz, 6H). LC-MS m/z: 293.1 [M+H]⁺. HPLC Purity (214 nm): 100%; t_{R}=5.23 min.

### Example 57—4-(tert-Butyl)-N-isopentyl-2-methoxy-1H-imidazole-1-carboxamide

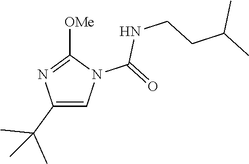 [SMILES: COc1nc(C(C)(C)C)cn1C(=O)NCCC(C)C]

Following general procedure C, 4-tert-butyl-2-methoxy-1H-imidazole (140 mg, 0.91 mmol) and isoamylamine (80 mg, 0.91 mmol) afforded the title compound (120.8 mg, 49.4%) as a white solid. ¹H NMR (400 MHz, CDCl₃) δ 6.91 (s, 1H), 6.84 (brs, 1H), 4.14 (s, 3H), 3.41-3.35 (m, 2H), 1.69-1.60 (m, 1H), 1.47 (dt, J=14.6, 7.1 Hz, 2H), 1.21 (s, 9H), 0.95 (d, J=6.6 Hz, 6H). LC-MS m/z: 268.2 [M+H]⁺. HPLC Purity (214 nm): >96%; t_{R}=9.91 min.

### Example 58—4-(tert-Butyl)-2-methoxy-N-(4-methylpentan-2-yl)-1H-imidazole-1-carboxamide

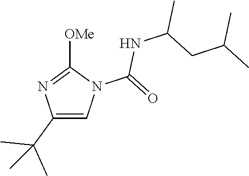 [SMILES: COc1nc(C(C)(C)C)cn1C(=O)NC(C)CC(C)C]

Following general procedure C, 4-(tert-butyl)-2-methoxy-1H-imidazole (224 mg, 1.5 mmol) and 4-methylpentan-2-amine (177 mg, 1.75 mmol) afforded the title compound 99.2 mg, 22.3%) as a colorless liquid. ¹H NMR (400 MHz, CDCl₃) δ 6.91 (s, 1H), 6.63 (brs, 1H), 4.15 (s, 3H), 4.11-4.03 (m, 1H), 1.73-1.65 (m, 1H), 1.44 (ddd, 1=14.7, 8.5, 6.2 Hz, 1H), 1.31 (ddd, J=13.8, 8.1, 5.9 Hz, 1H), 1.23 (s, 9H), 1.20 (d, J=7.3 Hz, 3H), 1.01-0.84 (dd, J=6.8, 2.0 Hz, 6H). LC-MS m/z: 282.7 [M+H]⁺. HPLC Purity (214 nm): 100%; t_{R}=9.37 min.

### Example 59—N-(4-Methylpentan-2-yl)-4-(pyridin-3-yl)-1H-imidazole-1-carboxamide

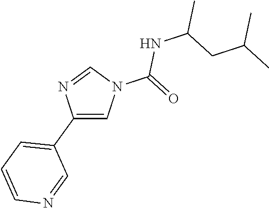 [SMILES: CC(C)CC(C)NC(=O)n1cnc(-c2cccnc2)c1]

Following general procedure C, 3-(1H-imidazol-4-yl)pyridine (122 mg, 0.84 mmol) and 4-methyl-2-pentanamine hydrochloride (85 mg, 0.84 mmol) afforded the title compound (13 mg, 5.7%) as a white oil. ¹H NMR (400 MHz, CDCl₃) δ 8.92 (s, 1H), 8.49 (d, J=4.2 Hz, 1H), 8.23 (s, 1H), 8.10 (d, J=7.8 Hz, 1H), 7.76 (s, 1H), 7.37-7.29 (m, 1H), 6.39 (brs, 1H), 4.12-4.05 (m, 1H), 1.75-1.65 (m, 1H), 1.54-49 (m, 1H), 1.41-1.30 (m, 1H), 1.28 (d, J=6.4 Hz, 3H), 0.95 (dd, J=10.0, 6.6 Hz, 6H). LC-MS m/z: 273 [M+H]⁺. HPLC Purity (214 nm): 99%; t_{R}=1.49 min.

### Example 60—N-(2-Methylbutyl)-4-(pyridin-3-yl)-1H-imidazole-1-carboxamide

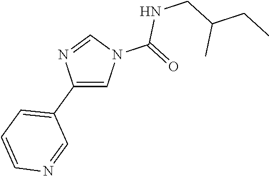 [SMILES: CCC(C)CNC(=O)n1cnc(-c2cccnc2)c1]

Following general procedure C, 3-(1H-imidazol-4-yl)pyridine (150 mg, 1.0 mmol) and 2-methylbutan-1-amine (104 mg, 1.2 mmol) afforded the title compound (85 mg, 32%) as a yellow solid. ¹H NMR (400 MHz, CDCl₃) δ 9.01 (d, J=2.2 Hz, 1H), 8.54 (dd, J=4.8, 1.6 Hz, 1H), 8.18 (d, J=1.3 Hz, 1H), 8.13 (dt, J=8.0, 1.9 Hz, 1H), 7.66 (d, J=1.3 Hz, 1H), 7.35 (dd, J=7.9, 4.8 Hz, 1H), 5.73 (s, 1H), 3.47-3.38 (m, 1H), 3.34-3.24 (m, 1H), 1.81-1.70 (m, 1H), 1.53-1.45 (m, 1H), 1.32-1.22 (m, 1H), 0.98 (dd, J=15.9, 7.1 Hz, 6H). LC-MS m/z: 259.0 [M+H]⁺. HPLC Purity (254 nm): 100%; t_{R}=1.52 min.

### Example 61—N-Cyclohexyl-4-(pyridin-3-yl)-1H-imidazole-1-carboxamide

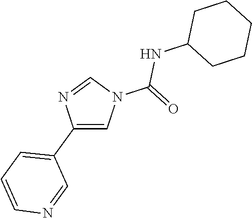 [SMILES: O=C(NC1CCCCC1)n1cnc(-c2cccnc2)c1]

Following general procedure C, 3-(1H-imidazol-4-yl)pyridine (120 mg, 0.8 mmol) and cyclohexylamine (95 mg, 0.96 mmol) afforded the title compound (60.6 mg, 27.1%) as a white solid. ¹H NMR (400 MHz, CDCl₃) δ 8.96 (d, J=1.8 Hz, 1H), 8.52 (dd, J=4.8, 1.2 Hz, 1H), 8.19 (s, 1H), 8.10 (d, J=7.9 Hz, 1H), 7.73 (s, 1H), 7.38-7.32 (m, 1H), 6.06 (d, J=7.6 Hz, 1H), 3.92-3.81 (m, 1H), 2.15-2.06 (m, 2H), 1.92-1.78 (m, 2H), 1.75-1.67 (m, 1H), 1.51-1.13 (m, 5H). LC-MS m/z: 271.1 [M+H]⁺. HPLC Purity (254 nm): 100%; t_{R}=5.49 min.

### Example 62—N-Cyclohexyl-4-(6-methoxypyridin-3-yl)-1H-imidazole-1-carboxamide

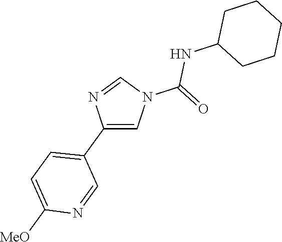 [SMILES: COc1ccc(-c2cn(C(=O)NC3CCCCC3)cn2)cn1]

Following general procedure C, 5-(1H-imidazol-4-yl)-2-methoxypyridine (246 mg, 1.4 mmol) and cyclohexylamine (167 mg, 1.7 mmol) afforded the title compound (55.9 mg, 13.3%) as a white solid. ¹H NMR (400 MHz, CDCl₃) δ 8.56 (d, J=2.1 Hz, 1H), 8.12 (s, 1H), 7.99 (dd, J=8.6, 2.3 Hz, 1H), 7.48 (s, 1H), 6.79 (d, J=8.6 Hz, 1H), 5.41 (d, J=7.5 Hz, 1H), 3.98 (s, 3H), 3.96-3.83 (m, 1H), 2.14-2.08 (m, 2H), 1.85-1.78 (m, 2H), 1.76-1.68 (m, 1H), 1.52-1.39 (m, 2H), 1.37-1.18 (m, 3H). LC-MS m/z: 301.7 [M+H]⁺. HPLC Purity (214 nm): 98.90%; t_{R}=7.27 min

### Example 63—N-(2-Cyclopropylethyl)-2-ethoxy-4-(pyridin-2-yl)-1H-imidazole-1-carboxamide

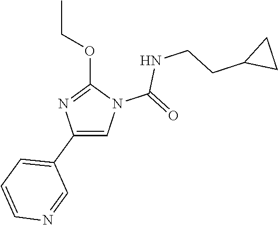 [SMILES: CCOc1nc(-c2cccnc2)cn1C(=O)NCCC1CC1]

A solution of 2-(bromoacetyl)pyridine hydrobromide (500 mg, 1.78 mmol), ethyl carbamimidate hydrobromide (330 mg, 2.65 mmol) and sodium bicarbonate (840 mg, 10.0 mmol) in EtOH (10 ml) was stirred for 16 h at 65° C. The mixture was concentrated and purified by SGC (5% MeOH in DCM) to give 2-(2-ethoxy-1H-imidazol-4-yl)pyridine (250 mg, 74.0%) as a white solid.

Following general procedure C, 2-(2-ethoxy-1H-imidazol-4-yl)pyridine (180 mg, 0.95 mmol), and 2-cyclopropylethylamine hydrochloride (170 mg, 1.43 mmol) afforded the title compound as a yellow solid (69.4 mg, 24.3%). ¹H NMR (400 MHz, CDCl₃) δ 8.59 (d, J=4.1 Hz, 1H), 7.89 (s, 1H), 7.77 (d, J=7.9 Hz, 1H), 7.68 (td, J=7.7, 1.8 Hz, 1H), 7.21-7.08 (m, 2H), 4.71 (q, J=7.1 Hz, 2H), 3.52 (dd, J=12.4, 6.8 Hz, 2H), 1.56-1.49 (m, 5H), 0.79-0.70 (m, 1H), 0.54-0.47 (m, 2H), 0.16-0.12 (m, 2H). LC-MS m/z: 310.3 [M+H]⁺. HPLC Purity (214 nm): >96%; t_{R}=9.22 min.

### Example 64—N-(2-Cyclopropylethyl)-2-ethoxy-4-(pyridin-3-yl)-1H-imidazole-1-carboxamide

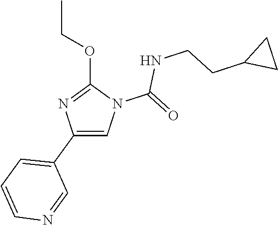 [SMILES: CCOc1nc(-c2cccnc2)cn1C(=O)NCCC1CC1]

To a solution of 2-amino-1-(pyridin-3-yl)ethanone (560 mg, 2 mmol) in EtOH (10 mL) was added ethyl carbamimidate (372 mg, 3 mmol) and NaHCO₃ (840 mg, 10 mmol) and the mixture was stirred at 100° C. for 16 h and purified by silica gel column chromatography (DCM/MeOH=10/1) to give 3-(2-ethoxy-1H-imidazol-4-yl)pyridine (70 mg, 18.5%) as a yellow solid. LC-MS m/z: 190.3 [M+H]⁺. HPLC Purity (214 nm): 95.47%; t_{R}=0.48 min.

Following general procedure C, 3-(2-ethoxy-1H-imidazol-4-yl)pyridine (70 mg, 0.37 mmol) and 2-cyclopropylethanamine (45 mg, 0.37 mmol) afforded the title compound (37.1 mg, 33.3%) as a white solid. ¹H NMR (400 MHz, CDCl₃) δ 8.97 (s, 1H), 8.47 (d, J=4.4 Hz, 1H), 7.99 (d, J=7.8 Hz, 1H), 7.61 (s, 1H), 7.33-7.28 (m, 1H), 7.16 (s, 1H), 4.68 (q, J=7.1 Hz, 2H), 3.52 (dd, J=12.4, 6.7 Hz, 2H), 1.60-1.52 (m, 5H), 0.81-0.73 (m, 1H), 0.57-0.50 (m, 2H), 0.18-0.14 (m, 2H). LC-MS m/z: 301.2 [M+H]⁺. HPLC Purity (214 nm): 98.46%; t_{R}=6.47 min.

### Example 65—N-(2-Cyclopropylethyl)-2-isopropoxy-4-(pyridin-3-yl)-1H-imidazole-1-carboxamide

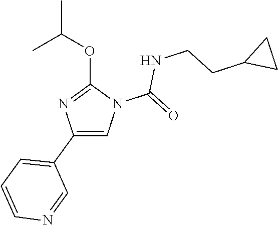 [SMILES: CC(C)Oc1nc(-c2cccnc2)cn1C(=O)NCCC1CC1]

To a solution of 2,4,5-tribromo-1H-imidazole (10.0 g, 32.81 mmol) in DMF (50 mL) was added NaH (60% in oil) (3.9 g, 98.43 mmol) at 0° C. The mixture was stirred at 0° C. for 1 h, then a solution of SEMCl (10.9 g, 65.62 mmol) in DMF (30 mL) was added and the mixture was stirred at RT for 15 h. The reaction was quenched with water, extracted with EA (50 mL×3), washed with water (40 mL×3) and brine, dried over Na₂SO₄, and purified by silica gel column chromatography (PE:EA=20:1) to give 2,4,5-tribromo-1-((2-(trimethylsilyl)ethoxy)methyl)-1H-imidazole (6.2 g, 43%) as a colorless oil. LC-MS m/z: 407.0 [M+H]⁺. HPLC Purity (214 nm): 80.0%; t_{R}=1.64 min.

To a solution of propan-2-ol (40 mL) was added NaH (60% in oil) (1.38 g, 34.48 mmol) at 0° C. The mixture was stirred at 0° C. for 2 h then 2,4,5-tribromo-1-((2-(trimethylsilyl)ethoxy)methyl)-1H-imidazole (3.0 g, 6.90 mmol) was added and the mixture was stirred at 110° C. for 8 h under microwave. The mixture was purified by silica gel column chromatography (PE:EA=40:1) to give 4,5-dibromo-2-isopropoxy-1-((2-(trimethylsilyl)ethoxy) methyl)-1H-imidazole (1.63 g, 57%) as a colorless oil. LC-MS m/z: 415.1 [M+H]⁺. HPLC Purity (214 nm): 100.0%; t_{R}=1.73 min.

To a solution of 4,5-dibromo-2-isopropoxy-1-((2-(trimethylsilyl)ethoxy)methyl)-1H-imidazole (1.64 g, 3.96 mmol) in THF (25 mL) was added n-BuLi (2.5 M in THF) (1.4 mL, 3.56 mmol) at −78° C. and stirred at for 1 h. The reaction was quenched with aq. NH₄Cl and extracted with EA (30 mL×4). The organic phase was washed with brine, dried over Na₂SO₄ and purified by silica gel column chromatography (PE:EA=4:1) to give 4-bromo-2-isopropoxy-1-((2-(trimethylsilyl)ethoxy)methyl)-1H-imidazole (1.32 g, 98%) as a light-yellow oil. LC-MS m/z: 335.2 [M+H]⁺. HPLC Purity (214 nm): 80%; t_{R}=1.62 min.

Following general procedure D, 4-bromo-2-isopropoxy-1-((2-(trimethylsilyl)ethoxy) methyl)-1H-imidazole (1.3 g, 3.88 mmol) and pyridin-3-ylboronic acid (715 mg, 5.81 mmol) afforded 3-(2-isopropoxy-1-((2-(trimethylsilyl)ethoxy)methyl)-1H-imidazol-4-yl)pyridine (700 mg, 53.8%) as a light brown oil. LC-MS m/z: 334.3 [M+H]⁺. HPLC Purity (214 nm): 100%; t_{R}=1.29 min.

Following general procedure F (method 2), 3-(2-isopropoxy-1-((2-(trimethylsilyl) ethoxy)methyl)-1H-imidazol-4-yl)-pyridine (680 mg, 3.88 mmol) afforded 3-(2-isopropoxy-1H-imidazol-4-yl)pyridine (300 mg, 38%) as a brown oil. LC-MS m/z: 204.1 [M+H]⁺. HPLC Purity (214 nm): 90.7%; t_{R}=1.54 min.

Following general procedure C, 3-(2-isopropoxy-1H-imidazol-4-yl)pyridine (280 mg, 1.38 mmol) and 2-cyclopropylethan-1-amine hydrogen chloride (280 mg, 1.38 mmol) afforded the title compound (14.5 mg, 3.1%) as a white solid. ¹H NMR (400 MHz, CDCl₃) δ 8.97 (d, J=1.7 Hz, 1H), 8.47 (dd, J=4.8, 1.6 Hz, 1H), 7.98 (dt, J=7.9, 1.9 Hz, 1H), 7.60 (s, 1H), 7.28 (dd, J=7.0, 3.3 Hz, 1H), 7.18 (brs, 1H), 5.43 (dq, J=12.4, 6.2 Hz, 1H), 3.52 (q, J=6.4 Hz, 2H), 1.59-1.52 (m, 2H), 1.53 (d, J=6.4 Hz, 6H), 0.79-0.72 (m, 1H), 0.56-0.50 (m, 2H), 0.18-14 (m, 2H). LC-MS m/z: 315.1 [M+H]⁺. HPLC Purity (214 nm): 100%; t_{R}=6.86 min.

### Example 66—2-Cyclopropoxy-N-(2-cyclopropylethyl)-4-(pyridin-3-yl)-1H-imidazole-1-carboxamide

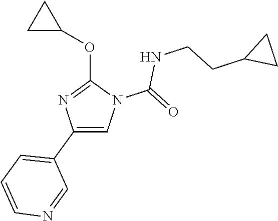 [SMILES: O=C(NCCC1CC1)n1cc(-c2cccnc2)nc1OC1CC1]

To a solution of cyanamide (714 mg, 17 mmol) in cyclopropanol (1.0 g, 17 mmol) at 0° C. was added methanesulfonic acid (1.6 g, 17 mmol). The mixture was stirred at RT for 16 hr and then concentrated in vacuo to give crude cyclopropyl carbamimidate (800 mg) as a red oil.

To a solution of cyclopropyl carbamimidate (800 mg, 8 mmol) in EtOH (15 mL) was added NaHCO₃ (3.4 g, 40 mmol) and 2-bromo-1-(pyridin-3-yl)ethanone (450 mg, 1.6 mmol). The mixture was stirred at 65° C. for 2 hr and purified by silica gel column chromatography (DCM/MeOH=20/1) to give 3-(2-cyclopropoxy-1H-imidazol-4-yl)pyridine (150 mg, 9.3%) as a yellow oil. LC-MS m/z: 202.1 [M+H]⁺. HPLC Purity (254 nm): 100.0%; t_{R}=1.46 min.

Following general procedure C, 3-(2-cyclopropoxy-1H-imidazol-4-yl)pyridine (150 mg, 0.75 mmol) and 2-cyclopropylethanamine (90 mg, 0.75 mmol) afforded the title compound (17.2 mg, 7.4%) as a white solid. ¹H NMR (400 MHz, CDCl₃) δ 9.01 (d, J=1.6 Hz, 1H), 8.51 (d, J=3.5 Hz, 1H), 8.04 (d, J=2.0 Hz, 1H), 7.65 (s, 1H), 6.93 (brs, 1H), 4.59 (brs, 1H), 3.51 (q, J=6.7 Hz, 2H), 1.60-1.51 (m, 3H), 1.02-0.93 (m, 4H), 0.77-0.68 (m, 1H), 0.56-0.50 (m, 2H), 0.16-0.12 (m, 2H). LC-MS m/z: 313.0 [M+H]⁺. HPLC Purity (214 nm): 100.0%; t_{R}=6.63 min.

### Example 67—2-Ethoxy-N-iso-pentyl-4-(pyridin-3-yl)-1H-imidazole-1-carboxamide

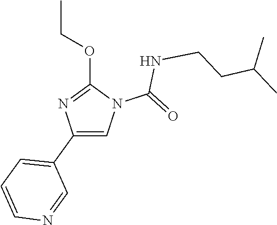 [SMILES: CCOc1nc(-c2cccnc2)cn1C(=O)NCCC(C)C]

Following general procedure C, 3-(2-ethoxy-1H-imidazol-4-yl)pyridine (26 mg, 0.14 mmol) and 3-methylbutan-1-amine (12 mg, 0.14 mmol) afforded the title compound (10.0 mg, 23.7%) as a white solid. ¹H NMR (400 MHz, CDCl₃) δ 8.99 (d, J=1.2 Hz, 1H), 8.50 (d, J=3.5 Hz, 1H), 8.07-7.95 (m, 1H), 7.63 (s, 1H), 7.31 (dd, J=7.4, 4.4 Hz, 1H), 7.02 (s, 1H), 4.69 (q, J=7.1 Hz, 2H), 3.46 (dd, J=12.9, 7.2 Hz, 2H), 1.78-1.60 (m, 1H), 1.57-1.50 (m, 5H), 1.00 (d, J=6.6 Hz, 6H). LC-MS m/z: 303.2 [M+H]⁺. HPLC Purity (214 nm): 100%; t_{R}=0.99 min.

### Example 68—N-(2-Cyclopropylethyl)-2-ethoxy-4-(6-methoxypyridin-3-yl)-1H-imidazole-1-carboxamide

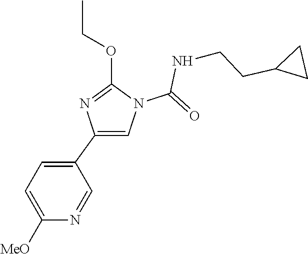 [SMILES: CCOc1nc(-c2ccc(OC)nc2)cn1C(=O)NCCC1CC1]

A solution of 1-(6-methoxypyridin-3-yl)ethanone (4.7 g, 31.1 mmol), NBS (5.5 g, 31.1 mmol) and PTSA (8.0 g, 46.6 mmol) in MCCN (50 mL) was stirred at 80° C. overnight under N₂. The mixture was triturated with MeCN to give 2-bromo-1-(6-methoxypyridin-3-yl)ethanone as a white solid (6 g, 84.3%). LC-MS m/z: 232.1 [M+H]⁺. HPLC Purity (214 nm): 41%; t_{R}=1.75 min.

A suspension of 2-bromo-1-(6-methoxypyridin-3-yl)ethanone (3.0 g, 13.1 mmol), ethyl carbamimidate hydrochloride (2.4 g, 19.7 mmol) and NaHCO₃ (3.3 g, 39.3 mmol) in EtOH (40 mL) was stirred at 65° C. for 5 h under N₂. The mixture was filtered and concentrated to give a residue which was purified by silica gel column chromatography (DCM:MeOH=19:1) and triturated with EA to give 5-(2-ethoxy-1H-imidazol-4-yl)-2-methoxypyridine (200 mg, 7.0%) as a yellow solid. LC-MS m/z: 220.1 [M+H]⁺. HPLC Purity (214 nm): 95%; t_{R}=1.73 min.

Following general procedure C, 5-(2-ethoxy-1H-imidazol-4-yl)-2-methoxypyridine (150 mg, 0.7 mmol) and 2-cyclopropylethanamine hydrochloride (97 mg, 0.8 mmol) afforded the title compound (107.5 mg, 47.6%) as a white solid. ¹H NMR (400 MHz, CDCl₃) δ 8.52 (d, J=2.0 Hz, 1H), 7.88 (dd, J=8.4, 2.4 Hz, 1H), 7.46 (s, 1H), 7.14 (brs, 1H), 6.75 (d, J=8.8 Hz, 1H), 4.66 (q, J=7.2 Hz, 2H), 3.95 (s, 3H), 3.51 (q, J=6.8 Hz, 2H), 1.55-1.49 (m, 5H), 0.75-0.69 (m, 1H), 0.53-0.49 (m, 2H), 0.15-0.11 (m, 2H). LC-MS m/z: 331.2 [M+H]⁺. HPLC Purity (214 nm): 100%; t_{R}=9.08 min.

### Example 69—N-(2-Cyclopropylethyl)-4-(thiazol-4-yl)-1H-imidazole-1-carboxamide

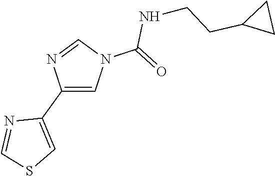 [SMILES: O=C(NCCC1CC1)n1cnc(-c2cscn2)c1]

A mixture of 1-(thiazol-4-yl)ethanone (2.0 g, 15.7 mmol) and pyridinium tribromide (6.0 g, 18.8 mmol) in 33% HBr caustic acid (20 mL) was stirred at RT for 16 hr under N₂. The mixture was filtered and concentrated to afford crude 2-bromo-1-(thiazol-4-yl)ethanone (1.9 g, 86%) as a white solid. LC-MS m/z: 207.3 [M+H]⁺. t_{R}=1.34 min.

Following general procedure A, 2-bromo-1-(thiazol-4-yl)ethanone (1.9 g, 9.2 mmol) afforded 4-(1H-imidazol-4-yl)thiazole (1.2, 89%) as a yellow oil. LC-MS m/z: 152.3 [M+H]⁺. t_{R}=0.48 min.

Following general procedure C, 4-(1H-imidazol-4-yl)thiazole (150 mg, 1.0 mmol) and 2-cyclopropylethanamine hydrochloride (146 mg, 1.2 mmol) afforded the title compound (46.2 mg, 17.8%) as a white solid. ¹H NMR (400 MHz, CDCl₃) δ 8.82 (d, J=1.6 Hz, 1H), 8.22 (s, 1H), 7.77 (d, J=2.0 Hz, 1H), 7.74 (s, 1H), 5.89 (brs, 1H), 3.55 (q, J=6.0 Hz, 2H), 1.55 (q, J=7.2 Hz, 2H), 0.75-0.69 (m, 1H), 0.55-0.49 (m, 2H), 0.15-0.10 (m, 2H). LC-MS m/z: 263.0 [M+H]⁺. HPLC Purity (214 nm): 100%; t_{R}=6.77 min.

### Example 70—N-(2-Cyclopropylethyl)-4-(6-methoxypyridin-3-yl)-1H-imidazole-1-carboxamide

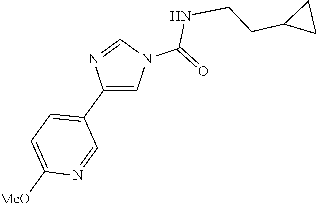 [SMILES: COc1ccc(-c2cn(C(=O)NCCC3CC3)cn2)cn1]

Following general procedure C, 5-(1H-imidazol-4-yl)-2-methoxypyridine (280 mg, 1.6 mmol) and 2-cyclopropylethylamine (163 mg, 1.9 mmol) afforded the title compound (82.5 mg, 18.6%) as a white solid. ¹H NMR (400 MHz, CDCl₃) δ 8.56 (d, J=2.3 Hz, 1H), 8.14 (d, J=1.3 Hz, 1H), 7.99 (dd, J=8.6, 2.4 Hz, 1H), 7.51 (d, J=1.3 Hz, 1H), 6.79 (d, J=8.6 Hz, 1H), 5.84 (s, 1H), 3.97 (s, 3H), 3.56 (dd, J=12.8, 6.9 Hz, 2H), 1.57 (q, J=7.0 Hz, 2H), 0.82-0.67 (m, 1H), 0.58-0.45 (m, 2H), 0.17-0.13 (m, 2H). LC-MS m/z: 287.7 [M+H]⁺. HPLC Purity (214 nm): 98.63%; t_{R}=6.80 min.

### Example 71—4-(6-Methoxypyridin-3-yl)-N-(2-(1-(trifluoromethyl)cyclopropyl)ethyl)-1H-imidazole-1-carboxamide

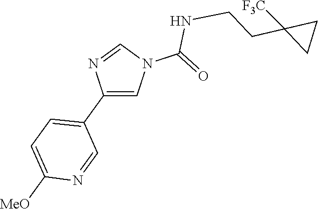 [SMILES: COc1ccc(-c2cn(C(=O)NCCC3(C(F)(F)F)CC3)cn2)cn1]

Following general procedure C, 5-(1H-imidazol-4-yl)-2-methoxypyridine (80 mg, 0.5 mmol) and 2-(1-(trifluoromethyl)cyclopropyl)ethanamine hydrochloride (95 mg, 0.5 mmol) afforded the title compound (119.2 mg, 73.7%) as a white solid. ¹H NMR (400 MHz, CDCl₃) δ 8.55 (d, J=2.4 Hz, 1H), 8.13 (d, J=1.2 Hz, 1H), 7.98 (dd, J=8.8, 2.4 Hz, 1H), 7.49 (d, J=0.8 Hz, 1H), 6.80 (d, J=8.8 Hz, 1H), 5.92 (brs, 1H), 3.97 (s, 3H), 3.65-3.58 (m, 2H), 1.95-1.90 (m, 2H), 1.06 (t, J=6.4 Hz, 2H), 0.72-0.68 (m, 2H) LC-MS m/z: 355.1 [M+H]⁺. HPLC Purity (214 nm): 100%; t_{R}=7.85 min.

### Example 72—N-(2-Cyclopropylethyl)-4-(iso-thiazol-5-yl)-1H-imidazole-1-carboxamide

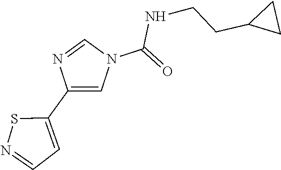 [SMILES: O=C(NCCC1CC1)n1cnc(-c2ccns2)c1]

To a solution of isothiazole-5-carboxylic acid (1.0 g, 7.8 mmol) in DCM (10 mL) were added Et₃N (3.1 g, 31.2 mmol), HATU (3.6 g, 9.4 mmol) and N,O-dimethylhydroxylamine hydrochloride (912 mg, 9.4 mmol). The mixture was stirred at RT for 16 h and purified by silica gel column chromatography (PE:EA=2:1) to give N-methoxy-N-methylisothiazole-5-carboxamide (1.2 g, 90%) as a yellow oil. LC-MS m/z: 173.0 [M+H]⁺. HPLC Purity (214 nm): 89%; t_{R}=1.39 min.

To a solution of N-methoxy-N-methylisothiazole-5-carboxamide (1.2 g, 7.0 mmol) in THF (10 mL) was added MgMeBr (7.0 mL, 3N in THF) at −50° C. The mixture was stirred at RT for 2 h and quenched with NH₄C₁ solution (15 mL). The mixture was extracted with EA (20 mL), the organic layers were dried, filtered, concentrated and purified by silica gel column chromatography (PE:EA=2:1) to give 1-(isothiazol-5-yl)ethan-1-one (650 mg, 73%) as a yellow oil. LC-MS m/z: 128.2 [M+H]⁺. HPLC Purity (214 nm): 95%; t_{R}=0.62 min.

To a solution of 1-(isothiazol-5-yl)ethan-1-one (635 mg, 5.0 mmol) in Et₂O (10 mL) was added Br₂ (960 mg, 6.0 mmol) at 0° C. The mixture was stirred at RT for 2 h and concentrated to give 2-bromo-1-(isothiazol-5-yl)ethan-1-one (850 mg, crude) as a yellow oil. LC-MS m/z: 206.1 [M+H]⁺. HPLC Purity (214 nm): 72%; t_{R}=0.84 min.

Following general procedure A, 2-bromo-1-(isothiazol-5-yl)ethan-1-one (820 mg, 4.0 mmol) afforded 5-(1H-imidazol-4-yl)isothiazole (210 mg, 34%) as a yellow oil. LC-MS m/z: 152.0 [M+H]⁺. HPLC Purity (214 nm): 81%; t_{R}=1.45 min.

Following general procedure C, 5-(1H-imidazol-4-yl)isothiazole (76.0 mg, 0.5 mmol) and 2-cyclopropylethan-1-amine (42.0 mg, 0.5 mmol) afforded the title compound (20.8 mg, 15.9%) as a white solid. ¹H NMR (400 MHz, CDCl₃) δ 8.45 (d, J=1.7 Hz, 1H), 8.13 (d, J=1.1 Hz, 1H), 7.65 (d, J=1.2 Hz, 1H), 7.44 (d, J=1.2 Hz, 1H), 5.85 (s, 1H), 3.57 (q, J=7.2 Hz, 2H), 1.59-1.45 (m, 2H), 0.79-0.63 (m, 1H), 0.61-0.39 (m, 2H), 0.14 (q, J=4.7 Hz, 2H). LC-MS m/z: 263.3 [M+H]⁺. HPLC Purity (214 nm): 100%; t_{R}=6.79 min.

### Example 73—N-(2-Cyclopropylethyl)-4-(2-methyloxazol-4-yl)-1H-imidazole-1-carboxamide

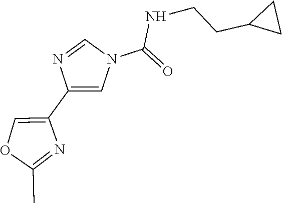 [SMILES: Cc1nc(-c2cn(C(=O)NCCC3CC3)cn2)co1]

A solution of 1-(1H-imidazol-5-yl)ethanone (1.0 g, 9 mmol), pyridinium tribromide (2.9 g, 9 mmol) and HBr (33% in AcOH, 8 mL) in AcOH (6 mL) was stirred for 3 h at RT. Then the reaction mixture was filtered to give 2-bromo-1-(1H-imidazol-5-yl)ethanone (1.0 g, 58%). LC-MS m/z: 189.1 [M+H]⁺. HPLC Purity (214 nm): 90%; t_{R}=0.192 min.

A mixture of 2-bromo-1-(1H-imidazol-5-yl)ethanone (500 mg, 2.6 mmol) and acetamine (800 mg) was heated to 140° C. for 1 h, concentrated and purified by SGC (DCM:MeOH=7:1) to give 4-(1H-imidazol-5-yl)-2-methyloxazole (20 mg, 7.6%) as a colorless oil. LC-MS m/z: 150.2 [M+H]⁺. HPLC Purity (214 nm): 97%; t_{R}=0.22 min.

Following general procedure C, 4-(1H-imidazol-5-yl)-2-methyloxazole (20 mg, 0.15 mmol) and 2-cyclopropylethanamine (16 mg, 0.13 mmol) afforded the title compound (18.3 mg, 17.4%) as a yellow solid. ¹H NMR (400 MHz, CDCl₃) δ 8.19 (s, 1H), 7.93 (s, 1H), 7.57 (s, 1H), 5.91 (brs, 1H), 3.53 (q, J=6.4 Hz, 2H), 2.50 (s, 3H), 1.55-1.50 (m, 2H), 0.77-0.72 (m, 1H), 0.56-0.48 (m, 2H), 0.16-0.10 (m, 2H). LC-MS m/z: 261.0 [M+H]⁺. HPLC Purity (214 nm): >96%; t_{R}=7.09 min.

### Example 74—N-(2-Cyclopropylethyl)-4-(3-fluoro-1H-pyrazol-1-yl)-1H-imidazole-1-carboxamide

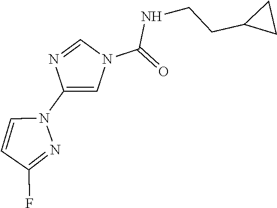 [SMILES: O=C(NCCC1CC1)n1cnc(-n2ccc(F)n2)c1]

A suspension of 4-fluoro-1H-imidazole (250 mg, 2.9 mmol) and 3-fluoro-1H-pyrazole (250 mg, 2.9 mmol) and K₂CO₃ (800 mg, 5.8 mmol) in DMSO (5 mL) was stirred at 110° C. for 3 h. Water was added and the mixture was extracted with DCM (×3). The combined organic layers were dried over Na₂SO₄, filtered and concentrated to give a residue which was purified by SGC (DCM:MeOH=15:1) to give 3-fluoro-1-(1H-imidazol-4-yl)-1H-pyrazole (30 mg, 6.8%) as a yellow oil. LC-MS m/z: 153.1 [M+H]⁺. HPLC Purity (214 nm): 70%; t_{R}=1.39 min.

Following general procedure C, 3-fluoro-1-(1H-imidazol-4-yl)-1H-pyrazole (30 mg, 0.20 mmol) and 2-cyclopropylethanamine (24 mg, 0.2 mmol) afforded the title compound (2.5 mg, 4.8%) as a colorless oil. ¹H NMR (400 MHz, CDCl₃) δ 8.07 (d, J=1.6 Hz, 1H), 8.01 (t, J=2.6 Hz, 1H), 7.33 (d, J=1.2 Hz, 1H), 5.98 (dd, J=5.8, 2.6 Hz, 1H), 5.84 (brs, 1H), 3.54 (q, J=6.8 Hz, 2H), 1.58-1.50 (m, 2H), 0.77-0.67 (m, 1H), 0.57-0.48 (m, 2H), 0.13 (q, J=5.0 Hz, 2H). LC-MS m/z: 264.1 [M+H]⁺. Purity (214 nm): 100%; t_{R}=1.36 min. HPLC Purity (214 nm): >97%; t_{R}=8.09 min.

### Example 75—4-Cyclopentyl-N-iso-pentyl-2-methoxy-1H-imidazole-1-carboxamide

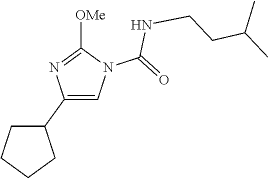 [SMILES: COc1nc(C2CCCC2)cn1C(=O)NCCC(C)C]

Following general procedure D, 5-bromo-2-methoxy-1-((2-(trimethylsilyl)ethoxy)methyl)-1H-imidazole (1.7 g, 5.6 mmol) and cyclopentenylboronic acid (933 mg, 8.3 mmol) afforded 4-cyclopentenyl-2-methoxy-1-((2-(trimethylsilyl)ethoxy)methyl)-1H-imidazole (672 mg, 70%) as a pale-yellow oil. LC-MS m/z: 295.1 [M+H]⁺. HPLC Purity (214 nm): 100%; t_{R}=2.23 min.

To a solution of 4-cyclopentenyl-2-methoxy-1-((2-(trimethylsilyl)ethoxy)methyl)-1H-imidazole (672 mg, 2.36 mmol) in MeOH (10 mL) was added Pd/C (10%, 70 mg) and the mixture stirred at RT for 2 h under H₂. The mixture was filtered and concentrated to give 4-cyclopentyl-2-methoxy-1-((2-(trimethylsilyl)ethoxy)methyl)-1H-imidazole (650 mg, 96.2%) as a pale-yellow oil. LC-MS m/z: 297.1 [M+H]⁺. HPLC Purity (214 nm): 100%; t_{R}=2.35 min.

Following general procedure A, 4-cyclopentyl-2-methoxy-1-((2-(trimethylsilyl)ethoxy)methyl)-1H-imidazole (650 mg, 2.2 mmol) afforded 4-cyclopentyl-2-methoxy-1H-imidazole (360 mg, 98.9%) as a pale-yellow oil which was used in the next step. LC-MS m/z: 167.1 [M+H]⁺. HPLC Purity (214 nm): 100%; t_{R}=1.66 min.

Following general procedure C, 4-cyclopentyl-2-methoxy-1H-imidazole (100 mg, 0.60 mmol) and 3-methylbutan-1-amine (105 mg, 2.0 mmol) afforded the title compound (32 mg, 19.0%) as a colorless oil. ¹H NMR (400 MHz, CDCl₃) δ 6.94 (s, 1H), 6.98 (brs, 1H), 4.14 (s, 3H), 3.37 (q, J=6.4 Hz, 2H), 2.96-2.91 (m, 1H), 2.01-1.96 (m, 2H), 1.77-1.58 (m, 6H), 1.48 (q, J=7.2 Hz, 2H), 1.30-1.21 (m, 1H), 0.94 (d, J=6.8 Hz, 6H). LC-MS m/z: 280.2 [M+H]⁺. HPLC Purity (214 nm): 100%; t_{R}=9.58 min.

### Example 76—1-iso-Pentyl-4-isopropyl-2-methoxy-1H-imidazole

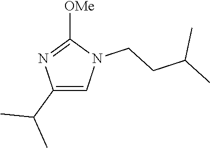

To a solution of 1-bromo-3-methylbutan-2-one (1.0 g, 6.1 mmol) and methyl carbamimidate (1.57 g, 9.15 mmol) in CH₃CN (6 mL) was added aq. NaHCO₃ (2 ml) and the mixture was stirred at 120° C. under N₂ for 20 min under microwave conditions. Purified by silica gel column chromatography (DCM/MeOH=10/1) to give crude 4-iso-propyl-2-methoxy-1H-imidazole (750 mg) as a solid. LC-MS m/z: 141.7 [M+H]⁺. HPLC Purity (214 nm): 86.55%; t_{R}=0.43 min.

Following general procedure C, 4-isopropyl-2-methoxy-1H-imidazole (150 mg, 1.07 mmol) and 3-methylbutan-1-amine (93 mg, 1.07 mmol) afforded the title compound (36.7 mg, 13%) as a colorless oil. ¹H NMR (400 MHz, CDCl₃) δ 6.92 (d, J=0.4 Hz, 1H), 6.83 (brs, 1H), 4.14 (s, 3H), 3.38 (q, J=6.2 Hz, 2H), 2.75-2.66 (m, 1H), 1.67-1.60 (m, 1H), 1.48 (q, J=7.2 Hz, 2H), 1.19 (d, J=6.8 Hz, 6H), 0.95 (d, J=6.6 Hz, 6H). LC-MS m/z: 254.2 [M+H]⁺. HPLC Purity (214 nm): 96.95%; t_{R}=9.05 min.

### Example 77—4-Ethyl-N-iso-pentyl-2-methoxy-1H-imidazole-1-carboxamide

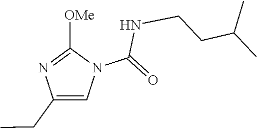 [SMILES: CCc1cn(C(=O)NCCC(C)C)c(OC)n1]

To a solution of 1-bromobutan-2-one (1.0 g, 6.6 mmol) in EtOH (30 mL) was added methyl carbamimidate (1.36 g, 7.9 mmol) and NaHCO₃ (2.2 g, 26.4 mmol). Then the mixture was stirred at 65° C. for 16 h, cooled and filtered. The filtrate was concentrated and purified by silica gel column chromatography (DCM/MeOH=25/1) to give 4-ethyl-2-methoxy-1H-imidazole (500 mg, 60%) as a yellow oil. LC-MS m/z: 127.1 [M+H]⁺. HPLC Purity (214 nm): 31.62%; t_{R}=1.45 min.

Following general procedure C, 4-ethyl-2-methoxy-1H-imidazole (500 mg, 4.0 mmol) and 3-methylbutan-1-amine (348 mg, 4.0 mmol) afforded the title compound (104.2 mg, 11.0%) as a yellow oil. ¹H NMR (400 MHz, CDCl₃) δ 6.94 (dd, J=1.2 Hz, 1H), 6.82 (brs, 1H), 4.14 (s, 3H), 3.43-3.37 (m, 2H), 2.47 (q, J=7.5 Hz, 2H), 1.73-1.63 (m, 1H), 1.49 (q, J=7.6 Hz, 2H), 1.19 (t, J=8.0 Hz, 3H), 0.95 (d, J=6.6 Hz, 6H). LC-MS m/z: 240.1 [M+H]⁺. HPLC Purity (214 nm): 96.7%; t_{R}=7.65 min.

### Example 78—N-iso-pentyl-2-methoxy-1H-imidazole-1-carboxamide

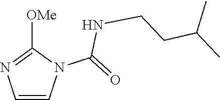

Following general procedure C, 2-methoxy-1H-imidazole (120 mg, 1.20 mmol) and 3-methylbutan-1-amine (104 mg, 1.20 mmol) afforded the title compound (25 mg, 9.7%) as a colorless oil. ¹H NMR (400 MHz, CDCl₃) δ 7.25 (d, J=2.0 Hz, 1H), 6.89 (brs, 1H), 6.57 (d, J=2.0 Hz, 1H), 4.16 (s, 3H), 3.40 (q, J=6.4 Hz, 2H), 1.78-1.60 (m, 1H), 1.50 (q, J=6.8 Hz, 2H), 0.95 (d, J=6.4 Hz, 6H). LC-MS m/z: 212.2 [M+H]⁺. HPLC Purity (214 nm): 96.00%; t_{R}=7.14 min.

### Example 79—4-(tert-Butyl)-N-(4-methylpentan-2-yl)-1H-imidazole-1-carboxamide

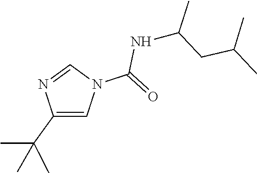 [SMILES: CC(C)CC(C)NC(=O)n1cnc(C(C)(C)C)c1]

A solution of 1-bromo-3,3-dimethylbutan-2-one (2.0 g, 11.2 mmol) in CHONH₂ (12 mL) was stirred at 180° C. under N₂ for 2 h and then cooled and the pH value was adjusted 9. The mixture was poured into water (100 mL) and extracted with EA (100 mL×3). The combined organic layers were washed with brine (100 mL), dried over Na₂SO₄, filtered and concentrated to give 4-(tert-butyl)-1H-imidazole as a brown solid. LC-MS m/z: 125.7 [M+H]⁺. HPLC Purity (214 nm): >70%.

Following general procedure C, 4-(tert-butyl)-1H-imidazole (150 mg, 1.21 mmol) and 4-methylpentan-2-amine (146 mg, 1.45 mmol) afforded the title compound (40.0 mg, 13.2%) as a white solid. ¹H NMR (400 MHz, CDCl₃) δ 8.06 (s, 1H), 6.99 (s, 1H), 5.63 (brs, 1H), 4.25-3.78 (m, 1H), 1.70-1.61 (m, 1H), 1.59-1.45 (m, 1H), 1.40-1.22 (m, 13H), 0.94 (t, J=6.4 Hz, 6H). LC-MS m/z: 252.1 [M+H]⁺. HPLC Purity (214 nm): 93.22%; t_{R}=6.92 min.

### Example 80—4-(tert-Butyl)-N-iso-pentyl-2-(methylthio)-1H-imidazole-1-carboxamide

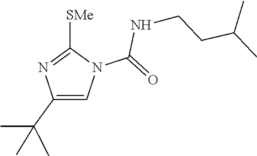 [SMILES: CSc1nc(C(C)(C)C)cn1C(=O)NCCC(C)C]

A mixture of 1-bromo-3,3-dimethylbutan-2-one (1.0 g, 5.6 mmol), NaHCO₃ (1.9 g 22.4 mmol) and methyl carbamimidothioate (0.76 g, 8.6 mmol) in EtOH (20 ml) was stirred at 66° C. under N₂ for 15 h and then cooled, filtered and concentrated. The residue was purified by silica gel column chromatography (DCM/MeOH=9/1) to give 4-(tert-butyl)-2-(methylthio)-1H-imidazole (800 mg, 90%) as an off white solid. LC-MS m/z: 171.7 [M+H]⁺. HPLC Purity (214 nm): 90%

Following general procedure C, 4-(tert-butyl)-2-(methylthio)-1H-imidazole (200 mg, 1.2 mmol) and 3-methylbutan-1-amine (125 mg, 1.44 mmol) afforded the title compound (21.3 mg, 6.4%) as a white solid. ¹H NMR (400 MHz, CDCl₃) δ 7.15 (s, 1H), 6.78 (s, 1H), 3.43 (dt, J=7.3, 5.9 Hz, 2H), 2.61 (s, 3H), 1.73-1.69 (m, 1H), 1.62-1.43 (m, 2H), 1.25 (s, 9H), 0.93 (d, J=6.8 Hz, 6H). LC-MS m/z: 284.1 [M+H]⁺. HPLC Purity (254 nm): 96.8%; t_{R}=8.69 min.

### Example 81—4-(2-Cyanopropan-2-yl)-N-iso-pentyl-2-methoxy-1H-imidazole-1-carboxamide

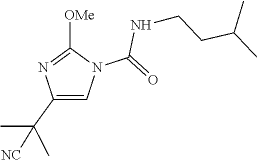 [SMILES: COc1nc(C(C)(C)C#N)cn1C(=O)NCCC(C)C]

A solution of 2,2-dimethyl-3-oxobutanenitrile (310 mg, 2.8 mmol), NBS (397 mg, 2.2 mmol) and PTSA (626 mg, 3.6 mmol) in MeCN (4 mL) was stirred at 80° C. overnight under N₂. The mixture was filtered and concentrated to give 4-bromo-2,2-dimethyl-3-oxobutanenitrile as a yellow solid (310 mg, 58.7%). LC-MS m/z: 192.1 [M+H]⁺. HPLC Purity (214 nm): 60%; t_{R}=1.07 min.

A suspension of 4-bromo-2,2-dimethyl-3-oxobutanenitrile (300 mg, 1.6 mmol), methyl carbamimidate sulfate (408 mg, 2.4 mmol) and NaHCO₃ (663 mg, 7.9 mmol) in EtOH (5 mL) was stirred at 65° C. overnight under N₂. The mixture was filtered, concentrated and purified by silica gel column chromatography (DCM:MeOH=19:1) and triturated with EA to give 2-(2-methoxy-1H-imidazol-4-yl)-2-methylpropanenitrile (310 mg, 119.0%) as a colorless oil. LC-MS m/z: 166.1 [M+H]⁺. HPLC Purity (214 nm): 40%; t_{R}=1.56 min.

Following general procedure C, 2-(2-methoxy-1H-imidazol-4-yl)-2-methyl propanenitrile (300 mg, 1.8 mmol) and 3-methylbutan-1-amine (235 mg, 2.7 mmol) afforded the title compound (5.9 mg, 1.2%) as a colorless oil. ¹H NMR (400 MHz, CDCl₃) δ 7.16 (s, 1H), 6.84 (brs, 1H), 4.17 (s, 3H), 3.42-3.36 (m, 2H), 1.69 (s, 6H), 1.67-1.63 (m, 1H), 1.49 (q, J=7.6 Hz, 2H), 0.95 (d, J=6.8 Hz, 6H). LC-MS m/z: 279.1 [M+H]⁺. HPLC Purity (214 nm): 95.88%; t_{R}=9.00 min.

### Example 82—4-(1,2-Dimethyl-5-oxopyrrolidin-2-yl)-N-iso-pentyl-2-methoxy-1H-imidazole-1-carboxamide

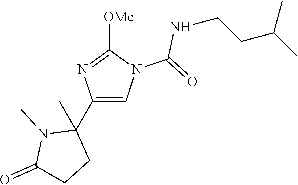 [SMILES: COc1nc(C2(C)CCC(=O)N2C)cn1C(=O)NCCC(C)C]

A solution of 2-aminopentanedioic acid (11 g, 74.8 mmol), DMAP (91.5 mg, 0.75 mmol) and Et₃N (15.1 g, 149.6 mmol) in acetic anhydride (50 ml) was stirred at 60° C. for 16 h. The reaction mixture was and then cooled to RT, diluted with DCM (10 mL), washed with H₂O (10 mL×2), brine (10 mL) and dried over Na₂SO₄. The combined organic layers were washed with brine (20 mL), dried over Na₄SO₄, filtered and concentrated to afford 1,1′-(5-oxopyrrolidine-1,2-diyl)diethanone (8.5 g, 83%) as a yellow oil. LC-MS m/z: 170.1 [M+H]⁺. HPLC Purity (214 nm): 83%; t_{R}=1.26 min.

A solution of 1,1′-(5-oxopyrrolidine-1,2-diyl)diethanone (8.5 g, 41.7 mmol) and K₂CO₃ (17.3 g, 125.1 mmol) in H₂O (30 mL) was stirred at RT under N₂ atmosphere for 3 h. The reaction mixture was diluted with DCM (10 mL), washed with H₂O (10 mL×2), brine (10 mL) and dried over Na₂SO₄. The combined organic layers were washed with brine (20 mL), dried over Na₄SO₄, filtered and concentrated to afford 5-acetylpyrrolidin-2-one (4.6 g, 90%) as a yellow oil. LC-MS m/z: 128.1 [M+H]⁺. HPLC Purity (214 nm): 90%; t_{R}=0.41 min.

To a solution of 5-acetylpyrrolidin-2-one (4.6 g, 32.6 mmol) in THF (30 mL) was added NaH (782.4 mg, 32.6 mmol) at 0° C. and the mixture was stirred at 0° C. for 30 min. CH₃I (5.5 g, 39.1 mmol) in THF (10 mL) was added and the reaction mixture was stirred at RT for 16 h. The reaction mixture was washed with H₂O (10 mL). The combined organic layers were washed with brine (20 mL), dried over Na₄SO₄, filtered and concentrated. The resulting residue was purified by Prep-HPLC to afford 5-acetyl-1,5-dimethylpyrrolidin-2-one (1.4 g, 100%) as a yellow oil. LC-MS m/z: 156.1 [M+H]⁺. HPLC Purity (214 nm): 100%; t_{R}=1.16 min.

A mixture of 5-acetyl-1,5-dimethylpyrrolidin-2-one (300 mg, 1.94 mmol), Br₂ (310.4 mg, 1.94 mmol) in Et₂O (10 mL) was stirred at RT for 2 h under N₂. The mixture was filtered and concentrated to afford 5-(2-bromoacetyl)-1,5-dimethylpyrrolidin-2-one (400 mg) as a white solid. LC-MS m/z: 235.4 [M+H]⁺. t_{R}=1.24 min.

A mixture of 5-(2-bromoacetyl)-1,5-dimethylpyrrolidin-2-one (380 mg, 1.62 mmol), NaHCO₃ (680.4 mg, 8.1 mmol) and methyl carbamimidate (179.8 mg, 2.43 mmol) in EtOH (10 mL) was stirred at 65° C. for 4 h under N₂. Then cooled and diluted with DCM (10 mL), washed with H₂O (10 mL×2), brine (10 mL), dried over Na₂SO₄, concentrated in vacuo to give 5-(2-methoxy-1H-imidazol-4-yl)-1,5-dimethylpyrrolidin-2-one (320 mg) as an off yellow oil. LC-MS m/z: 210.2 [M+H]⁺. t_{R}=0.56 min.

Following general procedure C, 5-(2-methoxy-1H-imidazol-4-yl)-1,5-dimethylpyrrolidin-2-one (320 mg, 1.52 mmol) and 3-methylbutan-1-amine (133.1 mg, 1.53 mmol) afforded the title compound (40.1 mg, 12.5%) as a brown solid. ¹H NMR (400 MHz, CDCl₃) δ 7.09 (s, 1H), 6.84 (s, 1H), 4.11 (s, 3H), 3.39 (q, J=6.0 Hz, 2H), 2.68-2.57 (m, 2H), 2.61 (s, 3H), 2.47-2.40 (m, 2H), 1.95-1.87 (m, 1H), 1.71-1.62 (m, 1H), 1.51 (s, 3H), 1.50-1.45 (m, 2H), 0.95 (d, J=6.6 Hz, 6H). LC-MS m/z: 323.2 [M+H]⁺. HPLC Purity (214 nm): 100%; t_{R}=7.28 min.

### Example 83—N-iso-Pentyl-2-methoxy-4-(pyridazin-3-yl)-1H-imidazole-1-carboxamide

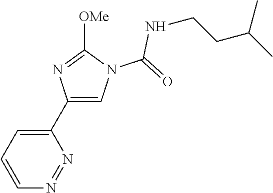 [SMILES: COc1nc(-c2cccnn2)cn1C(=O)NCCC(C)C]

A mixture of 1-(pyridazin-3-yl)ethanone (900 mg, 7.4 mmol) and pyridinium tribromide (2.3 g, 7.4 mmol) in 33% HBr caustic acid (10 mL) was stirred at RT for 16 h under N₂. The mixture was filtered and concentrated to afford 2-bromo-1-(pyridazin-3-yl)ethanone (1.1 g, 96%) as a white solid. LC-MS m/z: 202.3 [M+H]⁺. t_{R}=1.37 min.

A mixture of 2-bromo-1-(pyridazin-3-yl)ethanone (500 mg, 2.5 mmol), NaHCO₃ (1.05 g, 12.5 mmol) and methyl carbamimidate (2.8 g, 3.75 mmol) in EtOH (10 mL) was stirred at 65° C. for 4 h under N₂. Then cooled and diluted with DCM (10 mL), washed with H₂O (10 mL×2), brine (10 mL), dried over Na₂SO₄ and concentrated in vacuo to give 3-(2-methoxy-1H-imidazol-4-yl)pyridazine (150 mg, 86%) as an off yellow solid. LC-MS m/z: 177.4 [M+H]⁺. HPLC Purity (214 nm): 87%; t_{R}=0.42 min.

Following general procedure B (method 2), 3-(2-methoxy-1H-imidazol-4-yl)-pyridazine (150 mg, 0.85 mmol), triphosgene (171.3 mg, 0.85 mmol) and 3-methylbutan-1-amine (74.0 mg, 0.85 mmol) afforded the title compound (12 mg, 4.9%) as a brown solid. ¹H NMR (400 MHz, CDCl₃) δ 9.05 (dd, J=4.9, 1.6 Hz, 1H), 8.16 (s, 1H), 7.96 (dd, J=8.6, 1.5 Hz, 1H), 7.47 (dd, J=8.5, 5.0 Hz, 1H), 6.92 (brs, 1H), 4.27 (s, 3H), 3.44 (dt, J=14.7, 5.9 Hz, 2H), 1.72-1.62 (m, 1H), 1.55-1.49 (m, 2H), 0.97 (d, J=6.6 Hz, 6H). LC-MS m/z: 290.1 [M+H]⁺. HPLC Purity (214 nm): 100%; t_{R}=7.63 min.

### Example 84—N-iso-pentyl-2-methoxy-4-(tetrahydrofuran-2-yl)-1H-imidazole-1-carboxamide

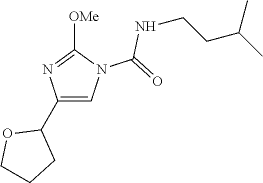 [SMILES: COc1nc(C2CCCO2)cn1C(=O)NCCC(C)C]

To a solution of 1-(tetrahydrofuran-2-yl)ethan-1-one (228 mg, 2.0 mmol) in Et₂O (10 mL) was added Br₂ (320 mg, 2.0 mmol) at 0° C. The mixture was stirred at RT for 2 h and then concentrated to give crude 2-bromo-1-(tetrahydrofuran-2-yl)-ethan-1-one (390 mg) as a yellow oil. LC-MS m/z: 193.1 [M+H]⁺. HPLC Purity (214 nm): 19%; t_{R}=0.49 min.

To a solution of 2-bromo-1-(tetrahydrofuran-2-yl)ethan-1-one (384 mg, 2.0 mmol) in EtOH (10 mL) was added NaHCO₃ (672 mg, 8.0 mmol) and methyl carbamimidate (688 mg, 4.0 mmol). The mixture was stirred at 70° C. for 12 h and purified by silica gel column chromatography (DCM:MeOH=20:1) to give crude 2-methoxy-4-(tetrahydrofuran-2-yl)-1H-imidazole (35 mg) as a yellow oil. LC-MS m/z: 169.1 [M+H]⁺. Purity (214 nm): 15%; t_{R}=1.09 min.

Following general procedure C, 2-methoxy-4-(tetrahydrofuran-2-yl)-1H-imidazole (34.0 mg, 0.2 mmol) and 3-methylbutan-1-amine (17.0 mg, 0.2 mmol) afforded the title compound 8.0 mg, 17.8%) as a white solid. ¹H NMR (400 MHz, CDCl₃) δ 7.27 (s, 1H), 6.84 (brs, 1H), 4.75 (t, J=6.3 Hz, 1H), 4.16 (s, 3H), 4.01 (dd, J=13.6, 7.6 Hz, 1H), 3.85 (dd, J=13.4, 7.8 Hz, 1H), 3.40 (q, J=6.0 Hz, 2H), 2.20-2.12 (m, 1H), 2.09-1.91 (m, 3H), 1.72-1.66 (m, 1H), 1.48 (q, J=7.6 Hz, 2H), 0.95 (d, J=6.6 Hz, 6H). LC-MS m/z: 282.1 [M+H]⁺. HPLC Purity (214 nm): 100%; t_{R}=8.11 min.

### Example 85—N-iso-Pentyl-2-methoxy-4-(tetrahydrofuran-3-yl)-1H-imidazole-1-carboxamide

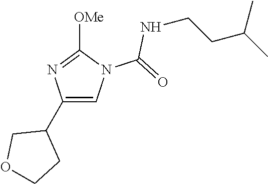 [SMILES: COc1nc(C2CCOC2)cn1C(=O)NCCC(C)C]

To a solution of 1-(tetrahydrofuran-3-yl)ethan-1-one (560 mg, 4.9 mmol) in Et₂O (10 mL) was added Br₂ (784 mg, 4.9 mmol) at 0° C. The mixture was stirred at RT for 2 h and concentrated to give crude 2-bromo-1-(tetrahydrofuran-3-yl)ethan-1-one (950 mg) as a yellow oil. LC-MS m/z: 193.1 [M+H]⁺. HPLC Purity (214 nm): 290%; t_{R}=0.49 min.

To a solution of 2-bromo-1-(tetrahydrofuran-3-yl)ethan-1-one (768 mg, 4.0 mmol) in EtOH (10 mL) was added NaHCO₃ (1.3 g, 16.0 mmol) and methyl carbamimidate (1.38 g, 8.0 mmol). The mixture was stirred at 70° C. for 12 h and purified by silica gel column chromatography (DCM:MeOH=20:1) to give crude 2-methoxy-4-(tetrahydrofuran-3-yl)-1H-imidazole (100 mg) as a yellow oil. LC-MS m/z: 169.3 [M+H]⁺. HPLC Purity (214 nm): 15%; t_{R}=0.22 min.

Following general procedure C, 2-methoxy-4-(tetrahydrofuran-3-yl)-1H-imidazole (101.0 mg, 0.6 mmol) and 3-methylbutan-1-amine (52.0 mg, 0.6 mmol) afforded the title compound (44.2 mg, 26.5%) as a white solid. ¹H NMR (400 MHz, CDCl₃) δ 7.03 (s, 1H), 6.83 (brs, 1H), 4.14 (s, 3H), 4.05 (t, J=7.7 Hz, 1H), 3.92 (dq, J=32.5, 7.3 Hz, 2H), 3.74 (t, J=7.4 Hz, 1H), 3.38 (q, J=6.8 Hz, 2H), 3.33-3.26 (m, 1H), 2.30-2.20 (m, 1H), 2.08-1.99 (m, 1H), 1.72-1.62 (m, 1H), 1.49 (q, J=7.2 Hz, 2H), 0.90 (d, J=6.4 Hz, 6H). LC-MS m/z: 282.1 [M+H]⁺. HPLC Purity (214 nm): 100%; t_{R}=7.78 min.

### Example 86—4-(tert-Butyl)-N-iso-butyl-2-methoxy-1H-imidazole-1-carboxamide

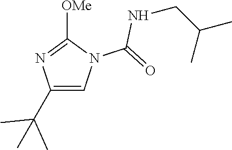 [SMILES: COc1nc(C(C)(C)C)cn1C(=O)NCC(C)C]

Following general procedure C, 4-(tert-butyl)-2-methoxy-1H-imidazole (289 mg, 2.4 mmol) and 2-methylpropan-1-amine (175 mg, 2.4 mmol) afforded the title compound (49.6 mg, 10.6%) as a white solid. ¹H NMR (400 MHz, CDCl₃) δ 6.95 (s, 1H), 6.91 (s, 1H), 4.15 (s, 3H), 3.21 (t, J=6.4 Hz, 2H), 1.92-1.86 (m, 1H), 1.22 (s, 9H), 0.95 (d, J=6.7 Hz, 6H). LC-MS m/z: 254.1 [M+H]⁺. HPLC Purity (214 nm): 100%; t_{R}=8.42 min.

### Example 87—4-(tert-Butyl)-N-iso-butyl-1H-imidazole-1-carboxamide

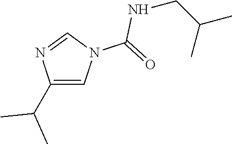

Following general procedure C, 4-(tert-butyl)-1H-imidazole (500 mg, 4.0 mmol) and 2-methylpropan-1-amine (350 mg, 4.8 mmol) afforded the title compound (35.1 mg, 3.9%) as a white solid. ¹H NMR (400 MHz, CDCl₃) δ 8.03 (s, 1H), 6.96 (s, 1H), 5.56 (s, 1H), 3.25 (t, J=6.4 Hz, 2H), 1.99-1.91 (m, 1H), 1.30 (s, 9H), 0.99 (d, J=6.6 Hz, 6H). LC-MS m/z: 224.1 [M+H]⁺. HPLC Purity (214 nm): 100%; t_{R}=6.16 min.

### Example 88—4-(4-Cyanophenyl)-N-iso-pentyl-1H-imidazole-1-carboxamide

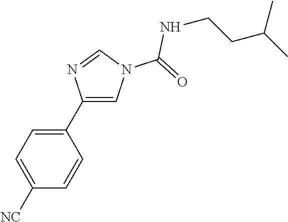 [SMILES: CC(C)CCNC(=O)n1cnc(-c2ccc(C#N)cc2)c1]

A solution of 4-(2-bromoacetyl)benzonitrile (2.24 g, 10 mmol) in formamide (6 mL) was stirred at 180° C. for 2 h. Then the volatiles were removed under vacuum at 50° C. to give 4-(1H-imidazol-4-yl)benzonitrile (1 g, 96%) as a yellow solid. LC-MS m/z: 170.1 [M+H]⁺. HPLC Purity (214 nm): >95%; t_{R}=1.60 min.

Following general procedure C, 4-(1H-imidazol-4-yl)benzonitrile (200 mg, 1.2 mmol) and 3-methylbutan-1-amine (157 mg, 1.8 mmol) afforded the title compound (99 mg, 29.7%) as a white solid. 1H NMR (400 MHz, CDCl₃) δ 8.14 (s, 1H), 7.89 (d, J=8.4 Hz, 2H), 7.73 (s, 1H), 7.68 (d, J=8.0 Hz, 2H), 5.73 (brs, 1H), 3.52-3.46 (m, 2H), 1.73-1.66 (m, 1H), 1.56 (q, J=7.2 Hz, 2H), 0.98 (d, J=6.8 Hz, 6H). LC-MS m/z: 283.1 [M+H]⁺. HPLC Purity (214 nm): 96.55%; t_{R}=8.40 min.

### Example 89—4-(tert-Butyl)-2-methoxy-N-(3-phenoxypropyl)-1H-imidazole-1-carboxamide

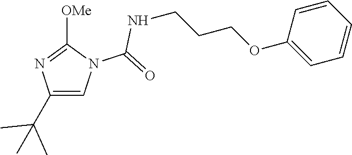 [SMILES: COc1nc(C(C)(C)C)cn1C(=O)NCCCOc1ccccc1]

Following general procedure C, 4-tert-butyl-2-methoxy-1H-imidazole (300 mg, 1.95 mmol) and 3-phenoxypropan-1-amine (440 mg, 2.9 mmol) afforded the title compound (87.0 mg, 13.5%) as a white solid. ¹H NMR (400 MHz, CDCl₃) δ 7.30 (t, J=8.0 Hz, 2H), 7.16 (brs, 1H), 6.97 (t, J=7.4 Hz, 1H), 6.92-6.90 (m, 3H), 4.07 (t, J=5.8 Hz, 2H), 4.04 (s, 3H), 3.60 (q, J=6.4 Hz, 2H), 2.09 (p, J=6.2 Hz, 2H), 1.21 (s, 9H). LC-MS m/z: 332.1 [M+H]⁺. HPLC Purity (214 nm): >96%; t_{R}=8.92 min.

### Example 90—4-(tert-Butyl)-2-methoxy-N-(2-(1-(trifluoromethyl)cyclopropyl)ethyl)-1H-imidazole-1-carboxamide

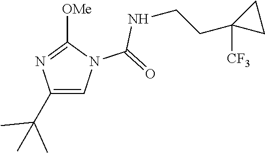 [SMILES: COc1nc(C(C)(C)C)cn1C(=O)NCCC1(C(F)(F)F)CC1]

Following general procedure C, 4-(tert-butyl)-2-methoxy-1H-imidazole (200 mg, 1.3 mmol) and 2-(1-(trifluoromethyl)cyclopropyl)ethan-1-amine hydrochloride (246 mg, 1.3 mmol) afforded the title compound (14.5 mg, 3.1%) as a colorless oil. ¹H NMR (400 MHz, CDCl₃) δ 7.10 (brs, 1H), 6.88 (s, 1H), 4.14 (s, 3H), 3.52 (q, J=6.7 Hz, 2H), 1.86 (t, J=7.2 Hz, 2H), 1.21 (s, 9H), 1.04-1.00 (m, 2H), 0.69-0.65 (m, 2H). LC-MS m/z: 334.2 [M+H]⁺. HPLC Purity (214 nm): 100%; t_{R}=9.01 min.

### Example 91—4-(tert-Butyl)-2-methoxy-N-(4,4,4-trifluorobutyl)-1H-imidazole-1-carboxamide

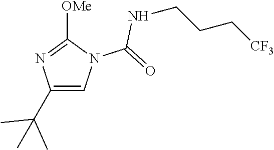 [SMILES: COc1nc(C(C)(C)C)cn1C(=O)NCCCC(F)(F)F]

Following general procedure C, 4-(tert-butyl)-2-methoxy-1H-imidazole (200 mg, 1.3 mmol) and 4,4,4-trifluorobutan-1-amine hydrochloride (212 mg, 1.3 mmol) afforded the title compound (14.5 mg, 3.1%) as a colorless oil. ¹H NMR (400 MHz, CDCl₃) δ 6.99 (brs, 1H), 6.89 (s, 1H), 4.15 (s, 3H), 3.45 (q, J=6.7 Hz, 2H), 2.26-2.09 (m, 2H), 1.88 (dt, J=14.6, 7.2 Hz, 2H), 1.22 (s, 9H). LC-MS m/z: 308.1 [M+H]⁺. HPLC Purity (214 nm): 100%; t_{R}=8.49 min.

### Example 92—4-(tert-Butyl)-N-(2-cyclopropylethyl)-2-ethoxy-1H-imidazole-1-carboxamide

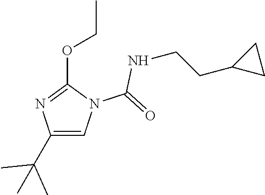 [SMILES: CCOc1nc(C(C)(C)C)cn1C(=O)NCCC1CC1]

To a mixture of 1-bromo-3,3-dimethylbutan-2-one (600 mg, 3.35 mmol) and ethyl carbamimidate hydrochloride (623 mg, 5.0 mmol) in EtOH (10 mL) was added NaHCO₃ (2.8 g, 13.4 mmol) and the mixture was stirred at 50° C. for 15 h. Then filtered, concentrated and the residue was purified by silica gel column chromatography (DCM/MeOH=9:1) to give 4-(tert-butyl)-2-ethoxy-1H-imidazole (230 mg, solid). LC-MS m/z: 169.1 [M+H]⁺. HPLC Purity (214 nm): 98%.

Following general procedure C, 4-(tert-butyl)-2-ethoxy-1H-imidazole (200 mg, 1.20 mmol) and 2-cyclopropylethan-1-amine (122 mg, 1.44 mmol) afforded the title compound (42.9 mg, 13.0%) as a colorless oil. ¹H NMR (400 MHz, DMSO-d₆) δ 7.50 (t, J=5.4 Hz, 1H), 6.75 (s, 1H), 4.43 (q, J=7.0 Hz, 2H), 3.44-3.16 (m, 2H), 1.42 (q, J=6.8 Hz, 3H), 1.37 (t, J=7.2 Hz, 2H), 1.16 (s, 9H), 0.79-0.63 (m, 1H), 0.53-0.35 (m, 2H), 0.13-0.02 (m, 2H). LC-MS m/z: 280.1 [M+H]⁺. HPLC Purity (214 nm): 100%; t_{R}=8.82 min.

### Example 93—4-(tert-Butyl)-N-cyclohexyl-2-methoxy-1H-imidazole-1-carboxamide

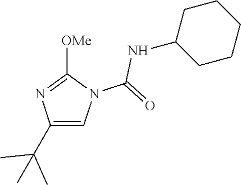 [SMILES: COc1nc(C(C)(C)C)cn1C(=O)NC1CCCCC1]

Following general procedure C, 4-tert-butyl)-2-ethoxy-1H-imidazole (200 mg, 1.20 mmol) cyclohexanamine (143 mg, 1.44 mmol) afforded the title compound (44.0 mg, 11.0%) as a colorless oil. ¹H NMR (400 MHz, CDCl₃) δ 6.90 (s, 1H), 6.79 (d, J=7.1 Hz, 1H), 4.13 (s, 3H), 3.85-3.75 (m, 1H), 2.05-1.95 (m, 2H), 1.77-1.62 (m, 2H), 1.66-1.63 (m, 1H), 1.49-1.35 (m, 2H), 1.33-1.24 (m, 3H), 1.21 (s, 9H). LC-MS m/z: 280.3 [M+H]⁺. HPLC Purity (214 nm): 96.71%; t_{R}=9.31 min.

### Example 94—2-Methoxy-4-(6-methoxypyridin-3-yl)-N-(2-(1-(trifluoromethyl)cyclopropyl) ethyl)-1H-imidazole-1-carboxamide

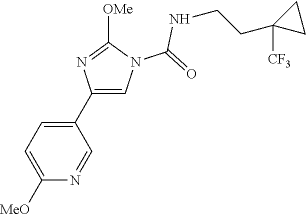 [SMILES: COc1ccc(-c2cn(C(=O)NCCC3(C(F)(F)F)CC3)c(OC)n2)cn1]

To a solution of 1-(6-methoxypyridin-3-yl)ethan-1-one (1.0 g, 6.6 mmol) in CH₃CN (15 ml) were added NBS (1.4 g, 7.9 mmol) and PTSA (1.36 g, 7.9 mmol). The reaction mixture was stirred at 80° C. for 15 h, cooled to 0° C. and MeOH (500 mL) was added. The reaction mixture was stirred for 0.5 h and diluted with water. The organic layer was dried over Na₂SO₄, filtered and concentrated in vacuo to give a residue which was purified by silica gel column chromatography (MeOH:DCM=1:9) to give 2-bromo-1-(6-methoxypyridin-3-yl)ethan-1-one (890 mg) as a yellow solid. LC-MS m/z: 230.7 [M+H]⁺. HPLC Purity (214 nm): 90%.

To a solution of 2-bromo-1-(6-methoxypyridin-3-yl)ethan-1-one (890 mg, 3.9 mmol) and methyl carbamimidate hydrochloride (635 mg, 5.9 mmol) in EtOH (12 mL) was added NaHCO₃ (1.3 g, 15.6 mmol) and the mixture was stirred at 66° C. for 15 h. Then filtered, concentrated and the residue was purified by silica gel column chromatography (DCM/MeOH=9:1) to give 2-methoxy-5-(2-methoxy-1H-imidazol-4-yl)pyridine (350 mg, 44%) LC-MS m/z: 206.1 [M+H]⁺. HPLC Purity (214 nm): 90%.

Following general procedure C, 2-methoxy-5-(2-methoxy-1H-imidazol-4-yl)pyridine (120 mg, 0.6 mmol) and 2-(1-(trifluoromethyl)cyclopropyl)ethan-1-amine (110 mg, 0.72 mmol) afforded the title compound (11.3 mg, 5.0%) as a white solid. ¹H NMR (400 MHz, CDCl₃) δ 8.52 (d, J=2.2 Hz, 1H), 7.89 (dd, J=8.6, 2.3 Hz, 1H), 7.44 (s, 1H), 7.15 (brs, 1H), 6.75 (d, J=8.6 Hz, 1H), 4.24 (s, 3H), 3.96 (s, 3H), 3.57 (q, J=6.7 Hz, 2H), 1.88 (t, 1=7.4 Hz, 2H), 1.13-0.87 (m, 2H), 0.68 (s, 2H). LC-MS m/z: 385.1 [M+H]⁺. Purity (214 nm): 100%; t_{R}=8.90 min.

### Example 95—4-(tert-Butyl)-N-(3-cyclopropylpropyl)-2-methoxy-1H-imidazole-1-carboxamide

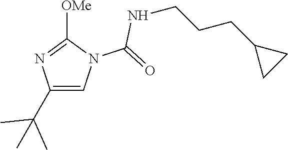 [SMILES: COc1nc(C(C)(C)C)cn1C(=O)NCCCC1CC1]

Following general procedure C, 4-tert-butyl-2-methoxy-1H-imidazole (200 mg, 1.3 mmol) and 3-cyclopropylpropan-1-amine (129 mg, 1.3 mmol) afforded the title compound (48.8 mg, 17.5%) as a colorless oil. ¹H NMR (500 MHz, CDCl₃) δ 6.89 (s, 1H), 6.87 (brs, 1H), 4.14 (s, 3H), 3.39 (q, J=7.1 Hz, 2H), 1.73-1.64 (m, 2H), 1.29-1.24 (m, 2H), 1.21 (s, 9H), 0.69 (s, 1H), 0.48-0.39 (m, 2H), 0.04-0.01 (m, 2H). LC-MS m/z: 280.3 [M+H]⁺. HPLC Purity (214 nm): 99.99%; t_{R}=9.84 min.

### Example 96—4-(tert-Butyl)-2-methoxy-N-(3-(1-(trifluoromethyl)cyclopropyl)propyl)-1H-imidazole-1-carboxamide

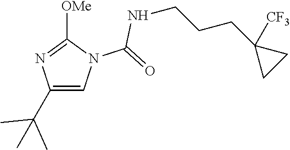 [SMILES: COc1nc(C(C)(C)C)cn1C(=O)NCCCC1(C(F)(F)F)CC1]

Following general procedure C, 4-tert-butyl-2-methoxy-1H-imidazole (150 mg, 1.0 mmol) and 3-(1-(trifluoromethyl)cyclopropyl)propan-1-amine (300 mg, 1.8 mmol) afforded the title compound (106.3 mg, 29.7%) as a colorless oil. ¹H NMR (400 MHz, DMSO-d₆) δ 7.65 (brs, 1H), 6.76 (s, 1H), 3.99 (s, 3H), 3.19 (q, J=6.4 Hz, 2H), 1.63-1.55 (m, 4H), 1.16 (s, 9H), 0.90-0.87 (m, 2H), 0.72-0.68 (m, 2H). LC-MS m/z: 348.1 [M+H]⁺. HPLC Purity (214 nm): 100%; t_{R}=9.03 min.

### Example 97—4-(tert-Butyl)-N-(4-cyclopropylbutyl)-2-methoxy-1H-imidazole-1-carboxamide

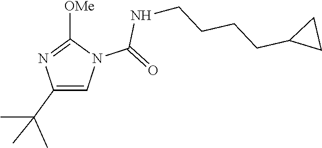 [SMILES: COc1nc(C(C)(C)C)cn1C(=O)NCCCCC1CC1]

Following general procedure C, 4-tert-butyl-2-methoxy-1H-imidazole (0.10 g, 0.65 mmol) and 4-cyclopropylbutan-1-amine hydrochloride (0.11 g, 0.71 mmol) afforded the title compound (67.7 mg, 39.5%) as a white solid. ¹H NMR (400 MHz, CDCl₃) δ 6.90 (s, 1H), 6.89 (brs, 1H), 4.14 (d, 3H), 3.36 (q, J=6.8 Hz, 2H), 1.65-1.57 (m, 2H), 1.49-1.42 (m, 2H), 1.28-1.21 (m, 2H), 1.21 (s, 9H), 0.67-0.64 (m, 1H), 0.43-0.38 (m, 2H), 0.02-0.00 (m, 2H). LC-MS m/z: 294.3 [M+H]⁺. HPLC Purity (214 nm): >99%; t_{R}=10.04 min.

### Example 98—4-(tert-Butyl)-2-methoxy-N-(4-methylpent-2-yn-1-yl)-1H-imidazole-1-carboxamide

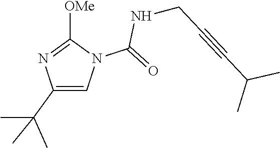 [SMILES: COc1nc(C(C)(C)C)cn1C(=O)NCC#CC(C)C]

To a solution of 4-methylpent-2-yn-1-ol (2.0 g, 20.38 mmol) in Et₂O (20 mL) was added TEA (6.19 g, 8.52 mL) at 0° C. and the reaction mixture was stirred at 0° C. for 10 min. Then MsCl (3.03 g, 2.05 mL) was added and the resulting mixture was stirred at RT for 2 h, diluted with Et₂O and washed with brine. The organic layer was dried over Na₂SO₄, filtered and concentrated to afford crude 4-methylpent-2-ynyl methanesulfonate (3.59 g, 100%) which was used directly in the next step. LC-MS m/z: 177.0 [M+H]⁺. t_{R}=1.81 min.

To a solution of crude 4-methylpent-2-ynyl methanesulfonate (3.59 g, 20.37 mmol) in DMF (40 mL) was added potassium 1,3-dioxoisoindolin-2-ide (3.77 g, 20.37 mmol) and the reaction mixture was stirred at 85° C. for 3 h, diluted with EA and washed with brine. The organic layer was dried over Na₂SO₄, filtered and concentrated. The residue was purified by flash column (PE:EA=9:1) to afford 2-(4-methylpent-2-ynyl)isoindoline-1,3-dione (2 g, 41.5%) as a white solid. ¹H NMR (400 MHz, CDCl₃) δ 7.90-7.85 (m, 2H), 7.75-7.72 (m, 2H), 4.43 (d, J=2.1 Hz, 2H), 2.57-2.45 (m, 1H), 1.12 (d, J=6.9 Hz, 6H). LC-MS m/z: 228.1 [M+H]⁺. t_{R}=1.40 min.

To a solution of 2-(4-methylpent-2-ynyl)isoindoline-1,3-dione (5.06 g, 22.3 mmol) in EtOH (100 mL), was added hydrazine hydrate (3.28 g, 55.76 mmol, 85%) and the resulting reaction mixture was stirred at 80° C. for 3 h, cooled to RT, quenched with concentrated HCl (12.5 mL) and concentrated under reduced pressure. The residue was neutralized with 5 N aq. NaOH and the mixture was extracted with DCM and the organic layers were washed with brine, dried over Na₂SO₄, filtered and concentrated. To the residue was added 4N HCl/dioxane (10 mL) and the solution was concentrated under reduced pressure and the solid washed with Et₂O to afford 4-methylpent-2-yn-1-amine hydrochloride (3.41 g, 90.2%) as a white solid. ¹H NMR (400 MHz, DMSO) δ 8.45 (s, 3H), 3.65 (d, J=2.1 Hz, 2H), 2.75-2.55 (m, 1H), 1.13 (d, J=6.9 Hz, 6H). LC-MS m/z: 98.2 [M+H]⁺. t_{R}=0.36 min.

Following general procedure C, 4-tert-butyl-2-methoxy-1H-imidazole (100 mg, 0.65 mmol) and 4-methylpent-2-yn-1-amine hydrochloride (69.30 mg, 0.71 mmol) afforded the title compound as a white solid (37 mg, 20.6%). ¹H NMR (400 MHz, CDCl₃) δ 7.00 (brs, 1H), 6.90 (s, 1H), 4.15 (s, 3H), 4.13 (dd, J=5.3, 2.1 Hz, 2H), 2.62-2.55 (m, 1H), 1.21 (s, 9H), 1.16 (d, 0.1=6.9 Hz, 6H). LC-MS m/z: 278.1 [M+H]⁺. HPLC Purity (214 nm): 100%; t_{R}=9.76 min.

### Example 99—4-(tert-Butyl)-N-(3,3-difluorocyclohexyl)-2-methoxy-1H-imidazole-1-carboxamide

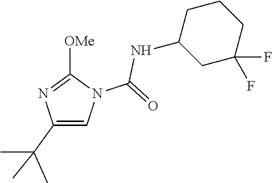 [SMILES: COc1nc(C(C)(C)C)cn1C(=O)NC1CCCC(F)(F)C1]

Following general procedure C, 4-tert-butyl-2-methoxy-1H-imidazole (100 mg, 0.65 mmol) and 3,3-difluorocyclohexanamine (88 mg, 0.65 mmol) afforded the title compound (26.8 mg, 13.4%) as a colorless oil. ¹H NMR (400 MHz, DMSO-d₆) δ 7.52 (d, J=8.0 Hz, 1H), 6.78 (s, 1H), 4.01 (s, 3H), 3.97-3.79 (m, 1H), 2.39-2.30 (m, 1H), 2.11-1.91 (m, 2H), 1.75-1.69 (s, 3H), 1.52-1.43 (m, 2H), 1.17 (s, 9H). LC-MS m/z: 316.2 [M+H]⁺. HPLC Purity (214 nm): 98%; t_{R}=9.71 min.

### Example 100—N-iso-Pentyl-2-methoxy-4-(tert-pentyl)-1H-imidazole-1-carboxamide

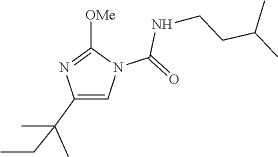 [SMILES: CCC(C)(C)c1cn(C(=O)NCCC(C)C)c(OC)n1]

A solution of 3,3-dimethylpentan-2-one (920 mg, 8.1 mmol) and Br₂ (1.3 g, 8.1 mmol) in Et₂O (10 mL) was stirred at RT for 2 h. The resulting solution was concentrated under vacuum to afford 1-bromo-3,3-dimethylpentan-2-one (1.4 g, 56.5%) as a colorless oil.

A suspension of 1-bromo-3,3-dimethylpentan-2-one (1.4 g, 7.3 mmol), methyl carbamimidate sulfate (1.87 g, 10.9 mmol) and NaHCO₃ (3.0 g, 36.3 mmol) in EtOH (15 mL) was stirred at 65° C. for 5 h under N₂. The mixture was filtered and concentrated to give a residue which was purified by silica gel column chromatography (DCM:MeOH=20:1) to afford 2-methoxy-4-tert-pentyl-1H-imidazole (500 mg, 41.0%) as a yellow oil. LC-MS m/z: 169.1 [M+H]⁺. HPLC Purity (214 nm): 65%; t_{R}=1.55 min.

Following general procedure C, 2-methoxy-4-tert-pentyl-1H-imidazole (100 mg, 0.6 mmol) and 3-methylbutan-1-amine (78 mg, 0.9 mmol) afforded the title compound (41.3 mg, 24.7%) as a colorless oil. ¹H NMR (400 MHz, DMSO-d₆) δ 7.60 (brs, 1H), 6.76 (s, 1H), 3.98 (s, 3H), 3.22 (q, J=8.0 Hz, 2H), 1.61-1.47 (m, 3H), 1.40 (q, J=7.6 Hz, 2H), 1.10 (s, 6H), 0.89 (d, J=6.4 Hz, 6H), 0.68 (t, J=7.6 Hz, 3H). LC-MS m/z: 282.2 [M+H]⁺. HPLC Purity (214 nm): 100%; t_{R}=9.20 min.

### Example 101—4-(tert-Butyl)-2-methoxy-N-(3-phenylpropyl)-1H-imidazole-1-carboxamide

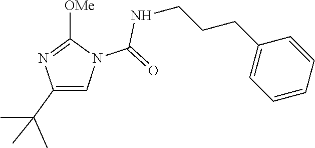 [SMILES: COc1nc(C(C)(C)C)cn1C(=O)NCCCc1ccccc1]

Following general procedure C, 4-(tert-butyl)-2-ethoxy-1H-imidazole (300 mg, 1.8 mmol) and 3-phenylpropan-1-amine (297 mg, 2.2 mmol) afforded the title compound (86.1 mg, 14.0%) as a colorless oil. ¹H NMR (400 MHz, CDCl₃) δ 7.28 (dd, J=13.5, 6.4 Hz, 2H), 7.25-7.19 (m, 3H), 6.90 (s, 1H), 6.89 (brs, 1H), 4.13 (s, 3H), 3.38 (q, J=6.9 Hz, 2H), 2.69 (t, J=7.6 HZ, 2H), 1.99-1.82 (m, 2H), 1.22 (s, 9H). LC-MS m/z: 316.1 [M+H]⁺. Purity (214 nm): 100%; t_{R}=8.95 min.

### Example 102—4-(tert-Butyl)-2-methoxy-N-(4-methylpentyl)-1H-imidazole-1-carboxamide

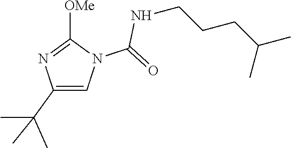 [SMILES: COc1nc(C(C)(C)C)cn1C(=O)NCCCC(C)C]

Following general procedure C, 4-tert-butyl-2-methoxy-1H-Imidazole (0.15 g, 0.97 mmol) and 4-methylpentan-1-amine (0.11 g, 1.07 mmol) afforded the title compound (18.3 mg, 6.8%) as a colorless oil. ¹H NMR (400 MHz, CDCl₃) δ 6.90 (s, 2H), 4.14 (s, 3H), 2.01 (q, J=6.0 Hz, 2H), 1.59-1.55 (m, 3H), 1.26-1.21 (m, 2H), 1.20 (s, 9H), 0.90 (d, J=12.5 Hz, 6H). LC-MS m/z: 282.3 [M+H]⁺. HPLC Purity (214 nm): >98%; t_{R}=10.13 min.

### Example 103—4-(2-(tert-Butyl)oxazol-4-yl)-N-(2-cyclopropylethyl)-2-methoxy-1H-imidazole-1-carboxamide

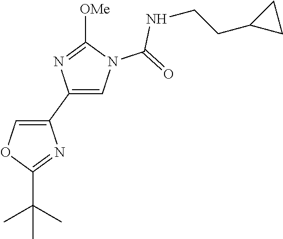 [SMILES: COc1nc(-c2coc(C(C)(C)C)n2)cn1C(=O)NCCC1CC1]

A solution of pivalamide (8.4 g, 83.3 mmol), oxetan-3-one (6.0 g, 83.3 mmol) methanesulfonic acid (797.7 mg, 8.3 mmol) in dimethyl carbonate (40 ml) was stirred at 120° C. for 80 min under microwave. The reaction mixture was diluted with DCM (10 mL), washed with H₂O (10 mL×2), brine (10 mL), dried over Na₂SO₄, filtered and concentrated. The resulting residue was purified by silica gel column chromatography (DCM:MeOH=10:1) to afford (2-tert-butyloxazol-4-yl)methanol (2.7 g, 90%) as a yellow oil. LC-MS m/z: 156.3 [M+H]⁺. Purity (214 nm): 90%; t_{R}=1.53 min.

A suspension of (2-tert-butyloxazol-4-yl)methanol (2.7 g, 17.4 mmol) and MnO₂ (15.1 g, 174 mmol) in DCM (30 mL) was stirred at RT under N₂ for 6 h. The reaction mixture was diluted with DCM (10 mL), washed with H₂O (10 mL×2), brine (10 mL), dried over Na₂SO₄, filtered and concentrated and purified by silica gel column chromatography (DCM:MeOH=10:1) to afford 2-tert-butyloxazole-4-carbaldehyde (800 mg, 92%) as a yellow oil. LC-MS m/z: 154.3 [M+H]⁺. Purity (214 nm): 92%; t_{R}=1.62 min.

To a solution of 2-tert-butyloxazole-4-carbaldehyde (800 mg, 5.2 mmol) in THF (5 mL) was added MeMgBr (618.8 mg, 5.2 mmol) at −65° C. The reaction mixture was then stirred at −65° C. for 1 h, diluted with DCM (10 mL), washed with H₂O (10 mL×2), brine (10 mL), dried over Na₂SO₄, filtered and concentrated. The resulting residue was purified by silica gel column chromatography (DCM:MeOH=10:1) to afford 1-(2-tert-butyloxazol-4-yl)ethanol (520 mg, 97%) as a yellow oil. LC-MS m/z: 170.3 [M+H]⁺. Purity (214 nm): 97%; t_{R}=1.57 min.

A suspension of 1-(2-tert-butyloxazol-4-yl)ethanol (520 mg, 3.1 mmol) and MnO₂ (2.7 g, 31 mmol) in DCM (10 mL) was stirred at RT under N₂ for 6 h. The reaction mixture was diluted with DCM (10 mL), washed with H₂O (10 mL×2), brine (10 mL), dried over Na₂SO₄, filtered and concentrated. The resulting residue was purified by silica gel column chromatography (DCM:MeOH=10:1) to afford 1-(2-tert-butyloxazol-4-yl)ethanone (300 mg, 94%) as a yellow oil. LC-MS m/z: 168.3 [M+H]⁺. Purity (214 nm): 94%; t_{R}=1.52 min.

A solution of 1-(2-tert-butyloxazol-4-yl)ethanone (300 mg, 1.80 mmol), Br₂ (288 mg, 1.80 mmol) in Et₂O (10 mL) was stirred at RT for 2 h under N₂. The mixture was filtered and concentrated to afford 2-bromo-1-(2-tert-butyloxazol-4-yl)-ethanone (1.0 g) as a white solid. LC-MS m/z: 247.2 [M+H]⁺. t_{R}=1.34 min.

A mixture of 2-bromo-1-(2-tert-butyloxazol-4-yl)ethanone (1.0 g, 4.1 mmol), NaHCO₃ (1.7 g, 20.5 mmol) and methyl carbamimidate (455.1 mg, 6.2 mmol) in EtOH (10 mL) was stirred at 65° C. for 4 h under N₂. Then cooled and diluted with DCM (10 mL), washed with H₂O (10 mL×2), brine (10 mL), dried over Na₂SO₄. concentrated in vacuo to give 2-tert-butyl-4-(2-methoxy-1H-imidazol-4-yl)oxazole (320 mg) as an off yellow oil. LC-MS m/z: 222.3 [M+H]⁺. t_{R}=0.48 min.

Following general procedure C, 2-tert-butyl-4-(2-methoxy-1H-imidazol-4-yl)oxazole (320 mg, 1.44 mmol) and 2-cyclopropylethanamine (122.4 mg, 1.44 mmol) afforded the title compound (27.1 mg, 8.5%) as a yellow oil. ¹H NMR (400 MHz, CDCl₃) δ 7.76 (s, 1H), 7.58 (s, 1H), 7.06 (s, 1H), 4.21 (s, 3H), 3.49 (q, J=6.7 Hz, 2H), 1.54-1.50 (m, 2H), 1.33 (s, 9H), 0.75-0.70 (m, 1H), 0.53-0.48 (m, 2H), 0.14-0.05 (m, 2H). LC-MS m/z: 333.0 [M+H]⁺. HPLC Purity (214 nm): 1000%; t_{R}=8.74 min.

### Example 104—N-(2-Cyclopropylethyl)-2-methoxy-4-(4-methyltetrahydro-2H-pyran-4-yl)-1H-imidazole-1-carboxamide

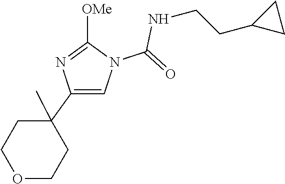 [SMILES: COc1nc(C2(C)CCOCC2)cn1C(=O)NCCC1CC1]

A mixture of 1-(4-methyltetrahydro-2H-pyran-4-yl)ethanone (800 mg, 5.65 mmol), pyridinium tribromide (1.8 g, 4.8 mmol) and HBr in AcOH (6 ml) was stirred at RT for 3 h. The reaction mixture was concentrated under vacuum at 50° C. to give 2-bromo-1-(4-methyltetrahydro-2H-pyran-4-yl)ethanone (1.2 g, 96%) as a brown oil. LC-MS m/z: 221.09 [M+H]⁺. Purity (214 nm): >70%; t_{R}=1.71 min.

To a solution of 2-bromo-1-(4-methyltetrahydro-2H-pyran-4-yl)ethanone (1.2 g, 5.4 mmol) in EtOH (10 mL) was added methyl carbamimidate (1.5 g), NaHCO₃ (1.85 g). The mixture was stirred at 80° C. under microwave for 1 h. Then cooled, concentrated and the residue was purified by silica gel column chromatography (DCM:MeOH=10:1) to give 2-methoxy-4-(4-methyltetrahydro-2H-pyran-4-yl)-1H-imidazole (145 mg, 13.7%) as a yellow oil. LC-MS m/z: 196.2 [M+H]⁺. Purity (214 nm): 95%; t_{R}=1.24 min.

Following general procedure C, 2-methoxy-4-(4-methyltetrahydro-2H-pyran-4-yl)-1H-imidazole (145 mg, 0.74 mmol) and 2-cyclopropylethanamine hydrochloride (100 mg, 0.81 mmol) afforded the title compound (62 mg, 27.3%) as a colorless oil. 1H NMR (400 MHz, CDCl₃) δ 7.01 (brs, 1H), 6.95 (s, 1H), 4.13 (s, 3H), 3.76-3.70 (m, 2H), 3.50 (ddd, J=11.5, 8.4, 3.1 Hz, 2H), 3.35 (q, J=6.7 Hz, 2H), 2.05-1.98 (m, 2H), 1.60-1.52 (m, 2H), 1.48 (q, J=6.8 Hz, 2H), 1.22 (s, 3H), 0.75-0.65 (m, 1H), 0.53-0.47 (m, 2H), 0.14-0.08 (m, 2H). LC-MS m/z: 308.2 [M+H]⁺. HPLC Purity (214 nm): 100%; t_{R}=8.37 min.

### Example 105—4-Cyclopentyl-2-methoxy-N-(3-phenoxypropyl)-1H-imidazole-1-carboxamide

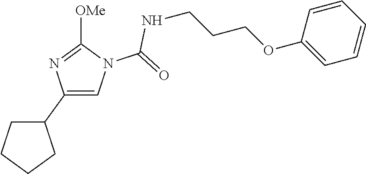 [SMILES: COc1nc(C2CCCC2)cn1C(=O)NCCCOc1ccccc1]

Following general procedure C, 4-cyclopentyl-2-methoxy-1H-imidazole (100 mg, 0.60 mmol) and 3-phenoxypropan-1-amine (302 mg, 2.0 mmol) afforded the title compound (23 mg, 11.1%) as a colorless oil. ¹H NMR (400 MHz, CDCl₃) δ 7.29 (t, J=7.6 Hz, 2H), 7.17 (brs, 1H), 6.98-6.89 (m, 4H), 4.06 (t, J=5.6 Hz, 2H), 4.03 (s, 3H), 3.59 (q, J=6.0 Hz, 2H), 2.91-2.85 (m, 1H), 2.09 (q, J=6.0 Hz, 2H), 1.91-1.95 (m, 2H), 1.73-1.66 (m, 2H), 1.65-1.55 (m, 4H). LC-MS m/z: 344.2 [M+H]⁺. HPLC Purity (214 nm): 100%; t_{R}=9.50 min.

### Example 106—N-Cyclohexyl-4-cyclopentyl-2-methoxy-1H-imidazole-1-carboxamide

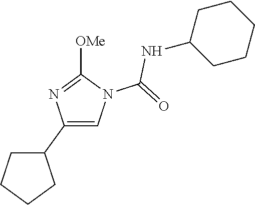 [SMILES: COc1nc(C2CCCC2)cn1C(=O)NC1CCCCC1]

Following general procedure C, 4-cyclopentyl-2-methoxy-1H-imidazole (100 mg, 0.60 mmol) and cyclohexanamine (200 mg, 2.0 mmol) afforded the title compound (29.8 mg, 17.3%) as a white solid. ¹H NMR (400 MHz, CDCl₃) δ 6.94 (s, 1H), 6.78 (d, J=7.2 Hz, 1H), 4.14 (s, 3H), 3.82-3.73 (m, 1H), 2.91-2.82 (m, 1H), 2.01-1.91 (m, 4H), 1.76-1.68 (m, 6H), 1.67-1.53 (m, 2H), 1.45-1.36 (m, 2H), 1.30-1.21 (m, 4H). LC-MS m/z: 292.2 [M+H]⁺. HPLC Purity (214 nm): 100%; t_{R}=8.85 min.

### Example 107—4-(tert-Butyl)-N-iso-butyl-2-iso-propoxy-1H-imidazole-1-carboxamide

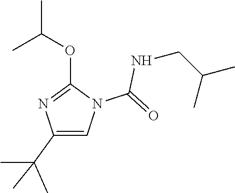 [SMILES: CC(C)CNC(=O)n1cc(C(C)(C)C)nc1OC(C)C]

To a solution of cyanamide (1.0 g, 23.80 mmol) in propan-2-ol (50.0 mL) was added methanesulfonic acid (2.3 g, 23.80 mmol) dropwise at 0° C. The mixture was stirred overnight at RT under N₂, then concentrated in vacuo to give crude isopropyl carbamimidate (2.0 g, 84%) as a white solid. ¹H NMR (400 MHz, CDCl₃) δ 3.81 (dd, 0.1=5.8, 3.3 Hz, 1H), 3.58-3.51 (m, 1H), 3.59-3.49 (m, 1H), 3.42 (s, 3H), 3.01 (s, 3H).

To a suspension of 4 isopropyl carbamimidate (500.0 mg, 4.9 mmol) and NaHCO₃ (2.1 g, 24.5 mmol) in EtOH (50.0 mL) was added 1-bromo-3,3-dimethylbutan-2-one (1.1 g, 5.88 mmol). The mixture was stirred at 70° C. for 2 h, then filtered and concentrated in vacuo to give crude 4-tert-butyl-2-isopropoxy-1H-imidazole (812.3 mg, 36.2%) as a yellow solid which was used directly in the next step. LC-MS m/z: 183.2 [M+H]⁺. Purity (214 nm): 40.3%, t_{R}=1.98 min.

Following general procedure C, crude 4-tert-butyl-2-isopropoxy-1H-imidazole (1.5 g, 3.3 mmol) and 2-methylpropan-1-amine (336.4, 4.6 mmol) afforded the title compound (52.0 mg, 5.6%) as a white solid. ¹H NMR (400 MHz, CDCl₃) δ 7.11 (brs, 1H), 6.88 (s, 1H), 5.28 (septet, J=6.4 Hz, 1H), 3.21 (dd, J=6.5, 5.8 Hz, 2H), 1.84 (tq, J=13.3, 6.6 Hz, 1H), 1.43 (d, J=6.2 Hz, 6H), 1.21 (s, 9H), 0.97 (d, J=6.7 Hz, 6H). LC-MS m/z: 282.2 [M+H]⁺. HPLC Purity (214 nm): 100.0%; t_{R}=10.37 min.

### Example 108—N-Cyclohexyl-2-methoxy-4-(tetrahydrofuran-3-yl)-1H-imidazole-1-carboxamide

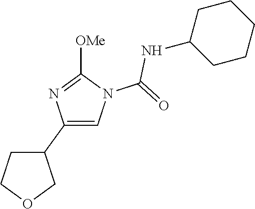 [SMILES: COc1nc(C2CCOC2)cn1C(=O)NC1CCCCC1]

Following general procedure C, 2-methoxy-4-(tetrahydrofuran-3-yl)-1H-imidazole (100 mg, 0.60 mmol) and cyclohexanamine (60 mg, 0.60 mmol) afforded the title compound (31.6 mg, 18.1%) as a white solid. ¹H NMR (400 MHz, CDCl₃) δ 7.02 (s, 1H), 6.76 (d, J=6.5 Hz, 1H), 4.14 (s, 3H), 4.05 (t, J=7.8 Hz, 1H), 3.95 (dd, J=13.8, 8.2 Hz, 1H), 3.87 (dd, J=15.2, 7.6 Hz, 1H), 3.79-3.69 (m, 2H), 3.27 (dt, J=14.9, 7.5 Hz, 1H), 2.26-2.16 (m, 1H), 2.05 (dd, J=13.5, 5.9 Hz, 1H), 1.97 (d, J=13.4 Hz, 2H), 1.72 (d, J=13.6 Hz, 2H), 1.68-1.58 (m, 1H), 1.43 (dd, J=13.6, 10.7 Hz, 2H), 1.26 (dd, 0.1=21.3, 11.0 Hz, 3H). LC-MS m/z: 294.1 [M+H]⁺. HPLC Purity (214 nm): >96%; t_{R}=8.40 min.

### Example 109—4-Cyclohexyl-N-iso-pentyl-2-methoxy-1H-imidazole-1-carboxamide

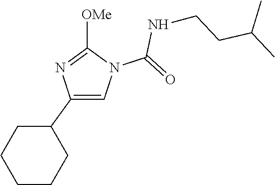 [SMILES: COc1nc(C2CCCCC2)cn1C(=O)NCCC(C)C]

Following general procedure D, 5-bromo-2-methoxy-1-((2-(trimethylsilyl)ethoxy)methyl)-1H-imidazole (657 mg, 2.1 mmol) and cyclohexenylboronic acid (270 mg, 3.22 mmol) afforded 4-cyclohexenyl-2-methoxy-1-((2-(trimethylsilyl)ethoxy)methyl)-1H-imidazole (332 mg, 70%) as a yellow oil. LC-MS m/z: 309.1 [M+H]⁺. Purity (214 nm): 82.97%; t_{R}=2.48 min.

To a solution of 4-cyclohexenyl-2-methoxy-1-((2-(trimethylsilyl)ethoxy)methyl)-1H-imidazole (332 mg, 1.13 mmol) in MeOH (4 mL) was added PtO₂ (10%, 26 mg) and the mixture was stirred at RT for 2 h under H₂. The mixture was filtered and concentrated to give 4-cyclohexyl-2-methoxy-1-((2-(trimethylsilyl)ethoxy)methyl)-1H-imidazole (200 mg, 57%) as a yellow oil which was used directly in the next step. LC-MS m/z: 311.1 [M+H]⁺.

Following general procedure F (method 2), 4-cyclohexyl-2-methoxy-1-((2-(trimethylsilyl)ethoxy)methyl)-1H-imidazole (200 mg, 0.65 mmol) afforded 4-cyclohexyl-2-methoxy-1H-imidazole (70 mg, 59.8%) as a yellow oil which was used directly in the next step. LC-MS m/z: 181.1 [M+H]⁺. Purity (214 nm): >99%; t_{R}=1.72 min.

Following general procedure C, 4-cyclohexyl-2-methoxy-1H-imidazole (60 mg, 0.33 mmol) and 3-methylbutan-1-amine (29 mg, 0.33 mmol) afforded the title compound (25.3 mg, 25.9%) as a colorless oil. ¹H NMR (400 MHz, CDCl₃) δ 6.90 (s, 1H), 6.83 (brs, 1H), 4.13 (s, 3H), 3.38 (q, J=6.3 Hz, 2H), 2.37 (t, J=11.1 Hz, 1H), 2.00 (d, J=12.2 Hz, 2H), 1.82-1.73 (m, 2H), 1.68 (dd, J=13.6, 7.2 Hz, 2H), 1.48 (q, J=7.0 Hz, 2H), 1.40-1.19 (m, 5H), 0.95 (d, J=6.6 Hz, 6H). LC-MS m/z: 294.2 [M+H]⁺. HPLC Purity (214 nm): >99%; t_{R}=10.13 min.

### Example 110—N-iso-Pentyl-2-methoxy-4-(tetrahydro-2H-pyran-4-yl)-1H-imidazole-1-carboxamide

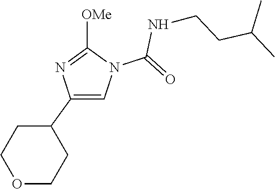 [SMILES: COc1nc(C2CCOCC2)cn1C(=O)NCCC(C)C]

A solution of 1-(tetrahydro-2H-pyran-4-yl)ethanone (350 mg 2.7 mmol) and Br₂ (440 mg 2.7 mmol) in Et₂O (10 ml) was stirred for 2 h at 0° C. The reaction mixture was than concentrated to give 2-bromo-1-(tetrahydro-2H-pyran-4-yl)ethanone (400 mg, 70.6%) which was used directly in the next step. LC-MS m/z: 193.1 [M+H]⁺. Purity (214 nm): 40%; t_{R}=1.63 min.

A suspension of 2-bromo-1-(tetrahydro-2H-pyran-4-yl)ethanone (400 mg, 1.9 mmol), O-methylisourea sulfate (350 mg, 2.9 mmol) and NaHCO₃ (800 mg, 9.5 mmol) in EtOH (10 ml) was stirred for 3 h at 65° C. The reaction was cooled, filtered and concentrated to give a residue which was purified by SGC (DCM:MeOH=20:1) to give 2-methoxy-4-(tetrahydro-2H-pyran-4-yl)-1H-imidazole (300 mg, 85%). LC-MS m/z: 182.1 [M+H]⁺. Purity (214 nm): 50%; t_{R}=1.35 min.

Following general procedure C, 2-methoxy-4-(tetrahydro-2H-pyran-4-yl)-1H-imidazole (400 mg, 2.2 mmol) and 3-methylbutan-1-amine (190 mg, 4.4 mmol) afforded the title compound (46.4 mg, 7.1%) as a colorless oil. ¹H NMR (400 MHz, CDCl₃) δ 6.94 (d, 0.1=1.2 Hz, 1H), 6.83 (s, 1H), 4.14 (s, 3H), 4.02 (dd, J=11.6, 2.1 Hz, 2H), 3.49 (dt, J=11.7, 2.1 Hz, 2H), 3.43-3.33 (m, 2H), 2.71-2.59 (m, 1H), 1.92-1.85 (m, 2H), 1.70-1.59 (m, 3H), 1.49 (q, J=7.1 Hz, 2H), 0.95 (d, J=6.6 Hz, 6H). LC-MS m/z: 296.2 [M+H]⁺. HPLC Purity (214 nm): >96%; t_{R}=7.64 min.

### Example 111—4-(4,4-Difluorocyclohexyl)-N-iso-pentyl-2-methoxy-1H-imidazole-1-carboxamide

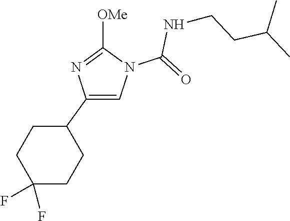 [SMILES: COc1nc(C2CCC(F)(F)CC2)cn1C(=O)NCCC(C)C]

Following general procedure D, 5-bromo-2-methoxy-1-((2-(trimethylsilyl)ethoxy)methyl)-1H-imidazole (1.5 g, 4.92 mmol) and 4,4-difluorocyclohex-1-enylboronic acid (800 mg, 3.28 mmol) afforded 4-(4,4-difluorocyclohex-1-enyl)-2-methoxy-1-((2-(trimethylsilyl)ethoxy)methyl)-1H-imidazole (660 mg, 58.4%) as a pale-yellow oil. LC-MS m/z: 344.47 [M+H]⁺. Purity (214 nm): 97.9%; t_{R}=2.12 min.

To a solution of 4-(4,4-difluorocyclohex-1-enyl)-2-methoxy-1-((2-(trimethylsilyl) ethoxy)methyl)-1H-imidazole (660 mg, 1.92 mmol) in MeOH (10 mL) was added PtO₂ (10%, 70 mg) and the mixture was stirred at RT for 1 h under H₂. The mixture was filtered and concentrated to give 4-(4,4-difluorocyclohexyl)-2-methoxy-1-((2-(trimethylsilyl)ethoxy)methyl)-1H-imidazole (620 mg, 93.37%) as a pale-yellow oil. LC-MS m/z: 346.49 [M+H]⁺. Purity (214 nm): 100%; t_{R}=2.11 min.

Following general procedure F (method 2), 4-(4,4-difluorocyclohexyl)-2-methoxy-1-((2-(trimethylsilyl)ethoxy)methyl)-1H-imidazole (560 mg, 1.62 mmol) afforded 4-(4,4-difluorocyclohexyl)-2-methoxy-1H-imidazole (340 mg, 97.1%) as a pale-yellow oil. LC-MS m/z: 216.26 [M+H]⁺. Purity (214 nm): 91.5%; t_{R}=1.66 min.

Following general procedure C, 4-(4,4-difluorocyclohexyl)-2-methoxy-1H-imidazole (300 mg, 1.39 mmol) and 3-methylbutan-1-amine (135 mg, 1.53 mmol) afforded the title compound (27.6 mg, 5.2%) as a white solid. 1H NMR (400 MHz, CDCl₃) δ 6.95 (d, J=1.0 Hz, 1H), 6.83 (brs, 1H), 4.14 (s, 3H), 3.38 (dt, J=7.3, 6.0 Hz, 2H), 2.52 (t, J=10.8 Hz, 1H), 2.22-2.05 (m, 4H), 1.92-1.64 (m, 5H), 1.49 (q, J=7.1 Hz, 2H), 0.95 (d, J=6.6 Hz, 6H). LC-MS m/z: 329.4 [M+H]⁺. HPLC Purity (214 nm): 96.74%, t_{R}=9.06 min.

### Example 112—4-(tert-Butyl)-N-(4,4-difluorocyclohexyl)-2-methoxy-1H-imidazole-1-carboxamide

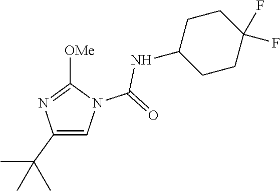 [SMILES: COc1nc(C(C)(C)C)cn1C(=O)NC1CCC(F)(F)CC1]

Following general procedure C, 4-tert-butyl-2-methoxy-1H-imidazole (300 mg, 1.95 mmol) and 4,4-difluorocyclohexanamine (263 mg, 1.95 mmol) afforded the title compound (196.1 mg, 65.0%) as a brown solid. ¹H NMR (400 MHz, DMSO) δ 7.45 (d, J=7.9 Hz, 1H), 6.77 (s, 1H), 3.98 (s, 3H), 3.80 (s, 1H), 2.08-1.53 (m, 8H), 1.16 (s, 9H). LC-MS m/z: 316.2 [M+H]⁺. HPLC Purity (214 nm): 100%; t_{R}=9.56 min.

### Example 113—4-(tert-Butyl)-N-cyclopropyl-2-methoxy-1H-imidazole-1-carboxamide

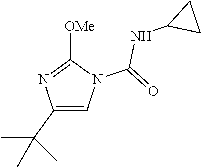 [SMILES: COc1nc(C(C)(C)C)cn1C(=O)NC1CC1]

Following general procedure C, 4-tert-butyl-2-methoxy-1H-imidazole (100 mg, 0.6 mmol) and cyclopropanamine (56 mg, 0.9 mmol) afforded the title compound (41.5 mg, 26.9%) as a white solid. ¹H NMR (400 MHz, CDCl₃) δ 6.98 (brs, 1H), 6.90 (s, 1H), 4.11 (s, 3H), 2.78-2.73 (m, 1H), 1.21 (s, 9H), 0.85-0.81 (m, 2H), 0.62-0.59 (m, 2H). LC-MS m/z: 238.1 [M+H]⁺. HPLC Purity (214 nm): 98.56%; t_{R}=8.19 min.

### Example 114—4-(tert-Butyl)-2-methoxy-N-(tetrahydro-2H-pyran-4-yl)-1H-imidazole-1-carboxamide

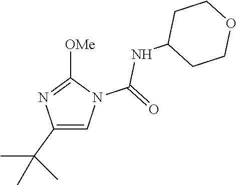 [SMILES: COc1nc(C(C)(C)C)cn1C(=O)NC1CCOCC1]

Following general procedure C, 4-tert-butyl-2-methoxy-1H-Imidazole (0.20 g, 1.30 mmol) and tetrahydro-2H-pyran-4-amine (0.11 g, 1.07 mmol) afforded the title compound (103.5 mg, 28.6%) as a colorless oil. ¹H NMR (400 MHz, CDCl₃) δ 6.89 (s, 11H), 6.81 (brs, 1H), 4.14 (s, 3H), 4.04-3.94 (m, 3H), 3.51 (dt, J=12.0, 2.0 Hz, 2H), 1.98 (dd, J=12.8, 2.0 Hz, 2H), 1.60-1.51 (m, 2H), 1.21 (s, 9H). LC-MS m/z: 282.1 [M+H]⁺. HPLC Purity (214 nm): >99%; t_{R}=7.76 min.

### Example 115—4-tert-Butyl-2-(2-fluorophenoxy)-N-iso-pentyl-1H-imidazole-1-carboxamide

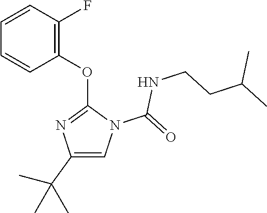 [SMILES: CC(C)CCNC(=O)n1cc(C(C)(C)C)nc1Oc1ccccc1F]

Following general procedure E, 2-bromo-4-tert-butyl-1-((2-(trimethylsilyl)ethoxy) methyl)-1H-imidazole (400 mg, 1.20 mmol) and 2-fluorophenol (2.69 g, 1.5 mL, 24 mmol) afforded 4-tert-butyl-2-(2-fluorophenoxy)-1-((2-(trimethylsilyl)ethoxy)methyl)-1H-imidazole (383 mg, 58.3%) as a yellow oil. LC-MS m/z: 365.0. [M+H]⁺. t_{R}=2.30 min.

Following general procedure F (method 2), 4-tert-butyl-2-(2-fluorophenoxy)-1-((2-(trimethylsilyl)ethoxy)methyl)-1H-imidazole (350 mg, 0.96 mmol) afforded 4-tert-butyl-2-(2-fluorophenoxy)-1H-imidazole (231 mg, 90%). LC-MS m/z: 235.0 [M+H]⁺. HPLC Purity (214 nm): >99%; t_{R}=8.38 min.

Following general procedure C, 4-tert-butyl-2-(2-fluorophenoxy)-1H-imidazole (231 mg, 0.97 mmol) and 3-methylbutan-1-amine (103.14 mg, 1.18 mmol) afforded the title compound (39 mg, 11.5%) as a white solid. ¹H NMR (400 MHz, CDCl₃) δ 7.93 (ddd, J=7.9, 5.2, 1.9 Hz, 1H), 7.24-7.13 (m, 3H), 7.04 (s, 1H), 7.00 (s, 1H), 3.47-3.40 (m, 2H), 1.66 (dd, J=13.3, 6.7 Hz, 1H), 1.53-1.46 (m, 2H), 1.23 (d, J=16.0 Hz, 9H), 0.94 (d, J=6.6 Hz, 6H). LC-MS m/z: 348.0 [M+H]⁺. HPLC Purity (214 nm): 100%; t_{R}=10.72 min.

### Example 116—4-tert-Butyl-2-(4-fluorophenoxy)-N-iso-pentyl-1H-imidazole-1-carboxamide

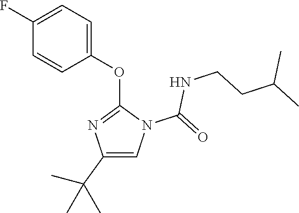 [SMILES: CC(C)CCNC(=O)n1cc(C(C)(C)C)nc1Oc1ccc(F)cc1]

Following general procedure C, 4-tert-butyl-2-(2,4-difluorophenoxy)-1H-imidazole (231 mg, 0.986 mmol) and 3-methylbutan-1-amine (103.14 mg, 1.18 mmol) afforded the title compound (30.6 mg, 8.9%) as a white solid. ¹H NMR (400 MHz, CDCl₃) δ 7.30-7.26 (m, 2H), 7.13-7.06 (m, 2H), 7.04 (s, 1H), 6.80 (s, 1H), 3.47-3.37 (m, 2H), 1.66-1.61 (m, 1H), 1.48 (dd, J=14.5, 7.1 Hz, 2H), 1.26-1.12 (m, 9H), 0.93 (d, J=6.6 Hz, 6H). LC-MS m/z: 348.3 [M+H]⁺. HPLC Purity (214 nm): 100%; t_{R}=11.58 min.

### Example 117—4-(tert-Butyl)-N-iso-pentyl-2-(2-methoxyethoxy)-1H-imidazole-1-carboxamide

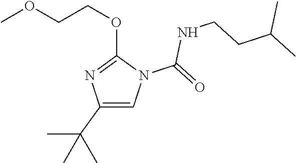 [SMILES: COCCOc1nc(C(C)(C)C)cn1C(=O)NCCC(C)C]

To a solution of cyanamide (1.0 g, 23.80 mmol) in 2-methoxyethanol (10.0 mL) was added methanesulfonic acid (2.3 g, 23.80 mmol) dropwise at 0° C. The mixture was than stirred overnight at RT under N₂ and concentrated in vacuo to give crude 2-methoxyethyl carbamimidate (2.0 g, 71.2%) as a white solid. ¹H NMR (400 MHz, DMSO) δ 4.40-4.34 (m, 2H), 3.64-3.59 (m, 2H), 3.29 (d, J=2.5 Hz, 3H), 2.44 (s, 2H).

To a suspension of 2-methoxyethyl carbamimidate (1.5 g, 12.7 mmol) and NaHCO₃ (3.2 g, 38.1 mmol) in EtOH (50.0 mL) was added 1-bromo-3,3-dimethylbutan-2-one (2.7 g, 15.3 mmol). The mixture was stirred at 70° C. for 2 h, then filtrated and concentrated in vacuo to give crude 4-tert-butyl-2-(2-methoxyethoxy)-1H-imidazole (2.0 g, 25.4%) as a yellow solid which was used directly in the next step. LC-MS m/z: 199.2 [M+H]⁺. Purity (214 nm): 32.8%, t_{R}=1.61 min.

Following general procedure C, crude 4-tert-butyl-2-(2-methoxyethoxy)-1H-imidazole (1.5 g, 2.5 mmol) and 3-methylbutan-1-amine (400.2 mg, 4.6 mmol) afforded the title compound (218.6 mg, 28.1%) as a white solid. ¹H NMR (400 MHz, CDCl₃) δ 7.07 (brs, 1H), 6.90 (s, 1H), 4.64-4.55 (m, 2H), 3.77-3.68 (m, 2H), 3.41 (s, 3H), 3.38 (q, J=7.2 Hz, 2H), 1.71-1.64 (m, 1H), 1.47 (q, J=7.1 Hz, 2H), 1.20 (s, 9H), 0.94 (d, J=6.6 Hz, 6H). LC-MS m/z: 312.2 [M+H]⁺. HPLC Purity (214 nm): 100.0%; t_{R}=10.98 min.

### Example 118—2-Methoxy-N-(3-phenylpropyl)-4-(tetrahydrofuran-3-yl)-1H-imidazole-1-carboxamide

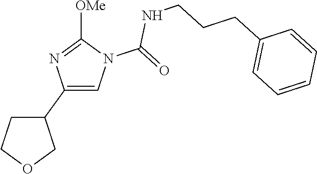 [SMILES: COc1nc(C2CCOC2)cn1C(=O)NCCCc1ccccc1]

Following general procedure C, 2-methoxy-4-(tetrahydrofuran-3-yl)-1H-imidazole (60 mg, 0.35 mmol) and 3-phenylpropan-1-amine (47 mg, 0.35 mmol) afforded the title compound (17.2 mg, 14.6%) as a colorless oil. ¹H NMR (400 MHz, CDCl₃) δ 7.32-7.25 (m, 2H), 7.22-7.17 (m, 3H), 7.03 (s, 1H), 6.87 (brs, 1H), 4.13 (s, 3H), 4.06 (t, J=7.8 Hz, 1H), 3.96 (dd, J=13.8, 8.1 Hz, 1H), 3.88 (dd, J=15.1, 7.6 Hz, 1H), 3.75 (t, J=7.6 Hz, 1H), 3.39 (q, J=6.8 Hz, 2H), 3.32-3.24 (m, 1H), 2.70 (t, J=7.6 Hz, 2H), 2.24-2.17 (m, 1H), 2.04-1.99 (m, 1H), 1.93 (q, J=7.2 Hz, 2H). LC-MS m/z: 330.1 [M+H]⁺. HPLC Purity (214 nm): >96%; t_{R}=8.10 min.

### Example 119—N-iso-Pentyl-2-methoxy-4-methyl-1H-imidazole-1-carboxamide

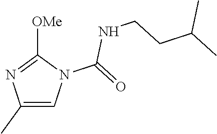 [SMILES: COc1nc(C)cn1C(=O)NCCC(C)C]

Following general procedure C, 2-methoxy-4-methyl-1H-imidazole (56 mg, 0.5 mmol) and 3-methylbutan-1-amine (45.0 mg, 0.5 mmol) afforded the title compound (30.1 mg, 26.8%) as a white solid. ¹H NMR (400 MHz, CDCl₃) δ 6.94 (d, J=1.1 Hz, 1H), 6.82 (brs, 1H), 4.14 (s, 3H), 3.42-3.35 (m, 2H), 2.10 (d, J=1.0 Hz, 3H), 1.72-1.61 (m, 1H), 1.49 (dq J=7.1 Hz, 2H), 0.95 (d, J=6.6 Hz, 6H). LC-MS m/z: 226.1 [M+H]⁺. HPLC Purity (214 nm): 100%; t_{R}=7.18 min.

### Example 120—N-Cyclohexyl-2-methoxy-1H-imidazole-1-carboxamide

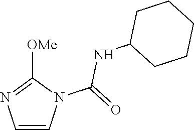 [SMILES: COc1nccn1C(=O)NC1CCCCC1]

Following general procedure C, 2-methoxy-1H-imidazole (100 mg, 0.98 mmol) and cyclohexanamine (97 mg, 0.98 mmol) afforded the title compound (26.9 mg, 12.3%) as a white solid. ¹H NMR (400 MHz, CDCl₃) δ 7.27 (d, J=1.9 Hz, 1H), 6.86 (brs, 1H), 6.59 (d, J=1.9 Hz, 1H), 4.19 (s, 3H), 3.88-3.78 (m, 1H), 2.06-1.97 (m, 2H), 1.79-1.70 (m, 2H), 1.69-1.60 (m, 2H), 1.50-1.38 (m, 2H), 1.36-1.24 (m, 2H). LC-MS m/z: 246.1 [M+23]⁺. HPLC Purity (214 nm): 100%; t_{R}=7.39 min.

### Example 121—2-Ethoxy-N-iso-pentyl-1H-imidazole-1-carboxamide

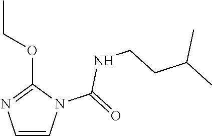 [SMILES: CCOc1nccn1C(=O)NCCC(C)C]

Following general procedure C, 2-ethoxy-1H-imidazole (224 mg, 2.0 mmol) and 3-methylbutan-1-amine (209 mg, 2.4 mmol) afforded the title compound (52.4 mg, 10.0%) as a colorless oil. ¹H NMR (400 MHz, CDCl₃) δ 7.22 (d, J=1.8 Hz, 1H), 6.99 (brs, 1H), 6.56 (d, J=1.8 Hz, 1H), 4.56 (q, J=7.1 Hz, 2H), 3.41 (q, J=7.0 Hz, 2H), 1.72-1.62 (m, 1H), 1.53-1.45 (m, 5H), 0.96 (d, J=6.6 Hz, 6H). LC-MS m/z: 226.1 [M+H]⁺. Purity (214 nm): 100%; t_{R}=7.69 min.

### Example 122—N-iso-Pentyl-2-iso-propoxy-1H-imidazole-1-carboxamide

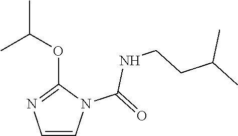 [SMILES: CC(C)CCNC(=O)n1ccnc1OC(C)C]

A mixture of 2,4,5-tribromo-1-((2-(trimethylsilyl)ethoxy)methyl)-1H-imidazole (4 g, 9.3 mmol) and tBuONa (4.4 g, 46.3 mmol) in i-PrOH (60 mL) in a sealed tube was stirred at 120° C. under microwave for 8 h. Then the reaction mixture was concentrated and the residue was purified by silica gel column chromatography (PE:EA=20:1) to give 4,5-dibromo-2-isopropoxy-1-((2-(trimethylsilyl)ethoxy)methyl)-1H-imidazole (2.8 g, 74%) as a white solid. LC-MS m/z: 412.8 [M+H]⁺. Purity (254 nm): >96%; t_{R}=2.44 min.

To a solution of 4,5-dibromo-2-isopropoxy-1-((2-(trimethylsilyl)ethoxy)methyl)-1H-imidazole (2.8 g, 6.8 mmol) in THF (20 mL) was added nBuLi (2.5 M, 13.6 mL) at −78° C. and the mixture was stirred for 4 h. Then the reaction mixture was quenched with aq. NH₄Cl (100 mL), extracted with EA (2×100 mL), concentrated and the residue was purified by silica gel column chromatography (PE:EA=1:1) to give 2-isopropoxy-1-((2-(trimethylsilyl)ethoxy)methyl)-1H-imidazole (580 mg, 26%) as a yellow oil. LC-MS m/z: 257.2 [M+H]⁺. Purity (254 nm): >46%; t_{R}=1.20 min.

Following general procedure F (method B), 2-isopropoxy-1-((2-(trimethylsilyl)ethoxy)methyl)-1H-imidazole (600 mg, 2.34 mmol) afforded 2-isopropoxy-1H-imidazole (280 mg, 95%) as a yellow oil. LC-MS m/z: 127.1 [M+H]⁺. Purity (254 nm): >98%; t_{R}=1.27 min.

Following general procedure C, 2-iso-propoxy-1H-imidazole (100 mg, 0.79 mmol) and 3-methylbutan-1-amine (76 mg, 0.87 mmol) afforded the title compound (30.6 mg, 16.1%) as a colorless oil. ¹H NMR (400 MHz, CDCl₃) δ 7.22 (d, J=2.0 Hz, 1H), 7.06 (brs, 1H), 6.56 (d, J=2.0 Hz, 1H), 5.26 (hept, J=6.2 Hz, 1H), 3.46-3.39 (m, 2H), 1.75-1.65 (m, 1H), 1.50 (q, J=7.2 Hz, 2H), 1.48 (d, J=6.4 Hz, 6H), 0.95 (dd, J=6.7 Hz, 6H). LC-MS m/z: 240.1 [M+H]⁺. HPLC Purity (214 nm): >99%; t_{R}=8.19 min.

### Example 123—2-Methoxy-N-(4-methylpentyl)-1H-imidazole-1-carboxamide

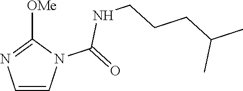 [SMILES: COc1nccn1C(=O)NCCCC(C)C]

Following general procedure C, 2-methoxy-1H-imidazole (120 mg, 1.2 mmol) and 4-methylpentan-1-amine hydrochloride (280 mg, 2.0 mmol) afforded 2-methoxy-N-(4-methylpentyl)-1H-imidazole-1-carboxamide (16.6 mg, 6.0%) as a white solid. ¹H NMR (400 MHz, CDCl₃) δ 7.27 (d, J=1.9 Hz, 1H), 6.95 (brs, 1H), 6.60 (d, J=1.9 Hz, 1H), 4.19 (s, 3H), 3.38 (q, J=7.1 Hz, 2H), 1.66-1.57 (m, 3H), 1.30-1.23 (m, 2H), 0.93 (d, J=6.6 Hz, 6H). LC-MS m/z: 226.1 [M+H]⁺. HPLC Purity (214 nm): 100.0%; t_{R}=7.90 min.

### Example 124—2-methoxy-N-(3-phenylpropyl)-1H-imidazole-1-carboxamide

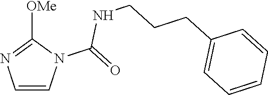 [SMILES: COc1nccn1C(=O)NCCCc1ccccc1]

Following general procedure C, 2-methoxy-1H-imidazole (98 mg, 1 mmol) and 3-phenylpropan-1-amine (135 mg, 1 mmol) afforded the title compound (60.8 mg, 25.5%) as a white solid. ¹H NMR (400 MHz, CDCl₃) δ 7.33-7.27 (m, 2H), 7.26 (d, J=3.1 Hz, 1H), 7.25-7.20 (m, 3H), 6.92 (brs, 1H), 6.59 (d, J=1.9 Hz, 1H), 4.15 (s, 3H), 3.41 (q, J=6.4 Hz, 2H), 2.71 (t, J=7.6 Hz, 2H), 1.98 (q, J=7.2 Hz, 2H). LC-MS m/z: 260.1 [M+H]⁺. HPLC Purity (214 nm): 100%; t_{R}=7.63 min.

### Example 125—N,4-Dicyclopropyl-2-methoxy-1H-imidazole-1-carboxamide

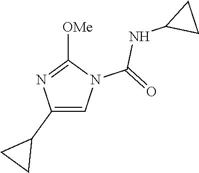 [SMILES: COc1nc(C2CC2)cn1C(=O)NC1CC1]

A suspension of 2-bromo-1-cyclopropylethanone (5 g, 30.7 mmol), NaHCO₃ (12.9 g, 153.5 mmol) and methyl carbamimidate (3.4 g, 46.1 mmol) in EtOH (100 mL) was stirred at 65° C. for 4 h under N₂. Then cooled and concentrated in vacuo to give a residue which was purified by silica gel column chromatography (PEEA=4/1) to give 4-cyclopropyl-2-methoxy-1H-imidazole (1.3 g, 89%) as an off yellow solid. LC-MS m/z: 139.2 [M+H]⁺. Purity (214 nm): 89%; t_{R}=1.46 min.

Following general procedure C, 4-cyclopropyl-2-methoxy-1H-imidazole (300 mg, 2.2 mmol) and cyclopropanamine (125.4 mg, 2.2 mmol) afforded the title compound (65.2 mg, 32.6%) as a brown solid. ¹H NMR (400 MHz, CDCl₃) δ 6.94 (brs, 1H), 6.90 (s, 1H), 4.11 (s, 3H), 2.80-7.72 (m, 1H), 1.75-1.62 (m, 1H), 0.92-0.71 (m, 4H), 0.68-0.54 (m, 4H). LC-MS m/z: 222.2 [M+H]⁺. HPLC Purity (214 nm): 97%; t_{R}=6.63 min.

### Example 126—4-Cyclopentyl-N-cyclopropyl-2-methoxy-1H-imidazole-1-carboxamide

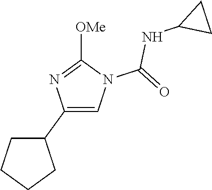 [SMILES: COc1nc(C2CCCC2)cn1C(=O)NC1CC1]

Following general procedure C, 4-cyclopentyl-2-methoxy-1H-imidazole (100 mg, 0.6 mmol) and cyclopropanamine (34 mg, 0.6 mmol) afforded the title compound (22.1 mg, 14.7%) as a colorless oil. ¹H NMR (400 MHz, CDCl₃) δ 6.98 (brs, 1H), 6.95 (d, 0.1=1.2 Hz, 1H), 4.12 (s, 3H), 2.94-2.84 (m, 1H), 2.81-2.75 (m, 1H), 2.03-1.94 (m, 2H), 1.77-1.47 (m, 6H), 0.90-0.84 (m, 2H), 0.66-0.60 (m, 2H). LC-MS m/z: 250.1 [M+H]⁺. HPLC Purity (214 nm): >99%; t_{R}=7.33 min.

### Example 127—4-Cyclopropyl-N-iso-pentyl-2-methoxy-1H-imidazole-1-carboxamide

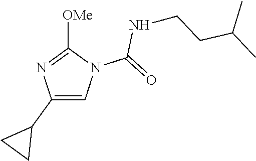 [SMILES: COc1nc(C2CC2)cn1C(=O)NCCC(C)C]

Following general procedure C, 4-cyclopropyl-2-methoxy-1H-imidazole (150.0 mg, 1.1 mmol) and 3-methylbutan-1-amine (113.1 mg, 1.3 mmol) afforded the title compound (218.6 mg, 28.1%) as a white solid. (138.7 mg, 50%). ¹H NMR (400 MHz, CDCl₃) δ 6.90 (d, J=0.7 Hz, 1H), 6.80 (brs, 1H), 4.13 (s, 3H), 3.47-3.28 (m, 2H), 1.76-1.57 (m, 2H), 1.48 (dt, J=8.6, 7.1 Hz, 2H), 0.94 (d, J=6.6 Hz, 6H), 0.82-0.73 (m, 2H), 0.69-0.56 (m, 2H). LC-MS m/z: 252.3 [M+H]⁺. HPLC Purity (214 nm): 100.0%; t_{R}=8.18 min.

### Example 128—N-Cyclopropyl-2-methoxy-4-(tetrahydrofuran-3-yl)-1H-imidazole-1-carboxamide

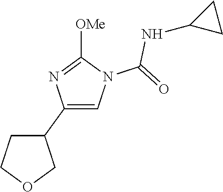 [SMILES: COc1nc(C2CCOC2)cn1C(=O)NC1CC1]

Following general procedure C, 2-methoxy-4-(tetrahydrofuran-3-yl)-1H-imidazole (40 mg, 0.24 mmol) and cyclopropanamine (10 mg, 0.24 mmol) afforded the title compound (14.4 mg, 24.1%) as a white solid. ¹H NMR (400 MHz, CDCl₃) δ 7.03 (s, 1H), 6.96 (brs, 1H), 4.12 (s, 3H), 4.04 (t, J=8.0 Hz, 1H), 3.93 (dq, J=15.0, 7.6 Hz, 2H), 3.77 (t, J=7.2 Hz, 1H), 3.26 (q, J=7.6 Hz, 1H), 2.79-2.72 (m, 1H), 2.24-2.18 (m, 1H), 2.05-1.98 (m, 1H), 0.85 (q, J=6.9 Hz, 2H), 0.64-0.58 (m, 2H). LC-MS m/z: 252.1 [M+H]⁺. HPLC Purity (214 nm): >96%; t_{R}=5.08 min.

### Example 129—4-(tert-Butyl)-N-(1-ethylcyclopropyl)-2-methoxy-1H-imidazole-1-carboxamide

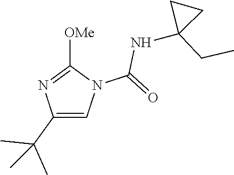 [SMILES: CCC1(NC(=O)n2cc(C(C)(C)C)nc2OC)CC1]

To a solution of propiononitrile (1.0 g, 18 mmol) and Ti(OiPr)₄ (6.2 g, 20 mmol) in Et₂O (20 mL) was added EtMgBr (40 mL, 40 mmol) at −78° C. and the reaction mixture was stirred at RT for 30 min. Then BF₃-Et₂O (10 mL, 48% solution) was added and the reaction mixture was stirred at RT for 30 min. The reaction mixture was quenched with saturated aq. NH₄Cl (20 mL), extracted with EA (20 mL×3), washed with brine (20 mL), dried over Na₄SO₄, filtered and concentrated to give crude 1-ethylcyclopropanamine (380 mg, 25.0%) as a colorless oil which was used directly in the next step.

To a solution of crude 1-ethylcyclopropanamine (380 g, 4.46 mmol) and TEA (900 mg, 8.92 mmol) in DCM (4 ml) was added Boc₂O (1.46 g, 6.7 mmol) and the reaction mixture was stirred at RT for 1 h. The reaction mixture was concentrated and purified by silica gel column chromatography (PE:EA=15:1) to give tert-butyl 1-ethylcyclopropylcarbamate (120 mg, 13.7%) as colorless crystals. LC-MS m/z: 185.26 [M+H]⁺. HPLC Purity (214 nm): 95%; t_{R}=2.00 min.

A mixture of tert-butyl 1-ethylcyclopropylcarbamate (120 mg, 0.65 mmol) in HCl dioxane solution (2 ml, 4M) was stirred at RT for 1 h. The reaction was concentrated to give 1-ethylcyclopropanamine (80 mg, 98%) as a colorless oil and used directly in the next step.

Following general procedure C, 4-tert-butyl-2-methoxy-1H-imidazole (155 mg, 1.0 mmol) and 1-ethylcyclopropanamine (80 mg, 0.66 mmol) afforded the title compound (39 mg, 22.4%) as a colorless oil. 1H NMR (400 MHz, CDCl₃) δ 7.11 (s, 1H), 6.89 (s, 1H), 4.13 (s, 3H), 1.67 (dq, J=7.6, 7.2 Hz, 2H), 1.21 (s, 9H), 0.97 (t, J=7.4 Hz, 3H), 0.81 (t, J=5.9 Hz, 2H), 0.72 (t, J=5.9 Hz, 2H). LC-MS m/z: 266.1 [M+H]⁺. HPLC Purity (214 nm): 100%; t_{R}=8.47 min.

### Example 130—4-Cyclopropyl-2-methoxy-N-phenethyl-1H-imidazole-1-carboxamide

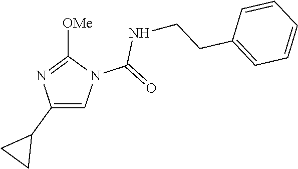 [SMILES: COc1nc(C2CC2)cn1C(=O)NCCc1ccccc1]

Following general procedure B (method 1), 4-cyclopropyl-2-methoxy-1H-imidazole (0.10 g, 0.72 mmol) and (2-isocyanatoethyl)benzene (0.12 g, 0.80 mmol) afforded the title compound (31.2 mg, 15.1%) as a colorless oil. ¹H NMR (400 MHz, CDCl₃) δ 7.33 (t, J=7.2 Hz, 2H), 7.27-7.25 (m, 1H), 7.24-7.20 (m, 2H), 6.88 (s, 1H), 6.85 (brs, 1H), 3.96 (s, 3H), 3.63 (q, J=6.4 Hz, 2H), 2.88 (t, J=6.8 Hz, 2H), 1.70-1.66 (m, 1H), 0.80-0.75 (m, 2H), 0.64-0.60 (m, 2H). LC-MS m/z: 286.2 [M+H]⁺. HPLC Purity (214 nm): >99%; t_{R}=8.61 min.

### Example 131—N-(4-Cyclopropylbutyl)-2-methoxy-4-(tetrahydrofuran-3-yl)-1H-imidazole-1-carboxamide

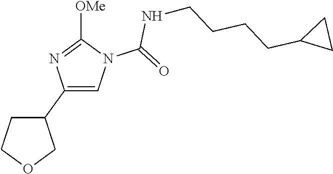 [SMILES: COc1nc(C2CCOC2)cn1C(=O)NCCCCC1CC1]

Following general procedure C, 2-methoxy-4-(tetrahydrofuran-3-yl)-1H-imidazole (100 mg, 0.60 mmol) and 4-cyclopropylbutan-1-amine (70 mg, 0.60 mmol) afforded the title compound (11.7 mg, 6.4%) as a colorless oil. ¹H NMR (400 MHz, CDCl₃) δ 7.03 (s, 1H), 6.87 (brs, 1H), 4.14 (s, 3H), 4.05 (t, J=8.0 Hz, 1H), 4.02 (m, 1H), 3.90 (dq, J=13.7, 8.0 Hz, 2H), 3.74 (t, J=5.6 Hz, 1H), 3.36 (q, J=7.1 Hz, 2H), 3.30-3.25 (m, 1H), 2.26-2.18 (m, 1H), 2.09-2.00 (m, 1H), 1.65-1.58 (m, 2H), 1.51-1.41 (m, 2H), 1.24-1.20 (m, 2H), 0.70-0.62 (m, 1H), 0.46-0.36 (m, 2H), 0.00 (brs, 2H). LC-MS m/z: 308.3 [M+H]⁺. HPLC Purity (214 nm): >96%; t_{R}=8.97 min.

### Example 132—N-iso-Pentyl-2-(methylthio)-1H-imidazole-1-carboxamide

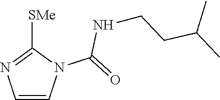

To a solution of 1H-imidazole-2-thiol (500 mg, 5.0 mmol) in DCM (20 mL) at 0° C. was added NaH (180 mg, 7.5 mmol) and the reaction mixture was stirred at 0° C. for 30 min. Then MeI (1.1 g, 7.5 mmol) was added and the reaction mixture was stirred at RT for 2 h. The mixture was quenched with water, extracted with DCM (×3), dried over anhydrous Na₂SO₄, filtered and concentrated to give 2-(methylthio)-1H-imidazole (350 mg) as a white solid. LC-MS m/z: 115.7 [M+H]⁺.

Following general procedure C, 2-(methylthio)-1H-imidazole (300 mg, 2.6 mmol) and 3-methylbutan-1-amine (270 mg, 3.1 mmol) afforded the title compound (71.8 mg, 12.0%) as a colorless oil. ¹H NMR (400 MHz, DMSO) δ 8.23 (brs, 1H), 7.70 (d, J=1.5 Hz, 1H), 6.97 (t, J=1.6 Hz, 1H), 3.22 (q, J=8.0 Hz, 2H), 2.46 (s, 3H), 1.61-1.52 (m, 1H), 1.37 (q, J=6.8 Hz, 2H), 0.85 (d, J=6.8 Hz, 6H). LC-MS m/z: 228.3 [M+H]⁺. Purity (214 nm): 100%; t_{R}=6.25 min.

### Example 133—N-Cyclopropyl-2-methoxy-1H-imidazole-1-carboxamide

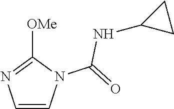 [SMILES: COc1nccn1C(=O)NC1CC1]

Following general procedure C, 2-methoxy-1H-imidazole (120 mg, 1.20 mmol) and cyclopropanamine (68 mg, 1.20 mmol) afforded the title compound (29 mg, 13.1%) as a white solid. ¹H NMR (400 MHz, CDCl₃) δ 7.25 (d, J=2.0 Hz, 1H), 7.03 (brs, 1H), 6.57 (d, J=1.9 Hz, 1H), 4.15 (s, 3H), 2.82-2.75 (m, 1H), 0.90-0.84 (m, 2H), 0.66-061 (m, 2H). LC-MS m/z: 182.2 [M+H]⁺. HPLC Purity (214 nm): 99.99%; t_{R}=4.40 min.

### Example 134—4-(tert-Butyl)-N-(4-(3-(tert-butyl)-1H-pyrazol-1-yl)butyl)-2-methoxy-1H-imidazole-1-carboxamide

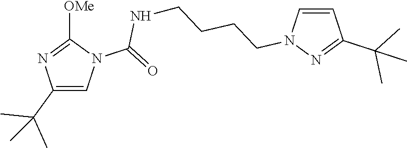 [SMILES: COc1nc(C(C)(C)C)cn1C(=O)NCCCCn1ccc(C(C)(C)C)n1]

Following general procedure C, 4-(tert-butyl)-2-methoxy-1H-imidazole (110 mg, 0.71 mmol) and 4-(3-(tert-butyl)-1H-pyrazol-1-yl)butan-1-amine (348 mg, 1.78 mmol) afforded the title compound (189 mg, 70.8%) as a clear oil. ¹H NMR (400 MHz, CDCl₃) δ 7.25 (d, J=2.0 Hz, 1H), 6.90 (brs, 1H), 6.88 (s, 1H), 6.06 (d, J=2.0 Hz, 1H), 4.13 (s, 3H), 4.10 (t, 0.1=7.2 Hz, 2H), 3.34 (q, J=6.8 Hz, 2H), 1.89 (p, J=7.2 Hz, 2H), 1.57 (p, J=7.2 Hz, 2H), 1.29 (s, 9H), 1.19 (s, 9H). LC-MS m/z: 398.0 [M+23]⁺. HPLC Purity (214 nm): 99%; t_{R}=9.49 min.

### Example 135—2-Methoxy-N-phenethyl-1H-imidazole-1-carboxamide

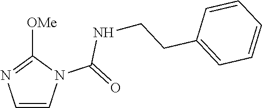 [SMILES: COc1nccn1C(=O)NCCc1ccccc1]

Following general procedure C, 2-methoxy-1H-imidazole (80 mg, 0.81 mmol) and 2-phenylethanamine (98 mg, 0.81 mmol) afforded the title compound (9 mg, 4.5%) as a yellow solid. ¹H NMR (400 MHz, CDCl₃) δ 7.35 (t, J=7.3 Hz, 2H), 7.30-7.22 (m, 4H), 6.93 (brs, 1H), 6.56 (d, J=1.6 Hz, 1H), 4.00 (s, 3H), 3.67 (q, J=6.8 Hz, 2H), 2.91 (t, J=6.8 Hz, 2H). LC-MS m/z: 246.1 [M+H]⁺. HPLC Purity (214 nm): 96.94%; t_{R}=6.86 min.

### Example 136—2-Methoxy-N-(4-phenylbutyl)-1H-imidazole-1-carboxamide

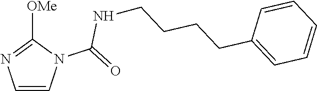 [SMILES: COc1nccn1C(=O)NCCCCc1ccccc1]

Following general procedure C, 2-methoxy-1H-imidazole (98 mg, 1.0 mmol) and 4-phenylbutan-1-amine (224 mg, 1.5 mmol) afforded the title compound (40.4 mg, 14.8%) as a colorless oil. ¹H NMR (400 MHz, CDCl₃) δ 7.32-7.25 (m, 2H), 7.24 (d, J=2.0 Hz, 1H), 7.21-7.15 (m, 3H), 6.91 (brs, 1H), 6.57 (d, J=2.0 Hz, 1H), 4.14 (s, 3H), 3.40 (q, J=6.8 Hz, 2H), 2.66 (t, J=7.6 Hz, 2H), 1.60-1.74 (m, 4H). LC-MS m/z: 274.1 [M+H]⁺. HPLC Purity (214 nm): 98%; t_{R}=8.85 min.

### Example 137—2-Methoxy-N-(3-(1-(trifluoromethyl)cyclopropyl)propyl)-1H-imidazole-1-carboxamide

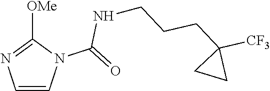 [SMILES: COc1nccn1C(=O)NCCCC1(C(F)(F)F)CC1]

Following general procedure C, 2-methoxy-1H-imidazole (98 mg, 1.0 mmol) and 3-(1-(trifluoromethyl)cyclopropyl)propan-1-amine (251 mg, 1.5 mmol) afforded the title compound (14.6 mg, 5.0%) as a white solid. ¹H NMR (400 MHz, CDCl₃) δ 7.24 (d, J=2.0 Hz, 1H), 6.94 (brs, 1H), 6.58 (d, J=2.4 Hz, 1H), 4.17 (s, 3H), 3.37 (q, J=7.2 Hz, 2H), 1.81-1.73 (m, 2H), 1.64-1.59 (m, 2H), 0.99-0.96 (m, 2H), 0.59 (brs, 2H). LC-MS m/z: 292.1 [M+H]⁺. HPLC Purity (214 nm): 100%; t_{R}=7.57 min.

### Example 138—2-Methoxy-N-(4-(1-(trifluoromethyl)cyclopropyl)butyl)-1H-imidazole-1-carboxamide

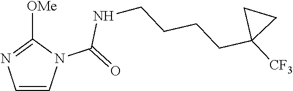 [SMILES: COc1nccn1C(=O)NCCCCC1(C(F)(F)F)CC1]

Following general procedure C, 2-methoxy-11H-imidazole (50 mg, 0.51 mmol) and 4-(1-(trifluoromethyl)cyclopropyl)butan-1-amine (92 mg, 0.51 mmol) afforded the title compound (15.8 mg, 12.2%) as a colorless oil. ¹H NMR (400 MHz, CDCl₃) δ 7.24 (d, J=1.9 Hz, 1H), 6.96 (brs, 1H), 6.58 (d, J=1.9 Hz, 1H), 4.17 (s, 3H), 3.38 (q, J=6.6 Hz, 2H), 1.63-1.57 (m, 4H), 1.561.50 (m, 2H), 0.98-0.93 (m, 2H), 0.56 (brs, 2H). LC-MS m/z: 306.2 [M+H]⁺. HPLC Purity (214 nm): >99%; t_{R}=8.91 min.

### Example 139—2-Methoxy-4-methyl-N-(4,4,4-trifluorobutyl)-1H-imidazole-1-carboxamide

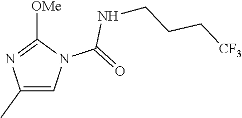 [SMILES: COc1nc(C)cn1C(=O)NCCCC(F)(F)F]

Following general procedure C, 2-methoxy-4-methyl-1H-imidazole (112 mg, 1.0 mmol) and 4,4,4-trifluorobutan-1-amine (191 mg, 1.5 mmol) afforded the title compound (57.0 mg, 26.4%) as a colorless oil. ¹H NMR (400 MHz, CDCl₃) δ 6.95 (brs, 1H), 6.93 (s, 1H), 4.15 (s, 3H), 3.45 (q, J=6.4 Hz, 2H), 2.09-2.10 (m, 2H), 1.91 (s, 3H), 1.88-1.90 (m, 2H). LC-MS m/z: 266.1 [M+H]⁺. HPLC Purity (214 nm): 95%; t_{R}=7.09 min.

### Example 140—N-Cyclopropyl-2-methoxy-4-phenyl-1H-imidazole-1-carboxamide

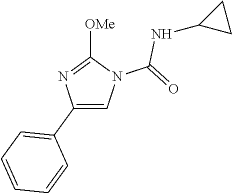 [SMILES: COc1nc(-c2ccccc2)cn1C(=O)NC1CC1]

Following general procedure C, 2-methoxy-4-phenyl-1H-imidazole (100 mg, 0.57 mmol) and cyclopropanamine (39.33 mg, 0.69 mmol) afforded the title compound (60 mg, 40.6%) as a white solid. ¹H NMR (400 MHz, CDCl₃) δ 7.75 (dd, J=3.2, 1.2 Hz, 2H), 7.54 (s, 1H), 7.42-7.36 (m, 2H), 7.26 (m, J=7.6 Hz, 1H), 7.02 (brs, 1H), 4.23 (s, 3H), 2.88-2.80 (m, 1H), 0.92-0.85 (m, 2H), 0.68-0.63 (m, 2H). LC-MS m/z: 258.0 [M+H]⁺. HPLC Purity (214 nm): 100%; t_{R}=8.44 min.

### Example 141—2-Methoxy-4-methyl-N-(3-phenylpropyl)-1H-imidazole-1-carboxamide

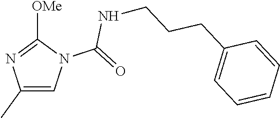 [SMILES: COc1nc(C)cn1C(=O)NCCCc1ccccc1]

Following general procedure C, 2-methoxy-4-methyl-1H-imidazole (112 mg, 1.0 mmol) and 3-phenylpropan-1-amine (134 mg, 1.0 mmol) afforded the title compound (86.1 mg, 35.3%) as a white solid. ¹H NMR (400 MHz, CDCl₃) δ 7.39-7.23 (m, 2H), 7.24-7.18 (m, 3H), 6.92 (d, J=1.2 Hz, 1H), 6.85 (brs, 1H), 4.12 (s, 3H), 3.39 (q, J=7.0 Hz, 2H), 2.69 (t, J=7.6 Hz, 2H), 2.10 (d, J=1.1 Hz, 3H), 1.94 (dt, J=14.7, 7.4 Hz, 2H). LC-MS m/z: 274.1 [M+H]⁺. HPLC Purity (214 nm): 98.91%; t_{R}=7.86 min.

### Example 142—2-Methoxy-4-methyl-N-(4-methylpentyl)-1H-imidazole-1-carboxamide

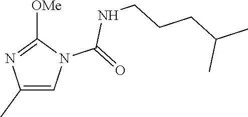 [SMILES: COc1nc(C)cn1C(=O)NCCCC(C)C]

Following general procedure C, 2-methoxy-4-methyl-1H-imidazole (100 mg, 0.89 mmol) and 4-methylpentan-1-amine (113 mg, 1.0 mmol) afforded the title compound (130 mg, 62.5%) as a yellow oil. ¹H NMR (400 MHz, DMSO-d₆) δ 7.62 (t, J=5.6 Hz, 1H), 6.84 (d, J=1.2 Hz, 1H), 3.98 (s, 3H), 3.17 (q, J=7.1 Hz, 2H), 1.98 (d, J=1.1 Hz, 3H), 1.69-1.41 (m, 3H), 1.20-1.12 (m, 2H), 0.86 (d, J=6.6 Hz, 6H). LC-MS m/z: 240.2 [M+H]⁺. HPLC Purity (214 nm): >99%; t_{R}=8.03 min.

### Example 143—2-Methoxy-4-methyl-N-phenethyl-1H-imidazole-1-carboxamide

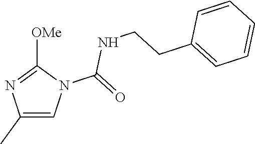 [SMILES: COc1nc(C)cn1C(=O)NCCc1ccccc1]

Following general procedure B (method 1), 2-methoxy-4-methyl-1H-imidazole (200 mg, 1.78 mmol) and (2-isocyanatoethyl)benzene (290 mg, 1.96 mmol) afforded the title compound (120 mg, 25.92%) as a colorless oil. ¹H NMR (400 MHz, CDCl₃) δ 7.34 (t, J=7.3 Hz, 2H), 7.31-7.23 (m, 3H), 6.93 (d, J=1.1 Hz, 1H), 6.85 (brs, 1H), 3.98 (s, 3H), 3.64 (q, J=6.7 Hz, 2H), 2.90 (t, J=6.8 Hz, 2H), 2.09 (d, J=1.1 Hz, 3H). LC-MS m/z: 260.1 [M+H]⁺. HPLC Purity (214 nm): 100%; t_{R}=7.23 min.

### Example 144—2-Methoxy-4-methyl-N-(3-phenoxypropyl)-1H-imidazole-1-carboxamide

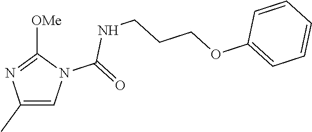 [SMILES: COc1nc(C)cn1C(=O)NCCCOc1ccccc1]

Following general procedure C, 2-methoxy-4-methyl-1H-imidazole (0.10 g, 0.89 mmol) and 3-phenoxypropan-1-amine (0.15 g, 0.98 mmol) afforded the title compound (21.8 mg, 8.7%) as a white solid. ¹H NMR (400 MHz, CDCl₃) δ 7.31-7.28 (m, 2H), 7.14 (br, 1H), 6.90 (t, J=7.6 Hz, 1H), 6.99-6.89 (m, 3H), 4.07 (t, J=5.2 Hz, 2H), 4.03 (s, 3H), 3.60 (q, J=6.4 Hz, 2H), 2.12-2.06 (m, 5H). LC-MS m/z: 290.1 [M+H]⁺. HPLC Purity (214 nm): >99%; t_{R}=7.50 min.

### Example 144—2-Methoxy-4-methyl-N-(3-(1-(trifluoromethyl)cyclopropyl)propyl)-1H-imidazole-1-carboxamide

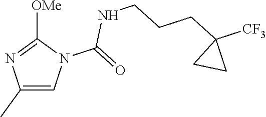 [SMILES: COc1nc(C)cn1C(=O)NCCCC1(C(F)(F)F)CC1]

Following general procedure C, 2-methoxy-4-methyl-1H-imidazole (300 mg, 2.7 mmol) and 3-(1-(trifluoromethyl)cyclopropyl)propan-1-amine (445 mg, 2.67 mmol) afforded the title compound (20.0 mg, 2.4%) as a colorless oil. ¹H NMR (400 MHz, CDCl₃) δ 6.93 (d, J=1.2 Hz, 1H), 6.87 (brs, 1H), 4.15 (s, 3H), 3.35 (q, J=7.0 Hz, 2H), 2.11 (d, J=1.2 Hz, 3H), 1.80-1.75 (m, 2H), 1.61-1.55 (m, 2H), 0.99-0.93 (m, 2H), 0.58 (brs, 2H). LC-MS m/z: 306.1 [M+H]⁺. HPLC Purity (214 nm): >96%; t_{R}=7.58 min.

### Example 146—N-iso-Pentyl-4-methyl-2-phenoxy-1H-imidazole-1-carboxamide

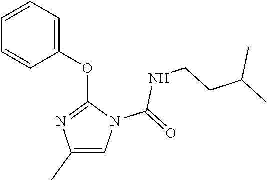 [SMILES: Cc1cn(C(=O)NCCC(C)C)c(Oc2ccccc2)n1]

Following procedure E, 2-bromo-4-methyl-1-((2-(trimethylsilyl)ethoxy)methyl)-1H-imidazole (900 mg, 3.1 mmol) in phenol (15 mL) afforded 4-methyl-2-phenoxy-1-((2-(trimethylsilyl) ethoxy)methyl)-1H-imidazole (500 mg, 53%) as a yellow oil. LC-MS m/z: 305.0 [M+H]⁺. Purity (214 nm): >21%; t_{R}=1.95 min.

Following procedure F (method 2), 4-methyl-2-phenoxy-1-((2-(trimethylsilyl)ethoxy) methyl)-1H-imidazole (500 mg, 1.52 mmol) afforded 4-methyl-2-phenoxy-1H-imidazole (250 mg, 87%) as a yellow oil. LC-MS m/z: 175.1 [M+H]⁺. Purity (254 nm): >99%; t_{R}=1.45 min.

Following general procedure C, 4-methyl-2-phenoxy-1H-imidazole (100 mg, 0.57 mmol) and 3-methylbutan-1-amine (52 mg, 0.6 mmol) afforded the title compound (46.1 mg, 27.9%) as a white solid. ¹H NMR (400 MHz, CDCl₃) δ 7.48-7.43 (m, 2H), 7.32-7.24 (m, 3H), 7.09-7.05 (m, 1H), 6.88 (brs, 1H), 3.42 (td, J=8.3, 2.6 Hz, 2H), 2.10 (d, J=1.6 Hz, 3H), 1.74-1.53 (m, 1H), 1.53-1.37 (m, 2H), 0.93 (dd, J=6.6, 2.7 Hz, 6H). LC-MS m/z: 288.1 [M+H]⁺. HPLC Purity (214 nm): >99%; t_{R}=8.87 min.

### Example 147—N-Cyclopropyl-4-methyl-2-phenoxy-1H-imidazole-1-carboxamide

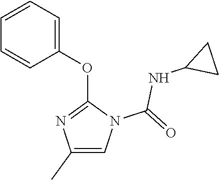 [SMILES: Cc1cn(C(=O)NC2CC2)c(Oc2ccccc2)n1]

Following general procedure C, 4-methyl-2-phenoxy-1H-imidazole (100 mg, 0.57 mmol) and cyclopropanamine (36 mg, 0.63 mmol) afforded the title compound (25.9 mg, 17.5%) as a white solid. ¹H NMR (400 MHz, CDCl₃) δ 7.43 (dt, J=7.6, 2.1 Hz, 2H), 7.31-7.23 (m, 3H), 7.06 (d, 0.1=1.1 Hz, 1H), 7.02 (brs, 1H), 2.87-2.79 (m, 1H), 2.07 (d, J=1.2 Hz, 3H), 0.94-0.80 (m, 2H), 0.67-0.54 (m, 2H). LC-MS m/z: 258.1 [M+H]⁺. Purity (214 nm): >99%; t_{R}=1.84 min.

### Example 148—N-Cyclopropyl-2-methoxy-4-methyl-1H-imidazole-1-carboxamide

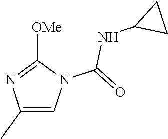

Following general procedure C, 2-methoxy-4-methyl-1H-imidazole (200 mg, 1.79 mmol) and cyclopropanamine (252 mg, 2.68 mmol) afforded the title compound (123 mg, 35.3%) as a white solid. ¹H NMR (400 MHz, CDCl₃) δ 6.94 (d, J=1.2 Hz, 2H), 4.12 (s, 3H), 2.82-2.75 (m, 1H), 2.10 (d, J=1.2 Hz, 3H), 0.91-0.80 (m, 2H), 0.66-056 (m, 2H). LC-MS m/z: 196.1 [M+H]⁺. HPLC Purity (214 nm): 100%; t_{R}=4.98 min.

### Examples 149 and 150—2-iso-Propoxy-4-methyl-N-phenethyl-1H-imidazole-1-carboxamide and 2-iso-Propoxy-5-methyl-N-phenethyl-1H-imidazole-1-carboxamide

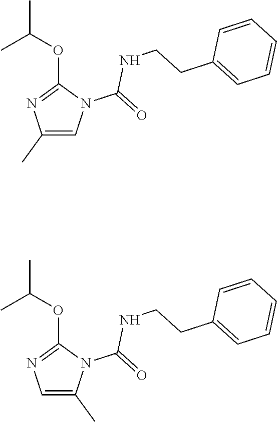 [SMILES: Cc1cn(C(=O)NCCc2ccccc2)c(OC(C)C)n1 ; Cc1cnc(OC(C)C)n1C(=O)NCCc1ccccc1]

To a solution of cyanamide (1.0 g, 23.80 mmol) in propan-2-ol (20.0 mL) was added methanesulfonic acid (2.3 g, 23.80 mmol) dropwise at 0° C. The mixture was stirred overnight at RT under N₂, then concentrated in vacuo to give the crude isopropyl carbamimidate (2.0 g, 82.4%) as a colorless solid which was used directly in the next step.

To a suspension of isopropyl carbamimidate (2.0 g, 19.6 mmol) and NaHCO₃ (4.9 g, 58.8 mmol) in EtOH (100.0 mL) was added 1-bromopropan-2-one (3.5 g, 25.4 mmol). The mixture was stirred at 70° C. for 2 h, then filtered and concentrated in vacuo to give crude 2-iso-propoxy-4-methyl-1H-imidazole (4.0 g, 95.0%) as a yellow oil. The crude product was not stable and was used directly in the next step. LC-MS m/z: 141.2 [M+H]⁺. Purity (214 nm): 65.2%, t_{R}=1.53 min.

Following general procedure B (method 1), crude 2-iso-propoxy-4-methyl-1H-imidazole (1.0 g, 4.7 mmol) and (2-isocyanatoethyl)benzene (837.9 g, 5.7 mmol) afforded 2-iso-propoxy-4-methyl-N-phenethyl-1H-imidazole-1-carboxamide (30.1 mg, 2.5%) and 2-iso-propoxy-5-methyl-N-phenethyl-1H-imidazole-1-carboxamide (3.0 mg, 0.3%) as white solids. 2-iso-propoxy-4-methyl-N-phenethyl-1H-imidazole-1-carboxamide: ¹H NMR (400 MHz, DMSO-d₆) δ 7.27 (tt, J=14.6, 7.2 Hz, 6H), 6.82 (d, J=1.3 Hz, 1H), 5.02 (sep, J=6.2 Hz, 1H), 3.53 (q, J=6.8 Hz, 2H), 2.83 (t, J=6.9 Hz, 2H), 1.97 (d, J=1.2 Hz, 3H), 1.24 (d, J=6.2 Hz, 6H). LC-MS m/z: 288.3 [M+H]⁺. HPLC Purity (214 nm): 100.0%; t_{R}=7.85 min. 2-iso-propoxy-5-methyl-N-phenethyl-1H-imidazole-1-carboxamide: ¹H NMR (400 MHz, CDCl₃) δ 7.33 (t, J=7.2 Hz, 2H), 7.26-7.21 (m, 3H), 7.10 (s, 1H), 6.25 (d, J=1.3 Hz, 1H), 5.12 (sep, J=6.0 Hz, 1H), 3.69 (q, J=6.8 Hz, 2H), 2.89 (t, J=6.8 Hz, 2H), 2.36 (d, J=1.2 Hz, 3H), 1.21 (d, J=6.2 Hz, 6H). LC-MS m/z: 288.1 [M+H]⁺. HPLC Purity (214 nm): 95.91%; t_{R}=8.38 min.

### Example 151—2-Methoxy-4-methyl-N-(3-(trifluoromethoxy)-propyl)-1H-imidazole-1-carboxamide

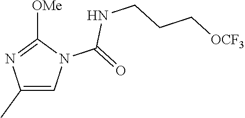 [SMILES: COc1nc(C)cn1C(=O)NCCCOC(F)(F)F]

Following general procedure C, 2-methoxy-4-methyl-1H-imidazole (24 mg, 0.22 mmol) and 3-(trifluoromethoxy)propan-1-amine (50 mg, 0.26 mmol) afforded the title compound (21.4 mg, 35.1%) as a clear oil. ¹H NMR (400 MHz, CDCl₃) δ 7.07 (s, 1H), 6.93-6.92 (m, 1H), 4.14 (s, 3H), 4.09 (t, J=6.0 Hz, 2H), 3.52 (q, J=6.4 Hz, 2H), 2.11 (d, J=1.2 Hz, 3H), 2.05-1.98 (m, 2H). LC-MS m/z: 282.0 [M+H]⁺. HPLC Purity (214 nm): 100%; t_{R}=7.60 min.

### Example 152—N-(4,4-difluorocyclohexyl)-2-methoxy-4-methyl-1H-imidazole-1-carboxamide

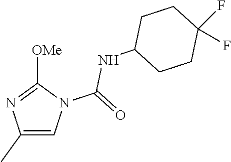 [SMILES: COc1nc(C)cn1C(=O)NC1CCC(F)(F)CC1]

Following general procedure C, to a solution of 2-methoxy-4-methyl-1H-imidazole (100 mg, 0.89 mmol) in DCM (5 mL) and 4,4-difluorocyclohexanamine (120 mg, 0.89 mmol) afforded the title compound (66.4 mg, 27.2%) as a white solid. ¹H NMR (400 MHz, DMSO-d₆) δ 7.42 (d, J=7.6 Hz, 1H), 6.85 (d, J=1.2 Hz, 1H), 3.97 (s, 3H), 3.80 (brs, 1H), 2.05-1.97 (m, 6H), 1.95-1.81 (m, 3H), 1.74-1.61 (m, 2H). LC-MS m/z: 274.0 [M+H]⁺. HPLC Purity (214 nm): 97%; t_{R}=7.26 min.

### Example 153—N-(3,3-difluorocyclohexyl)-2-methoxy-4-methyl-1H-imidazole-1-carboxamide

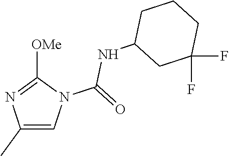 [SMILES: COc1nc(C)cn1C(=O)NC1CCCC(F)(F)C1]

Following general procedure C, 2-methoxy-4-methyl-1H-imidazole (100 mg, 0.89 mmol) and 3,3-difluorocyclohexanamine (120 mg, 0.89 mmol) afforded the title compound (70.5 mg, 29%) as a white solid. ¹H NMR (400 MHz, CDCl₃) δ 6.92 (d, J=1.1 Hz, 1H), 4.26 (brs, 1H), 4.13 (s, 3H), 2.31-2.19 (m, 1H), 2.11 (d, J=1.1 Hz, 3H), 2.01-1.62 (m, 8H). LC-MS m/z: 274.1 [M+H]⁺. HPLC Purity (214 nm): 98%; t_{R}=7.16 min.

### Example 154—N-(2-(3,3-Difluorocyclopentyl)ethyl)-2-methoxy-4-methyl-1H-imidazole-1-carboxamide

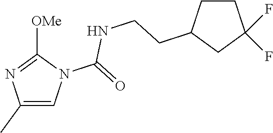 [SMILES: COc1nc(C)cn1C(=O)NCCC1CCC(F)(F)C1]

Following general procedure C, 2-methoxy-4-methyl-1H-imidazole (50 mg, 0.44 mmol) and 2-(3,3-difluorocyclopentyl)ethanamine (65 mg, 0.44 mmol) afforded the title compound (47 mg, 37.0%) as a white solid. ¹H NMR (400 MHz, CDCl₃) δ 6.94 (d, J=1.6 Hz, 1H), 6.88 (s, 1H), 4.15 (s, 3H), 3.40-3.55 (m, 2H), 2.34-2.28 (m, 2H), 2.21-2.15 (m, 2H), 2.11-2.08 (m, 3H), 2.07-1.99 (m, 2H), 1.75-1.67 (m, 3H), 1.49-1.43 (m, 1H). LC-MS m/z: 288.2 [M+H]⁺. HPLC Purity (214 nm): 100%; t_{R}=7.65 min.

### Example 155—2-Methoxy-4-methyl-N-(4-(1-(trifluoromethyl)-cyclopropyl)butyl)-1H-imidazole-1-carboxamide

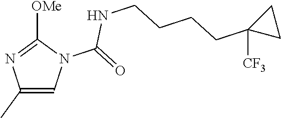 [SMILES: COc1nc(C)cn1C(=O)NCCCCC1(C(F)(F)F)CC1]

Following general procedure C, 2-methoxy-4-methyl-1H-imidazole (56 mg, 0.5 mmol) and 4-(1-(trifluoromethyl)cyclopropyl)butan-1-amine (78 mg, 0.4 mmol) afforded the title compound (34.5 mg, 32.3%) as a colorless liquid. ¹H NMR (400 MHz, CDCl₃) δ 6.94 (d, J=1.3 Hz, 1H), 6.89 (s, 1H), 4.14 (s, 3H), 3.46-3.20 (m, 2H), 2.11 (d, J=1.2 Hz, 3H), 1.67 (s, 1H), 1.63-1.43 (m, 5H), 1.00-0.88 (m, 2H), 0.59 (d, J=21.9 Hz, 2H). LC-MS m/z=319.7 [M+H]⁺. HPLC Purity (214 nm): 100%; t_{R}=8.48 min.

### Example 156—2-Methoxy-4-methyl-N-(4-phenylbutyl)-1H-imidazole-1-carboxamide

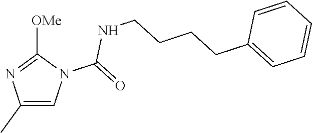 [SMILES: COc1nc(C)cn1C(=O)NCCCCc1ccccc1]

Following general procedure C, 2-methoxy-4-methyl-1H-imidazole (0.15 g, 1.34 mmol) and 4-phenylbutan-1-amine (0.22 g, 1.47 mmol) afforded the title compound (100.3 mg, 26.3%) as a white solid. ¹H NMR (400 MHz, CDCl₃) δ 7.30-7.26 (m, 2H), 7.20-7.16 (m, 3H), 6.93 (s, 1H), 6.83 (brs, 1H), 4.11 (s, 3H), 3.40-3.35 (m, 2H), 2.65 (t, J=7.2 Hz, 2H), 2.10 (s, 3H), 1.70-1.60 (m, 4H). LC-MS m/z: 288.2 [M+H]⁺. HPLC Purity (214 nm): >97%; t_{R}=8.25 min.

### Example 157—N-(4,4-Difluorocyclohexyl)-2-methoxy-4-phenyl-1H-imidazole-1-carboxamide

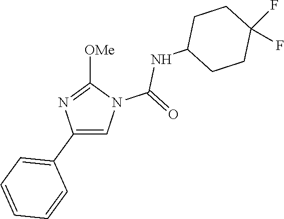 [SMILES: COc1nc(-c2ccccc2)cn1C(=O)NC1CCC(F)(F)CC1]

Following general procedure C, 2-methoxy-4-phenyl-1H-imidazole (80 mg, 0.46 mmol) and 4,4-difluorocyclohexanamine (74.48 mg, 0.55 mmol) afforded the title compound (34.6 mg, 22.5%) as a white solid. ¹H NMR (400 MHz, DMSO-d₆) δ 7.76 (d, J=8.0 Hz, 2H), 7.69 (s, 1H), 7.64 (d, J=8.0 Hz, 1H), 7.36 (t, J=7.6 Hz, 2H), 7.23 (t, J=7.4 Hz, 1H), 4.10 (s, 3H), 3.86 (brs, 1H), 2.06-2.00 (m, 3H), 1.95-1.89 (m, 3H), 1.79-1.73 (m, 2H). LC-MS m/z: 336.0 [M+H]⁺. HPLC Purity (214 nm): 100%; t_{R}=11.21 min.

### Example 158—N-(3,3-Difluorocyclohexyl)-2-methoxy-4-phenyl-1H-imidazole-1-carboxamide

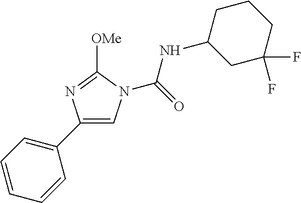 [SMILES: COc1nc(-c2ccccc2)cn1C(=O)NC1CCCC(F)(F)C1]

Following general procedure C, 2-methoxy-4-phenyl-1H-imidazole (80 mg, 0.46 mmol) and 3-difluorocyclohexanamine (74.48 mg, 0.55 mmol) afforded the title compound (34.6 mg, 22.5%) as a white solid. ¹H NMR (400 MHz, CDCl₃) δ 7.73 (d, J=7.9 Hz, 2H), 7.53 (s, 1H), 7.37 (t, J=7.6 Hz, 3H), 7.24 (s, 1H), 4.31 (brs, 1H), 4.24 (s, 3H), 2.32-2.21 (m, 1H), 2.04-1.86 (m, 3H), 1.83-1.72 (m, 4H). LC-MS m/z: 336.2 [M+H]⁺. HPLC Purity (214 nm): 100%; t_{R}=10.89 min.

### Example 159—4-Methyl-2-(2-morpholinoethoxy)-N-phenethyl-1H-imidazole-1-carboxamide

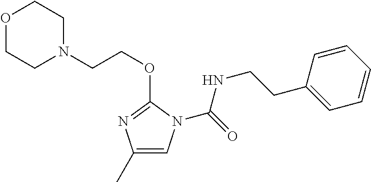 [SMILES: Cc1cn(C(=O)NCCc2ccccc2)c(OCCN2CCOCC2)n1]

Following general procedure E, 2-bromo-4-methyl-1-((2-(trimethylsilyl)ethoxy)methyl)-1H-imidazole (1.0 g, 3.4 mmol) in 2-morpholinoethanol (5.0 mL) afforded 4-(2-(4-methyl-1-((2-(trimethylsilyl)ethoxy)methyl)-1H-imidazol-2-yloxy)ethyl)morpholine (1.2 g, 97.8%) as a yellow oil. LC-MS m/z: 342.2 [M+H]⁺. Purity (254 nm): 100.0%; t_{R}=2.01 min.

Following general procedure F (method 2), 4-(2-(4-methyl-1-((2-(trimethylsilyl)ethoxy)methyl)-1H-imidazol-2-yloxy)ethyl)morpholine (1.2 g, 3.5 mmol) afforded 4-(2-(4-methyl-1H-imidazol-2-yloxy)ethyl)morpholine (700.0 mg, 94.8%) as a yellow oil. LC-MS m/z: 212.2 [M+H]⁺. Purity (254 nm): 100.0%; t_{R}=1.32 min.

Following general procedure B (method 1), 4-(2-(4-methyl-1H-imidazol-2-yloxy)ethyl)morpholine (200.0 mg, 0.9 mmol) and (2-isocyanatoethyl)benzene (200.0 mg, 1.4 mmol) afforded the title compound (102.0 mg, 43.9%) as a colorless oil. ¹H NMR (400 MHz, CDCl₃) δ 7.32 (t, J=7.2 Hz, 2H), 7.23 (dd, J=8.7, 7.3 Hz, 3H), 6.93 (d, J=1.2 Hz, 1H), 4.44 (t, J=5.6 Hz, 2H), 3.63 (dt, J=9.1, 5.7 Hz, 6H), 2.90 (t, J=6.8 Hz, 2H), 2.55 (t, J=5.6 Hz, 2H), 2.39-2.33 (m, 4H), 2.09 (d, J=1.2 Hz, 3H). LC-MS m/z: 359.1 [M+H]⁺. HPLC Purity (214 nm): 98.46%; t_{R}=6.40 min.

### Example 160—N-iso-Pentyl-2-methoxy-4-methyl-1H-imidazole-1-carboxamide

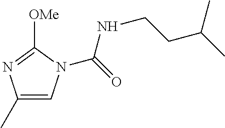 [SMILES: COc1nc(C)cn1C(=O)NCCC(C)C]

Following general procedure B (method 1), 2-methoxy-4-methyl-1H-imidazole (200 mg, 1.76 mmol) and 1-isocyanato-3-methylbutane (200 mg, 1.76 mmol) afforded the title compound (94.4 mg, 15.6%) as a colorless oil. ¹H NMR (400 MHz, CDCl₃) δ 6.88 (brs, 1H), 6.28 (d, J=1.2 Hz, 1H), 4.11 (s, 3H), 3.42-3.33 (m, 2H), 2.36 (d, J=1.2 Hz, 3H), 1.71-1.61 (m, 1H), 1.50 (q, J=6.8 Hz, 2H), 0.95 (d, J=6.4 Hz, 6H). LC-MS m/z: 226.2 [M+H]⁺. HPLC Purity (214 nm): >95%; t_{R}=7.52 min.

### Example 161—N-iso-Pentyl-2-phenoxy-1H-imidazole-1-carboxamide

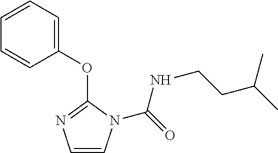 [SMILES: CC(C)CCNC(=O)n1ccnc1Oc1ccccc1]

Following general procedure E, 2-bromo-1H-imidazole (150 mg, 1.02 mmol in phenol (2 ml) afforded 2-phenoxy-1H-imidazole (60 mg, 36.8%) as a yellow solid. LC-MS m/z: 160.17 [M+H]r. Purity (214 nm): 95%; t_{R}=1.52 min.

Following general procedure C, 2-phenoxy-1H-imidazole (60 mg, 0.56 mmol) and 3-methylbutan-1-amine (80 mg, 0.76 mmol) afforded the title compound (46.6 mg, 45.5%) as a white solid. ¹H NMR (400 MHz, CDCl₃) δ 7.45 (t, J=6.9 Hz, 2H), 7.35 (d, J=1.9 Hz, 1H), 7.33-7.26 (m, 3H), 6.96 (brs, 1H), 6.61 (d, J=1.9 Hz, 1H), 3.47 (dt, J=6.0, 1.6 Hz, 2H), 1.67 (sep, J=6.8 Hz, 1H), 1.52 (q, J=7.2 Hz, 2H), 0.95 (d, J=6.6 Hz, 6H). LC-MS m/z: 274.2 [M+H]⁺. HPLC Purity (214 nm): 100%; t_{R}=9.01 min.

### Example 162—N-(4,4-Difluorocyclohexyl)-4-methyl-2-(3-(1-methylazetidin-3-yl)phenoxy)-1H-imidazole-1-carboxamide

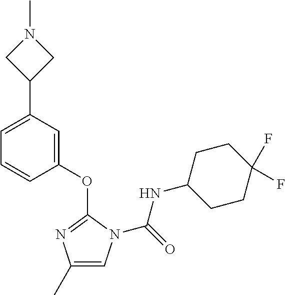 [SMILES: Cc1cn(C(=O)NC2CCC(F)(F)CC2)c(Oc2cccc(C3CN(C)C3)c2)n1]

To a solution of tert-butyl 3-(2-tosylhydrazineylidene)azetidine-1-carboxylate (20 g, 60 mmol) in dioxane (300 mL) were added (3-nitrophenyl)boronic acid (20.5 g, 90 mmol) and Cs₂CO₃ (29.3 g, 90 mmol). The mixture was stirred at 110° C. for 30 h and filtered. The filtrate was concentrated and purified by silica gel column chromatography (PE:EA=10:1) to give tert-butyl 3-(3-nitrophenyl)azetidine-1-carboxylate (7.0 g, 39.2%) as a yellow oil. LC-MS m/z: 284.2 [M−55]⁺. Purity (214 nm): 91%; t_{R}=1.59 min.

To a solution of tert-butyl 3-(3-nitrophenyl)azetidine-1-carboxylate (10.2 g, 30 mmol) in THF (10 mL) was added LAH (60 mL, 60 mmol). The mixture was stirred at 40° C. for 2 h and quenched with Na₂SO₄.10H₂O (3.0 g). The mixture was then filtered and the filtrate was concentrated and purified by silica gel column chromatography (DCM:MeOH=10:1) to give 3-(3-(benzyloxy)phenyl)-1-methylazetidine (7.5 g, 99%) as a yellow oil. LC-MS m/z: 254.1 [M+H]⁺. Purity (214 nm): 91%; t_{R}=1.56 min.

To a solution of 3-(3-(benzyloxy)phenyl)-1-methylazetidine (2.5 g, 10 mmol) in MeOH (10 mL) was added Pd/C (100 mg, 0.2 mmol). The mixture was stirred at RT for 2 h and filtered. The filtrate was concentrated to give 3-(1-methylazetidin-3-yl)phenol (1.6 g, 100%) as a yellow oil. LC-MS m/z: 164.2 [M+H]⁺. Purity (214 nm): 99%; t_{R}=1.08 min.

To a solution of 3-(1-methylazetidin-3-yl)phenol (163 mg, 1 mmol) in dioxane (8 mL) were added 2-bromo-4-methyl-1-((2-(trimethylsilyl)ethoxy)methyl)-1H-imidazole (290 mg, 1 mmol), CuI (19 mg, 0.1 mmol), DMEDA (18 mg, 0.2 mmol) and K₂CO₃ (276 mg, 2 mmol). The mixture was stirred at 90° C. for 3 h. The reaction mixture was filtered, concentrated and purified by silica gel column chromatography (DCM:MeOH=10:1) to give 4-methyl-2-(3-(1-methylazetidin-3-yl)phenoxy)-1-((2-(trimethylsilyl)ethoxy)methyl)-1H-imidazole (380 mg, crude) as a yellow oil. LC-MS m/z: 374.2 [M+H]⁺. Purity (214 nm): 65%; t_{R}=2.03 min.

Following general procedure F (method 2), 4-methyl-2-(3-(1-methylazetidin-3-yl)phenoxy)-1-((2-(trimethylsilyl)-ethoxy)methyl)-1H-imidazole (373 mg, 1.0 mmol) afforded 4-methyl-2-(3-(1-methylazetidin-3-yl)phenoxy)-1H-imidazole (95 mg, 39%) as a yellow oil. LC-MS m/z: 244.2 [M+H]⁺. Purity (214 nm): 76%; t_{R}=1.23 min.

Following general procedure C, 4-methyl-2-(3-(1-methylazetidin-3-yl)-phenoxy)-1H-imidazole (94 mg, 0.4 mmol) and 4,4-difluorocyclohexan-1-amine (55.0 mg, 0.4 mmol) afforded the title compound (3.4 mg, 1.7%) as a yellow solid. ¹H NMR (400 MHz, CDCl₃) δ 7.47 (t, J=8.0 Hz, 1H), 7.28-7.20 (m, 3H), 7.04 (d, J=0.8 Hz, 1H), 6.77 (d, J=7.4 Hz, 1H), 4.34 (t, J=8.8 Hz, 2H), 4.21-4.13 (m, 1H), 3.99-3.91 (m, 1H), 3.80 (t, 0.1=8.4 Hz, 2H), 2.76 (s, 3H), 2.18-2.07 (m, 7H), 1.98-1.86 (m, 2H), 1.68-1.65 (m, 2H). LC-MS m/z: 405.1 [M+H]⁺. HPLC Purity (214 nm): 100%; t_{R}=5.95 min.

### Example 163—2-(4-Cyanophenoxy)-N-(4,4-difluorocyclohexyl)-4-methyl-1H-imidazole-1-carboxamide

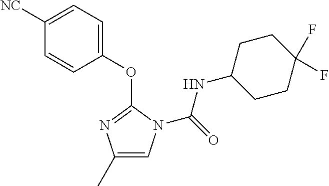 [SMILES: Cc1cn(C(=O)NC2CCC(F)(F)CC2)c(Oc2ccc(C#N)cc2)n1]

Following general procedure E, 2-bromo-4-methyl-1-((2-(trimethylsilyl)ethoxy)methyl)-1H-imidazole (870 mg, 3.0 mmol) and 4-hydroxybenzonitrile (7.14 g, 60.0 mmol) afforded 4-(4-methyl-1-((2-(trimethylsilyl)ethoxy)methyl)-1H-imidazol-2-yloxy)benzonitrile (500 mg, 51%) as a yellow solid. LC-MS m/z: 330.1 [M+H]⁺. Purity (214 nm): >14%; t_{R}=1.52 min.

Following general procedure F (method 2), 4-(4-methyl-1-((2-(trimethylsilyl)ethoxy)methyl)-1H-imidazol-2-yloxy)benzonitrile (500 mg, 1.52 mmol) afforded 4-(4-methyl-1H-imidazol-2-yloxy)benzonitrile (200 mg, 66%) as a yellow oil. LC-MS m/z: 200.1 [M+H]⁺. Purity (254 nm): 75%; t_{R}=1.57 min.

Following general procedure C, 4-(4-methyl-1H-imidazol-2-yloxy)benzonitrile (150 mg, 0.75 mmol) and 4,4-difluorocyclohexanamine hydrochloride (129 mg, 0.75 mmol) afforded the title compound (24.4 mg, 9.0%) as a white solid. ¹H NMR (400 MHz, CDCl₃) δ 7.76 (d, J=8.8 Hz, 2H), 7.48 (d, J=8.8 Hz, 2H), 7.04 (d, J=1.2 Hz, 1H), 6.56 (d, J=7.1 Hz, 1H), 3.98-3.93 (m, 1H), 2.19-2.03 (m, 7H), 2.00-1.77 (m, 2H), 1.70-1.59 (m, 2H). LC-MS m/z: 361.0 [M+H]⁺. Purity (214 nm): 95.17%; t_{R}=7.94 min.

### Example 164—2-(3-Cyanophenoxy)-N-(4,4-difluorocyclohexyl)-4-methyl-1H-imidazole-1-carboxamide

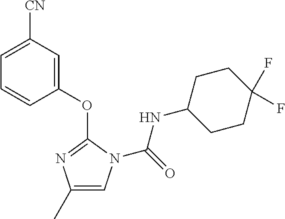 [SMILES: Cc1cn(C(=O)NC2CCC(F)(F)CC2)c(Oc2cccc(C#N)c2)n1]

Following general procedure C, 3-(4-methyl-1H-imidazol-2-yloxy)benzonitrile (150 mg, 0.75 mmol) and 4,4-difluorocyclohexanamine hydrochloride (129 mg, 0.75 mmol) afforded the title compound (9.4 mg, 3.5%) as a white solid. ¹H NMR (400 MHz, CDCl₃) δ 7.68 (dd, J=3.2, 1.5 Hz, 1H), 7.63-7.52 (m, 3H), 7.07 (d, J=1.2 Hz, 1H), 6.59 (d, J=7.0 Hz, 1H), 3.98-3.94 (m, 1H), 2.18-2.03 (m, 4H), 2.11 (d, J=1.2 Hz, 3H), 1.98-1.89 (m, 2H), 1.71-1.60 (m, 2H). LC-MS m/z: 361.0 [M+H]⁺. Purity (214 nm): 90.39%; t_{R}=8.00 min.

### Example 165—N-(4,4-Difluorocyclohexyl)-4-methyl-2-(4-morpholinophenoxy)-1H-imidazole-1-carboxamide

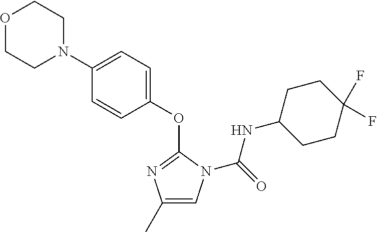 [SMILES: Cc1cn(C(=O)NC2CCC(F)(F)CC2)c(Oc2ccc(N3CCOCC3)cc2)n1]

A mixture of 4-morpholinophenol (500 mg, 2.79 mmol), diboc-thiourea (770 mg, 2.79 mmol), HgCl₂ (835 mg, 3.07 mmol) and TEA (901 mg, 8.93 mmol) in anhydrous DCM (6 mL) was stirred at 0° C. for 1 h and then stirred at RT for 1 h. The resulting mixture was filtered, concentrated and purified by silica gel column chromatography (PE:EA=3:1) to give the desired intermediate (900 mg, 77.1%) as a white solid. LC-MS m/z: 422.0 [M+H]⁺. Purity (214 nm): 99%; t_{R}=2.16 min. The intermediate was dissolved in DCM (4 mL) and TFA (6 mL) was added and the solution was stirred at RT for 1 h. The reaction mixture was concentrated to give 4-morpholinophenyl carbamimidate (400 mg, 84.7%) as a clear oil. LC-MS m/z: 222.2 [M+H]⁺. Purity (214 nm): 94%; t_{R}=1.26 min.

A suspension of 1-bromopropan-2-one (200 mg, 1.45 mmol), 4-morpholinophenyl carbamimidate (400 mg, 1.80 mmol) and NaHCO₃ (609 mg, 7.25 mmol) in EtOH (10 mL) was stirred at 78° C. for 16 h and then cooled. The mixture was filtered, concentrated and purified to give 4-(4-((4-methyl-1H-imidazol-2-yl)oxy)phenyl)morpholine (60 mg, 15.9%) as a white solid. LC-MS m/z: 260.2 [M+H]⁺. Purity (214 nm): 99%; t_{R}=1.56 min.

Following general procedure C, 4-(4-((4-methyl-1H-imidazol-2-yl)oxy)phenyl)morpholine (60 mg, 0.23 mmol) and 4,4-difluorocyclohexan-1-amine (59 mg, 0.34 mmol) afforded the title compound (41.0 mg, 42.8%) as a grey solid. ¹H NMR (400 MHz, CDCl₃) δ 7.19 (d, J=9.2 Hz, 2H), 7.02 (s, 1H), 6.95-6.89 (m, 3H), 3.95-3.94 (m, 1H), 3.88-3.85 (m, 4H), 3.15-3.13 (m, 4H), 2.16-2.07 (m, 7H), 1.97-1.82 (m, 2H), 1.68-1.55 (m, 2H). LC-MS m/z: 421.0 [M+H]⁺. HPLC Purity (214 nm): 100%; t_{R}=8.31 min.

### Example 166—N-(2-(3, 3-Difluorocyclobutyl)ethyl)-2-methoxy-4-methyl-1H-imidazole-1-carboxamide

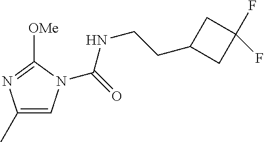 [SMILES: COc1nc(C)cn1C(=O)NCCC1CC(F)(F)C1]

Following general procedure C, 2-methoxy-4-methyl-1H-imidazole (34 mg, 0.3 mmol) and 2-(3,3-difluorocyclobutyl)ethan-1-amine (44.5 mg, 0.33 mmol) afforded the title compound (45.6 mg, 55.0%) as a white solid. ¹H NMR (400 MHz, CDCl₃) δ 6.93 (d, J=1.2 Hz, 1H), 6.85 (brs, 1H), 4.15 (s, 3H), 3.34 (dd, J=13.7, 6.4 Hz, 2H), 2.72 (ddt, J=13.1, 9.8, 5.4 Hz, 2H), 2.31-2.13 (m, 3H), 2.11 (d, J=1.1 Hz, 3H), 1.81 (p, J=7.1 Hz, 2H). LC-MS m/z: 274.1 [M+H]⁺. HPLC Purity (214 nm): 100%; t_{R}=7.48 min.

### Example 167—N-(2-(2,2-Difluorocyclopentyl)ethyl)-2-methoxy-4-methyl-1H-imidazole-1-carboxamide

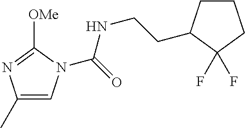 [SMILES: COc1nc(C)cn1C(=O)NCCC1CCCC1(F)F]

Following general procedure C, 2-methoxy-4-methyl-1H-imidazole (50 mg, 0.44 mmol) and 2-(2,2-difluorocyclopentyl)ethanamine (65 mg, 0.44 mmol) afforded the title compound 62 mg, 49%) as a colorless oil. ¹H NMR (400 MHz, CDCl₃) δ 6.98 (brs, 1H), 6.94 (d, J=1.2 Hz, 1H), 4.14 (s, 3H), 3.48-3.43 (m, 2H), 2.16-2.00 (m, 7H), 1.91-1.67 (m, 4H), 1.51-1.41 (m, 1H). LC-MS m/z: 288.2 [M+H]⁺. HPLC Purity (214 nm): 98.50%; t_{R}=7.73 min.

### Example 168—4-Chloro-2-methoxy-N-(4,4,4-trifluorobutyl)-1H-imidazole-1-carboxamide

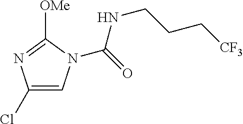 [SMILES: COc1nc(Cl)cn1C(=O)NCCCC(F)(F)F]

To a stirred solution of NaH (588 mg, 14.7 mmol) in THF (10 mL) was added 4-chloro-1H-imidazole (1.0 g, 9.80 mmol) at 0° C. and the resulting reaction mixture was stirred for 30 min and then SEMCl (1.96 g, 11.8 mmol) was added and the mixture was stirred at RT for 16 h. The reaction mixture was diluted with EA (200 mL), washed with water (30 mL×3), brine (30 mL), dried over Na₂SO₄, filtered and concentrated to give a residue which was purified by silica gel column chromatography (EA:PE=1:6) to give 4-chloro-1-((2-(trimethylsilyl)ethoxy)methyl)-1H-imidazole (1.4 g, 99.1%) as a clear oil. LC-MS m/z: 233.0 [M+H]⁺. Purity (214 nm): 99%; t_{R}=1.88 min.

A solution of 4-chloro-1-((2-(trimethylsilyl)ethoxy)methyl)-1H-imidazole (1.26 g, 5.43 mmol) and NBS (966 mg, 5.43 mmol) in MeCN (10 mL) was stirred at RT for 2 h. Then the reaction mixture was concentrated and purified by silica gel column chromatography (EA:PE=1:9) to give 2-bromo-4-chloro-1-((2-(trimethylsilyl)ethoxy)methyl)-1H-imidazole (600 mg, 35.7%) as a yellow oil. LC-MS m/z: 312.9 [M+H]⁺. Purity (214 nm): 97%; t_{R}=2.19 min.

A suspension of 2-bromo-4-chloro-1-((2-(trimethylsilyl)ethoxy)methyl)-1H-imidazole (600 mg, 1.94 mmol), NaOMe (627 mg, 11.6 mmol) and CuBr (30 mg, 0.19 mmol) in MeOH (4 mL) was stirred at 110° C. for 2.5 h under microwave condition. The reaction mixture was concentrated and purified by silica gel column chromatography (EA:PE=1:6) to give 4-chloro-2-methoxy-1-((2-(trimethylsilyl)ethoxy)methyl)-1H-imidazole (187 mg, 36.7%) as a clear oil. LC-MS m/z: 263.1 [M+H]⁺. Purity (214 nm): 75%; t_{R}=1.48 min.

Following general procedure F (method 1), 4-chloro-2-methoxy-1-((2-(trimethylsilyl)ethoxy)methyl)-1H-imidazole (187 mg, 0.71 mmol) afforded 4-chloro-2-methoxy-1H-imidazole (69.0 mg, 74.2%) as a white solid. LC-MS m/z: 132.1 [M+H]⁺. Purity (214 nm): 99%; t_{R}=1.28 min.

Following general procedure C, 4-chloro-2-methoxy-1H-imidazole (69 mg, 0.52 mmol) and 4,4,4-trifluorobutan-1-amine (99 mg, 0.78 mmol) afforded the title compound (54.4 mg, 37.4%) as a clear oil. ¹H NMR (400 MHz, CDCl₃) δ 7.13 (s, 1H), 6.92 (brs, 1H), 4.18 (s, 3H), 3.46 (q, J=6.8 Hz, 2H), 2.24-2.12 (m, 2H), 1.90 (p, J=7.6 Hz, 2H). LC-MS m/z: 286.0 [M+H]⁺. HPLC Purity (214 nm): 100%; t_{R}=8.29 min.

### Example 169—N-iso-Pentyl-2-methoxy-4,5-dimethyl-1H-imidazole-1-carboxamide

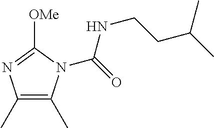 [SMILES: COc1nc(C)c(C)n1C(=O)NCCC(C)C]

A suspension of 3-bromobutan-2-one (5.0 g, 33.1 mmol), methyl carbamimidate (6.1 g, 49.7 mmol) and NaHCO₃ (13.9 g, 165.5 mmol) in EtOH (100 ml) was stirred at 65° C. under N₂ overnight. The mixture was concentrated, poured into water (20 ml), extracted with DCM (30 ml×2), dried over Na₂SO₄, filtered, concentrated and purified by silica gel chromatography (PE:EA=4:1) to give 2-methoxy-4,5-dimethyl-1H-imidazole (800 mg, 92%) as an off-white solid. LC-MS m/z: 127.1 [M+H]⁺. Purity (214 nm): 93%; t_{R}=1.24 min.

Following general procedure B (method 1), 2-methoxy-4,5-dimethyl-1H-imidazole (100 mg, 0.79 mmol) and crude 2-(isopentylimino)ethenone (300 mg) afforded the title compound (29.0 mg, 3.6%) as a white solid. ¹H NMR (400 MHz, DMSO-d₆) δ 8.00 (brs, 1H), 3.88 (s, 3H), 3.18 (dd, J=14.1, 6.2 Hz, 2H), 2.07 (s, 3H), 1.92 (s, 3H), 1.68-1.54 (m, 1H), 1.38 (dd, J=14.4, 7.0 Hz, 2H), 0.89 (d, J=6.6 Hz, 6H). LC-MS m/z: 240.3 [M+H]⁺. HPLC Purity (214 nm): 100%; t_{R}=6.68 min.

### Example 170—2-Methoxy-4,5-dimethyl-N-phenethyl-1H-imidazole-1-carboxamide

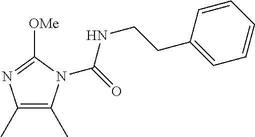 [SMILES: COc1nc(C)c(C)n1C(=O)NCCc1ccccc1]

Following general procedure B (method 1), 2-methoxy-4,5-dimethyl-1H-imidazole (0.12 g, 0.95 mmol) and 1-(2-isocyanatoethyl)benzene (0.28 g, 1.14 mmol) afforded the title compound 25.2 mg, 9.7%) as a colorless oil. ¹H NMR (400 MHz, DMSO-d₆) δ 8.00 (brs, 1H), 7.31-7.21 (m, 5H), 3.85 (s, 3H), 3.45-3.38 (m, 2H), 2.81 (t, J=6.0 Hz, 2H), 2.03 (s, 3H), 1.91 (s, 3H). LC-MS m/z: 274.2 [M+H]⁺. HPLC Purity (214 nm): 100%; t_{R}=7.64 min.

### Example 171—4-Methyl-2-phenoxy-N-(4,4,4-trifluorobutyl)-1H-imidazole-1-carboxamide

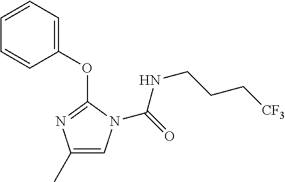 [SMILES: Cc1cn(C(=O)NCCCC(F)(F)F)c(Oc2ccccc2)n1]

Following general procedure C, 4-methyl-2-phenoxy-1H-imidazole (100 mg, 0.57 mmol) and 4,4,4-trifluorobutan-1-amine (110 mg, 0.86 mmol) afforded the title compound (68.2 mg, 36.3%) as a white solid. ¹H NMR (400 MHz, CDCl₃) δ 7.46-7.42 (m, 2H), 7.30-7.27 (m, 3H), 7.05 (d, J=1.2 Hz, 1H), 7.02 (brs, 1H), 3.50 (q, J=7.6 Hz, 2H), 2.20-2.12 (m, 2H), 2.08 (d, J=1.2 Hz, 3H), 1.91 (p, J=6.8 Hz, 2H). LC-MS m/z: 328.2 [M+H]⁺. HPLC Purity (254 nm): 96.29%; t_{R}=8.54 min.

### Example 172—N-(4,4-Difluorocyclohexyl)-4-methyl-2-phenoxy-1H-imidazole-1-carboxamide

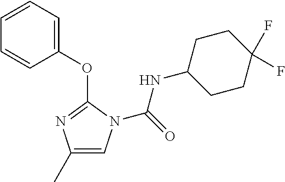 [SMILES: Cc1cn(C(=O)NC2CCC(F)(F)CC2)c(Oc2ccccc2)n1]

Following general procedure C, 4-methyl-2-phenoxy-1H-imidazole (150 mg, 0.86 mmol) and 4,4-difluorocyclohexanamine (130 mg, 0.95 mmol) afforded the title compound (58.3 mg, 20.2%) as a white solid. ¹H NMR (400 MHz, CDCb3) δ 7.46-7.40 (m, 2H), 7.32-7.25 (m, 3H), 7.04 (d, J=1.6 Hz, 1H), 6.83 (brs, 1H), 3.93 (brs, 1H), 2.15-2.04 (m, 4H), 2.09 (d, J=1.2 Hz, 3H), 1.96-1.83 (m, 2H), 1.67-1.59 (m, 2H). LC-MS m/z: 336.0 [M+H]⁺. HPLC Purity (214 nm): 100%; t_{R}=8.24 min.

### Example 173—N-(4-Methylpent-2-yn-1-yl)-2-phenoxy-1H-imidazole-1-carboxamide

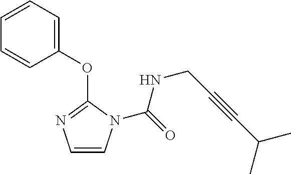 [SMILES: CC(C)C#CCNC(=O)n1ccnc1Oc1ccccc1]

Following general procedure E, 2-bromo-1H-imidazole (500 mg, 3.4 mmol) in phenol (10 mL) afforded 2-phenoxy-1H-imidazole (320 mg, 58.8%) as a brown solid. LC-MS m/z: 161.0 [M+H]⁺. Purity (254 nm): no absorption, t_{R}=0.53 min.

Following general procedure C, 2-phenoxy-1H-imidazole (50 mg, 0.31 mmol) and 4-methyl pent-2-yn-1-amine hydrogen chloride (42 mg, 0.31 mmol) afforded the title compound (13.4 mg, 15.1%) as a white solid. ¹H NMR (400 MHz, CDCl₃) δ 7.48-7.44 (m, 2H), 7.37-7.28 (m, 4H), 7.13 (brs, 1H), 6.61 (d, J=4.0 Hz, 1H), 4.20 (dd, J=5.2, 2.4 Hz, 2H), 2.57-2.53 (m, 1H), 1.14 (d, J=6.8 Hz, 6H). LC-MS m/z: 284.1 [M+H]⁺. HPLC Purity (214 nm): 97.41%; t_{R}=8.63 min.

### Example 174—4-(1-Methylpiperidin-4-yl)-N-(3-(1-(trifluoromethyl)cyclopropyl)propyl)-1H-imidazole-1-carboxamide

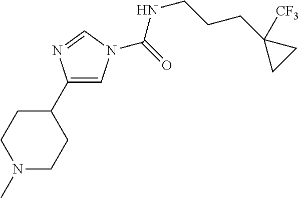 [SMILES: CN1CCC(c2cn(C(=O)NCCCC3(C(F)(F)F)CC3)cn2)CC1]

Following general procedure C, 4-(1H-imidazol-4-yl)-1-methylpiperidine (50 mg, 0.30 mmol) and 3-(1-(trifluoromethyl)cyclopropyl)propan-1-amine (100 mg, 0.60 mmol) afforded the title compound (30 mg, 28%) as a white solid. ¹H NMR (400 MHz, MeOD) δ 8.19 (s, 1H), 7.38 (s, 1H), 3.36-3.34 (m, 2H), 3.03 (d, J=12.0 Hz, 4H), 2.62-2.56 (m, 1H), 2.38 (s, 3H), 2.30-2.24 (m, 2H), 2.09 (d, J=11.6 Hz, 2H), 1.81-1.61 (m, 6H), 0.97-0.94 (m, 2H), 0.69 (brs, 2H). LC-MS m/z: 359.2 [M+H]⁺. HPLC Purity (214 nm): 96.19%, t_{R}=5.26 min.

### Example 175—4-(2-(Piperidin-1-yl)pyridin-3-yl)-N-(3-(1-(trifluoromethyl)cyclopropyl) propyl)-1H-imidazole-1-carboxamide

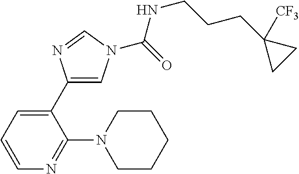 [SMILES: O=C(NCCCC1(C(F)(F)F)CC1)n1cnc(-c2cccnc2N2CCCCC2)c1]

Following general procedure C. 3-(1H-imidazol-4-yl)-2-(piperidin-1-yl)pyridine (50 mg, 0.22 mmol) and 3-(1-(trifluoromethyl)cyclopropyl)propan-1-amine (55 mg, 0.33 mmol) afforded the title compound (9.7 mg, 10%) as a white solid. ¹H NMR (400 MHz, CDCl₃) δ 8.31-8.25 (m, 2H), 8.16 (d, J=7.6 Hz, 1H), 7.04-7.01 (m, 1H), 5.69 (brs, 1H), 3.49 (q, J=6.8 Hz, 2H), 3.14-3.09 (m, 4H), 1.90-1.83 (m, 2H), 1.71-1.55 (m, 8H), 1.03-1.00 (m, 2H), 0.62 (brs, 2H). LC-MS m/z: 422.0 [M+H]⁺. HPLC Purity (214 nm): 98.30%; t_{R}=6.39 min.

### Example 176—4-(2-(4-Methylpiperazin-1-yl)pyridin-3-yl)-N-(3-(1-(trifluoromethyl)cyclo

propyl)propyl)-1H-imidazole-1-carboxamide

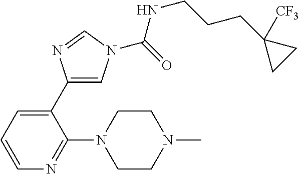 [SMILES: CN1CCN(c2ncccc2-c2cn(C(=O)NCCCC3(C(F)(F)F)CC3)cn2)CC1]

Following general procedure C, 1-(3-(1H-imidazol-4-yl)pyridin-2-yl)-4-methylpiperazine (50 mg, 0.20 mmol) and 3-(1-(trifluoromethyl)cyclopropyl)propan-1-amine (67 mg, 0.4 mmol) afforded the title compound (12.8 mg, 15%) as a white solid. ¹H NMR (400 MHz, CDCl₃) δ 8.34 (s, 1H), 8.24 (dd, J=7.2, 2.0 Hz, 1H), 8.20-8.18 (m, 1H), 8.12 (s, 1H), 8.04 (brs, 1H), 7.07-7.03 (m, 1H), 3.53-3.48 (m, 4H), 3.41 (q, J=6.8 Hz, 2H), 3.20-3.11 (m, 4H), 2.70 (s, 3H), 1.84-1.80 (m, 2H), 1.67-1.63 (m, 2H), 0.98-0.95 (m, 2H), 0.60 (brs, 2H). LC-MS m/z: 437.2 [M+H]⁺. HPLC Purity (214 nm): 98.35%; t_{R}=5.53 min.

### Example 177—2-Phenoxy-N-(3-(1-(trifluoromethyl)cyclopropyl)propyl)-1H-imidazole-1-carboxamide

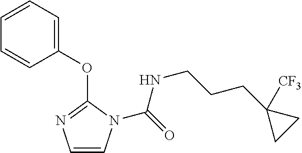 [SMILES: O=C(NCCCC1(C(F)(F)F)CC1)n1ccnc1Oc1ccccc1]

Following general procedure C, 2-phenoxy-1H-imidazole (100 mg, 0.625 mmol) and 3-(1-(trifluoromethyl)cyclopropyl)propan-1-amine (125 mg, 0.75 mmol) afforded the title compound (94.8 mg, 43.0%) as a white solid. ¹H NMR (400 MHz, CDCl₃) δ 7.52-7.45 (m, 2H), 7.37 (d, J=2.0 Hz, 1H), 7.35-7.29 (m, 3H), 7.04 (brs, 1H), 6.63 (d, J=2.0 Hz, 1H), 3.44 (q, J=5.3 Hz, 2H), 1.85-1.77 (m, 2H), 1.64 (dd, J=10.5, 5.9 Hz, 2H), 0.99 (t, J=5.8 Hz, 2H), 0.61-0.55 (m, 2H). LC-MS m/z: 354.1 [M+H]⁺. HPLC Purity (254 nm): 100%; t_{R}=2.06 min.

### Example 178—2-Phenoxy-N-(4,4,4-trifluorobutyl)-1H-imidazole-1-carboxamide

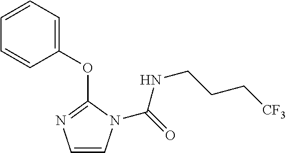 [SMILES: O=C(NCCCC(F)(F)F)n1ccnc1Oc1ccccc1]

Following general procedure C, 2-phenoxy-1H-imidazole (100 mg, 0.63 mmol) and 4,4,4-trifluorobutan-1-amine hydrogen chloride (102 mg, 0.63 mmol) afforded the title compound (27.4 mg, 14%) as a white solid. ¹H NMR (400 MHz, CDCl₃) δ 7.48-7.44 (m, 2H), 7.34-7.27 (m, 4H), 7.11 (brs, 1H), 6.61 (d, J=4.0 Hz, 1H), 3.52 (q, J=6.8 Hz, 2H), 2.23-2.13 (m, 2H), 1.96-1.89 (m, 2H). LC-MS m/z: 314.1 [M+H]⁺. HPLC Purity (214 nm): 100%; t_{R}=8.33 min.

### Example 179—N-(3-(3-(tert-Butyl)-1H-pyrazol-1-yl)propyl)-2-methoxy-4-methyl-1H-imidazole-1-carboxamide

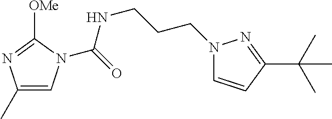 [SMILES: COc1nc(C)cn1C(=O)NCCCn1ccc(C(C)(C)C)n1]

Following general procedure C, 2-methoxy-4-methyl-1H-imidazole (0.34 g, 2.71 mmol) and 3-(3-(tert-butyl)-1H-pyrazol-1-yl)propan-1-amine (0.98 g, 5.41 mmol) afforded the title compound (400 mg, 41° %) as a colorless oil. ¹H NMR (500 MHz, CDCl₃) δ 7.29 (brs, 1H), 7.26 (d, J=3.0 Hz, 1H), 6.93 (d, J=1.5 Hz, 1H), 6.09 (d, J=3.0 Hz, 1H), 4.18-4.12 (m, 2H), 4.15 (s, 3H), 3.28 (q, J=8.0 Hz, 2H), 2.11 (s, 3H), 2.04 (p, J=8.0 Hz, 2H), 1.30 (s, 9H). LC-MS m/z: 342.1 [M+Na]⁺. HPLC Purity (214 nm): 100%; t_{R}=7.70 min.

### Example 180—2-Methoxy-4-methyl-N-(4-methylpent-2-yn-1-yl)-1H-imidazole-1-carboxamide

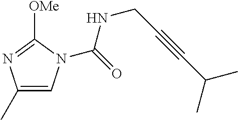 [SMILES: COc1nc(C)cn1C(=O)NCC#CC(C)C]

Following general procedure C, 2-methoxy-4-methyl-1H-imidazole (100 mg, 0.89 mmol) and 4-methylpent-2-yn-1-amine hydrogen chloride (269 mg, 1.34 mmol) afforded the title compound (60.8 mg, 29%) as a white solid. ¹H NMR (400 MHz, CDCl₃) δ 6.97 (brs, 1H), 6.94 (d, J=1.2 Hz, 1H), 4.16 (s, 3H), 4.13 (dd, J=8.4, 2.0 Hz, 2H), 2.58-2.54 (m, 1H), 2.10 (d, J=1.2 Hz, 3H), 1.16 (dd, J=7.2, 2.4 Hz, 6H). LC-MS m/z: 236.2 [M+H]⁺. HPLC Purity (214 nm): 96.11%, t_{R}=7.54 min.

### Example 181—2-Methoxy-4-methyl-N-(4-phenylbut-2-yn-1-yl)-1H-imidazole-1-carboxamide

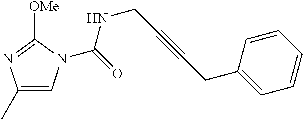 [SMILES: COc1nc(C)cn1C(=O)NCC#CCc1ccccc1]

Following general procedure C, 2-methoxy-4-methyl-1H-imidazole (20 mg, 0.18 mmol) and 4-phenylbut-2-yn-1-amine (64 mg, 0.45 mmol) afforded the title compound (28.8 mg, 57.6%) as a yellow oil. ¹H NMR (400 MHz, CDCl₃) δ 7.33 (d, J=4.4 Hz, 4H), 7.30-7.23 (m, 1H), 7.04 (brs, 1H), 6.95 (d, J=1.2 Hz, 1H), 4.22-4.19 (m, 2H), 4.14 (s, 3H), 3.62 (t, J=2.0 Hz, 2H), 2.11 (d, J 1.2 Hz, 3H). LC-MS m/z: 284.0 [M+H]⁺. HPLC Purity (214 nm): 97.03; t_{R}=8.31 min.

### Example 182—2-Methoxy-4-methyl-N-(4-(1-(trifluoromethyl)cyclopropyl)but-2-ynyl)-1H-imidazole-1-carboxamide

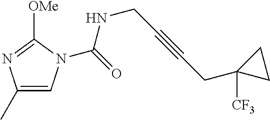 [SMILES: COc1nc(C)cn1C(=O)NCC#CCC1(C(F)(F)F)CC1]

Following general procedure C, 2-methoxy-4-methyl-1H-imidazole (100 mg, 0.89 mmol) and 4-(1-(trifluoromethyl)cyclopropyl)but-2-yn-1-amine (150 mg, 0.89 mmol) afforded the title compound (9.3 mg, 3.3%) as a white solid. ¹H NMR (400 MHz, CDCl₃) δ 6.99 (brs, 1H), 6.93 (d, J=1.3 Hz, 1H), 4.18 (s, 3H), 4.14-4.09 (m, 2H), 2.69 (t, J=2.2 Hz, 2H), 2.11 (d, J=1.3 Hz, 3H), 1.01-0.95 (m, 2H), 0.87-0.83 (m, 2H). LC-MS m/z: 316.2 [M+H]⁺. HPLC Purity (214 nm): 100%; t_{R}=8.11 min.

### Example 183—N-Cyclopropyl-2-(4,4-difluorocyclohexyloxy)-4-methyl-1H-imidazole-1-carboxamide

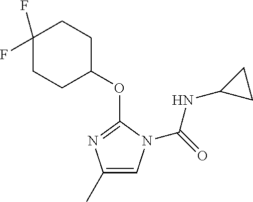 [SMILES: Cc1cn(C(=O)NC2CC2)c(OC2CCC(F)(F)CC2)n1]

Following general procedure E, 2-bromo-4-methyl-1-((2-(trimethylsilyl)ethoxy)methyl)-1H-imidazole (1.2 g, 4.1 mmol) and 4,4-difluorocyclohexanol (500 mg, 3.7 mmol) afforded 2-(4,4-difluorocyclohexyloxy)-4-methyl-1-((2-(trimethylsilyl)ethoxy)methyl)-1H-imidazole (250 mg, 19.7%) as a yellow oil. LC-MS m/z: 347.0 [M+H]⁺. Purity (254 nm): 100.0%; t_{R}=2.26 min

Following general procedure F (method A), 2-(4,4-difluorocyclohexyloxy)-4-methyl-1-((2-(trimethylsilyl)ethoxy)methyl-1H-imidazole (250 mg, 0.72 mmol) afforded 2-(4,4-difluorocyclohexyloxy)-4-methyl-1H-imidazole (60 mg, 19.7%) as a white solid. LC-MS m/z: 217.1 [M+H]⁺. Purity (254 nm): 100.0%; t_{R}=1.74 min.

Following general procedure C, 2-(4,4-difluorocyclohexyloxy)-4-methyl-1H-imidazole (60 mg, 0.28 mmol) and cyclopropanamine (16 mg, 0.28 mmol) afforded the title compound (25.1 mg, 30.2%) as a white solid. ¹H NMR (400 MHz, CDCl₃) δ 6.96 (d, J=1.2 Hz, 2H), 5.22 (brs, 1H), 2.82 (qd, J=7.0, 3.7 Hz, 1H), 2.11 (d, J=1.2 Hz, 3H), 2.18-1.98 (m, 8H), 0.88 (dd, J=6.9, 5.7 Hz, 2H), 0.63-0.56 (m, 2H). LC-MS m/z: 300.0 [M+H]⁺. HPLC Purity (254 nm): 100%; t_{R}=7.96 min.

### Example 184—4-Bromo-2-methoxy-N-(4,4,4-trifluorobutyl)-1H-imidazole-1-carboxamide

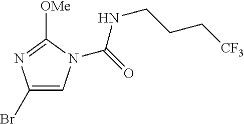 [SMILES: COc1nc(Br)cn1C(=O)NCCCC(F)(F)F]

To a solution of 4,5-dibromo-2-methoxy-1-((2-(trimethylsilyl)ethoxy)methyl)-1H-imidazole (1.2 g, 3.1 mmol) in THF (10 mL) was added n-BuLi (2.5 mol/L, 1.38 mL) at −78° C. and the mixture was stirred at −78° C. for 2 h. The mixture was poured into ice-water (20 mL) and extracted with EA (3×30 mL), dried over anhydrous Na₂SO₄, filtered, concentrated and purified by silica gel column chromatography (PE:EA=5:1) to give 4-bromo-2-methoxy-1-((2-(trimethylsilyl)ethoxy)methyl)-1H-imidazole (800 mg, 84%) as a yellow oil. LC-MS m/z: 306.9. [M+H]⁺. Purity (214 nm): 89%; t_{R}=2.16 min.

Following general procedure F (method 1), 4-bromo-2-methoxy-1-((2-(trimethylsilyl)ethoxy)methyl)-1H-imidazole (400 mg, 1.3 mmol) afforded 4-bromo-2-methoxy-1H-imidazole (200 mg, 87%) as a yellow oil. LC-MS m/z: 177.0 [M+H]⁺. Purity (214 nm): 96%; t_{R}=1.02 min.

Following general procedure C, 4-bromo-2-methoxy-1H-imidazole (100 mg, 0.57 mmol) and 4,4,4-trifluorobutan-1-amine (93 mg, 0.57 mmol) afforded the title compound (41.5 mg, 22.2%) as a white solid. ¹H NMR (400 MHz, CDCl₃) δ 7.22 (s, 1H), 6.91 (brs, 1H), 4.19 (s, 3H), 3.46 (dd, J=13.4, 6.8 Hz, 2H), 2.24-2.15 (m, 2H), 1.91 (p, J=7.6 Hz, 2H). LC-MS m/z: 330.0 [M+H]⁺. Purity (214 nm): 100%; t_{R}=8.29 min.

### Example 185—4-Chloro-N-(2-(2,2-difluorocyclopentyl)ethyl)-2-methoxy-1H-imidazole-1-carboxamide

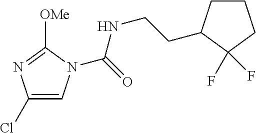 [SMILES: COc1nc(Cl)cn1C(=O)NCCC1CCCC1(F)F]

Following general procedure C, 4-chloro-2-methoxy-1H-imidazole (20 mg, 0.15 mmol) and 2-(2,2-difluorocyclopentyl)ethan-1-amine (30 mg, 0.16 mmol) afforded the title compound (11.0 mg, 23.9%) as a white solid. ¹H NMR (400 MHz, CDCl₃) δ 7.13 (s, 1H), 6.94 (brs, 1H), 4.17 (s, 3H), 3.46 (q, J=6.8 Hz, 2H), 2.09-2.01 (m, 4H), 1.98-1.73 (m, 4H), 1.51-1.44 (m, 1H). LC-MS m/z: 308.1 [M+H]⁺. HPLC Purity (214 nm): 96.65%; t_{R}=9.50 min.

### Example 186—4-Chloro-2-methoxy-N-(4-methylpent-2-yn-1-yl)-1H-imidazole-1-carboxamide

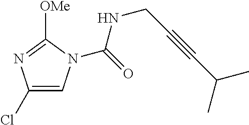 [SMILES: COc1nc(Cl)cn1C(=O)NCC#CC(C)C]

Following general procedure C, 4-chloro-2-methoxy-1H-imidazole (30 mg, 0.23 mmol) and 4-methylpent-2-yn-1-amine (60 mg, 0.46 mmol) afforded the title compound (3.5 mg, 6.0%) as a white solid. ¹H NMR (400 MHz, CDCl₃) δ 7.14 (s, 1H), 6.94 (brs, 1H), 4.19 (s, 3H), 4.14 (dd, J=5.2 Hz, 2.0 Hz, 2H), 2.58-2.55 (m, 1H), 1.16 (d, J=6.8 Hz, 6H). LC-MS m/z: 278.1 [M+Na]⁺. HPLC Purity (214 nm): 100.00%; t_{R}=8.61 min.

### Example 187—4-Chloro-2-isopropoxy-N-(4,4,4-trifluorobutyl)-1H-imidazole-1-carboxamide

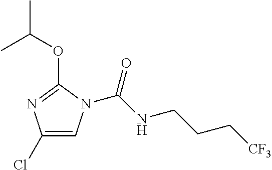 [SMILES: CC(C)Oc1nc(Cl)cn1C(=O)NCCCC(F)(F)F]

To a solution of 2-bromo-4-chloro-1-((2-(trimethylsilyl)ethoxy)methyl)-1H-imidazole (400 mg, 1.3 mmol) in THF (5 mL) was added sodium propan-2-olate (640 mg, 7.8 mmol). The reaction mixture was stirred at 90° C. for 3 h under microwave and then cooled to RT. The mixture was poured into H₂O (10 mL), with EA (30 mL×2), washed with brine (50 mL×1), dried over Na₂SO₄, concentrated and purified by silica gel chromatography (DCM/MeOH=30/1) to give 4-chloro-2-isopropoxy-1-((2-(trimethylsilyl)ethoxy)methyl)-1H-imidazole (120 mg, 32.1%) as a yellow oil. LC-MS m/z: 291.1 [M+H]⁺. Purity (214 nm): 79.69%; t_{R}=1.62 min.

Following general procedure F (method 1), 4-chloro-2-isopropoxy-1-((2-(trimethylsilyl)ethoxy)methyl)-1H-imidazole (120 mg, 0.4 mmol) afforded 4-chloro-2-isopropoxy-1H-imidazole (55 mg, 83.3%) as a white solid. LC-MS m/z: 161.1 [M+H]⁺. Purity (214 nm): 100.0%; t_{R}=0.85 min.

Following general procedure C, 4-chloro-2-isopropoxy-1H-imidazole (55 mg, 0.34 mmol) and 4,4,4-trifluorobutan-1-amine (43 mg, 0.34 mmol) afforded the title compound (8.5 mg, 7.9%) as a white solid. ¹H NMR (400 MHz, CDCl₃) δ 7.29 (s, 1H), 7.12 (s, 1H), 5.31 (septet, J=6.2 Hz, 1H), 3.49 (q, J=6.6 Hz, 2H), 2.23-2.18 (m, 2H), 1.98-1.84 (m, 2H), 1.49 (d, J=6.2 Hz, 6H). LC-MS m/z: 272.1 [M+H]⁺. HPLC Purity (254 nm): 100%; t_{R}=9.14 min.

### Example 188—4-Chloro-2-((1-methylpiperidin-4-yl)oxy)-N-(4,4,4-trifluorobutyl)-1H-imidazole-1-carboxamide

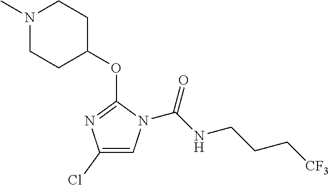 [SMILES: CN1CCC(Oc2nc(Cl)cn2C(=O)NCCCC(F)(F)F)CC1]

Following general procedure E, 2-bromo-4-chloro-1-((2-(trimethylsilyl)ethoxy)methyl)-1H-imidazole (800 mg, 2.6 mmol) and 1-methylpiperidin-4-ol (10 mL) afforded 4-((4-chloro-1-((2-(trimethylsilyl)ethoxy)methyl)-1H-imidazol-2-yl)oxy)-1-methylpiperidine (518 mg, 57.7%) as a yellow oil. LC-MS m/z: 346.1 [M+H]⁺. Purity (214 nm): 100%; t_{R}=1.5 min.

Following general procedure F (method 1), 4-((4-chloro-1-((2-(trimethylsilyl)ethoxy) methyl)-1H-imidazol-2-yl)oxy)-1-methylpiperidine (500 mg, 1.45 mmol) afforded 4-((4-chloro-1H-imidazol-2-yl)oxy)-1methylpiperidine (270 mg, 86.5%) as a yellow solid. LC-MS m/z: 216.1 [M+H]⁺. Purity (214 nm): 64.35%; t_{R}=1.37 min.

Following general procedure C, 4-((4-chloro-1H-imidazol-2-yl)oxy)-1-methylpiperidine (215 mg, 1 mmol) and 4,4,4-trifluorobutan-1-amine (163 mg, 1 mmol) afforded the title compound (13.1 mg, 5.9%) as a colorless oil. ¹H NMR (400 MHz, CDCl₃) δ 7.11 (s, 1H), 7.00 (brs, 1H), 5.18-5.01 (m, 1H), 3.47 (q, J=6.7 Hz, 2H), 2.68-2.59 (m, 2H), 2.42-2.31 (m, 2H), 2.28 (s, 3H), 2.23-2.13 (m, 4H), 1.98-1.86 (m, 4H). LC-MS m/z: 369.1 [M+H]⁺. HPLC Purity (214 nm): 100%; t_{R}=6.52 min.

### Example 189—4-Chloro-N-cyclopropyl-2-methoxy-1H-imidazole-1-carboxamide

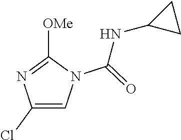 [SMILES: COc1nc(Cl)cn1C(=O)NC1CC1]

Following general procedure C, 4-chloro-2-methoxy-1H-imidazole (10 mg, 0.075 mmol) and cyclopropanamine (5 mg, 0.09 mmol) afforded the title compound (3.9 mg, 24.3%) as a yellow solid. ¹H NMR (400 MHz, CDCl₃) δ 7.14 (s, 1H), 6.92 (s, 1H), 4.15 (s, 3H), 2.79-2.75 (m, 1H), 0.90-0.85 (m, 2H), 0.66-0.62 (m, 2H). LC-MS m/z: 133.0 [M−83]⁺. HPLC Purity (214 nm): 96.07%; t_{R}=7.13 min.

### Example 190—4-Methyl-2-(2-(4-methylpiperazin-1-yl)ethoxy)-N-phenethyl-1H-imidazole-1-carboxamide

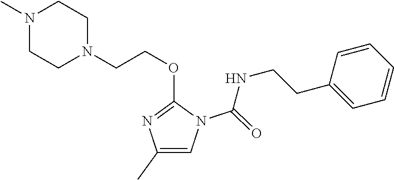 [SMILES: Cc1cn(C(=O)NCCc2ccccc2)c(OCCN2CCN(C)CC2)n1]

Following general procedure E, 2-bromo-4-methyl-1-((2-(trimethylsilyl)ethoxy)methyl)-1H-imidazole (870 mg, 3 mmol) and 2-(4-methylpiperazin-1-yl)ethan-1-ol (4 mL) afforded 1-methyl-4-(2-((4-methyl-1-((2-(trimethylsilyl)ethoxy)methyl)-1H-imidazol-2-yl)oxy)ethyl) piperazine imidazole (820 mg, 77%) as a yellow oil. LC-MS m/z: 355.2 [M+H]⁺. Purity (214 nm): 91%; t_{R}=0.96 min.

Following general procedure F (method 1), 1-methyl-4-(2-((4-methyl-1-((2-(trimethylsilyl)ethoxy)methyl)-1H-imidazol-2-yl)oxy)ethyl)piperazine imidazole (708 mg, 2 mmol) afforded 1-methyl-4-(2-((4-methyl-1H-imidazol-2-yl)oxy)ethyl)piperazine (310 mg, 69%) as a yellow oil. LC-MS m/z: 225.2 [M+H]⁺. Purity (214 nm): 92° %; t_{R}=0.93 min.

Following general procedure C, 1-methyl-4-(2-((4-methyl-1H-imidazol-2-yl)oxy)ethyl)piperazine (88.0 mg, 0.4 mmol) and 2-phenylethan-1-amine (62.0 mg, 0.5 mmol) afforded the title compound (48.9 mg, 33%) as a yellow solid. ¹H NMR (400 MHz, CDCl₃) δ 7.41-7.28 (m, 3H), 7.25-7.22 (m, 3H), 6.93 (s, 1H), 4.44 (t, J=5.6 Hz, 2H), 3.64 (dd, J=12.9, 6.8 Hz, 2H), 2.91 (t, J=6.8 Hz, 2H), 2.56 (t, J=5.6 Hz, 2H), 2.48-2.30 (m, 8H), 2.28 (s, 3H), 2.09 (s, 3H). LC-MS m/z: 372.3 [M+H]⁺. HPLC Purity (214 nm): 95.05%, t_{R}=4.77 min.

### Example 191—4-Chloro-N-(2-(2,2-difluorocyclopentyl)ethyl)-2-(2-morpholinoethoxy)-1H-imidazole-1-carboxamide

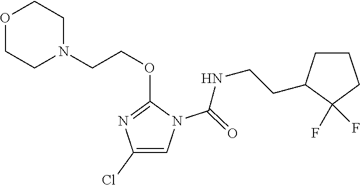 [SMILES: O=C(NCCC1CCCC1(F)F)n1cc(Cl)nc1OCCN1CCOCC1]

Following general procedure E, 2-bromo-4-chloro-1-((2-(trimethylsilyl)ethoxy)methyl)-1H-imidazole (1.0 g, 3.22 mmol) and 2-morpholinoethan-1-ol (5 mL) afforded 4-(2-((4-chloro-1-((2-(trimethylsilyl)ethoxy)methyl)-1H-imidazol-2-yl)oxy)ethyl)morpholine (61 mg, 5.2%) as a yellow oil. LC-MS m/z: 362.0 [M+H]⁺. Purity (214 nm): 94.43%; t_{R}=1.58 min.

Following general procedure F (method 1), 4-(2-((4-chloro-1-((2-(trimethylsilyl)ethoxy)methyl)-1H-imidazol-2-yl)oxy)ethyl)morpholine (60 mg, 0.17 mmol) afforded 4-(2-((4-chloro-1H-imidazol-2-yl)oxy)ethyl)morpholine (30.0 mg, 76.9%) as a white solid. LC-MS m/z: 232.1 [M+H]⁺. Purity (214 nm): 100.00%; t_{R}=1.23 min.

Following general procedure C, 4-(2-((4-chloro-1H-imidazol-2-yl)oxy)ethyl)morpholine (30 mg, 0.13 mmol) and 2-(2,2-difluorocyclopentyl)ethan-1-amine (29 mg, 0.16 mmol) afforded the title compound (6.1 mg, 11.5%) as a white solid. ¹H NMR (500 MHz, CDCl₃) δ 7.26 (s, 1H), 7.12 (s, 1H), 4.58 (t, J=5.5 Hz, 2H), 3.70 (t, J=4.5 Hz, 4H), 3.49-3.44 (m, 2H), 2.80-2.76 (m, 2H), 2.52 (brs, 4H), 2.18-2.00 (m, 4H), 1.88-1.72 (m, 4H), 1.49-1.41 (m, 1H). LC-MS m/z: 407.0 [M+H]⁺. HPLC Purity (214 nm): 100.00%; t_{R}=5.94 min.

### Example 192—4-Methyl-2-(2-morpholinoethoxy)-N-(4-phenylbut-2-ynyl)-1H-imidazole-1-carboxamide

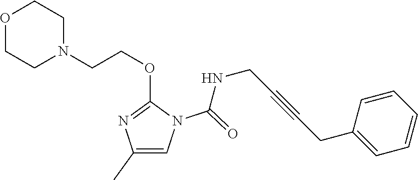 [SMILES: Cc1cn(C(=O)NCC#CCc2ccccc2)c(OCCN2CCOCC2)n1]

Following general procedure C, 4-(2-(4-methyl-1H-imidazol-2-yloxy)ethyl)morpholine (120 mg, 0.57 mmol) and 4-phenylbut-2-yn-1-amine (125 mg, 0.86 mmol) afforded the title compound (21 mg, 9.7%) as a white solid. ¹H NMR (400 MHz, CDCl₃) δ 7.52 (brs, 1H), 7.34 (d, J=4.5 Hz, 4H), 7.29-7.24 (m, 1H), 6.95 (d, J=1.2 Hz, 1H), 4.55 (t, J=5.3 Hz, 2H), 4.27-4.22 (m, 2H), 3.72-3.68 (m, 4H), 3.62 (t, J=1.2 Hz, 2H), 2.75 (t, J=5.3 Hz, 2H), 2.49-2.43 (m, 4H), 2.11 (d, J=1.2 Hz, 3H). LC-MS m/z: 383.3 [M+H]⁺. HPLC Purity (254 nm): 100%; t_{R}=5.41 min.

### Example 193—4-Methyl-N-(4-methylpent-2-yn-1-yl)-2-(2-morpholinoethoxy)-1H-imidazole-1-carboxamide

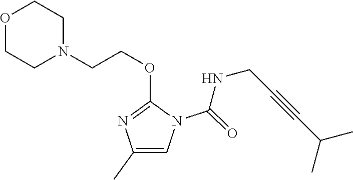 [SMILES: Cc1cn(C(=O)NCC#CC(C)C)c(OCCN2CCOCC2)n1]

Following general procedure C, 4-(2-((4-methyl-1H-imidazol-2-yl)oxy)ethyl)morpholine (105 mg, 0.5 mmol) and 4-methylpent-2-yn-1-amine (50 mg, 0.5 mmol) afforded the title compound (7.4 mg, 4.9%) as a white solid. ¹H NMR (400 MHz, CDCl₃) δ 7.42 (brs, 1H), 6.93 (d, J=1.3 Hz, 1H), 4.60-4.52 (dt, J=5.2, 2.4 Hz, 2H), 4.15 (dd, J=5.3, 2.1 Hz, 2H), 3.79-3.71 (m, 4H), 2.81 (dt, J=5.2, 2.4 Hz, 2H), 2.59-2.49 (m, 5H), 2.09 (d, J=1.2 Hz, 3H), 1.17 (dd, J=7.2, 3.2 Hz, 6H). LC-MS m/z: 335.3 [M+H]⁺. HPLC Purity (214 nm): 100%; t_{R}=4.85 min.

### Example 194—2-Methoxy-N-(4-methylpent-2-yn-1-yl)-4-(2-(4-methylpiperazin-1-yl)pyridin-3-yl)-1H-imidazole-1-carboxamide

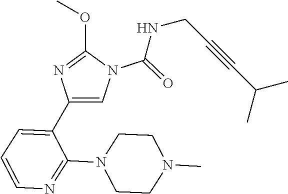 [SMILES: COc1nc(-c2cccnc2N2CCN(C)CC2)cn1C(=O)NCC#CC(C)C]

Following general procedure D, 4-bromo-2-methoxy-1-((2-(trimethylsilyl)ethoxy)methyl)-1H-imidazole (612 mg, 2.0 mmol) and (2-fluoropyridin-3-yl)boronic acid (310 mg, 2.2 mmol) afforded 2-fluoro-3-(2-methoxy-1-((2-(trimethylsilyl)ethoxy)methyl)-1H-imidazol-4-yl)pyridine (560 mg, 86%) as a yellow solid. LC-MS m/z: 324.0 [M+H]⁺. Purity (214 nm): 96%; t_{R}=2.23 min.

To a solution of 2-fluoro-3-(2-methoxy-1-((2-(trimethylsilyl)ethoxy)methyl)-1H-imidazol-4-yl)pyridine (560 mg, 1.7 mmol) in CH₃CN (10 mL) was added 1-methylpiperazine (340 mg, 3.4 mmol) and K₂CO₃ (470 mg, 3.4 mmol). The mixture was stirred at 80° C. for 16 h and then filtered. The filtrate was concentrated to give a residue which was purified by silica gel column chromatography (DCM:MeOH=10:1) to give 1-(3-(2-methoxy-1-((2-(trimethylsilyl)ethoxy)methyl)-1H-imidazol-4-yl)pyridine-2-yl)-4-methylpiperazine (410 mg, 60%) as a yellow oil. LC-MS m/z: 404.2 [M+H]⁺. Purity (214 nm): 87%; t_{R}=1.17 min.

Following general procedure F (method 1), 1-(3-(2-methoxy-1-((2-(trimethylsilyl) ethoxy)methyl)-1H-imidazol-4-yl)pyridin-2-yl)-4-methylpiperazine (403 mg, 1 mmol) afforded 1-(3-(2-methoxy-1H-imidazol-4-yl)pyridine-2-yl)-4-methylpiperazine (140 mg, 51%) as a yellow oil. LC-MS m/z: 274.2 [M+H]⁺. Purity (214 nm): 82%; t_{R}=1.26 min.

Following general procedure C, 1-(3-(2-methoxy-1H-imidazol-4-yl)pyridine-2-yl)-4-methylpiperazine (109 mg, 0.4 mmol) and 4-methylpent-2-yn-1-amine (62.0 mg, 0.5 mmol) afforded the title compound (18.5 mg, 12%) as a white solid. ¹H NMR (400 MHz, CDCl₃) δ 8.19 (ddd, J=9.5, 6.2, 1.9 Hz, 2H), 7.94 (s, 1H), 7.07 (brs, 1H), 6.98 (dd, J=6.0, 3.6 Hz, 1H), 4.26 (s, 3H), 4.19 (dd, J=5.2, 2.1 Hz, 2H), 3.23 (brs, 4H), 2.63-2.58 (m, 5H), 2.36 (s, 3H), 1.17 (d, J=7.2 Hz, 6H). LC-MS m/z: 397.2 [M+H]⁺. HPLC Purity (214 nm): 99.52%; t_{R}=5.24 min

### Example 195-2-Methoxy-4-(2-(4-methylpiperazine-1-yl)pyridin-3-yl)-N-(3-(1-trifluoromethyl)cyclopropyl-1H-imidazole-1-carboxamide

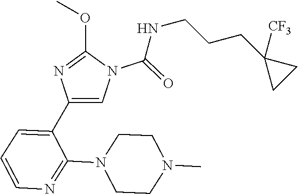 [SMILES: COc1nc(-c2cccnc2N2CCN(C)CC2)cn1C(=O)NCCCC1(C(F)(F)F)CC1]

Following general procedure C, 1-(3-(2-methoxy-1H-imidazol-4-yl)pyridin-2-yl)-4-methylpiperazine (109 mg, 0.4 mmol) and 3-(1-trifluoromethyl)cyclopropyl)propan-1-amine (83.0 mg, 0.5 mmol) afforded the title compound (4.7 mg, 4.6%) as a white solid. ¹H NMR (500 MHz, CDCl₃) δ 8.44 (s, 1H), 8.17 (ddd, J=9.4, 6.2, 1.8 Hz, 2H), 7.84 (s, 1H), 7.06-6.95 (m, 2H), 4.25 (s, 3H), 3.48-3.33 (m, 6H), 2.93 (brs, 4H), 2.56 (s, 3H), 1.90-1.78 (m, 2H), 1.70-1.62 (m, 2H), 1.01 (t, J=5.5 Hz, 2H), 0.58 (brs, 2H). LC-MS m/z: 467.1 [M+H]⁺. HPLC Purity (214 nm): 100%; t_{R}=6.40 min.

### Example 196—4-Methyl-2-(2-morpholinoethoxy)-N-(3-phenylprop-2-yn-1-yl)-1H-imidazole-1-carboxamide

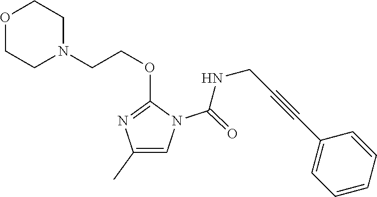 [SMILES: Cc1cn(C(=O)NCC#Cc2ccccc2)c(OCCN2CCOCC2)n1]

Following general procedure C, 4-(2-((4-methyl-1H-imidazol-2-yl)oxy)ethyl)morpholine (70 mg, 0.3 mmol) and 3-phenylprop-2-yn-1-amine (40 mg, 0.3 mmol) afforded the title compound (10.1 mg, 8.3%) as a white solid. ¹H NMR (400 MHz, CDCl₃) δ 7.60 (brs, 1H), 7.45-7.41 (m, 2H), 7.37-7.30 (m, 3H), 6.95 (d, J=1.3 Hz, 1H), 4.58 (t, J=5.3 Hz, 2H), 4.42 (d, 0.1=5.3 Hz, 2H), 3.70-3.63 (m, 4H), 2.80 (t, J=5.3 Hz, 2H), 2.55-2.46 (m, 4H), 2.10 (d, J=1.2 Hz, 3H). LC-MS m/z: 369.2 [M+H]⁺. HPLC Purity (214 nm): 100%; t_{R}=6.52 min.

### Example 197—4-Methyl-2-(2-(4-methylpiperazin-1-yl)ethoxy)-N-(3-phenylprop-2-yn-1-yl)-1H-imidazole-1-carboxamide

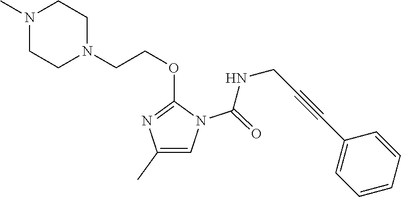 [SMILES: Cc1cn(C(=O)NCC#Cc2ccccc2)c(OCCN2CCN(C)CC2)n1]

Following general procedure C, 1-methyl-4-(2-((4-methyl-1H-imidazol-2-yl)oxy)ethyl)piperazine (88.0 mg, 0.4 mmol) and 3-phenylprop-2-yn-1-amine (65.0 mg, 0.5 mmol) afforded the title compound (61.4 mg, 40%0) as a yellow oil. ¹H NMR (400 MHz, CDCl₃) δ 7.67 (t, J=4.9 Hz, 1H), 7.48-7.41 (m, 2H), 7.34-7.28 (m, 3H), 6.94 (d, J=1.2 Hz, 1H), 4.55 (t, J=5.4 Hz, 2H), 4.41 (d, J=5.4 Hz, 2H), 2.79 (t, J=5.4 Hz, 2H), 2.63-2.50 (m, 4H), 2.50-2.38 (m, 4H), 2.21 (s, 3H), 2.10 (s, 3H). LC-MS m/z: 382.1 [M+H]⁺. HPLC Purity (214 nm): 97.35%; t_{R}=5.64 min.

### Example 198—4-Methyl-2-(2-(4-methylpiperazin-1-yl)ethoxy)-N-(4,4,4-trifluorobutyl)-1H-imidazole-1-carboxamide

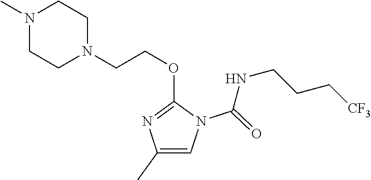 [SMILES: Cc1cn(C(=O)NCCCC(F)(F)F)c(OCCN2CCN(C)CC2)n1]

Following general procedure C, 1-methyl-4-(2-(4-methyl-1H-imidazol-2-yloxy)ethyl)piperazine (150 mg, 0.67 mmol) and 4,4,4-trifluorobutan-1-amine (60 mg, 0.67 mmol) afforded the title compound (9.2 mg, 3.5%) as a white solid. ¹H NMR (400 MHz, CDCl₃) δ 7.43 (brs, 1H), 6.93 (d, J=1.2 Hz, 1H), 4.56 (t, J=5.4 Hz, 2H), 3.47 (dd, J=13.3, 6.8 Hz, 2H), 2.79 (t, J=5.4 Hz, 2H), 2.65 (m, 8H), 2.31 (s, 3H), 2.24-2.16 (m, 2H), 2.12 (d, J=1.0 Hz, 3H), 1.96-1.87 (m, 2H). LC-MS m/z: 378.2 [M+H]⁺. HPLC Purity (254 nm): 100%; t_{R}=6.51 min.

### Example 199—4-Methyl-2-(2-(4-methylpiperazin-1-yl)ethoxy)-N-(2-phenoxyethyl)-1H-imidazole-1-carboxamide

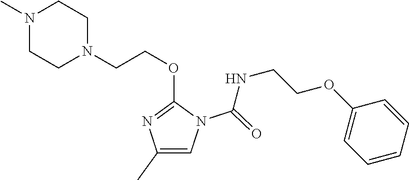 [SMILES: Cc1cn(C(=O)NCCOc2ccccc2)c(OCCN2CCN(C)CC2)n1]

Following general procedure C, 1-methyl-4-(2-((4-methyl-1H-imidazol-2-yl)oxy) ethyl)piperazine (88.0 mg, 0.4 mmol) and 2-phenoxyethan-1-amine (65.0 mg, 0.5 mmol) afforded the title compound (37.6 mg, 36%) as a yellow oil. ¹H NMR (500 MHz, CDCl₃) δ 7.64 (t, J=5.5 Hz, 1H), 7.33-7.29 (m, 2H), 6.97 (t, J=7.4 Hz, 1H), 6.91 (dd, J=11.4, 4.5 Hz, 3H), 4.56 (t, J=6.0 Hz, 2H), 4.16 (t, J=5.0 Hz, 2H), 3.78 (dd, J=10.6, 5.3 Hz, 2H), 2.77 (t, J=6.0 Hz, 2H), 2.52-2.27 (m, 8H), 2.27 (s, 3H), 2.11 (s, 3H). LC-MS m/z: 388.3 [M+H]⁺. HPLC Purity (214 nm): 95.34%; t_{R}=7.96 min.

### Example 200—4-Methyl-2-(2-(4-methylpiperazin-1-yl)ethoxy)-N-(3-(1-(trifluoromethyl) cyclopropyl)propyl)-1H-imidazole-1-carboxamide

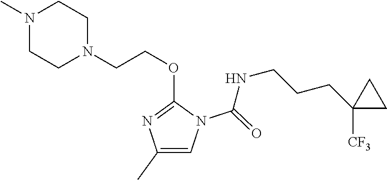 [SMILES: Cc1cn(C(=O)NCCCC2(C(F)(F)F)CC2)c(OCCN2CCN(C)CC2)n1]

Following general procedure C, 1-methyl-4-(2-((4-methyl-1H-imidazol-2-yl)oxy)ethyl)piperazine (88.0 mg, 0.4 mmol) and 3-(1-(trifluoromethyl)cyclopropyl)propan-1-amine (84.0 mg, 0.5 mmol) afforded the title compound (37.1 mg, 33%) as a yellow oil. ¹H NMR (500 MHz, CDCl₃) δ 7.33 (t, J=5.0 Hz, 1H), 6.93 (d, J=1.0 Hz, 1H), 4.54 (t, J=5.4 Hz, 2H), 3.36 (dd, J=13.1, 7.0 Hz, 2H), 2.77 (t, J=5.4 Hz, 2H), 2.69-2.32 (m, 8H), 2.29 (s, 3H), 2.09 (s, 3H), 1.80-1.74 (m, 2H), 1.65-1.61 (m, 2H), 0.99 (t, J=5.8 Hz, 2H), 0.59 (t, J=5.8 Hz, 2H). LC-MS m/z: 418.2 [M+H]⁺. HPLC Purity (214 nm): 98.70%; t_{R}=6.56 min.

### Example 201—4-Methyl-2-(2-(4-methylpiperazin-1-yl)ethoxy)-N-(4-phenylbut-2-ynyl)-1H-imidazole-1-carboxamide

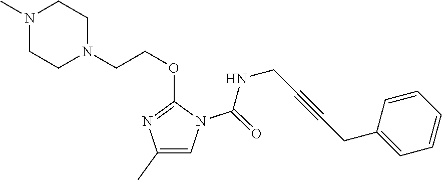 [SMILES: Cc1cn(C(=O)NCC#CCc2ccccc2)c(OCCN2CCN(C)CC2)n1]

Following general procedure C, 1-methyl-4-(2-(4-methyl-1H-imidazol-2-yloxy)ethyl)piperazine (150 mg, 0.67 mmol) and 4-phenylbut-2-yn-1-amine (97 mg, 0.67 mmol) afforded the title compound (5.0 mg, 1.9%) as a colorless oil. ¹H NMR (400 MHz, CDCl₃) δ 7.56 (brs, 1H), 7.34 (d, J=4.5 Hz, 4H), 7.28-7.24 (m, 1H), 6.95 (d, J=1.3 Hz, 1H), 4.55 (t, J=5.4 Hz, 2H), 4.23 (dd, J=5.2, 2.5 Hz, 2H), 3.63 (t, J=2.1 Hz, 2H), 2.77 (t, J=5.4 Hz, 2H), 2.71-2.35 (m, 8H), 2.28 (s, 3H), 2.11 (d, J=1.2 Hz, 3H). LC-MS m/z: 396.2 [M+H]⁺. HPLC Purity (254 nm): 100%; t_{R}=7.34 min.

### Example 202—2-(2-(4-Methylpiperazin-1-yl)ethoxy)-4-phenyl-N-(4-phenylbut-2-yn-1-yl)-1H-imidazole-1-carboxamide

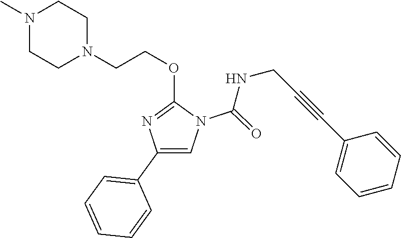 [SMILES: CN1CCN(CCOc2nc(-c3ccccc3)cn2C(=O)NCC#Cc2ccccc2)CC1]

Following general procedure E, 2-bromo-4-phenyl-1-((2-(trimethylsilyl)ethoxy)methyl)-1H-imidazole (1.9 g, 5.4 mmol) in 2-(4-methylpiperazin-1-yl)ethanol (4 mL) afforded 1-methyl-4-(2-(4-phenyl-1-((2-(trimethylsilyl)ethoxy)methyl)-1H-imidazol-2-yloxy)ethyl)piperazine (1.7 g, 77.3%) as a yellow oil. LC-MS m/z: 417.1 [M+H]⁺. Purity (214 nm): 96.6%; t_{R}=1.17 min.

Following general procedure F (method 1), 1-methyl-4-(2-(4-phenyl-1-((2-(trimethylsilyl)ethoxy)methyl)-1H-imidazol-2-yloxy)ethyl)piperazine (1.7 g, 4.1 mmol) afforded 1-methyl-4-(2-(4-phenyl-1H-imidazol-2-yloxy)ethyl)piperazine (700 mg, 77.3%) as a yellow oil. LC-MS m/z: 287.3 [M+H]⁺. Purity (254 nm): 92.7%; t_{R}=1.52 min.

Following general procedure C, 1-methyl-4-(2-(4-phenyl-1H-imidazol-2-yloxy)ethyl)piperazine (100 mg, 0.35 mmol) and 4-phenylbut-2-yn-1-amine (45 mg, 0.35 mmol) afforded the title compound (8.3 mg, 5.4%) as a white solid. ¹H NMR (400 MHz, CDCl₃) δ 7.78 (brs, 1H), 7.75 (d, J=7.3 Hz, 2H), 7.57 (s, 1H), 7.46 (dd, J=7.3, 2.4 Hz, 2H), 7.44-7.30 (m, 6H), 4.71 (t, J=7.4 Hz, 2H), 4.47 (d, J=5.4 Hz, 2H), 2.85-2.74 (m, 4H), 2.51-2.41 (m 4H), 2.24 (s, 3H). LC-MS m/z: 444.2 [M+H]⁺. HPLC Purity (254 nm): 100%; t_{R}=9.38 min.

### Example 203—N-(Biphenyl-4-ylmethyl)-4-methyl-2-(2-(4-methylpiperazin-1-yl)ethoxy)-1H-imidazole-1-carboxamide

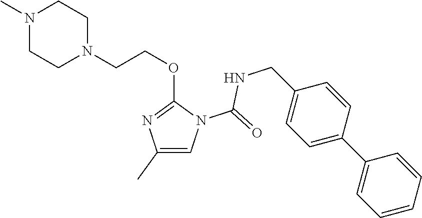 [SMILES: Cc1cn(C(=O)NCc2ccc(-c3ccccc3)cc2)c(OCCN2CCN(C)CC2)n1]

Following general procedure C, 1-methyl-4-(2-(4-methyl-1H-imidazol-2-yloxy)ethyl)piperazine (140 mg, 0.63 mmol) and biphenyl-4-ylmethanamine (115 mg, 0.63 mmol) afforded the title compound (47.1 mg, 15.7%) as a white solid. ¹H NMR (400 MHz, CDCl₃) δ 7.75-7.61 (brs, 1H), 7.62-7.58 (m, 4H), 7.49-7.35 (m, 5H), 6.99 (d, J=1.3 Hz, 1H), 4.65 (dd, J=5.7, 1.3 Hz, 2H), 4.55 (t, J=7.3 Hz, 2H), 2.72 (t, J=7.3 Hz, 2H), 2.51 t, J=7.3 Hz, 2.41 (m, 4H), 2.34-2.16 (m, 4H), 2.27 (s, 3H), 2.13 (d, J=1.2 Hz, 3H). LC-MS m/z: 434.2 [M+H]⁺. HPLC Purity (254 nm): 95.02%; t_{R}=8.57 min.

### Example 204—4-(1-Methylpiperidin-4-yl)-N-(4-phenylbut-3-yn-1-yl)-1H-imidazole-1-carboxamide

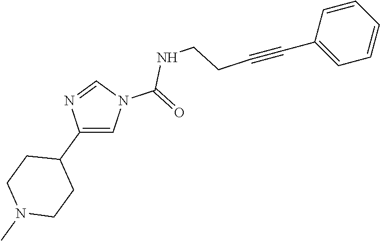 [SMILES: CN1CCC(c2cn(C(=O)NCCC#Cc3ccccc3)cn2)CC1]

Following general procedure C, 4-(1H-imidazol-4-yl)-1-methylpiperidine (165 mg, 1.0 mmol) and 4-phenylbut-3-yn-1-amine (145 mg, 1.0 mmol) afforded the title compound (68.6 mg, 20%) as a white solid. ¹H NMR (500 MHz, CDCl₃) δ 8.05 (d, J=1.2 Hz, 1H), 7.39 (dd, J=7.0, 2.7 Hz, 2H), 7.34-7.29 (m, 3H), 7.02 (s, 1H), 6.01 (s, 1H), 3.65 (q, J=6.2 Hz, 2H), 2.91 (d, J=11.9 Hz, 2H), 2.77 (t, J=6.4 Hz, 2H), 2.53 (t, J=11.7 Hz, 1H), 2.30 (s, 3H), 2.13-1.95 (m, 4H), 1.74-1.65 (m, 2H). LC-MS m/z: 337.2 [M+H]⁺. HPLC Purity (214 nm): 95.61%; t_{R}=6.43 min.

Compound 67a in Table 1 was prepared according to the methods described in the above Examples.

### Example 205—Biological Activity Evaluation

The ability of exemplary compounds to inhibit acid ceramidase was measured. Experimental procedures and results are provided below.

### Part I: Assay Procedure

Cell lysates overexpressing acid ceramidase were used as the enzyme source for compound potency determination in a biochemical fluorescent assay. Briefly, compounds were preincubated with 10 μg protein of cell lysates in a dose-response manner for 1 hr at RT in the assay buffer containing 25 mM NaAC and 100 mM NaCl, pH 4.5. The reaction was initiated by the addition of substrate Rbm14-12 at a final concentration of 6.3 μM. The reaction was run at RT for 1 hr before it was stopped by the addition of the stopping buffer containing 20% methanol (v/v), 1 mg/ml NaIO₄, 0.1 M glycine, pH 10.6. The samples were incubated with the stopping buffer at RT for 1 hr to allow the fluorescent product to be formed. Finally the plate was read with SpectraMax i3 plate reader (Molecular Devices) at ex360 nm and em446 nm. Data were collected and used to determine the IC₅₀ values of compounds by curve fitting to the four-parameter inhibition equation.

### Part II: Results

Acid ceramidase inhibition values for tested compounds are provided in Table 1 below, along with c Log P and compound solubility in water. The symbol “A” indicates inhibition of less than 0.2 WM; the symbol “B” indicates inhibition in the range of 0.2 μM up to 1 μM; and the symbol “C” indicates inhibition of greater than 1 μM.

TABLE 1

| | | | Compound | |
|---|---|---|---|---|
| | | | Solubility | |
| | | | in Water | IC₅₀ |
| Example | Compound Structure | cLogP | (μg/mL) | hACR |
| 2 | 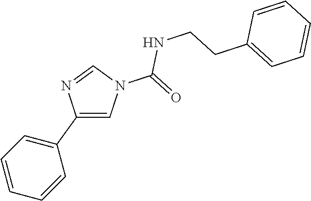 [SMILES: O=C(NCCc1ccccc1)n1cnc(-c2ccccc2)c1] | 2.7 | 0.2 | A |
| 3 | 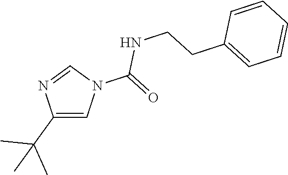 [SMILES: CC(C)(C)c1cn(C(=O)NCCc2ccccc2)cn1] | 2.2 | 5.2 | A |
| 4 | 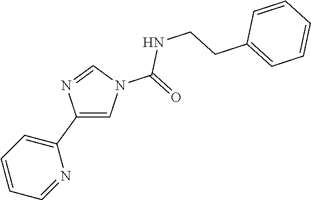 [SMILES: O=C(NCCc1ccccc1)n1cnc(-c2ccccn2)c1] | 1.4 | 21.1 | B |
| 5 | 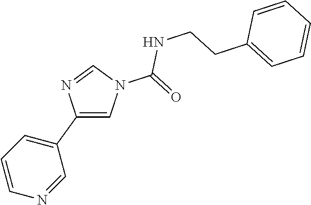 [SMILES: O=C(NCCc1ccccc1)n1cnc(-c2cccnc2)c1] | 1.2 | 16.2 | B |
| 6 | 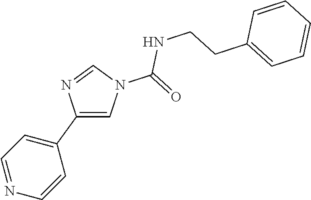 [SMILES: O=C(NCCc1ccccc1)n1cnc(-c2ccncc2)c1] | 1.2 | 23.9 | C |
| 7 | 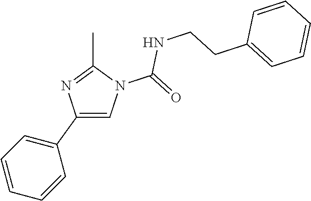 [SMILES: Cc1nc(-c2ccccc2)cn1C(=O)NCCc1ccccc1] | 2.9 | 1.1 | A |
| 8 | 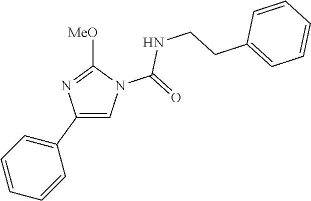 [SMILES: COc1nc(-c2ccccc2)cn1C(=O)NCCc1ccccc1] | 3.4 | 0.1 | B |
| 9 | 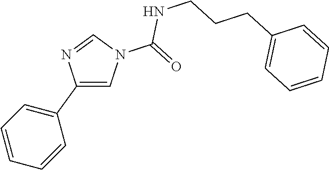 [SMILES: O=C(NCCCc1ccccc1)n1cnc(-c2ccccc2)c1] | 3.0 | 1.3 | A |
| 10 | 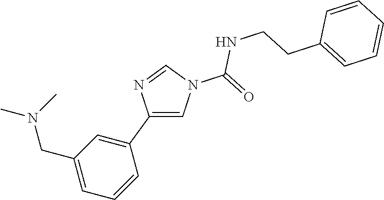 [SMILES: CN(C)Cc1cccc(-c2cn(C(=O)NCCc3ccccc3)cn2)c1] | 1.3 | 27.4 | A |
| 11 | 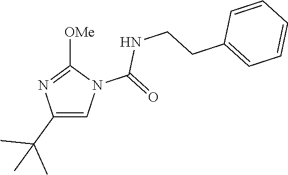 [SMILES: COc1nc(C(C)(C)C)cn1C(=O)NCCc1ccccc1] | 2.9 | 15.1 | B |
| 12 | 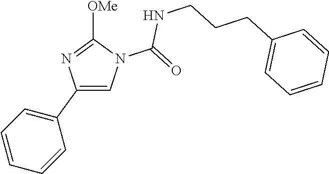 [SMILES: COc1nc(-c2ccccc2)cn1C(=O)NCCCc1ccccc1] | 3.8 | 1.0 | B |
| 13 | 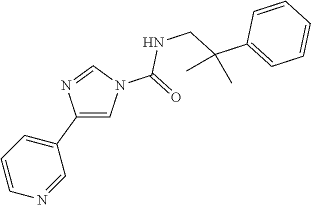 [SMILES: CC(C)(CNC(=O)n1cnc(-c2cccnc2)c1)c1ccccc1] | 2.0 | 1.9 | A |
| 14 | 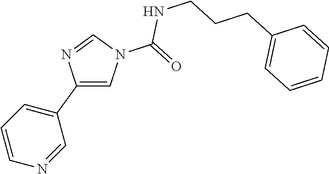 [SMILES: O=C(NCCCc1ccccc1)n1cnc(-c2cccnc2)c1] | 1.6 | 14.3 | A |
| 15 | 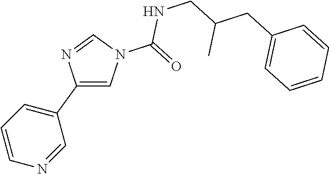 [SMILES: CC(CNC(=O)n1cnc(-c2cccnc2)c1)Cc1ccccc1] | 2.0 | 16.1 | A |
| 16 | 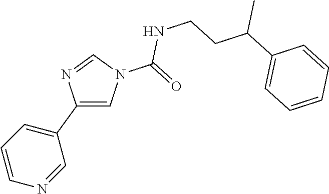 [SMILES: CC(CCNC(=O)n1cnc(-c2cccnc2)c1)c1ccccc1] | 2.0 | 3.8 | A |
| 17 | 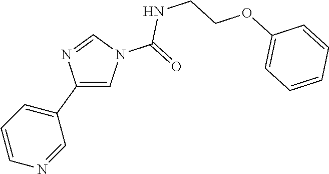 [SMILES: O=C(NCCOc1ccccc1)n1cnc(-c2cccnc2)c1] | 1.3 | 2.6 | A |
| 18 | 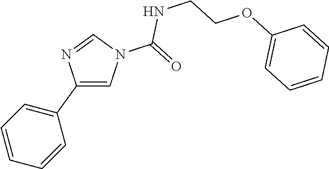 [SMILES: O=C(NCCOc1ccccc1)n1cnc(-c2ccccc2)c1] | 2.7 | 0.06 | A |
| 19 | 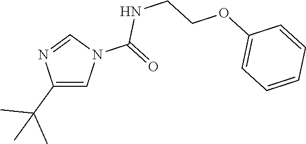 [SMILES: CC(C)(C)c1cn(C(=O)NCCOc2ccccc2)cn1] | 2.2 | 0.8 | A |
| 20 | 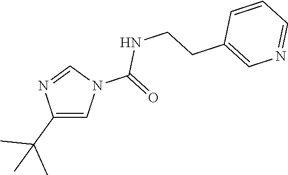 [SMILES: CC(C)(C)c1cn(C(=O)NCCc2cccnc2)cn1] | 0.7 | 0.2 | B |
| 21 | 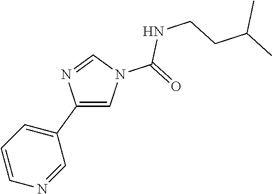 [SMILES: CC(C)CCNC(=O)n1cnc(-c2cccnc2)c1] | 1.1 | 21.5 | B |
| 22 | 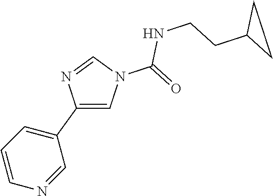 [SMILES: O=C(NCCC1CC1)n1cnc(-c2cccnc2)c1] | 0.6 | 23.0 | B |
| 23 | 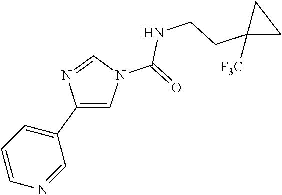 [SMILES: O=C(NCCC1(C(F)(F)F)CC1)n1cnc(-c2cccnc2)c1] | 0.6 | 23.7 | B |
| 24 | 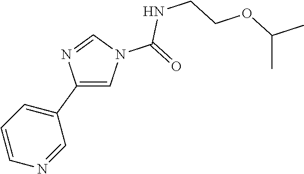 [SMILES: CC(C)OCCNC(=O)n1cnc(-c2cccnc2)c1] | 0.2 | 12.6 | C |
| 25 | 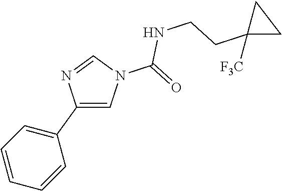 [SMILES: O=C(NCCC1(C(F)(F)F)CC1)n1cnc(-c2ccccc2)c1] | 2.0 | 0.8 | A |
| 26 | 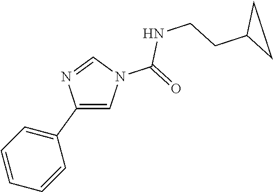 [SMILES: O=C(NCCC1CC1)n1cnc(-c2ccccc2)c1] | 2.1 | 1.9 | B |
| 27 | 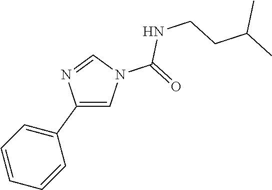 [SMILES: CC(C)CCNC(=O)n1cnc(-c2ccccc2)c1] | 2.5 | 3.1 | A |
| 28 | 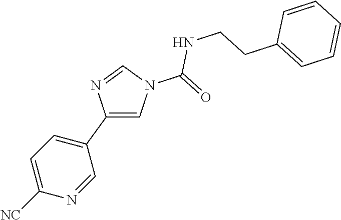 [SMILES: N#Cc1ccc(-c2cn(C(=O)NCCc3ccccc3)cn2)cn1] | 1.0 | 5 | A |
| 29 | 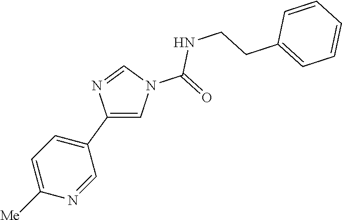 [SMILES: Cc1ccc(-c2cn(C(=O)NCCc3ccccc3)cn2)cn1] | 1.7 | 3.0 | A |
| 30 | 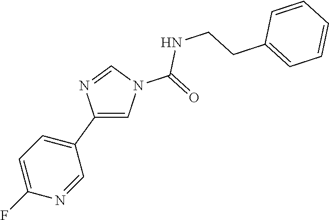 [SMILES: O=C(NCCc1ccccc1)n1cnc(-c2ccc(F)nc2)c1] | 1.4 | 3.4 | A |
| 31 | 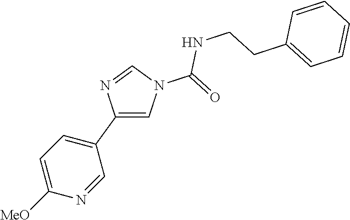 [SMILES: COc1ccc(-c2cn(C(=O)NCCc3ccccc3)cn2)cn1] | 2.0 | 25.1 | A |
| 32 | 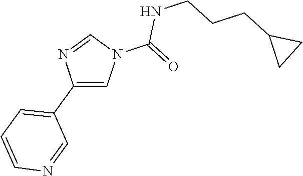 [SMILES: O=C(NCCCC1CC1)n1cnc(-c2cccnc2)c1] | 1.2 | 27.6 | B |
| 33 | 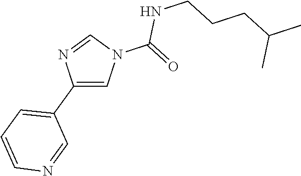 [SMILES: CC(C)CCCNC(=O)n1cnc(-c2cccnc2)c1] | 1.6 | 18.9 | B |
| 34 | 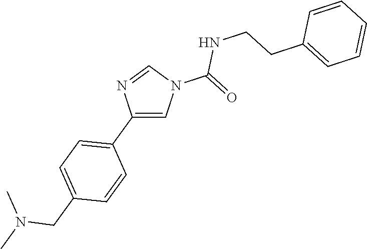 [SMILES: CN(C)Cc1ccc(-c2cn(C(=O)NCCc3ccccc3)cn2)cc1] | 2.5 | 25.6 | A |
| 35 | 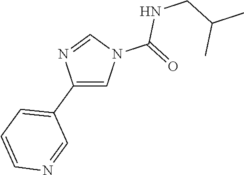 [SMILES: CC(C)CNC(=O)n1cnc(-c2cccnc2)c1] | 0.6 | 19.1 | C |
| 36 | 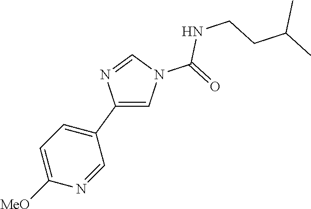 [SMILES: COc1ccc(-c2cn(C(=O)NCCC(C)C)cn2)cn1] | 1.9 | 2.9 | B |
| 37 | 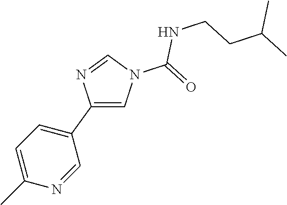 [SMILES: Cc1ccc(-c2cn(C(=O)NCCC(C)C)cn2)cn1] | 1.6 | 7.3 | C |
| 38 | 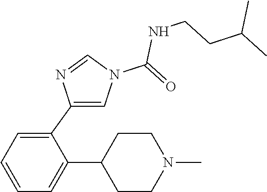 [SMILES: CC(C)CCNC(=O)n1cnc(-c2ccccc2C2CCN(C)CC2)c1] | 2.7 | 35.3 | B |
| 39 | 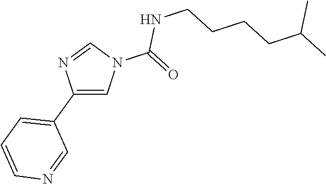 [SMILES: CC(C)CCCCNC(=O)n1cnc(-c2cccnc2)c1] | 2.2 | 2.7 | A |
| 40 | 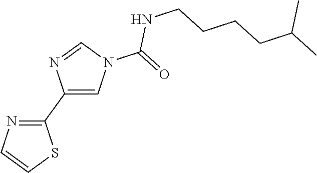 [SMILES: CC(C)CCCCNC(=O)n1cnc(-c2nccs2)c1] | 1.7 | 19 | A |
| 41 | 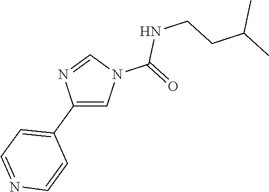 [SMILES: CC(C)CCNC(=O)n1cnc(-c2ccncc2)c1] | 1.1 | 22.1 | C |
| 42 | 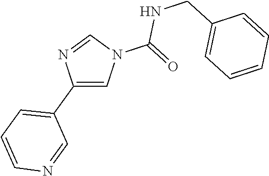 [SMILES: O=C(NCc1ccccc1)n1cnc(-c2cccnc2)c1] | 0.9 | 13.6 | B |
| 43 | 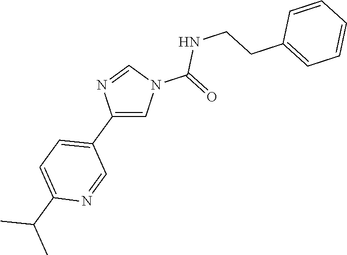 [SMILES: CC(C)c1ccc(-c2cn(C(=O)NCCc3ccccc3)cn2)cn1] | 2.7 | 0.8 | A |
| 44 | 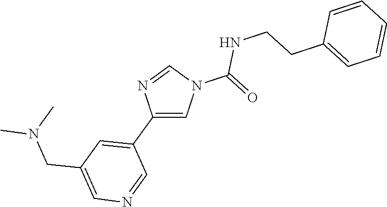 [SMILES: CN(C)Cc1cncc(-c2cn(C(=O)NCCc3ccccc3)cn2)c1] | 1.1 | 30.8 | B |
| 45 | 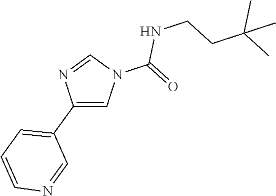 [SMILES: CC(C)(C)CCNC(=O)n1cnc(-c2cccnc2)c1] | 1.5 | 12.1 | B |
| 46 | 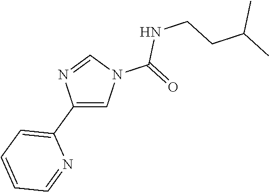 [SMILES: CC(C)CCNC(=O)n1cnc(-c2ccccn2)c1] | 1.3 | 13.8 | B |
| 47 | 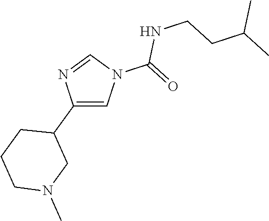 [SMILES: CC(C)CCNC(=O)n1cnc(C2CCCN(C)C2)c1] | 0.8 | 32.2 | B |
| 48 | 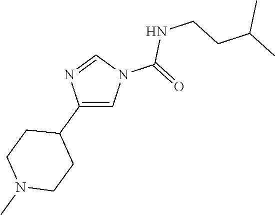 [SMILES: CC(C)CCNC(=O)n1cnc(C2CCN(C)CC2)c1] | 0.7 | 34.2 | B |
| 49 | 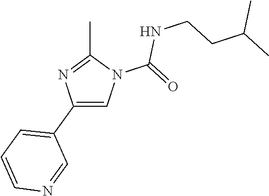 [SMILES: Cc1nc(-c2cccnc2)cn1C(=O)NCCC(C)C] | 1.4 | 7.9 | A |
| 50 | 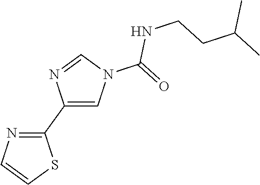 [SMILES: CC(C)CCNC(=O)n1cnc(-c2nccs2)c1] | 1.1 | 23.5 | B |
| 51 | 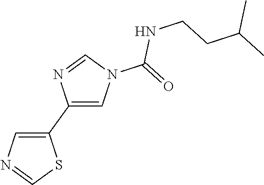 [SMILES: CC(C)CCNC(=O)n1cnc(-c2cncs2)c1] | 1.1 | 25.1 | B |
| 52 | 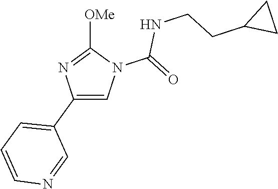 [SMILES: COc1nc(-c2cccnc2)cn1C(=O)NCCC1CC1] | 1.9 | 8.5 | C |
| 53 | 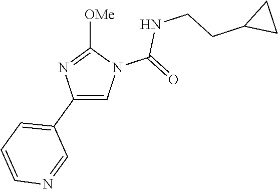 [SMILES: COc1nc(-c2cccnc2)cn1C(=O)NCCC1CC1] | 1.4 | 26.5 | c |
| 54 | 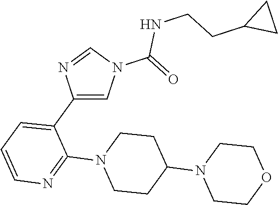 [SMILES: O=C(NCCC1CC1)n1cnc(-c2cccnc2N2CCC(N3CCOCC3)CC2)c1] | 0.8 | 32.8 | A |
| 55 | 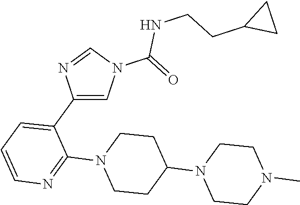 [SMILES: CN1CCN(C2CCN(c3ncccc3-c3cn(C(=O)NCCC4CC4)cn3)CC2)CC1] | 0.1 | 41.5 | B |
| 56 | 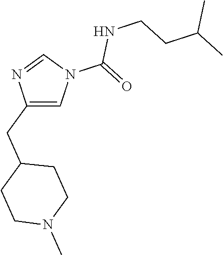 [SMILES: CC(C)CCNC(=O)n1cnc(CC2CCN(C)CC2)c1] | 1.2 | 21.7 | B |
| 57 | 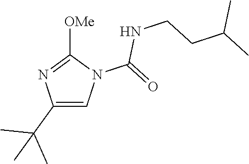 [SMILES: COc1nc(C(C)(C)C)cn1C(=O)NCCC(C)C] | 2.8 | 13.3 | B |
| 58 | 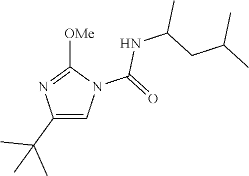 [SMILES: COc1nc(C(C)(C)C)cn1C(=O)NC(C)CC(C)C] | 3.1 | 15.1 | B |
| 59 | 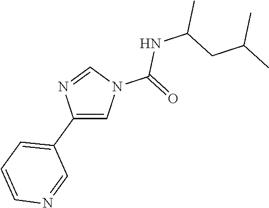 [SMILES: CC(C)CC(C)NC(=O)n1cnc(-c2cccnc2)c1] | 1.4 | 22.3 | B |
| 60 | 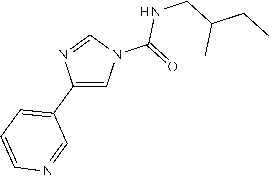 [SMILES: CCC(C)CNC(=O)n1cnc(-c2cccnc2)c1] | 1.1 | 25.6 | B |
| 61 | 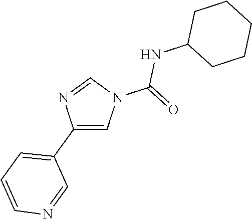 [SMILES: O=C(NC1CCCCC1)n1cnc(-c2cccnc2)c1] | 1.2 | 23.0 | C |
| 62 | 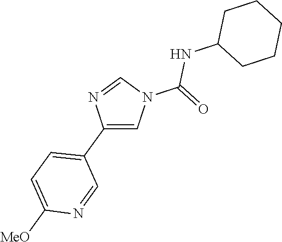 [SMILES: COc1ccc(-c2cn(C(=O)NC3CCCCC3)cn2)cn1] | 2.0 | 0.6 | C |
| 63 | 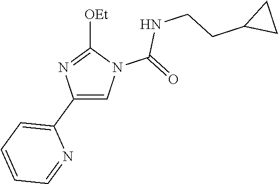 [SMILES: CCOc1nc(-c2ccccn2)cn1C(=O)NCCC1CC1] | 2.1 | 24.1 | C |
| 64 | 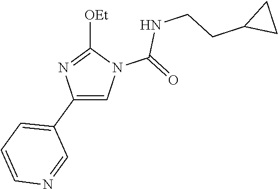 [SMILES: CCOc1nc(-c2cccnc2)cn1C(=O)NCCC1CC1] | 1.9 | 16.8 | C |
| 65 | 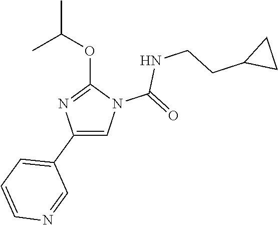 [SMILES: CC(C)Oc1nc(-c2cccnc2)cn1C(=O)NCCC1CC1] | 2.2 | 25 | B |
| 66 | 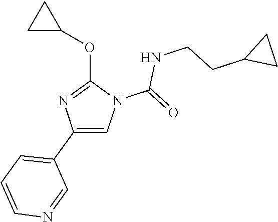 [SMILES: O=C(NCCC1CC1)n1cc(-c2cccnc2)nc1OC1CC1] | 2.0 | 25 | C |
| 67 | 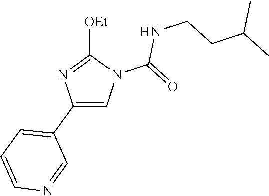 [SMILES: CCOc1nc(-c2cccnc2)cn1C(=O)NCCC(C)C] | 2.4 | 8 | C |
| 67a | 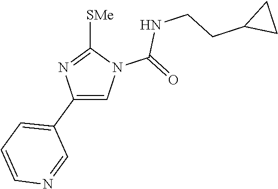 [SMILES: CSc1nc(-c2cccnc2)cn1C(=O)NCCC1CC1] | 1.8 | 23 | B |
| 68 | 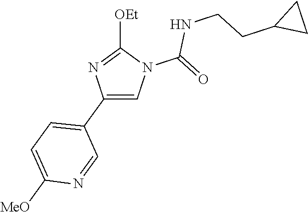 [SMILES: CCOc1nc(-c2ccc(OC)nc2)cn1C(=O)NCCC1CC1] | 2.8 | 2 | C |
| 69 | 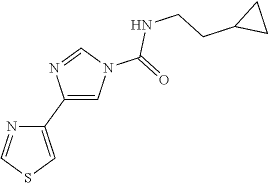 [SMILES: O=C(NCCC1CC1)n1cnc(-c2cscn2)c1] | 0.7 | 32 | B |
| 70 | 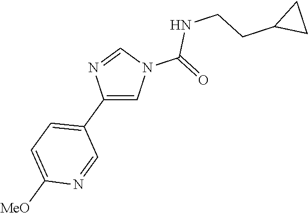 [SMILES: COc1ccc(-c2cn(C(=O)NCCC3CC3)cn2)cn1] | 1.5 | 1.3 | B |
| 71 | 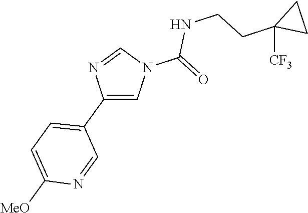 [SMILES: COc1ccc(-c2cn(C(=O)NCCC3(C(F)(F)F)CC3)cn2)cn1] | 1.4 | 22 | A |
| 72 | 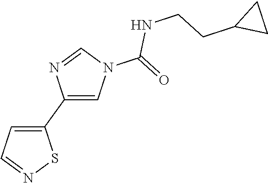 [SMILES: O=C(NCCC1CC1)n1cnc(-c2ccns2)c1] | 1.0 | 22 | B |
| 73 | 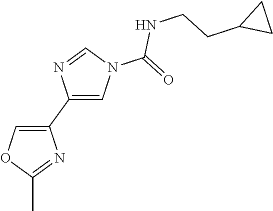 [SMILES: Cc1nc(-c2cn(C(=O)NCCC3CC3)cn2)co1] | 0.3 | 26 | B |
| 74 | 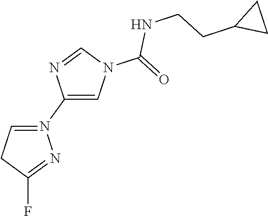 | 0.4 | 34 | C |
| 75 | 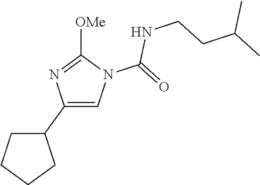 [SMILES: COc1nc(C2CCCC2)cn1C(=O)NCCC(C)C] | 3.0 | 13 | B |
| 76 | 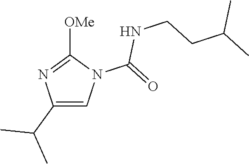 [SMILES: COc1nc(C(C)C)cn1C(=O)NCCC(C)C] | 2.4 | 20 | B |
| 77 | 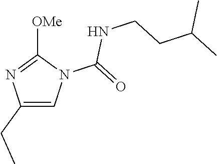 [SMILES: CCc1cn(C(=O)NCCC(C)C)c(OC)n1] | 2.0 | 23 | B |
| 78 | 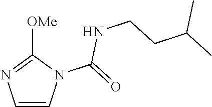 | 1.2 | 28 | B |
| 79 | 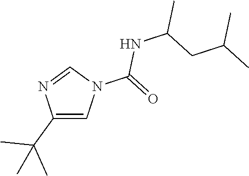 [SMILES: CC(C)CC(C)NC(=O)n1cnc(C(C)(C)C)c1] | 2.3 | 11 | A |
| 80 | 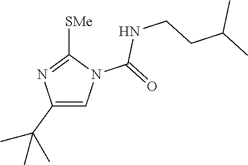 [SMILES: CSc1nc(C(C)(C)C)cn1C(=O)NCCC(C)C] | 3.2 | 0.2 | A |
| 81 | 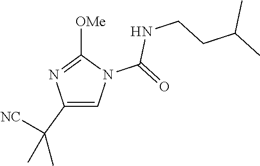 [SMILES: COc1nc(C(C)(C)C#N)cn1C(=O)NCCC(C)C] | 1.3 | 23 | C |
| 82 | 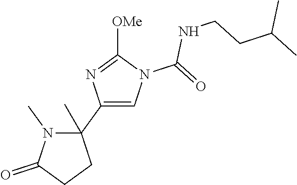 [SMILES: COc1nc(C2(C)CCC(=O)N2C)cn1C(=O)NCCC(C)C] | 0.8 | 31 | C |
| 83 | 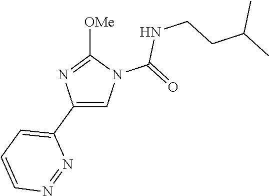 [SMILES: COc1nc(-c2cccnn2)cn1C(=O)NCCC(C)C] | 0.9 | 33 | B |
| 84 | 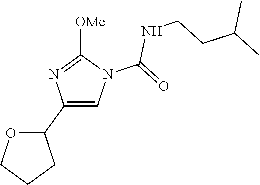 [SMILES: COc1nc(C2CCCO2)cn1C(=O)NCCC(C)C] | 1.2 | 25 | C |
| 85 | 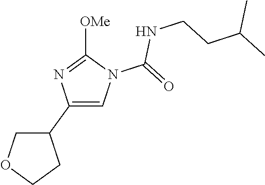 [SMILES: COc1nc(C2CCOC2)cn1C(=O)NCCC(C)C] | 0.9 | 28 | B |
| 86 | 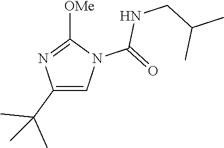 [SMILES: COc1nc(C(C)(C)C)cn1C(=O)NCC(C)C] | 2.3 | 15 | B |
| 87 | 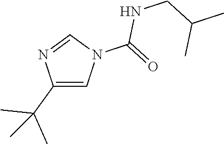 [SMILES: CC(C)CNC(=O)n1cnc(C(C)(C)C)c1] | 1.5 | 17 | B |
| 88 | 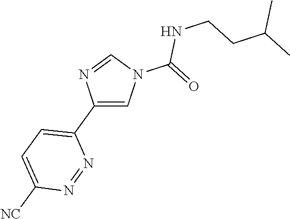 [SMILES: CC(C)CCNC(=O)n1cnc(-c2ccc(C#N)nn2)c1] | 2.0 | 6 | B |
| 89 | 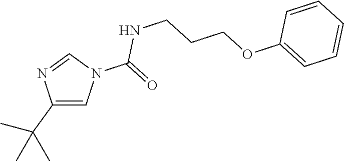 [SMILES: CC(C)(C)c1cn(C(=O)NCCCOc2ccccc2)cn1] | 3.3 | 4 | A |
| 90 | 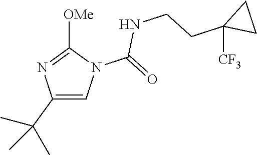 [SMILES: COc1nc(C(C)(C)C)cn1C(=O)NCCC1(C(F)(F)F)CC1] | 2.3 | 17 | B |
| 91 | 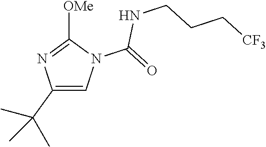 [SMILES: COc1nc(C(C)(C)C)cn1C(=O)NCCCC(F)(F)F] | 1.9 | 22 | B |
| 92 | 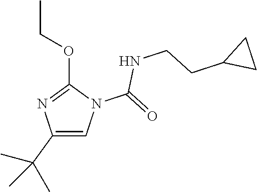 [SMILES: CCOc1nc(C(C)(C)C)cn1C(=O)NCCC1CC1] | 2.9 | 12 | B |
| 93 | 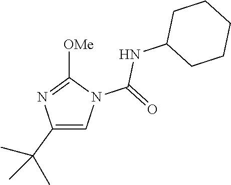 [SMILES: COc1nc(C(C)(C)C)cn1C(=O)NC1CCCCC1] | 2.9 | 8 | B |
| 94 | 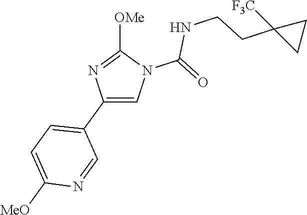 [SMILES: COc1ccc(-c2cn(C(=O)NCCC3(C(F)(F)F)CC3)c(OC)n2)cn1] | 2.2 | 6 | B |
| 95 | 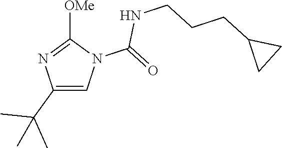 [SMILES: COc1nc(C(C)(C)C)cn1C(=O)NCCCC1CC1] | 2.9 | 12 | A |
| 96 | 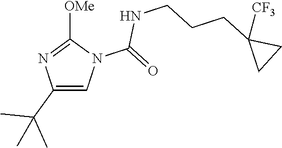 [SMILES: COc1nc(C(C)(C)C)cn1C(=O)NCCCC1(C(F)(F)F)CC1] | 2.6 | 12 | A |
| 97 | 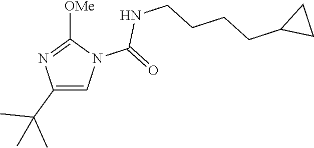 [SMILES: COc1nc(C(C)(C)C)cn1C(=O)NCCCCC1CC1] | 3.4 | 3 | A |
| 98 | 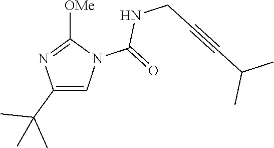 [SMILES: COc1nc(C(C)(C)C)cn1C(=O)NCC#CC(C)C] | 2.4 | 5 | A |
| 99 | 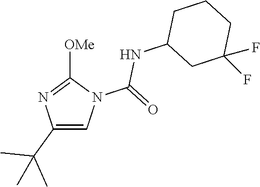 [SMILES: COc1nc(C(C)(C)C)cn1C(=O)NC1CCCC(F)(F)C1] | 2.2 | 14 | C |
| 100 | 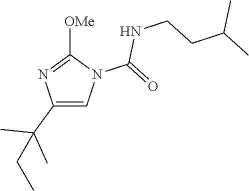 [SMILES: CCC(C)(C)c1cn(C(=O)NCCC(C)C)c(OC)n1] | 3.3 | 14 | B |
| 101 | 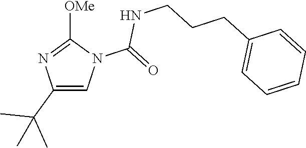 [SMILES: COc1nc(C(C)(C)C)cn1C(=O)NCCCc1ccccc1] | 3.3 | 10 | A |
| 102 | 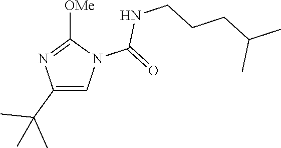 [SMILES: COc1nc(C(C)(C)C)cn1C(=O)NCCCC(C)C] | 3.3 | 10 | A |
| 103 | 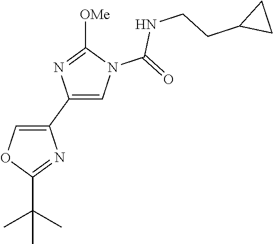 [SMILES: COc1nc(-c2coc(C(C)(C)C)n2)cn1C(=O)NCCC1CC1] | 2.4 | 6 | C |
| 104 | 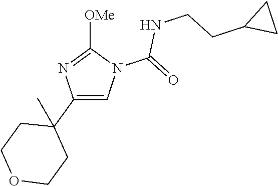 [SMILES: COc1nc(C2(C)CCOCC2)cn1C(=O)NCCC1CC1] | 1.2 | 28 | B |
| 105 | 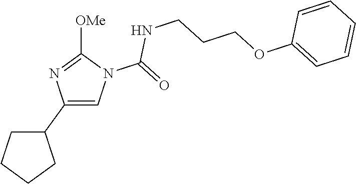 [SMILES: COc1nc(C2CCCC2)cn1C(=O)NCCCOc1ccccc1] | 3.6 | 14 | A |
| 106 | 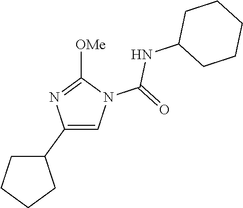 [SMILES: COc1nc(C2CCCC2)cn1C(=O)NC1CCCCC1] | 3.1 | 6 | C |
| 107 | 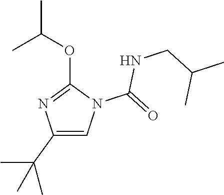 [SMILES: CC(C)CNC(=O)n1cc(C(C)(C)C)nc1OC(C)C] | 3.1 | 7 | B |
| 108 | 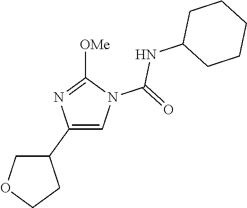 [SMILES: COc1nc(C2CCOC2)cn1C(=O)NC1CCCCC1] | 1.0 | 32 | C |
| 109 | 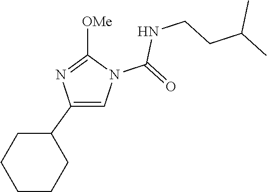 [SMILES: COc1nc(C2CCCCC2)cn1C(=O)NCCC(C)C] | 3.6 | 13 | B |
| 110 | 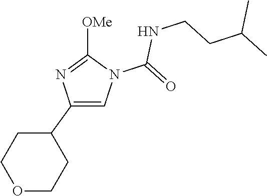 [SMILES: COc1nc(C2CCOCC2)cn1C(=O)NCCC(C)C] | 1.2 | 29 | B |
| 111 | 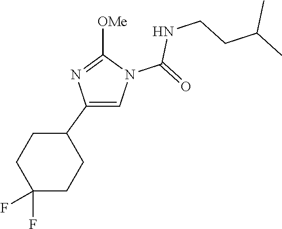 [SMILES: COc1nc(C2CCC(F)(F)CC2)cn1C(=O)NCCC(C)C] | 3.0 | 15 | B |
| 112 | 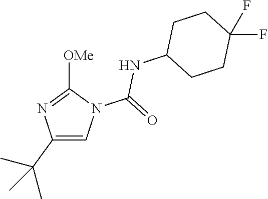 [SMILES: COc1nc(C(C)(C)C)cn1C(=O)NC1CCC(F)(F)CC1] | 2.2 | 12 | B |
| 113 | 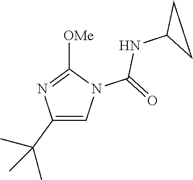 [SMILES: COc1nc(C(C)(C)C)cn1C(=O)NC1CC1] | 1.4 | 24 | B |
| 114 | 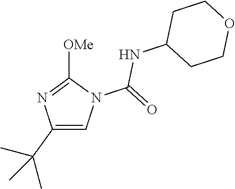 [SMILES: COc1nc(C(C)(C)C)cn1C(=O)NC1CCOCC1] | 0.5 | 29 | B |
| 115 | 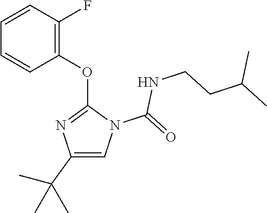 [SMILES: CC(C)CCNC(=O)n1cc(C(C)(C)C)nc1Oc1ccccc1F] | 4.1 | 0.4 | B |
| 116 | 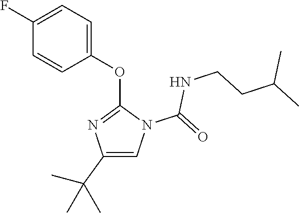 [SMILES: CC(C)CCNC(=O)n1cc(C(C)(C)C)nc1Oc1ccc(F)cc1] | 4.3 | 1.0 | A |
| 117 | 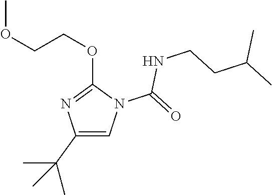 [SMILES: COCCOc1nc(C(C)(C)C)cn1C(=O)NCCC(C)C] | 2.7 | 10 | B |
| 118 | 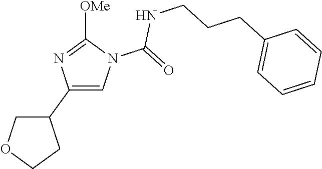 [SMILES: COc1nc(C2CCOC2)cn1C(=O)NCCCc1ccccc1] | 1.4 | 17 | A |
| 119 | 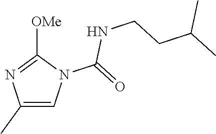 [SMILES: COc1nc(C)cn1C(=O)NCCC(C)C] | 1.5 | 14 | B |
| 120 | 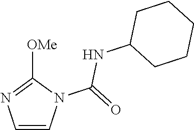 [SMILES: COc1nccn1C(=O)NC1CCCCC1] | 1.3 | 14 | C |
| 121 | 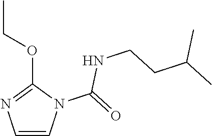 [SMILES: CCOc1nccn1C(=O)NCCC(C)C] | 1.7 | 23 | B |
| 122 | 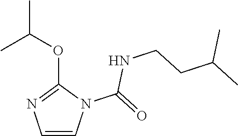 [SMILES: CC(C)CCNC(=O)n1ccnc1OC(C)C] | 2.1 | 21 | B |
| 123 | 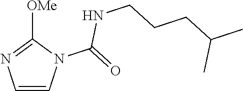 [SMILES: COc1nccn1C(=O)NCCCC(C)C] | 1.7 | 16 | A |
| 124 | 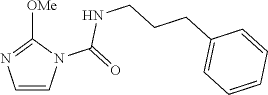 [SMILES: COc1nccn1C(=O)NCCCc1ccccc1] | 1.7 | 20 | A |
| 125 | 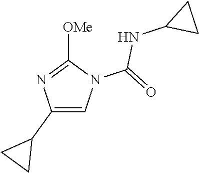 [SMILES: COc1nc(C2CC2)cn1C(=O)NC1CC1] | 0.5 | 14 | C |
| 126 | 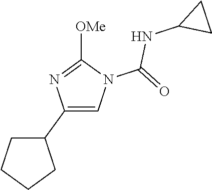 [SMILES: COc1nc(C2CCCC2)cn1C(=O)NC1CC1] | 1.6 | 13 | B |
| 127 | 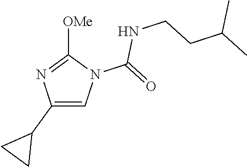 [SMILES: COc1nc(C2CC2)cn1C(=O)NCCC(C)C] | 1.9 | 23 | C |
| 128 | 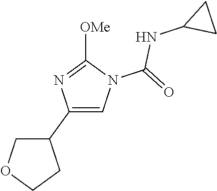 [SMILES: COc1nc(C2CCOC2)cn1C(=O)NC1CC1] | −0.5 | 15 | C |
| 129 | 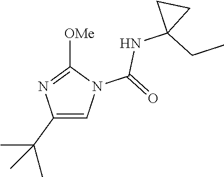 [SMILES: CCC1(NC(=O)n2cc(C(C)(C)C)nc2OC)CC1] | 2.2 | 16 | C |
| 130 | 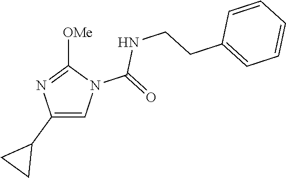 [SMILES: COc1nc(C2CC2)cn1C(=O)NCCc1ccccc1] | 2.0 | 12 | B |
| 131 | 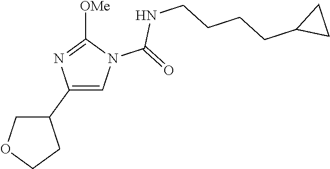 [SMILES: COc1nc(C2CCOC2)cn1C(=O)NCCCCC1CC1] | 1.5 | 23 | B |
| 132 | 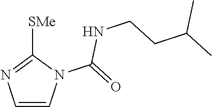 | 1.6 | 10 | A |
| 133 | 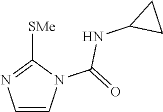 [SMILES: CSc1nccn1C(=O)NC1CC1] | −0.2 | 22 | B |
| 134 | 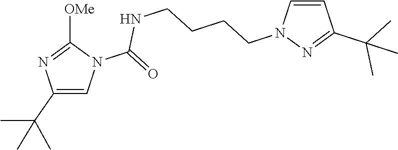 [SMILES: COc1nc(C(C)(C)C)cn1C(=O)NCCCCn1ccc(C(C)(C)C)n1] | 2.0 | 18 | A |
| 135 | 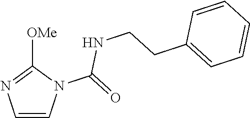 [SMILES: COc1nccn1C(=O)NCCc1ccccc1] | 1.3 | 26 | A |
| 136 | 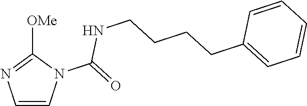 [SMILES: COc1nccn1C(=O)NCCCCc1ccccc1] | 2.2 | 22 | A |
| 137 | 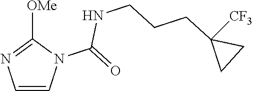 [SMILES: COc1nccn1C(=O)NCCCC1(C(F)(F)F)CC1] | 1.1 | 29 | A |
| 138 | 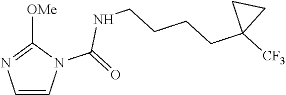 [SMILES: COc1nccn1C(=O)NCCCCC1(C(F)(F)F)CC1] | 1.6 | 27 | A |
| 139 | 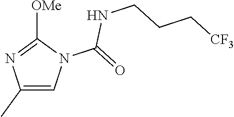 [SMILES: COc1nc(C)cn1C(=O)NCCCC(F)(F)F] | 0.6 | 32 | B |
| 140 | 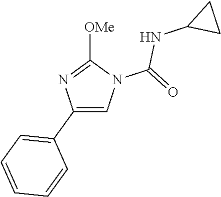 [SMILES: COc1nc(-c2ccccc2)cn1C(=O)NC1CC1] | 1.9 | 27 | C |
| 141 | 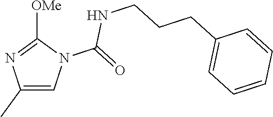 [SMILES: COc1nc(C)cn1C(=O)NCCCc1ccccc1] | 2.0 | 24 | A. |
| 142 | 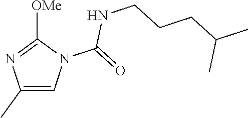 [SMILES: COc1nc(C)cn1C(=O)NCCCC(C)C] | 2.0 | 22 | B |
| 143 | 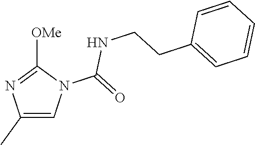 [SMILES: COc1nc(C)cn1C(=O)NCCc1ccccc1] | 1.6 | 27 | A |
| 144 | 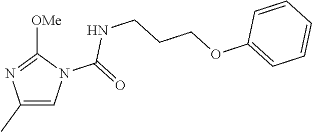 [SMILES: COc1nc(C)cn1C(=O)NCCCOc1ccccc1] | 2.0 | 26 | A |
| 145 | 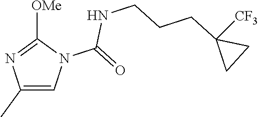 [SMILES: COc1nc(C)cn1C(=O)NCCCC1(C(F)(F)F)CC1] | 1.3 | 28 | A |
| 146 | 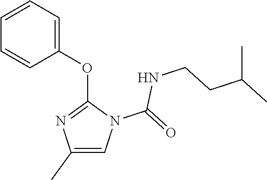 [SMILES: Cc1cn(C(=O)NCCC(C)C)c(Oc2ccccc2)n1] | 2.8 | 19 | A |
| 147 | 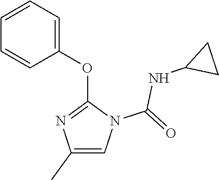 [SMILES: Cc1cn(C(=O)NC2CC2)c(Oc2ccccc2)n1] | 1.4 | 29 | A |
| 148 | 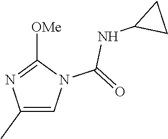 | 0.1 | 23 | B |
| 149 | 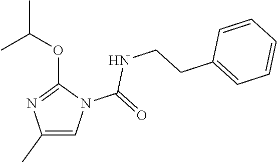 [SMILES: Cc1cn(C(=O)NCCc2ccccc2)c(OC(C)C)n1] | 2.4 | 21 | A |
| 150 | 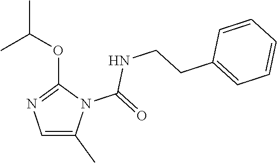 [SMILES: Cc1cnc(OC(C)C)n1C(=O)NCCc1ccccc1] | 2.4 | 5 | A |
| 151 | 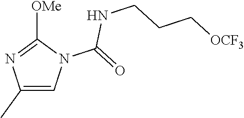 [SMILES: COc1nc(C)cn1C(=O)NCCCOC(F)(F)F] | 1.1 | 30 | B |
| 152 | 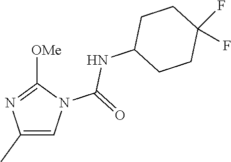 [SMILES: COc1nc(C)cn1C(=O)NC1CCC(F)(F)CC1] | 0.9 | 30 | C |
| 153 | 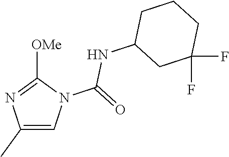 [SMILES: COc1nc(C)cn1C(=O)NC1CCCC(F)(F)C1] | 0.9 | 29 | C |
| 154 | 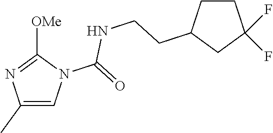 [SMILES: COc1nc(C)cn1C(=O)NCCC1CCC(F)(F)C1] | 1.5 | 30 | B |
| 155 | 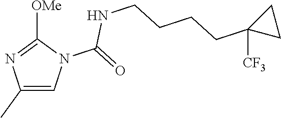 [SMILES: COc1nc(C)cn1C(=O)NCCCCC1(C(F)(F)F)CC1] | 1.8 | 22 | A |
| 156 | 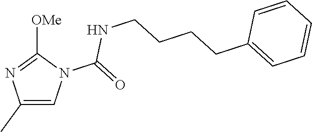 [SMILES: COc1nc(C)cn1C(=O)NCCCCc1ccccc1] | 2.5 | 23 | A |
| 157 | 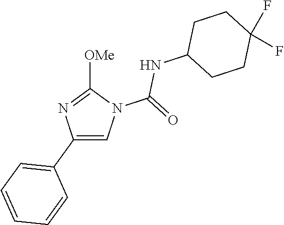 [SMILES: COc1nc(-c2ccccc2)cn1C(=O)NC1CCC(F)(F)CC1] | 2.7 | 1 | B |
| 158 | 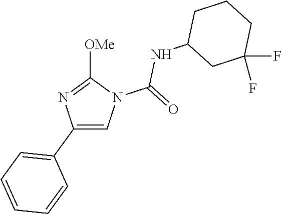 [SMILES: COc1nc(-c2ccccc2)cn1C(=O)NC1CCCC(F)(F)C1] | 2.7 | 1 | B |
| 159 | 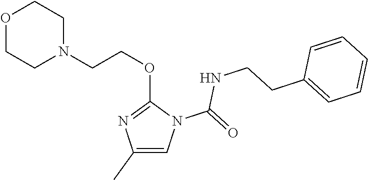 [SMILES: Cc1cn(C(=O)NCCc2ccccc2)c(OCCN2CCOCC2)n1] | 1.7 | 35 | A |
| 160 | 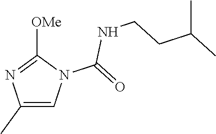 [SMILES: COc1nc(C)cn1C(=O)NCCC(C)C] | 1.5 | 13 | A |
| 161 | 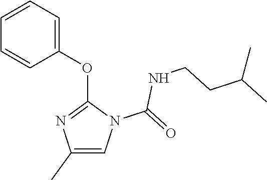 [SMILES: Cc1cn(C(=O)NCCC(C)C)c(Oc2ccccc2)n1] | 1.6 | 19 | A |
| 162 | 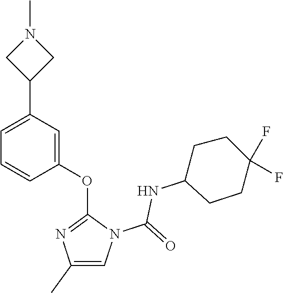 [SMILES: Cc1cn(C(=O)NC2CCC(F)(F)CC2)c(Oc2cccc(C3CN(C)C3)c2)n1] | 1.9 | 17 | B |
| 163 | 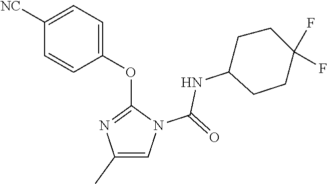 [SMILES: Cc1cn(C(=O)NC2CCC(F)(F)CC2)c(Oc2ccc(C#N)cc2)n1] | 1.7 | 13 | A |
| 164 | 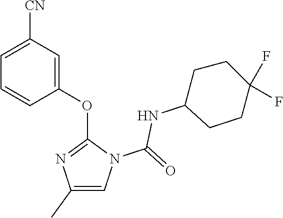 [SMILES: Cc1cn(C(=O)NC2CCC(F)(F)CC2)c(Oc2cccc(C#N)c2)n1] | 1.7 | 8 | A |
| 165 | 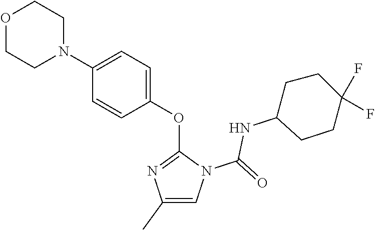 [SMILES: Cc1cn(C(=O)NC2CCC(F)(F)CC2)c(Oc2ccc(N3CCOCC3)cc2)n1] | 1.7 | 21 | A |
| 166 | 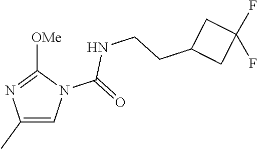 [SMILES: COc1nc(C)cn1C(=O)NCCC1CC(F)(F)C1] | 0.9 | 30 | B |
| 167 | 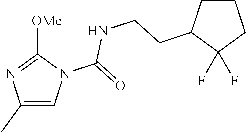 [SMILES: COc1nc(C)cn1C(=O)NCCC1CCCC1(F)F] | 1.5 | 32 | A |
| 168 | 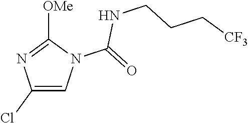 [SMILES: COc1nc(Cl)cn1C(=O)NCCCC(F)(F)F] | 1.1 | 34 | A |
| 169 | 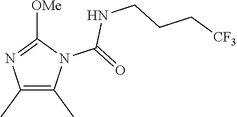 [SMILES: COc1nc(C)c(C)n1C(=O)NCCCC(F)(F)F] | 1.7 | 16 | A |
| 170 | 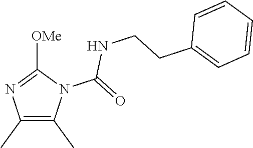 [SMILES: COc1nc(C)c(C)n1C(=O)NCCc1ccccc1] | 1.8 | 10 | A |
| 171 | 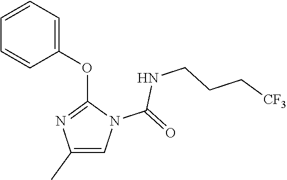 [SMILES: Cc1cn(C(=O)NCCCC(F)(F)F)c(Oc2ccccc2)n1] | 1.9 | 24 | A |
| 172 | 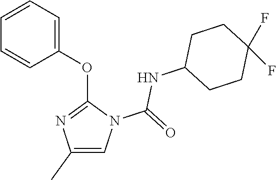 [SMILES: Cc1cn(C(=O)NC2CCC(F)(F)CC2)c(Oc2ccccc2)n1] | 2.2 | 4 | A |
| 173 | 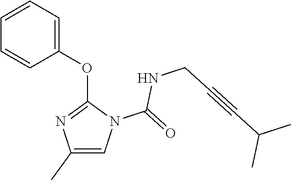 [SMILES: Cc1cn(C(=O)NCC#CC(C)C)c(Oc2ccccc2)n1] | 2.1 | 14 | A |
| 174 | 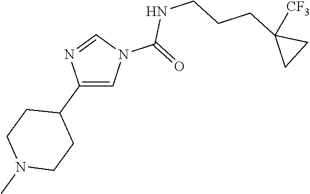 [SMILES: CN1CCC(c2cn(C(=O)NCCCC3(C(F)(F)F)CC3)cn2)CC1] | 0.7 | 25 | A |
| 175 | 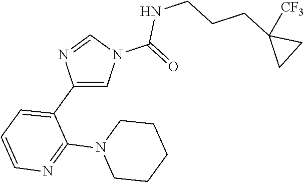 [SMILES: O=C(NCCCC1(C(F)(F)F)CC1)n1cnc(-c2cccnc2N2CCCCC2)c1] | 2.4 | 1 | A |
| 176 | 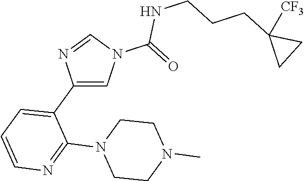 [SMILES: CN1CCN(c2ncccc2-c2cn(C(=O)NCCCC3(C(F)(F)F)CC3)cn2)CC1] | 1.6 | 29 | A |
| 177 | 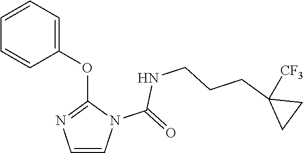 [SMILES: O=C(NCCCC1(C(F)(F)F)CC1)n1ccnc1Oc1ccccc1] | 2.4 | 8 | A |
| 178 | 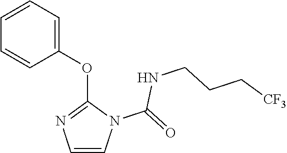 [SMILES: O=C(NCCCC(F)(F)F)n1ccnc1Oc1ccccc1] | 1.7 | 32 | A |
| 179 | 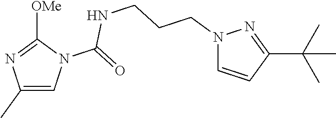 [SMILES: COc1nc(C)cn1C(=O)NCCCn1ccc(C(C)(C)C)n1] | 1.8 | 22 | A |
| 180 | 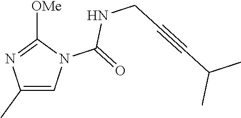 [SMILES: COc1nc(C)cn1C(=O)NCC#CC(C)C] | 1.0 | 17 | A |
| 181 | 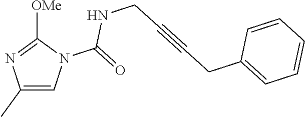 [SMILES: COc1nc(C)cn1C(=O)NCC#CCc1ccccc1] | 1.5 | 22 | A |
| 182 | 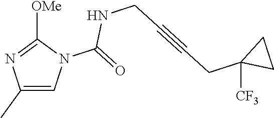 [SMILES: COc1nc(C)cn1C(=O)NCC#CCC1(C(F)(F)F)CC1] | 0.9 | 25 | A |
| 183 | 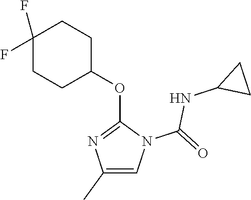 [SMILES: Cc1cn(C(=O)NC2CC2)c(OC2CCC(F)(F)CC2)n1] | 1.5 | 31 | B |
| 184 | 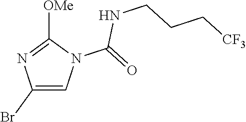 [SMILES: COc1nc(Br)cn1C(=O)NCCCC(F)(F)F] | 1.2 | 31 | A |
| 185 | 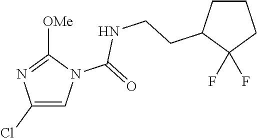 [SMILES: COc1nc(Cl)cn1C(=O)NCCC1CCCC1(F)F] | 2.0 | 29 | A |
| 186 | 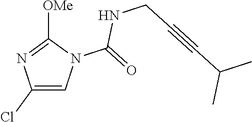 [SMILES: COc1nc(Cl)cn1C(=O)NCC#CC(C)C] | 1.5 | | B |
| 187 | 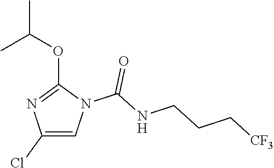 [SMILES: CC(C)Oc1nc(Cl)cn1C(=O)NCCCC(F)(F)F] | 1.9 | 29 | B |
| 188 | 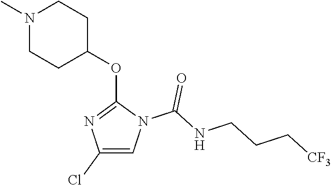 [SMILES: CN1CCC(Oc2nc(Cl)cn2C(=O)NCCCC(F)(F)F)CC1] | 1.1 | 36 | B |
| 189 | 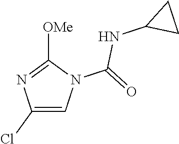 [SMILES: COc1nc(Cl)cn1C(=O)NC1CC1] | 0.5 | 26 | C |
| 190 | 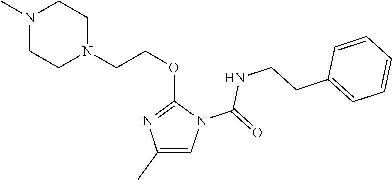 [SMILES: Cc1cn(C(=O)NCCc2ccccc2)c(OCCN2CCN(C)CC2)n1] | 0.8 | 39 | A |
| 191 | 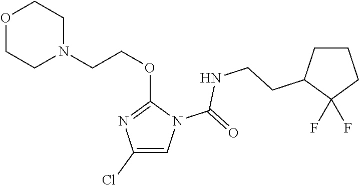 [SMILES: O=C(NCCC1CCCC1(F)F)n1cc(Cl)nc1OCCN1CCOCC1] | 2.1 | 33 | B |
| 192 | 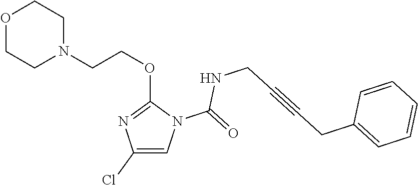 [SMILES: O=C(NCC#CCc1ccccc1)n1cc(Cl)nc1OCCN1CCOCC1] | 1.7 | 37 | A |
| 193 | 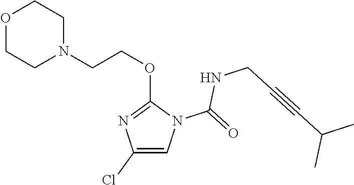 [SMILES: CC(C)C#CCNC(=O)n1cc(Cl)nc1OCCN1CCOCC1] | 1.2 | 27 | A |
| 194 | 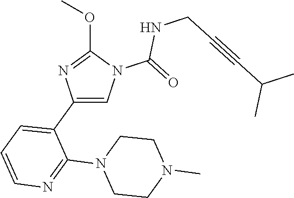 [SMILES: COc1nc(-c2cccnc2N2CCN(C)CC2)cn1C(=O)NCC#CC(C)C] | 1.9 | 45 | C |
| 195 | 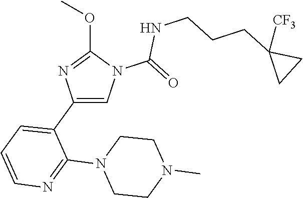 [SMILES: COc1nc(-c2cccnc2N2CCN(C)CC2)cn1C(=O)NCCCC1(C(F)(F)F)CC1] | 2.2 | 35 | C |
| 196 | 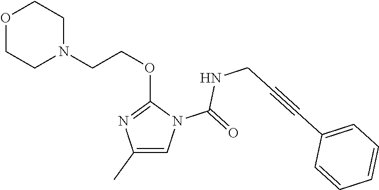 [SMILES: Cc1cn(C(=O)NCC#Cc2ccccc2)c(OCCN2CCOCC2)n1] | 1.3 | 31 | A |
| 197 | 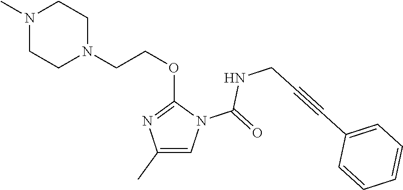 [SMILES: Cc1cn(C(=O)NCC#Cc2ccccc2)c(OCCN2CCN(C)CC2)n1] | 0.4 | 38 | A |
| 198 | 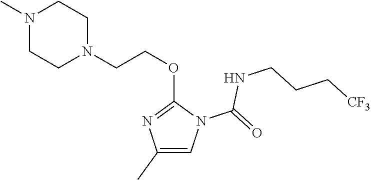 [SMILES: Cc1cn(C(=O)NCCCC(F)(F)F)c(OCCN2CCN(C)CC2)n1] | 0.2 | 38 | B |
| 199 | 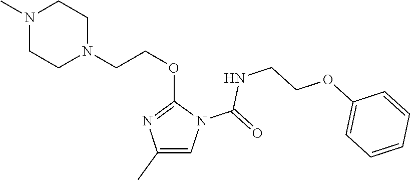 [SMILES: Cc1cn(C(=O)NCCOc2ccccc2)c(OCCN2CCN(C)CC2)n1] | 0.9 | 50 | A |
| 200 | 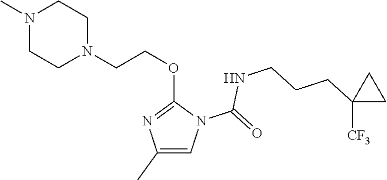 [SMILES: Cc1cn(C(=O)NCCCC2(C(F)(F)F)CC2)c(OCCN2CCN(C)CC2)n1] | 0.5 | 49 | A |
| 201 | 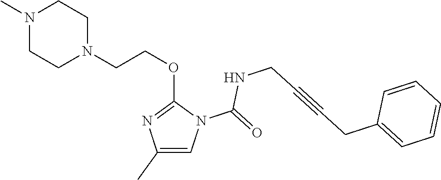 [SMILES: Cc1cn(C(=O)NCC#CCc2ccccc2)c(OCCN2CCN(C)CC2)n1] | 0.8 | 39 | A |
| 202 | 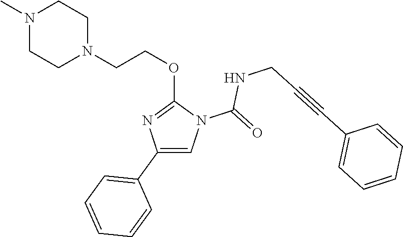 [SMILES: CN1CCN(CCOc2nc(-c3ccccc3)cn2C(=O)NCC#Cc2ccccc2)CC1] | 2.3 | 7 | A |
| 203 | 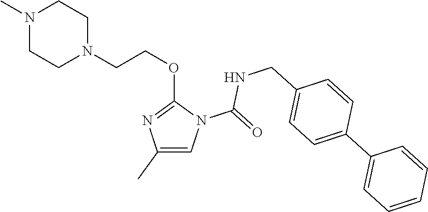 [SMILES: Cc1cn(C(=O)NCc2ccc(-c3ccccc3)cc2)c(OCCN2CCN(C)CC2)n1] | 2.4 | 30 | A |
| 204 | 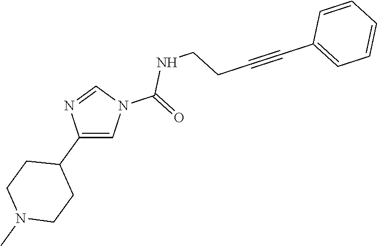 [SMILES: CN1CCC(c2cn(C(=O)NCCC#Cc3ccccc3)cn2)CC1] | 1.1 | 31 | A |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound of formula (I): 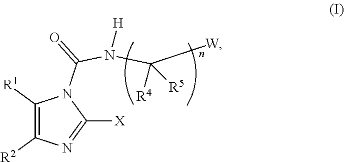
or a pharmaceutically acceptable salt thereof, wherein:
one of R¹ and R² is selected from the group consisting of hydrogen, C₁₋₆alkyl, halogen, cyano, phenyl, 3-12 membered heterocyclyl, C₃₋₇cycloalkyl, C₅₋₁₀ bicyclic carbocyclyl, 5-6 membered heteroaryl, C₁₋₆alkylene-cyano, C₁₋₆alkylene-N(R^{a})₂, —O—R^{b}, C₁₋₆alkylene-OR^{b}, C₁₋₆alkylene-(5-6 membered heteroaryl), C₁₋₆alkylene-(3-12 membered heterocyclyl), C₁₋₆alkylene-phenyl, C₁₋₆alkylene-C₃₋₇cycloalkyl, (3-7 membered heterocyclylene)-(3-7 membered heterocyclyl), (5-6 membered heteroarylene)-(3-7 membered heterocyclyl), phenylene-(3-7 membered heterocyclyl), phenylene-(3-7 membered heterocyclylene)-(3-7 membered heterocyclyl), and (5-6 membered heteroarylene)-(3-7 membered heterocyclylene)-(3-7 membered heterocyclyl), and the other is selected from the group consisting of hydrogen, C₁₋₆alkyl, and C₁₋₆alkylene-N(R^{a})₂;
R⁴ and R⁵ are independently, for each occurrence, selected from the group consisting of hydrogen, C₁₋₆alkyl, C₁₋₆haloalkyl, and halogen, or R⁴ and R⁵ can be taken together to form C₃₋₇cycloalkylene;
n is integer selected from 0 to 6;
X is selected from the group consisting of hydrogen, —OR^{c}, —S—C₁₋₆alkyl, C₁₋₆alkyl, and phenyl;
R^{a} is independently, for each occurrence, hydrogen or C₁₋₆alkyl;
R^{b} is independently, for each occurrence, selected from the group consisting of C₁₋₆alkyl, C₁₋₆alkylene-NR^{a}₂, C₁₋₆haloalkyl, C₃₋₆cycloalkyl, 3-7 membered heterocyclyl, 5-6 membered heteroaryl, and phenyl;
R^{c} is independently, for each occurrence, selected from the group consisting of C₁₋₆alkyl, —C₁₋₆alkylene-O—R^{a}, —C₁₋₆alkylene-N(R^{a})₂, C₁₋₆alkylene-(3-7 membered heterocyclyl), C₁₋₆haloalkyl, C₃₋₇cycloalkyl, 3-7 membered heterocyclyl, 5-6 membered heteroaryl, phenyl, and phenylene-(3-7 membered heterocyclyl); and
W is selected from the group consisting of methyl, halogen, phenyl, phenylene-phenyl, C₃₋₇cycloalkyl, 3-7 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₆alkynylene, —(C₂₋₆alkynylene)-phenyl, and —(C₂₋₆alkynylene)-C₃₋₇cycloalkyl; and
wherein any aforementioned phenyl, C₃₋₇cycloalkyl, 3-12 or 3-7 membered heterocyclyl, or 5-6 membered heteroaryl is optionally substituted (e.g., with one or more substituents each independently selected from the group consisting of C₁₋₆alkyl, C₁₋₆haloalkyl, —CN, halogen, C₁₋₆alkylene-N(R^{a})₂, —O—C₁₋₆alkyl, and oxo, wherein R^{a} is hydrogen or C₁₋₆alkyl), wherein
when (i) n is 0, or (ii) each of R⁴ and R⁵ is hydrogen, W is not methyl; and when each of R⁴ and R⁵ is independently selected from hydrogen and halogen and W is halogen, R² is not pyridyl.

2. The compound of claim 1, wherein X is selected from the group consisting of hydrogen, methyl, —OR^{c}, and —SCH₃.

3. The compound of claim 1 or 2, wherein X is —OR^{c}.

4. The compound of claim 1, wherein the compound is a compound of formula (I-a): 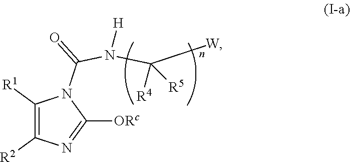
or a pharmaceutically acceptable salt thereof, wherein the variables are as defined in claim 1.

5. The compound of any one of claims 1-4, wherein R^{c} is selected from the group consisting of methyl, ethyl, —CH₂(CH₃)₂, phenyl,

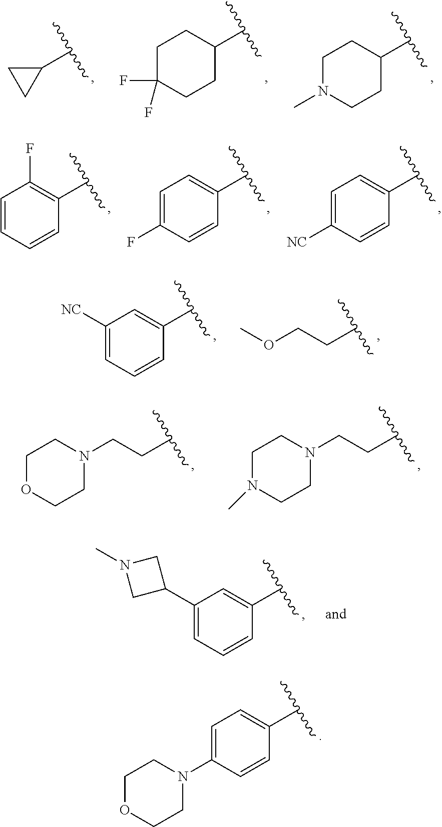

6. The compound of any one of claims 1-5, wherein R^{c} is methyl.

7. The compound of any one of claims 1-6, wherein R² is selected from the group consisting of hydrogen, C₁₋₄alkyl, halogen, cyano, —O—R^{b}, phenyl, 3-12 membered heterocyclyl, C₃₋₇cycloalkyl, C₅₋₁₀ bicyclic carbocyclyl, 5-6 membered heteroaryl, C₁₋₆alkylene-cyano, C₁₋₆alkylene-OR^{b}, C₁₋₆alkylene-N(R^{a})₂, C₁₋₆alkylene-(5-6 membered heteroaryl), C₁₋₆alkylene-(3-12 membered heterocyclyl), C₁₋₆alkylene-phenyl, C₁₋₆alkylene-C₃₋₇cycloalkyl, (3-7 membered heterocyclylene)-(3-7 membered heterocyclyl), phenylene-(3-7 membered heterocyclyl), phenylene-(3-7 membered heterocyclylene)-(3-7 membered heterocyclyl), (5-6 membered heteroarylene)-(3-7 membered heterocyclyl), and (5-6 membered heteroarylene)-(3-7 membered heterocyclylene)-(3-7 membered heterocyclyl), and R¹ is selected from the group consisting of hydrogen, C₁₋₆alkyl, and C₁₋₆alkylene-N(R^{a})₂, wherein the 3-12 membered heterocyclyl, C₃₋₇cycloalkyl, phenyl, 5-6 membered heteroaryl, C₁₋₆alkylene-(3-12 membered heterocyclyl) (5-6 membered heteroarylene)-(3-7 membered heterocyclyl), and (5-6 membered heteroarylene)-(3-7 membered heterocyclylene)-(3-7 membered heterocyclyl) are optionally substituted.

8. The compound of any one of claims 1-7, wherein R² is selected from the group consisting of hydrogen, C₁₋₆alkyl, halogen, phenyl, 3-12 membered heterocyclyl, C₃₋₇cycloalkyl, 5-6 membered heteroaryl, C₁₋₆alkylene-cyano, C₁₋₆alkylene-(3-12 membered heterocyclyl), (5-6 membered heteroarylene)-(3-7 membered heterocyclyl), and (5-6 membered heteroarylene)-(3-7 membered heterocyclylene)-(3-7 membered heterocyclyl), and R¹ is hydrogen or methyl, wherein the 3-12 membered heterocyclyl, C₃₋₇cycloalkyl, phenyl, 5-6 membered heteroaryl, C₁₋₆alkylene-(3-12 membered heterocyclyl), (5-6 membered heteroarylene)-(3-7 membered heterocyclyl), and (5-6 membered heteroarylene)-(3-7 membered heterocyclylene)-(3-7 membered heterocyclyl) are optionally substituted with 1-3 substituents independently, for each occurrence, selected from the group consisting of —CH₂N(CH₃)₂, cyano, C₁₋₆alkyl, halogen, methoxy, oxo, and combinations thereof.

9. The compound of any one of claims 1-8, wherein R² is selected from the group consisting of hydrogen, methyl, ethyl, isopropyl, t-butyl, —C(CH₃)₂CN, bromine, chlorine, phenyl,

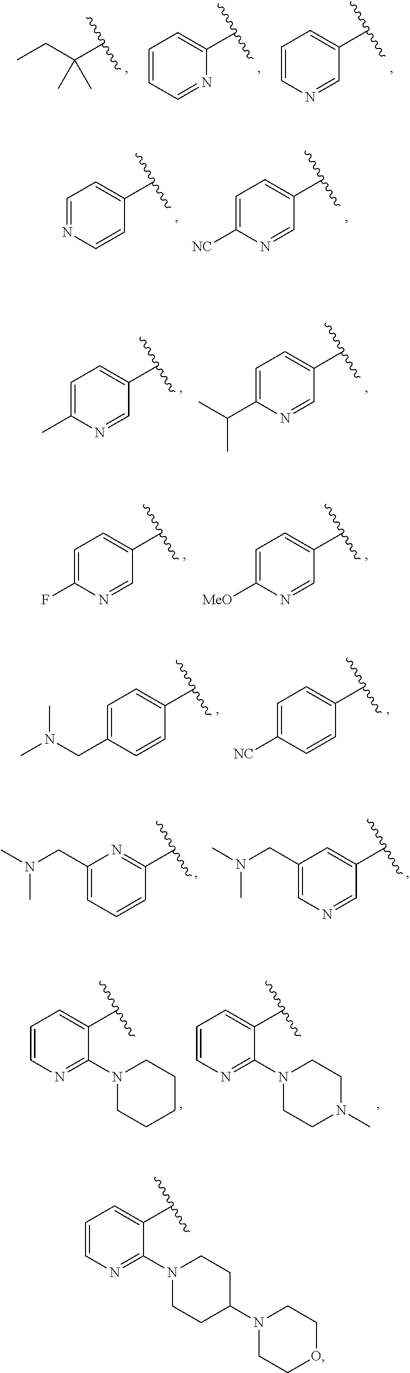

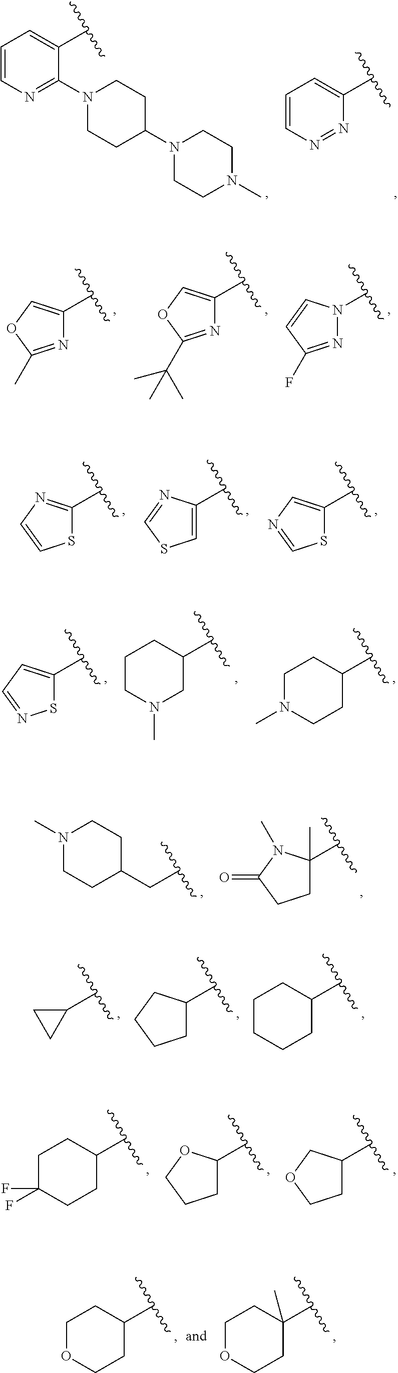

10. The compound of any one of claims 1-8, wherein R² is

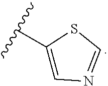 [SMILES: *c1cncs1]

11. The compound of any one of claims 1-8, wherein R² is

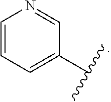 [SMILES: *c1cccnc1]

12. The compound of any one of claims 1-8, wherein R² is t-butyl.

13. The compound of any one of claims 1-8, wherein R² is 5-6 membered heteroaryl.

14. The compound of any one of claims 1-13, wherein R¹ is hydrogen.

15. The compound of any one of claims 1-14, wherein R⁴ and R⁵ are independently, for each occurrence, hydrogen or methyl, or R⁴ and R⁵ can be taken together to form a cyclopropylene.

16. The compound of any one of claims 1-15, wherein R⁴ and R⁵ are hydrogen.

17. The compound of any one of claims 1-16, wherein n is 0, 1, 2, 3, 4, or 5.

18. The compound of any one of claims 1-17, wherein n is 0.

19. The compound of any one of claims 1-17, wherein n is 1.

20. The compound of any one of claims 1-17, wherein n is 2.

21. The compound of any one of claims 1-17, wherein n is 3.

22. The compound of any one of claims 1-17, wherein n is 4.

23. The compound of any one of claims 1-17, wherein n is 5.

24. The compound of any one of claims 1-23, wherein W is selected from the group consisting of methyl, halogen, —O—C₁₋₆alkyl, C₂₋₆alkynylene, phenyl, C₃₋₇cycloalkyl, 5-6 membered heteroaryl, —(C₂₋₆alkynylene)-phenyl, —(C₂₋₆alkynylene)-C₃₋₇cycloalkyl, and —O— phenyl, wherein phenyl, C₃₋₇cycloalkyl, 5-6 membered heteroaryl, and —(C₂₋₆alkynylene)-C₃₋₇cycloalkyl is optionally substituted with 1-3 substituents independently, for each occurrence, selected from the group consisting of C₁₋₆alkyl, halogen, CF₃, phenyl, and combinations thereof.

25. The compound of any one of claims 1-24, wherein W is selected from the group consisting of methyl, —CF₃, —OCF₃,

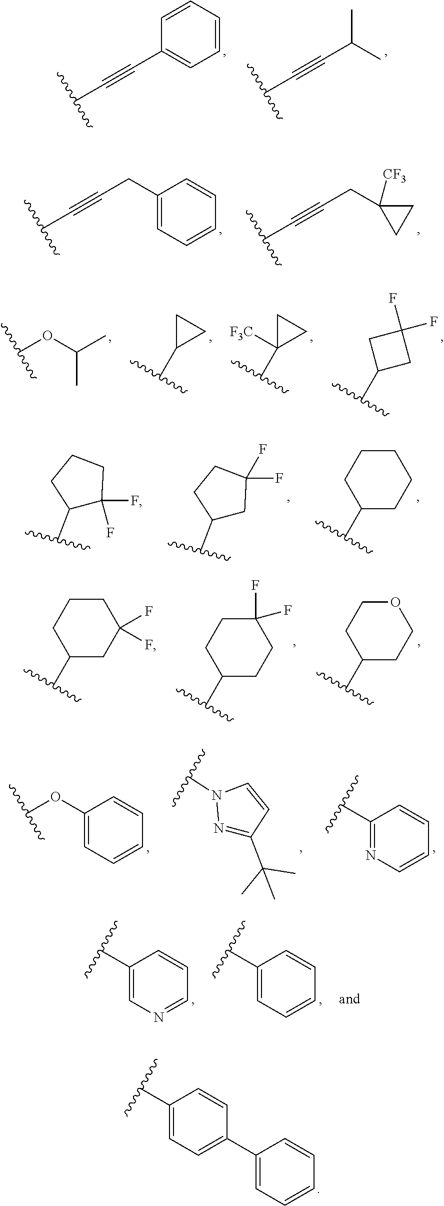

26. The compound of any one of claims 1-25, wherein W is methyl or phenyl.

27. The compound of any one of claims 1-26, wherein W is methyl.

28. The compound of any one of claims 1-26, wherein W is phenyl.

29. The compound of any one of claims 1-25, wherein W is

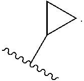 [SMILES: *C1CC1]

30. The compound of any one of claims 1-29, wherein any aforementioned phenyl, 3-12 membered heterocyclyl, or 5-6 membered heteroaryl is independently, for each occurrence, optionally substituted with 1-3 substituents independently, for each occurrence, selected from the group consisting of —CH₂N(R^{a})₂, cyano, C₁₋₆alkyl, halogen, and —O—C₁₋₆alkyl, wherein R^{a} is as defined in claim 1.

31. The compound of any one of claims 1-29, wherein any aforementioned phenyl is optionally substituted with 1-3 of —CH₂N(CH₃)₂, halogen, or —CN, any aforementioned 3-12 membered heterocyclyl is independently for each occurrence optionally substituted with 1-3 substituents each independently selected from methyl and oxo, any aforementioned 5-6 membered heteroaryl is independently for each occurrence optionally substituted with 1-3 substituents independently, for each occurrence, selected from the group consisting of —CH₂N(CH₃)₂, cyano, C₁₋₆alkyl, halogen, and methoxy, and any aforementioned C₃₋₇cyclohexyl is independently for each occurrence optionally substituted with 1-3 substituents each independently halogen or trifluoromethyl.

32. The compound of any one of claims 1-30, wherein any phenyl at R² is independently, for each occurrence, optionally substituted with 1-2 of —CH₂N(CH₃)₂, any 3-12 membered heterocyclyl of R² is independently, for each occurrence, optionally substituted with 1-3 substituents each independently selected from methyl or oxo, any 5-6 membered heteroaryl of R² is independently, for each occurrence, optionally substituted with 1 or 2 substituents independently, for each occurrence, selected from the group consisting of —CH₂N(CH₃)₂, cyano, C₁₋₆alkyl, halogen, and methoxy, and any C₃₋₇cyclohexyl at R² is independently for each occurrence optionally substituted with 1-3 halogen.

33. The compound of claim 1, wherein the compound is a compound of formula (I-b): 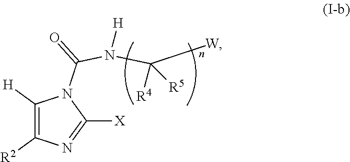
or a pharmaceutically acceptable salt thereof, wherein:
R² is selected from the group consisting of hydrogen, C₁₋₆alkyl, halogen, cyano, phenyl, 3-12 membered heterocyclyl, C₃₋₇cycloalkyl, C₅₋₁₀ bicyclic carbocyclyl, 5-6 membered heteroaryl, C₁₋₆alkylene-cyano, C₁₋₆alkylene-N(R^{a})₂, —O—R^{b}, C₁₋₆alkylene-OR^{b}, C₁₋₆alkylene-(5-6 membered heteroaryl), C₁₋₆alkylene-(3-12 membered heterocyclyl), C₁₋₆alkylene-phenyl, C₁₋₆alkylene-C₃₋₇cycloalkyl, (3-7 membered heterocyclylene)-(3-7 membered heterocyclyl), (5-6 membered heteroarylene)-(3-7 membered heterocyclyl), phenylene-(3-7 membered heterocyclyl), phenylene-(3-7 membered heterocyclylene)-(3-7 membered heterocyclyl), and (5-6 membered heteroarylene)-(3-7 membered heterocyclylene)-(3-7 membered heterocyclyl);
n is an integer selected from 0 to 6; wherein,
when n is selected from 2 to 6, R⁴ and R⁵ are independently, for each occurrence, selected from the group consisting of hydrogen, C₁₋₆alkyl, C₁₋₆haloalkyl, and halogen, or R⁴ and R⁵ can be taken together to form C₃₋₇cycloalkylene;
when n is 1, R⁴ and R⁵ are independently selected from the group consisting of hydrogen, C₁₋₆alkyl, C₁₋₆haloalkyl, and halogen;
X is selected from the group consisting of hydrogen, —OR^{c}, —S—C₁₋₆alkyl, C₁₋₆alkyl, and phenyl;
R is independently, for each occurrence, hydrogen or C₁₋₆alkyl,
R^{b} is independently, for each occurrence, selected from the group consisting of C₁₋₆alkyl, C₁₋₆alkylene-NR^{a}₂, C₁₋₆haloalkyl, C₃₋₇cycloalkyl, 3-7 membered heterocyclyl, 5-6 membered heteroaryl, and phenyl;
R^{c} is independently, for each occurrence, selected from the group consisting of C₁₋₆alkyl, —C₁₋₆alkylene-O—R^{a}, —C₁₋₆alkylene-N(R^{a})₂, C₁₋₆alkylene-(3-7 membered heterocyclyl), C₁₋₆haloalkyl, C₃₋₇cycloalkyl, 3-7 membered heterocyclyl, 5-6 membered heteroaryl, phenyl, and phenylene-(3-7 membered heterocyclyl); and
W is selected from the group consisting of methyl, halogen, phenyl, phenylene-phenyl, C₃₋₇cycloalkyl, 3-7 membered heterocyclyl, 5-6 membered heteroaryl, —O—C₁₋₆alkyl, —O—C₁₋₆haloalkyl, —O-phenyl, —O—(C₁₋₆alkylene)-phenyl, C₂₋₆alkynyl, —(C₂₋₆alkynylene)-phenyl, and —(C₂₋₆alkynylene)-C₃₋₇cycloalkyl; and
wherein any aforementioned phenyl, C₃₋₇cycloalkyl, 3-12 or 3-7 membered heterocyclyl, or 5-6 membered heteroaryl is optionally substituted with one or more substituents each independently selected from the group consisting of C₁₋₆alkyl, C₁₋₆haloalkyl, —CN, halogen, C₁₋₆alkylene-N(R^{a})₂, —O—C₁₋₆alkyl, and oxo, wherein R^{a} is hydrogen or C₁₋₆alkyl; wherein
when (i) n is 0, or (ii) each of R⁴ and R⁵ is hydrogen, W is not methyl; and when each of R⁴ and R⁵ is independently selected from hydrogen and halogen and W is halogen, R² is not pyridyl.

34. The compound of claim 33, wherein the compound is a compound of formula (I-c): 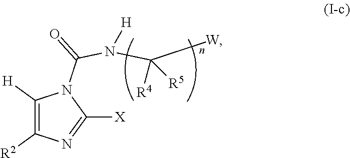
or a pharmaceutically acceptable salt thereof, wherein:
R² is selected from the group consisting of C₁₋₆alkyl, phenyl, and 3-7 membered heterocyclyl;
R⁴ and R⁵ are hydrogen;
n is an integer selected from 1 to 3;
X is hydrogen or —O—C₁₋₆alkylene-(3-7 membered heterocyclyl); and
W is selected from the group consisting of phenylene-phenyl, C₃₋₇cycloalkyl, and —(C₂₋₆alkynylene)-phenyl; and
wherein any aforementioned C₃₋₇cycloalkyl or 3-7 membered heterocyclyl is optionally substituted with one or more substituents each independently selected from C₁₋₆alkyl and C₁₋₆haloalkyl.

35. The compound of claim 33 or 34, wherein R² is methyl.

36. The compound of claim 33 or 34, wherein R² is phenyl.

37. The compound of claim 33 or 34, wherein R² is

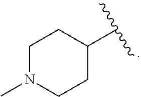 [SMILES: *C1CCN(C)CC1]

38. The compound of any one of claims 33-37, wherein n is 1.

39. The compound of any one of claims 33-37, wherein n is 3.

40. The compound of any one of claims 33-39, wherein X is hydrogen.

41. The compound of any one of claims 33-39, wherein X is

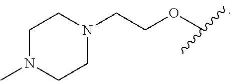 [SMILES: *OCCN1CCN(C)CC1]

42. The compound of any one of claims 33-41, wherein W is

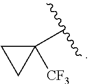 [SMILES: *C1(C(F)(F)F)CC1]

43. The compound ofany one of claims 33-41, wherein W is

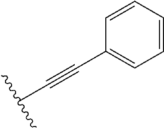 [SMILES: *C#Cc1ccccc1]

44. The compound of any one of claims 33-41, wherein W is

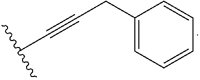 [SMILES: *C#CCc1ccccc1]

45. The compound of any one of claims 33-41, wherein W is

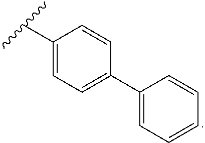 [SMILES: *c1ccc(-c2ccccc2)cc1]

46. A pharmaceutical composition comprising the compound of any one of claims 1-45 and a pharmaceutically acceptable carrier.

47. A method of treating a subject with cancer and in need thereof, the method comprising administering to the subject a therapeutically effective amount of the compound of any one of claims 1-45 or a pharmaceutical composition of claim 46.

48. The method of claim 47, wherein the cancer is glioblastoma.

49. A method of treating a subject with a lysosomal storage disorder and in need thereof, the method comprising administering to the subject a therapeutically effective amount of the compound of any one of claims 1-45 or a pharmaceutical composition of claim 46.

50. The method of claim 49, wherein the lysosomal storage disorder is selected from the group consisting of: Krabbe disease, Fabry disease, Tay-Sachs disease, Pompe disease, Hunter's syndrome, Niemann Pick disease Types A and B, and Gaucher disease.

51. The method of claim 50, wherein the lysosomal storage disorder is Gaucher disease.

52. A method of treating a subject with a neurodegenerative disorder and in need thereof, the method comprising administering to the subject a therapeutically effective amount of the compound of any one of claims 1-45 or a pharmaceutical composition of claim 46.

53. The method of claim 52, wherein the neurodegenerative disorder is selected from the group consisting of: Alzheimer's disease, Parkinson's disease, Huntington's disease, amyotrophic lateral sclerosis, Lewy body disease, dementia, and multiple system atrophy.

54. The method of claim 53, wherein the neurodegenerative disorder is Parkinson's disease.

55. The method of claim 53, wherein the neurodegenerative disorder is Lewy body disease.

56. The method of claim 53, wherein the neurodegenerative disorder is dementia.

57. The method of claim 53, wherein the neurodegenerative disorder is multiple system atrophy.

58. A method of treating a subject with an inflammatory disorder and in need thereof, the method comprising administering to the subject a therapeutically effective amount of the compound of any one of claims 1-45 or a pharmaceutical composition of claim 46.

59. The method of any one of claims 47-58, wherein the subject is human.

60. A compound of any one of claims 1-45 or a pharmaceutical composition of claim 46 for use in a method of treating a subject with cancer and in need thereof, the method comprising administering to the subject a therapeutically effective amount of the compound or the pharmaceutical composition.

61. A compound of any one of claims 1-45 or a pharmaceutical composition of claim 46 for use in a method of treating a subject with a lysosomal storage disorder and in need thereof, the method comprising administering to the subject a therapeutically effective amount of the compound or the pharmaceutical composition.

62. A compound of any one of claims 1-45 or a pharmaceutical composition of claim 46 for use in a method of treating a subject with a neurodegenerative disorder and in need thereof, the method comprising administering to the subject a therapeutically effective amount of the compound or the pharmaceutical composition.

63. A compound of any one of claims 1-45 or a pharmaceutical composition of claim 46 for use in a method of treating a subject with an inflammatory disorder and in need thereof, the method comprising administering to the subject a therapeutically effective amount of the compound or the pharmaceutical composition.

64. A compound of any one of claims 1-45 or a pharmaceutical composition of claim 46 for the manufacture of a medicament for treating a subject with cancer and in need thereof, the method comprising administering to the subject a therapeutically effective amount of the compound or the pharmaceutical composition.

65. A compound of any one of claims 1-45 or a pharmaceutical composition of claim 46 for the manufacture of a medicament for treating a subject with a lysosomal storage disorder and in need thereof, the method comprising administering to the subject a therapeutically effective amount of the compound or the pharmaceutical composition.

66. A compound of any one of claims 1-45 or a pharmaceutical composition of claim 46 for the manufacture of a medicament for treating a subject with a neurodegenerative disorder and in need thereof, the method comprising administering to the subject a therapeutically effective amount of the compound or the pharmaceutical composition.

67. A compound of any one of claims 1-45 or a pharmaceutical composition of claim 46 for the manufacture of a medicament for treating a subject with an inflammatory disorder and in need thereof, the method comprising administering to the subject a therapeutically effective amount of the compound or the pharmaceutical composition.
